SEQUENCE LISTING (161 sequences)

SEQ ID NO: 1 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 4 - protein, 9 aa, Human cytomegalovirus
VTEHDTLLY

SEQ ID NO: 5 - protein, 9 aa, Human cytomegalovirus
GLDRNSGNY

SEQ ID NO: 6 - protein, 9 aa, Human cytomegalovirus
NLVPMVATV

SEQ ID NO: 7 - protein, 9 aa, Human cytomegalovirus
GVMTRGRLK

SEQ ID NO: 8 - protein, 9 aa, Human cytomegalovirus
VYALPLKML

SEQ ID NO: 9 - protein, 9 aa, Human cytomegalovirus
QYDPVAALF

SEQ ID NO: 10 - protein, 9 aa, Influenza A virus
GILGFVFTL

SEQ ID NO: 11 - protein, 9 aa, Influenza A virus
ILRGSVAHK

SEQ ID NO: 12 - protein, 9 aa, Epstein-Barr virus
CLGGLLTMV

SEQ ID NO: 13 - protein, 507 aa, Artificial Sequence
  source: /note=“Description of Artificial Sequence Synthetic polypeptide”
VTEHDTLLYKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 14 - protein, 507 aa, Artificial Sequence
  source: /note=“Description of Artificial Sequence Synthetic polypeptide”
GLDRNSGNYKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 15 - protein, 506 aa, Artificial Sequence
  source: /note=“Description of Artificial Sequence Synthetic polypeptide”
GVMTRGRLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 16 - protein, 507 aa, Artificial Sequence
  source: /note=“Description of Artificial Sequence Synthetic polypeptide”
VYALPLKMLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 17 - protein, 520 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 18 - protein, 519 aa, Artificial Sequence
  source: /note=“Description of Artificial Sequence Synthetic polypeptide”
GILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVS

SEQ ID NO: 19 - protein, 520 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 20 - protein, 520 aa, Artificial Sequence
  source: /note=“Description of Artificial Sequence Synthetic polypeptide”
QVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 21 - protein, 507 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSVTEHDTLLY

SEQ ID NO: 22 - protein, 507 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSNLVPMVATV

SEQ ID NO: 23 - protein, 507 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSQYDPVAALF

SEQ ID NO: 24 - protein, 507 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSCLGGLLTMV

SEQ ID NO: 25 - protein, 507 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSILRGSVAHK

SEQ ID NO: 26 - protein, 520 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSNLVPMVATV

SEQ ID NO: 27 - protein, 508 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSNLVPMVATV

SEQ ID NO: 28 - protein, 526 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGILGFVFTL

SEQ ID NO: 29 - protein, 506 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSNLVPMVATV

SEQ ID NO: 30 - protein, 507 aa, Artificial Sequence; identical to SEQ ID NO: 22
  source: /note=“Description of Artificial Sequence Synthetic polypeptide”

SEQ ID NO: 31 - protein, 520 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKNLVPMVATV

SEQ ID NO: 32 - protein, 508 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSSGGGGSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELKNLVPMVATV

SEQ ID NO: 33 - protein, 521 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGSTSGSGKPGSGEGSTKGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKNLVPMVATV

SEQ ID NO: 34 - protein, 400 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAHHSEDNLVPMVATV

SEQ ID NO: 35 - protein, 516 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSSNLVPMVATV

SEQ ID NO: 36 - protein, 393 aa, Artificial Sequence
  source: /note=“Description of Artificial Sequence Synthetic polypeptide”
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAAHHSEDPSSKAPKAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSNLVPMVATV

SEQ ID NO: 37 - protein, 373 aa, Artificial Sequence
  source: /note=“Description of Artificial Sequence Synthetic polypeptide”
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPASVSDVPRDLEVVAATPTSLLISWCRQRCADSYRITYGETGGNSPVQEFTVPGSWKTATISGLKPGVDYTITVYVVTHYYGWDRYSHPISINYRTGSNLVPMVATV

SEQ ID NO: 38 - protein, 373 aa, Artificial Sequence
  source: /note=“Description of Artificial Sequence Synthetic polypeptide”
ASVSDVPRDLEVVAATPTSLLISWCRQRCADSYRITYGETGGNSPVQEFTVPGSWKTATISGLKPGVDYTITVYVVTHYYGWDRYSHPISINYRTGSEFPKPSTPPGSSGGAPKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAANLVPMVATV

SEQ ID NO: 39 - protein, 409 aa, Artificial Sequence
  source: /note=“Description of Artificial Sequence Synthetic polypeptide”
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTNLVPMVATV

SEQ ID NO: 40 - protein, 498 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAGGGGSGGDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 41 - protein, 520 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 42 - protein, 508 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 43 - protein, 526 aa, Artificial Sequence
  source: /note=“Description of Artificial Sequence Synthetic polypeptide”
GILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 44 - protein, 497 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAGGGGSGGDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 45 - protein, 507 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 46 - protein, 520 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELK

SEQ ID NO: 47 - protein, 519 aa, Artificial Sequence
  source: /note=“Description of Artificial Sequence Synthetic polypeptide”
NLVPMVATVKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAGGGGSGGQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSSGGGGSGGGGSGGGGSGGGGSGGGGSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELK

SEQ ID NO: 48 - protein, 409 aa, Artificial Sequence
  source: /note=“Description of Artificial Sequence Synthetic polypeptide”
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEFPKPSTPPGSSGGAPNLVPMVATVKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 49 - protein, 409 aa, Artificial Sequence
  source: /note=“Description of Artificial Sequence Synthetic polypeptide”
GILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAAEFPKPSTPPGSSGGAPAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 50 - protein, 521 aa, Artificial Sequence
  source: /note=“Description of Artificial Sequence Synthetic polypeptide”
MNLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 51 - protein, 521 aa, Artificial Sequence
  source: /note=“Description of Artificial Sequence Synthetic polypeptide”
MNLVPMVATVKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 52 - protein, 521 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSNLVPMVATV

SEQ ID NO: 53 - protein, 521 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSNLVPMVATV

SEQ ID NO: 54 - protein, 521 aa, Artificial Sequence
  source: /note=“Description of Artificial Sequence Synthetic polypeptide”
MGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 55 - protein, 521 aa, Artificial Sequence
  source: /note=“Description of Artificial Sequence Synthetic polypeptide”
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 56 - protein, 521 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 57 - protein, 509 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSNLVPMVATV

SEQ ID NO: 58 - protein, 527 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGILGFVFTL

SEQ ID NO: 59 - protein, 527 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGILGFVFTL

SEQ ID NO: 60 - protein, 507 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSSNLVPMVATV

SEQ ID NO: 61 - protein, 508 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSSNLVPMVATV

SEQ ID NO: 62 - protein, 251 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 63 - protein, 499 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGGGGSQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 64 - protein, 512 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 65 - protein, 512 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGSTSGSGKPGSGEGSTKGQVQLQESGPGLVRPSQTLSLTCTVSGFSLTSYGVHWVRQPPGRGLEWIGVMWRGGSTDYNAAFMSRLNITKDNSKNQVSLRLSSVTAADTAVYYCAKSMITTGFVMDSWGQGSLVTVSS

SEQ ID NO: 66 - protein, 500 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 67 - protein, 518 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 68 - protein, 518 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 69 - protein, 498 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKGGGGSQVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 70 - protein, 512 aa, Artificial Sequence; identical to SEQ ID NO: 64
  source: /note=“Description of Artificial Sequence Synthetic polypeptide”

SEQ ID NO: 71 - protein, 25 aa, Artificial Sequence
  source: /note=“Description of Artificial Sequence Synthetic peptide”
EFPKPSTPPGSSGGAPGILGFVFTL

SEQ ID NO: 72 - protein, 520 aa, Artificial Sequence; identical to SEQ ID NO: 20
  source: /note=“Description of Artificial Sequence Synthetic polypeptide”

SEQ ID NO: 73 - protein, 521 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISIGGTSLLMIDSGIGDNLFAVDVRGIAPEEGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAR

SEQ ID NO: 74 - protein, 525 aa, Artificial Sequence
  source: /note=“Description of Artificial Sequence Synthetic polypeptide”
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 75 - protein, 525 aa, Artificial Sequence; identical to SEQ ID NO: 74
  source: /note=“Description of Artificial Sequence Synthetic polypeptide”

SEQ ID NO: 76 - protein, 521 aa, Artificial Sequence
  source: /note=“Description of Artificial Sequence Synthetic polypeptide”
MDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 77 - protein, 521 aa, Artificial Sequence; identical to SEQ ID NO: 76
  source: /note=“Description of Artificial Sequence Synthetic polypeptide”

SEQ ID NO: 78 - protein, 530 aa, Artificial Sequence
  source: /note=“Description of Artificial Sequence Synthetic polypeptide”
MDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSAEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 79 - protein, 530 aa, Artificial Sequence; identical to SEQ ID NO: 78
  source: /note=“Description of Artificial Sequence Synthetic polypeptide”

SEQ ID NO: 80 - protein, 531 aa, Artificial Sequence
  source: /note=“Description of Artificial Sequence Synthetic polypeptide”
MDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 81 - protein, 531 aa, Artificial Sequence; identical to SEQ ID NO: 80
  source: /note=“Description of Artificial Sequence Synthetic polypeptide”

SEQ ID NO: 82 - protein, 398 aa, Artificial Sequence
  source: /note=“Description of Artificial Sequence Synthetic polypeptide”
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAHHSEDPSSKAPKAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 83 - protein, 399 aa, Artificial Sequence
  source: /note=“Description of Artificial Sequence Synthetic polypeptide”
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 84 - protein, 407 aa, Artificial Sequence
  source: /note=“Description of Artificial Sequence Synthetic polypeptide”
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAHHSEDPSSKAPKAPGILGFVFTLGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 85 - protein, 408 aa, Artificial Sequence
  source: /note=“Description of Artificial Sequence Synthetic polypeptide”
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSEFPKPSTPPGSSGGAPGILGFVFTLGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVARKDEL

SEQ ID NO: 86 - protein, 414 aa, Artificial Sequence
  source: /note=“Description of Artificial Sequence Synthetic polypeptide”
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 87 - protein, 414 aa, Artificial Sequence
  source: /note=“Description of Artificial Sequence Synthetic polypeptide”
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEFPKPSTPPGSSGGAPGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVARKDEL

SEQ ID NO: 88 - protein, 519 aa, Artificial Sequence
  source: /note=“Description of Artificial Sequence Synthetic polypeptide”
MQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSSGSTSGSGKPGSGEGSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 89 - protein, 521 aa, Artificial Sequence; identical to SEQ ID NO: 56
  source: /note=“Description of Artificial Sequence Synthetic polypeptide”

SEQ ID NO: 90 - protein, 521 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 91 - protein, 521 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 92 - protein, 522 aa, Artificial Sequence
  source: /note=“Description of Artificial Sequence Synthetic polypeptide”
MQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGSTSGSGKPGSGEGSTKGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 93 - protein, 521 aa, Artificial Sequence
  source: /note=“Description of Artificial Sequence Synthetic polypeptide”
MEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGSTSGSGKPGSGEGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKEFPKPSTPPGSSGGAPGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 94 - protein, 531 aa, Artificial Sequence
  source: /note=“Description of Artificial Sequence Synthetic polypeptide”
MEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSEFPKPSTPPGSSGGAPGILGFVFTLKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 95 - protein, 530 aa, Artificial Sequence
  source: /note=“Description of Artificial Sequence Synthetic polypeptide”
MQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKSGGGGSGGGGSGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSEFPKPSTPPGSSGGILGFVFTLGAPKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 96 - protein, 520 aa, Artificial Sequence
  source: /note=“Description of Artificial Sequence Synthetic polypeptide”
MQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGSTSGSGKPGSGEGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 97 - protein, 373 aa, Artificial Sequence
  source: /note=“Description of Artificial Sequence Synthetic polypeptide”
ASVSDVPRDLEVVAATPTSLLISWCRQRCADSYRITYGETGGNSPVQEFTVPGSWKTATISGLKPGVDYTITVYVVTHYYGWDRYSHPISINYRTGSEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAR

SEQ ID NO: 98 - protein, 781 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQ

SEQ ID NO: 99 - protein, 781 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVARCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQ

SEQ ID NO: 100 - protein, 525 aa, Artificial Sequence
  source: /note=“Description of Artificial Sequence Synthetic polypeptide”
MQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELKEFPKPSTPPGSSGGAPGILGFVFTLKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARKDEL

SEQ ID NO: 101 - protein, 522 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPGILGFVFTLMQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTVTVSSGSTSGSGKPGSGEGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELK

SEQ ID NO: 102 - protein, 517 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 103 - protein, 517 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 104 - protein, 521 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 105 - protein, 521 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 106 - protein, 525 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 107 - protein, 525 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 108 - protein, 527 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 109 - protein, 527 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 110 - protein, 394 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARAHHSEDPSSKAPKAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 111 - protein, 395 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 112 - protein, 394 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVARAHHSEDPSSKAPKAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 113 - protein, 395 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 114 - protein, 374 aa, Artificial Sequence
  source: /note=“Description of Artificial Sequence Synthetic polypeptide”
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 115 - protein, 374 aa, Artificial Sequence
  source: /note=“Description of Artificial Sequence Synthetic polypeptide”
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 116 - protein, 4 aa, Artificial Sequence
  source: /note=“Description of Artificial Sequence Synthetic peptide”
KDEL

SEQ ID NO: 117 - protein, 4 aa, Artificial Sequence
  source: /note=“Description of Artificial Sequence Synthetic peptide”
HDEF

SEQ ID NO: 118 - protein, 4 aa, Artificial Sequence
  source: /note=“Description of Artificial Sequence Synthetic peptide”
HDEL

SEQ ID NO: 119 - protein, 4 aa, Artificial Sequence
  source: /note=“Description of Artificial Sequence Synthetic peptide”
RDEF

SEQ ID NO: 120 - protein, 4 aa, Artificial Sequence
  source: /note=“Description of Artificial Sequence Synthetic peptide”
RDEL

SEQ ID NO: 121 - protein, 4 aa, Artificial Sequence
  source: /note=“Description of Artificial Sequence Synthetic peptide”
WDEL

SEQ ID NO: 122 - protein, 4 aa, Artificial Sequence
  source: /note=“Description of Artificial Sequence Synthetic peptide”
YDEL

SEQ ID NO: 123 - protein, 4 aa, Artificial Sequence
  source: /note=“Description of Artificial Sequence Synthetic peptide”
HEEF

SEQ ID NO: 124 - protein, 4 aa, Artificial Sequence
  source: /note=“Description of Artificial Sequence Synthetic peptide”
HEEL

SEQ ID NO: 125 - protein, 4 aa, Artificial Sequence
  source: /note=“Description of Artificial Sequence Synthetic peptide”
KEEL

SEQ ID NO: 126 - protein, 4 aa, Artificial Sequence
  source: /note=“Description of Artificial Sequence Synthetic peptide”
REEL

SEQ ID NO: 127 - protein, 4 aa, Artificial Sequence
  source: /note=“Description of Artificial Sequence Synthetic peptide”
KAEL

SEQ ID NO: 128 - protein, 4 aa, Artificial Sequence
  source: /note=“Description of Artificial Sequence Synthetic peptide”
KCEL

SEQ ID NO: 129 - protein, 4 aa, Artificial Sequence
  source: /note=“Description of Artificial Sequence Synthetic peptide”
KFEL

SEQ ID NO: 130 - protein, 4 aa, Artificial Sequence
  source: /note=“Description of Artificial Sequence Synthetic peptide”
KGEL

SEQ ID NO: 131 - protein, 4 aa, Artificial Sequence
  source: /note=“Description of Artificial Sequence Synthetic peptide”
KHEL

SEQ ID NO: 132 - protein, 4 aa, Artificial Sequence
  source: /note=“Description of Artificial Sequence Synthetic peptide”
KLEL

SEQ ID NO: 133 - protein, 4 aa, Artificial Sequence
  source: /note=“Description of Artificial Sequence Synthetic peptide”
KNEL

SEQ ID NO: 134 - protein, 4 aa, Artificial Sequence
  source: /note=“Description of Artificial Sequence Synthetic peptide”
KQEL

SEQ ID NO: 135 - protein, 4 aa, Artificial Sequence
  source: /note=“Description of Artificial Sequence Synthetic peptide”
KREL

SEQ ID NO: 136 - protein, 4 aa, Artificial Sequence
  source: /note=“Description of Artificial Sequence Synthetic peptide”
KSEL

SEQ ID NO: 137 - protein, 4 aa, Artificial Sequence
  source: /note=“Description of Artificial Sequence Synthetic peptide”
KVEL

SEQ ID NO: 138 - protein, 4 aa, Artificial Sequence
  source: /note=“Description of Artificial Sequence Synthetic peptide”
KWEL

SEQ ID NO: 139 - protein, 4 aa, Artificial Sequence
  source: /note=“Description of Artificial Sequence Synthetic peptide”
KYEL

SEQ ID NO: 140 - protein, 4 aa, Artificial Sequence
  source: /note=“Description of Artificial Sequence Synthetic peptide”
KEDL

SEQ ID NO: 141 - protein, 4 aa, Artificial Sequence
  source: /note=“Description of Artificial Sequence Synthetic peptide”
KIEL

SEQ ID NO: 142 - protein, 4 aa, Artificial Sequence
  source: /note=“Description of Artificial Sequence Synthetic peptide”
DKEL

SEQ ID NO: 143 - protein, 4 aa, Artificial Sequence
  source: /note=“Description of Artificial Sequence Synthetic peptide”
FDEL

SEQ ID NO: 144 - protein, 4 aa, Artificial Sequence
  source: /note=“Description of Artificial Sequence Synthetic peptide”
KDEF

SEQ ID NO: 145 - protein, 4 aa, Artificial Sequence
  source: /note=“Description of Artificial Sequence Synthetic peptide”
KKEL

SEQ ID NO: 146 - protein, 4 aa, Artificial Sequence
  source: /note=“Description of Artificial Sequence Synthetic peptide”
HADL

SEQ ID NO: 147 - protein, 4 aa, Artificial Sequence
  source: /note=“Description of Artificial Sequence Synthetic peptide”
HAEL

SEQ ID NO: 148 - protein, 4 aa, Artificial Sequence
  source: /note=“Description of Artificial Sequence Synthetic peptide”
HIEL

SEQ ID NO: 149 - protein, 4 aa, Artificial Sequence
  source: /note=“Description of Artificial Sequence Synthetic peptide”
HNEL

SEQ ID NO: 150 - protein, 4 aa, Artificial Sequence
  source: /note=“Description of Artificial Sequence Synthetic peptide”
HTEL

SEQ ID NO: 151 - protein, 4 aa, Artificial Sequence
  source: /note=“Description of Artificial Sequence Synthetic peptide”
KTEL

SEQ ID NO: 152 - protein, 4 aa, Artificial Sequence
  source: /note=“Description of Artificial Sequence Synthetic peptide”
HVEL

SEQ ID NO: 153 - protein, 4 aa, Artificial Sequence
  source: /note=“Description of Artificial Sequence Synthetic peptide”
NDEL

SEQ ID NO: 154 - protein, 4 aa, Artificial Sequence
  source: /note=“Description of Artificial Sequence Synthetic peptide”
QDEL

SEQ ID NO: 155 - protein, 4 aa, Artificial Sequence
  source: /note=“Description of Artificial Sequence Synthetic peptide”
REDL

SEQ ID NO: 156 - protein, 4 aa, Artificial Sequence
  source: /note=“Description of Artificial Sequence Synthetic peptide”
RNEL

SEQ ID NO: 157 - protein, 4 aa, Artificial Sequence
  source: /note=“Description of Artificial Sequence Synthetic peptide”
RTDL

SEQ ID NO: 158 - protein, 4 aa, Artificial Sequence
  source: /note=“Description of Artificial Sequence Synthetic peptide”
RTEL

SEQ ID NO: 159 - protein, 4 aa, Artificial Sequence
  source: /note=“Description of Artificial Sequence Synthetic peptide”
SDEL

SEQ ID NO: 160 - protein, 4 aa, Artificial Sequence
  source: /note=“Description of Artificial Sequence Synthetic peptide”
TDEL

SEQ ID NO: 161 - protein, 4 aa, Artificial Sequence
  source: /note=“Description of Artificial Sequence Synthetic peptide”
SKEL

(19) United States
(12) Patent Application Publication
(10) Pub. No.: US 2021/0138076 A2
(43) Pub. Date: May 13, 2021

(54) CELL-TARGETING MOLECULES COMPRISING SHIGA TOXIN A SUBUNIT EFFECTORS AND CD8+ T-CELL EPITOPES

(71) Applicant: Molecular Templates, Inc., Austin, TX (US)

(72) Inventors: Eric Poma, New York, NY (US); Erin Willert, Round Rock, TX (US); Jason Kim, Austin, TX (US)

(73) Assignee: Molecular Templates, Inc., Austin, TX (US)

(21) Appl. No.: 15/747,501

(22) PCT Filed: Jul. 25, 2016
(86) PCT No.: PCT/US2016/043902

§ 371 Date: Jan. 25, 2018

Related U.S. Application Data
Application No. PCT/US2016/043902 is a continuation-in-part of application No. 14/965,882, filed on Dec. 10, 2015, now abandoned.
Application No. 14/965,882 is a continuation-in-part of application No. PCT/US2015/019708, filed on Mar. 10, 2015, now abandoned.
Application No. 14/965,882 is a continuation-in-part of application No. PCT/US2015/035179, filed on Jun. 10, 2015, now abandoned.
Application No. PCT/US2015/035179 is a continuation of application No. PCT/US2015/012970, filed on Jan. 26, 2015, now abandoned.
(60) Provisional application No. 62/197,048, filed on Jul. 26, 2015.
(60) Provisional application No. 61/951,121, filed on Mar. 11, 2014.
(60) Provisional application No. 62/010,918, filed on Jun. 11, 2014.
(60) Provisional application No. 62/049,325, filed on Sep. 11, 2014.
(60) Provisional application No. 61/932,000, filed on Jan. 27, 2014.

(51) Int. Cl. A61K 47/64; A61K 47/68; C07K 16/28; C07K 16/30; C07K 16/32

(52) U.S. Cl. CPC A61K 47/6415; A61K 47/6851; C07K 16/2863; C07K 16/2866; C07K 2319/55; C07K 16/3007; C07K 16/32; C07K 2317/622; C07K 2319/33; C07K 16/2896

(57) ABSTRACT

The present invention provides cell-targeting molecules which can deliver a CD8+ T-cell epitope cargo to the MHC class I presentation pathway of the cell. The cell-targeting molecules of the invention can be used to deliver virtually any CD8+ T-cell epitope from an extracellular space to the MHC class I pathway of a target cell, which may be a malignant cell and/or non-immune cell. The target cell can then display on a cell-surface the delivered CD8+ T-cell epitope complexed with MHC I molecule. The cell-targeting molecules of the invention have uses which include the targeted labeling and/or killing of specific cell-types within a mixture of cell-types, including within a chordate, as well as the stimulation of beneficial immune responses. The cell-targeting molecules of the invention have uses, e.g., in the treatment of a variety of diseases, disorders, and conditions, including cancers, tumors, growth abnormalities, immune disorders, and microbial infections.

### TECHNICAL FIELD

The present invention relates to cell-targeting molecules which each comprise (1) a binding region for cell-targeting, (2) a Shiga toxin A Subunit effector polypeptide region for subcellular delivery, and (3) one or more, heterologous, CD8+ T-cell epitopes; wherein the cell-targeting molecule is capable of delivering a heterologous, CD8+ T-cell epitope to the MHC class I presentation pathway of a target cell, such as, e.g. a malignant cell. In certain embodiments, the cell-targeting molecule of the present invention can deliver to the MHC class I presentation pathway of a target cell the heterologous, CD8+ T-cell epitope that is linked, either directly or indirectly, to the Shiga toxin A Subunit effector polypeptide at a position carboxy-terminal to the carboxy terminus of a Shiga toxin A1 fragment derived region. The cell-targeting molecules of the present invention have uses, e.g., for the delivery from an extracellular location of a CD8+ T-cell epitope to the MHC class I presentation pathway of a target cell; the cell-surface labeling of a target cell with a displayed CD8+ T-cell epitope; the selective killing of specific cell-types; the stimulation of beneficial immune responses in vivo; the elicitation of a cytotoxic T lymphocyte cell response to the target cell; the repression of detrimental immune responses in vivo; the creation of memory immune cells, and the diagnosis and treatment of a variety of diseases, disorders, and conditions, such as, e.g., cancers, tumors, other growth abnormalities, immune disorders, and microbial infections.

### BACKGROUND

The immune system protects the body from potentially harmful intrusions by discerning self from non-self. Immunosurveillance systems of chordates, which include amphibians, birds, fish, mammals, reptiles, and sharks, scan within the body for foreign molecules to identify invading pathogens, foreign cells, and malignant cells in order to mount protective immune responses. The immune systems of jawed vertebrates (Gnathostomata) constantly scan both the extracellular and intracellular environments for foreign epitopes in an attempt to detect threatening molecules, pathogens, and/or cells. In such vertebrates, the major histocompatibility (MHC) system functions to display peptides on cellular surfaces for recognition by T lymphocytes (T-cells) of the immune system {see Elliot T et al., Nature 348: 195-7 (1990)). The MHC system functions in vertebrates as part of the adaptive immune system to differentiate self from non-self, which contributes to the immune system's ability to eliminate pathogens, neutralize foreign molecules, kill infected or damaged cells, and reject transformed cells {Janeway's Immunobiology (Murphy K, ed., Garland Science, 8th ed., 2011)).

The MHC class I system plays an essential role in the immune system by providing epitope presentation of intracellular antigens {Cellular and Molecular Immunology (Abbas A, ed., Saunders, 8^{th} ed., 2014)). This process is thought to be an important part of the adaptive immune system, a system which evolved in chordates primarily to protect against intracellular pathogens as well as malignant cells expressing intracellular antigens, such as, e.g., cancer cells. For example, human infections involving intracellular pathogens may only be overcome by the combined actions of both the MHC class I and class II systems {see e.g. Chiu C, Openshaw P, Nat Immunol 16: 18-26 (2015)). The MHC class I system's contribution is to identify and kill malignant cells based on the identification of intracellular antigens.

The MHC class I system functions in any nucleated cell of a vertebrate to present intracellular (or endogenous) antigens, whereas the MHC class II pathway functions in professional antigen-presenting cells (APCs) to present extracellular (or exogenous) antigens (Neefjes J et al., Nat Rev Immunol 11: 823-36 (2011)). Intracellular or “endogenous” epitopes recognized by the MHC class I system are typically fragments of molecules encountered in the cytosol or lumen of the endoplasmic reticulum (ER) of a cell, and these molecules are typically proteolytically processed by the proteasome and/or another protease(s) in the cytosol. When present in the ER, these endogenous epitopes are loaded onto MHC class I molecules and presented on the surface of the cell as pMHC Is. In contrast, the MHC class II system functions only in specialized cells to recognize exogenous epitopes derived from extracellularly encountered molecules processed only in specific endosomal compartments, such as, e.g., late endosomes, lysosomes, phagosomes, and phagolysosomes, and including intracellular pathogens residing in endocytotic organelles.

Peptide presentation by the MHC class I system involves five main steps: 1) generation of cytosolic peptides, 2) transport of these peptides to the lumen of the ER, 3) stable complex formation of MHC class I molecules bound to certain peptides, 4) display of those stable pMHC Is on the cell surface, and 5) recognition of certain presented pMHC Is by specific CD8+ immune cells. The recognition of presented pMHC Is by a CD8+ T-cell can lead to CD8+ T-cell activation, clonal expansion, and differentiation into CD8+ effector T-cells, including cytotoxic T lymphocytes (CTLs) which target specific pMHC I presenting cells for destruction. This leads to the creation of a population of specific CD8+ effector T-cells, some of which can travel systematically throughout the body to seek and destroy cells displaying a specific epitope-MHC class I complex as well as a population of memory T-cells. If a CTL, which recognizes the specific pMHC I being presented (e.g. a recall antigen), is already present, then this CTL may immediately kill the pMHC I presenting cell and release cytokines.

In general, the MHC class I pathway begins with a cytosolic peptide. The existence of peptides in the cytosol can occur in multiple ways. In general, peptides presented by MHC class I molecules are derived from the proteasomal degradation of intracellular proteins. The MHC class I pathway can begin with transporters associated with antigen processing (TAPs) which are associated with the ER membrane. TAPs translocate peptides from the cytosol to the lumen of the ER, where they can then associate with empty MHC class I molecules. TAPs commonly translocate peptides that are 8-12 amino acid residues in length, but TAPs can also transport peptides as small as 6 and as large as 40 amino acid residues in length (Koopmann J et al., Eur J Immunol 26: 1720-8 (1996)).

The MHC class I pathway can also be initiated in the lumen of the ER by a pathway involving transport of a protein or peptide into the cytosol for processing and then transporting certain degraded fragments back into the ER via TAP-mediated translocation.

The peptides transported from the cytosol into the lumen of the ER by TAP are then available to be bound by MHC class I molecules. In the ER, a complex, peptide-loading, molecular machine helps assemble stable peptide-MHC class I molecule complexes (pMHC Is) and, in some instances, further processes the peptides by cleaving them into optimal sizes in a process called trimming (see Mayerhofer P, Tampe R, J Mol Biol pii S0022-2835 (2014)). In the ER, MHC class I molecules tightly bind specific peptide-epitopes using highly specific immunoglobulin-type, antigen-binding domains, each of which has strong binding affinity only to a certain peptide-epitopes. Then the peptide-MHC class I complex is transported via the secretory pathway to the plasma membrane for presentation to the extracellular environment and inspection by CD8+ immune cells. Then, specific CD8+ CTLs are targeted to kill cells presenting specific pMHC Is to protect the organism.

The presentation of specific epitope-peptides complexed with MHC class I molecules by nucleated cells in chordates plays a major role in stimulating and maintaining immune responses to intracellular pathogens, tumors, and cancers. Intercellular CD8+ T-cell engagement of a cell presenting a specific epitope-MHC class I complex by a CD8+ T-cell initiates protective immune responses that can result in the rejection of the presenting cell, i.e. death of the presenting cell due to the cytotoxic activity of one or more CTLs. The specificity of this intercellular engagement is determined by multiple factors. CD8+ T-cells recognize pMHC Is on the cell surface of another cell via their TCRs. CD8+ T-cells express different T-cell receptors (TCRs) with differing binding specificities to different cognate pMHC Is. CD8+ T-cell specificity depends on each individual T-cell's specific TCR and that TCR's binding affinity to the presented epitope-MHC complex as well as the overall TCR binding occupancy to the presenting cell. In addition, there are diverse variants of MHC class I molecules that influence intercellular CD8+ T-cell recognition in at least in two ways: by affecting the specificity of peptides loaded and displayed {i.e. the pMHC I repertoire) and by affecting the contact regions between TCRs and pMHC Is involved in epitope recognition.

The presentation of certain epitopes complexed with MHC class I molecules can sensitize the presenting cell to targeted killing by lysis, induced apoptosis, and/or necrosis. CTL killing of pMHC I-presenting cells occurs primarily via cytolytic activities mediated by the delivery of perforin and/or granzyme into the presenting cell via cytotoxic granules {see e.g. Russell J, Ley T, Annu Rev Immunol 20: 323-70 (2002); Cullen S, Martin S, Cell Death Diff\5: 251-62 (2008)). Other CTL-mediated target cell killing mechanisms involve inducing apoptosis in the presenting cell via TNF signaling, such as, e.g., via FasL/Fas and TRAIL/TRAIL-DR signaling {see e.g. Topham D et al., J Immunol 159: 5197-200 (1997); Ishikawa E et al., J Virol 79: 7658-63 (2005); Brincks E et al., J Immunol 181: 4918-25 (2008); Cullen S, Martin S, Cell Death Diff 15: 251-62 (2008)). Furthermore, activated CTLs can indiscriminately kill other cells in proximity to the recognized, pMHC I-presenting cell regardless of the peptide-MHC class I complex repertoires being presented by the other proximal cells (Wiedemann A et al., Proc Natl Acad Sci USA 103: 10985-90 (2006)). In addition, activated CTLs can release immuno-stimulatory cytokines, interleukins, and other molecules to influence the immuno-activation of the microenvironment.

This MHC class I and CTL immunosurveillance system could conceivably be harnessed by certain therapies to guide a subject's adaptive immune system into rejecting and specifically killing certain cell types. In particular, the MHC class I presentation pathway could be exploited by various therapeutic molecules to force certain targeted cells to display certain epitopes on cell surfaces in order to induce desired immune responses including the killing of specifically targeted cells. Such therapeutic molecules could specifically deliver CD8+ T-cell epitopes to the MHC class I pathway for presentation by malignant cells (e.g. tumor or infected cells) to signal their own destruction. However, there are several barriers to developing such therapeutic molecules, including, e.g., cell-type targeting of the therapeutic molecule; delivery of the therapeutic molecule through the target cell's plasma membrane; providing a therapeutic molecule that can escape the endocytotic pathway and avoid destruction in the lysosome; and providing a therapeutic molecule that can generally protect its CD8+ T-cell epitope cargo from the sequestration, modification, and/or destruction of exogenous, foreign molecules by target cells while delivering its cargo to a desired subcellular location (Sahay G et al., J Control Release 145: 182-195 (2010); Fuchs H et al., Antibodies 2: 209-35 (2013)).

Generally, the exogenous administration of a foreign molecule to a cell results in the degradation of the molecule, sometimes after sequestration and/or modification. First, the administration of exogenous peptides (e.g. an immunogenic epitopes) or proteins (e.g. an antigenic protein) to a cell results in these molecules not entering the cell due to the physical barrier of the plasma membrane. In addition, these molecules are often degraded into smaller molecules (e.g. proteins into peptides) by extracellular enzymatic activities on the surfaces of cells and/or in the extracellular milieu. Proteins that are internalized from the extracellular environment by endocytosis are commonly degraded by lysosomal proteolysis as part of an endocytotic pathway involving early endosomes, late endosomes, and lysosomes. Proteins that are internalized from the extracellular environment by phagocytosis are commonly degraded by a similar pathway ending in phagolysosomes. Thus, exogenously administered peptides and proteins, or fragments thereof, generally do not reach an intracellular compartment competent for entry into the MHC class I pathway, such as, e.g., the cytosol or ER.

It would be desirable to have cell-targeting molecules capable, when exogenously administered, of delivering a CD8+ T-cell epitope to the MHC class I presentation pathway of a chosen target cell, where the target cell may be chosen from a wide variety of cells, such as, e.g., malignant and/or infected cells, particularly cells other than professional APCs like dendritic cells. Such cell-targeting molecules, which preferentially target malignant cells over healthy cells, may be administered to a chordate for the in vivo delivery of a CD8+ T-cell epitope for MHC class I presentation by targeted cells, such as, e.g., infected, neoplastic, or otherwise malignant cells.

### SUMMARY OF THE INVENTION

The present invention provides Shiga toxin A Subunit derived, cell-targeting molecules comprising CD8+ T-cell epitope-peptides heterologous to Shiga toxin A Subunits; wherein each cell-targeting molecule has the ability to deliver its CD8+ T-cell epitope-peptide cargo to the MHC class I presentation pathway of a target cell. Cell-targeting molecules of the present invention may be used for targeted delivery of various CD8+ T-cell epitopes to any nucleated, target cell within a chordate in order to cause the delivered CD8+ T-cell epitope to be presented on the target cell surface complexed with a MHC class I molecule. The target cells can be of various types, such as, e.g., neoplastic cells, infected cells, cells harboring intracellular pathogens, and other undesirable cells, and the target cell can be targeted by cell-targeting molecules of the invention either in vitro or in vivo. In addition, the present invention provides various cell-targeted molecules comprising protease-cleavage resistant, Shiga toxin effector polypeptides capable of intracellular delivery of heterologous, CD8+ T-cell epitopes to the MHC class I presentation pathways of target cells while simultaneously improving extracellular, in vivo tolerability of these cell-targeting molecules. Certain cell-targeting molecules of the present invention have improved usefulness for administration to chordates as either a therapeutic and/or diagnostic agent because of the reduced likelihood of producing nonspecific toxicities at a given dosage.

The cell-targeting molecule of the present invention comprises three distinct components: (i) a Shiga toxin effector polypeptide, (ii) a binding region capable of specifically binding at least one extracellular target biomolecule, and (iii) a CD8+ T-cell epitope; whereby administration of the cell-targeting molecule to a cell results in the cell presenting on a cellular surface the CD8+ T-cell epitope-peptide complexed with a MHC class I molecule. In certain further embodiments, the CD8+ T-cell epitope is fused, either directly or indirectly, to the Shiga toxin effector polypeptide and/or the binding region. In certain further embodiments, the cell-targeting molecule comprises a single-chain polypeptide comprising the binding region, the Shiga toxin effector polypeptide, and the CD8+ T-cell epitope-peptide.

In certain embodiments, the cell-targeting molecule of the present invention comprises (i) a Shiga toxin effector polypeptide having a Shiga toxin A1 fragment region, (ii) a heterologous binding region comprising a cell-targeting moiety or agent capable of specifically binding at least one extracellular target biomolecule, and (iii) a heterologous, CD8+ T-cell epitope-peptide; whereby administration of the cell-targeting molecule to a cell results in the cell presenting on a cellular surface the CD8+ T-cell epitope-peptide complexed with a MHC class I molecule. In certain further embodiments, the heterologous, CD8+ T-cell epitope is not embedded or inserted in the Shiga toxin A 1 fragment region. In certain further embodiments, the heterologous, CD8+ T-cell epitope-peptide is fused, either directly or indirectly, to the Shiga toxin effector polypeptide and/or the binding region. In certain further embodiments, the cell-targeting molecule comprises a single-chain polypeptide comprising the binding region, the Shiga toxin effector polypeptide, and the heterologous, CD8+ T-cell epitope-peptide.

In certain embodiments, the cell-targeting molecule of the present invention comprises (i) a Shiga toxin effector polypeptide having a Shiga toxin A1 fragment region, (ii) a heterologous binding region comprising a cell-targeting moiety or agent capable of specifically binding at least one extracellular target biomolecule, and (iii) a heterologous, CD8+ T-cell epitope-peptide; whereby administration of the cell-targeting molecule to a cell results in the cell presenting on a cellular surface the CD8+ T-cell epitope-peptide complexed with a MHC class I molecule; and with the proviso that the cell-targeting molecule does not comprise or consist of SEQ ID NOs: 71-72. In certain further embodiments, the heterologous, CD8+ T-cell epitope is not embedded or inserted in the Shiga toxin A 1 fragment region. In certain further embodiments, the heterologous, CD8+ T-cell epitope-peptide is fused, either directly or indirectly, to the Shiga toxin effector polypeptide and/or the binding region. In certain further embodiments, the cell-targeting molecule comprises a single-chain polypeptide comprising the binding region, the Shiga toxin effector polypeptide, and the heterologous, CD8+ T-cell epitope-peptide.

For certain embodiments, administration of the cell-targeting molecule to a cell results in the CD8+ T-cell epitope-peptide becoming complexed with a MHC class I molecule at an intracellular location before the cell presenting on a cellular surface the CD8+ T-cell epitope-peptide complexed with a MHC class I molecule.

In certain embodiments of the cell-targeting molecules of the present invention, the binding region comprises two or more polypeptide chains and the heterologous, CD8+ T-cell epitope-peptide is fused either directly or indirectly, to a polypeptide comprising the Shiga toxin effector polypeptide and one of the two or more polypeptide chains of the binding region.

In certain embodiments of the cell-targeting molecules of the present invention, the binding region comprises a polypeptide selected from the group consisting of: an autonomous VH domain, single-domain antibody fragment (sdAb), nanobody, heavy chain-antibody domain derived from a camelid (VHH or VH domain fragment), heavy-chain antibody domain derived from a cartilaginous fish (VHH or VH domain fragment), immunoglobulin new antigen receptor (IgNAR), VNAR fragment, single-chain variable fragment (scFv), antibody variable fragment (Fv), complementary determining region 3 fragment (CDR3), constrained FR3-CDR3-FR4 polypeptide (FR3-CDR3-FR4), Fd fragment, small modular immunopharmaceutical (SMIP) domain, antigen-binding fragment (Fab), Armadillo repeat polypeptide (ArmRP), fibronectin-derived 10^{th} fibronectin type III domain (10Fn3), tenascin type III domain (TNfn3), ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality.

In certain embodiments of the cell-targeting molecules of the present invention, the Shiga toxin effector polypeptide comprises or consists essentially of the polypeptide sequence selected from the group consisting of: (i) amino acids 75 to 251 of SEQ ID NO:1, SEQ ID NO:2, or SEQ ID NO:3; (ii) amino acids 1 to 241 of SEQ ID NO:1, SEQ ID NO:2, or SEQ ID NO:3; (iii) amino acids 1 to 251 of SEQ ID NO: 1, SEQ ID NO:2, or SEQ ID NO:3; and (iv) amino acids 1 to 261 of SEQ ID NO:1, SEQ ID NO:2, or SEQ ID NO:3.

In certain embodiments of the cell-targeting molecules of the present invention, the binding region is capable of binding to the extracellular target biomolecule selected from the group consisting of: CD20, CD22, CD40, CD74, CD79, CD25, CD30, HER2/neu/ErbB2, EGFR, EpCAM, EphB2, prostate-specific membrane antigen (PSMA), Cripto, CDCP1, endoglin, fibroblast activated protein (FAP), Lewis-Y, CD19, CD21, CSl/SLAMF7, CD33, CD52, CD133, CEA, gpA33, mucin, TAG-72, tyrosine-protein kinase transmembrane receptor (ROR1 or NTRKR1), carbonic anhydrase IX (CA9), folate binding protein (FBP), ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gplOO, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen (CEA), prostate specific antigen (PSA), prostate stem cell antigen (PSCA), human aspartyl (asparaginyl) beta-hydroxylase, EphA2, HER3/ErbB-3, MUC1, MART-1/MelanA, gplOO, tyrosinase associated antigen, HPV-E7, Epstein-Barr virus antigen, Bcr-Abl, alpha-fetoprotein antigen, 17-A1, bladder tumor antigen (BTA), CD38, CD15, CD23, CD45 (protein tyrosine phosphatase receptor type C), CD53, CD88, CD129, CD183, CD191, CD193, CD244, CD294, CD305, C3AR, FceRIa, galectin-9, IL-1R (interleukin-1 receptor), mrp-14, NKG2D ligand, programmed death-ligand 1 (PD-L1), Siglec-8, Siglec-10, CD49d, CD 13, CD44, CD54, CD63, CD69, CD 123, TLR4, FceRIa, IgE, CD 107a, CD203c, CD14, CD68, CD80, CD86, CD105, CD115, F4/80, ILT-3, galectin-3, CD1 la-c, GITRL, MHC class I molecule (optionally complexed with a polypeptide), MHC class II molecule (optionally complexed with a peptide), CD284 (TLR4), CD107-Mac3, CD195 (CCR5), HLA-DR, CD16/32, CD282 (TLR2), CD1 lc, and any immunogenic fragment of any of the foregoing.

In certain embodiments, the cell-targeting molecule of the present invention comprises a carboxy-terminal endoplasmic reticulum retention/retrieval signal motif of a member of the KDEL family. In certain further embodiments, the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL.

In certain embodiments, the cell-targeting molecule of the present invention comprises a heterologous, CD8+ T-cell epitope-peptide which is positioned within the cell-targeting molecule carboxy-terminal to the Shiga toxin effector polypeptide and/or binding region. In certain further embodiments, the cell-targeting molecule comprises two, three, four, five, or more heterologous, CD8+ T-cell epitope-peptides positioned within the cell-targeting molecule carboxy-terminal to the Shiga toxin effector polypeptide and/or binding region.

In certain embodiments, the cell-targeting molecule comprises a carboxy-terminal, heterologous, CD8+ T-cell epitope-peptide.

For certain embodiments of the cell-targeting molecules of the present invention, upon administration of the cell-targeting molecule to a target cell physically coupled with an extracellular target biomolecule of the binding region, the cell-targeting molecule is capable of causing intercellular engagement of the target cell by a CD8+ immune cell.

For certain embodiments of the cell-targeting molecules of the present invention, upon administration of the cell-targeting molecule to a target cell physically coupled with an extracellular target biomolecule of the binding region, the cell-targeting molecule is capable of causing death of the target cell. For certain further embodiments, upon administration of the cell-targeting molecule of the present invention to a first population of cells whose members are physically coupled to extracellular target biomolecules of the binding region, and a second population of cells whose members are not physically coupled to any extracellular target biomolecule of the binding region, the cytotoxic effect of the cell-targeting molecule to members of said first population of cells relative to members of said second population of cells is at least 3-fold greater.

In certain embodiments of the cell-targeting molecules of the present invention, the Shiga toxin effector polypeptide comprises a mutation relative to a naturally occurring A Subunit of a member of the Shiga toxin family which changes the enzymatic activity of the Shiga toxin effector polypeptide, the mutation selected from at least one amino acid residue deletion, insertion, or substitution. In certain further embodiments, the mutation is selected from at least one amino acid residue deletion, insertion, or substitution that reduces or eliminates cytotoxicity of the toxin effector polypeptide.

In certain embodiments, the cell-targeting molecule of the present invention does not consist of nor comprise any one of SEQ ID NOs: 71-115.

In certain embodiments, the cell-targeting molecule of the present invention comprises or consists essentially of the polypeptide of any one of SEQ ID NOs: 13-61 and 72-115.

In certain embodiments, the cell-targeting molecule of the present invention comprises (i) a binding region comprising a cell-targeting moiety or agent capable of specifically binding at least one extracellular target biomolecule, (ii) a Shiga toxin effector polypeptide comprising a Shiga toxin A1 fragment derived region having a carboxy terminus, and (iii) a heterologous, CD8+ T-cell epitope-peptide linked to a proteinaceous component of the cell-targeting molecule; whereby the heterologous, CD8+ T-cell epitope-peptide is carboxy-terminal to the carboxy terminus of the Shiga toxin A1 fragment derived region; and whereby administration of cell-targeting molecule to a cell results in the cell presenting on a cellular surface the CD8+ T-cell epitope-peptide complexed with a MHC class I molecule (see e.g. FIG. 1-B). In certain further embodiments, the heterologous, CD8+ T-cell epitope-peptide is fused, either directly or indirectly, to the Shiga toxin effector polypeptide and/or the binding region. In certain further embodiments, the cell-targeting molecule comprises a single-chain polypeptide comprising the binding region, the Shiga toxin effector polypeptide, and the heterologous, CD8+ T-cell epitope-peptide. In certain embodiments, the binding region comprises two or more polypeptide chains and the heterologous, CD8+ T-cell epitope-peptide is fused, either directly or indirectly, to a polypeptide comprising the Shiga toxin effector polypeptide and one of the two or more polypeptide chains of the binding region. In certain further embodiments, the binding region comprises a polypeptide selected from the group consisting of: an autonomous V H domain, single-domain antibody fragment (sdAb), nanobody, heavy chain-antibody domain derived from a camelid (VHH or V H domain fragment), heavy-chain antibody domain derived from a cartilaginous fish (VHH or V H domain fragment), immunoglobulin new antigen receptor (IgNAR), VNAR fragment, single-chain variable fragment (scFv), antibody variable fragment (Fv), complementary determining region 3 fragment (CDR3), constrained FR3-CDR3-FR4 polypeptide (FR3-CDR3-FR4), Fd fragment, small modular immunopharmaceutical (SMIP) domain, antigen-binding fragment (Fab), Armadillo repeat polypeptide (ArmRP), fibronectin-derived 10^{th} fibronectin type III domain (10Fn3), tenascin type III domain (TNfn3), ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality. In certain further embodiments, the Shiga toxin effector polypeptide comprises or consists essentially of the polypeptide sequence selected from the group consisting of: (i) amino acids 75 to 251 of SEQ ID NO:1, SEQ ID NO:2, or SEQ ID NO:3; (ii) amino acids 1 to 241 of SEQ ID NO:1, SEQ ID NO:2, or SEQ ID NO:3; (iii) amino acids 1 to 251 of SEQ ID NO:1, SEQ ID NO:2, or SEQ ID NO:3; and (iv) amino acids 1 to 261 of SEQ ID NO:1, SEQ ID NO:2, or SEQ ID NO:3. In certain further embodiments, the cell-targeting molecule comprises or consists essentially of the polypeptide of any one of SEQ ID NOs: 21-39, 52-53, 57-61, and 101-115. In certain further embodiments of the cell-targeting molecule of the present invention, the Shiga toxin effector polypeptide comprises a Shiga toxin A 1 fragment derived region having a carboxy terminus and the carboxy terminus of the Shiga toxin A 1 fragment derived region comprises a disrupted furin-cleavage motif. In certain further embodiments, the disrupted furin-cleavage motif comprises one or more mutations, relative to a wild-type Shiga toxin A Subunit, in a minimal furin cleavage site of the furin-cleavage motif. In certain further embodiments the minimal furin cleavage site is represented by the consensus amino acid sequence R/Y-x-x-R and/or R-x-x-R. In certain further embodiments, the disrupted furin-cleavage motif comprises one or more mutations, relative to a wild-type Shiga toxin A Subunit, the mutation altering at least one amino acid residue in a region natively positioned at 248-251 of the A Subunit of Shiga-like toxin 1 (SEQ ID NO: 1) or Shiga toxin (SEQ ID NO:2), or at 247-250 of the A Subunit of Shiga-like toxin 2 (SEQ ID NO:3) or the equivalent region in a Shiga toxin A Subunit or derivative thereof. In certain further embodiments, the disrupted furin-cleave motif comprises an amino acid residue substitution in the furin-cleavage motif relative to a wild-type Shiga toxin A Subunit. In certain further embodiments, the substitution of the amino acid residue in the furin-cleavage motif is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In certain further embodiments, the binding region is capable of binding to the extracellular target biomolecule selected from the group consisting of: CD20, CD22, CD40, CD74, CD79, CD25, CD30, HER2/neu/ErbB2, EGFR, EpCAM, EphB2, prostate-specific membrane antigen (PSMA), Cripto, CDCP1, endoglin, fibroblast activated protein (FAP), Lewis-Y, CD 19, CD21, CS1/SLAMF7, CD33, CD52, CD 133, CEA, gpA33, mucin, TAG-72, tyrosine-protein kinase transmembrane receptor (ROR1 or NTRKR1), carbonic anhydrase IX (CA9), folate binding protein (FBP), ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gplOO, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen (CEA), prostate specific antigen (PSA), prostate stem cell antigen (PSCA), human aspartyl (asparaginyl) beta-hydroxylase, EphA2, HER3/ErbB-3, MUC1, MART-1/MelanA, gplOO, tyrosinase associated antigen, HPV-E7, Epstein-Barr virus antigen, Bcr-Abl, alpha-fetoprotein antigen, 17-A1, bladder tumor antigen (BTA), CD38, CD15, CD23, CD45 (protein tyrosine phosphatase receptor type C), CD53, CD88, CD129, CD183, CD191, CD193, CD244, CD294, CD305, C3AR, FceRIa, galectin-9, IL-1R (interleukin-1 receptor), mrp-14, NKG2D ligand, programmed death-ligand 1 (PD-L1), Siglec-8, Siglec-10, CD49d, CD 13, CD44, CD54, CD63, CD69, CD 123, TLR4, FceRIa, IgE, CD 107a, CD203c, CD 14, CD68, CD80, CD86, CD105, CD115, F4/80, ILT-3, galectin-3, CD1 la-c, GITRL, MHC class I molecule (optionally complexed with a polypeptide), MHC class II molecule (optionally complexed with a peptide), CD284 (TLR4), CD107-Mac3, CD 195 (CCR5), HLA-DR, CD 16/32, CD282 (TLR2), CD 11c, and any immunogenic fragment of any of the foregoing. In certain further embodiments, the cell-targeting molecule of the present invention comprises a carboxy-terminal endoplasmic reticulum retention/retrieval signal motif of a member of the KDEL family. In certain further embodiments, the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. For certain embodiments, upon administration of the cell-targeting molecule of the present invention to a target cell physically coupled with an extracellular target biomolecule of the binding region, the cell-targeting molecule is capable of causing intercellular engagement of the target cell by a CD8+ immune cell. For certain further embodiments, upon administration of the cell-targeting molecule of the present invention to a target cell physically coupled with an extracellular target biomolecule of the binding region, the cell-targeting molecule is capable of causing death of the target cell. For certain further embodiments, upon administration of the cell-targeting molecule of the present invention to a first population of cells whose members are physically coupled to extracellular target biomolecules of the binding region, and a second population of cells whose members are not physically coupled to any extracellular target biomolecule of the binding region, the cytotoxic effect of the cell-targeting molecule to members of said first population of cells relative to members of said second population of cells is at least 3-fold greater. In certain further embodiments, the cell-targeting molecule comprises or consists essentially of the polypeptide of any one of SEQ ID NOs: 21-39, 52, 57-61, and 101-115. In certain embodiments, the Shiga toxin effector polypeptide comprises a mutation relative to a naturally occurring A Subunit of a member of the Shiga toxin family which changes the enzymatic activity of the Shiga toxin effector polypeptide, the mutation selected from at least one amino acid residue deletion, insertion, or substitution. In certain further embodiments, the mutation is selected from at least one amino acid residue deletion, insertion, or substitution that reduces or eliminates cytotoxicity of the toxin effector polypeptide. In certain further embodiments, the cell-targeting molecule comprises or consists essentially of the polypeptide shown in SEQ ID NO:53. In certain embodiments, the binding region comprises the heterologous, CD8+ T-cell epitope, whether the CD8+ epitope-peptide is autogenous or heterologous with respect to the binding region.

In certain embodiments of the cell-targeting molecules of the present invention, the heterologous, CD8+ T-cell epitope-peptide is fused, either directly or indirectly, to the Shiga toxin effector polypeptide and/or the binding region. In certain further embodiments, the cell-targeting molecule comprises a single-chain polypeptide comprising the binding region, the Shiga toxin effector polypeptide, and the heterologous, CD8+ T-cell epitope-peptide.

In certain embodiments of the cell-targeting molecule of the present invention, the carboxy terminus of the Shiga toxin A1 fragment derived region comprises a disrupted furin-cleavage motif. In certain further embodiments, the disrupted furin-cleavage motif comprises one or more mutations, relative to a wild-type Shiga toxin A Subunit, in a minimal furin cleavage site of the furin-cleavage motif. In certain further embodiments the minimal furin cleavage site is represented by the consensus amino acid sequence R/Y-x-x-R and/or R-x-x-R. In certain further embodiments, the disrupted furin-cleavage motif comprises one or more mutations, relative to a wild-type Shiga toxin A Subunit, the mutation altering at least one amino acid residue in a region natively positioned at 248-251 of the A Subunit of Shiga-like toxin 1 (SEQ ID NO: 1) or Shiga toxin (SEQ ID NO:2), or at 247-250 of the A Subunit of Shiga-like toxin 2 (SEQ ID NO:3) or the equivalent region in a Shiga toxin A Subunit or derivative thereof. In certain further embodiments, the disrupted furin-cleave motif comprises an amino acid residue substitution in the furin-cleavage motif relative to a wild-type Shiga toxin A Subunit.

In certain embodiments of the cell-targeting molecule of the present invention, the heterologous, CD8+ T-cell epitope-peptide is embedded or inserted in the binding region.

For certain embodiments, administration of the cell-targeting molecule to a cell results in the CD8+ T-cell epitope-peptide becoming complexed with a MHC class I molecule at an intracellular location before the cell presenting on a cellular surface the CD8+ T-cell epitope-peptide complexed with a MHC class I molecule. For certain embodiments, the cell-targeting molecule of the present invention and/or its Shiga toxin effector polypeptide is capable of exhibiting subcellular routing efficiency comparable to a reference cell-targeting molecule comprising a wild-type Shiga toxin A1 fragment or wild-type Shiga toxin effector polypeptide and/or capable of exhibiting a significant level of intracellular routing activity to the endoplasmic reticulum and/or cytosol from an endosomal starting location of a cell.

For certain embodiments, the cell-targeting molecule of the present invention is capable when introduced to a chordate of exhibiting improved, in vivo tolerability compared to a second cell-targeting molecule consisting of the first cell-targeting molecule except for all of its Shiga toxin effector polypeptide component(s) each comprise a wild-type Shiga toxin A 1 fragment and/or wild-type Shiga toxin furin-cleavage site at the carboxy terminus of its A 1 fragment region. This means the second cell-targeting molecule comprises a Shiga toxin A Subunit effector polypeptide linked in the same way as in the cell-targeting molecule of the invention to the same binding region and the same heterologous, CD8+ epitope-peptide(s) as the cell-targeting molecule of the invention, but the Shiga toxin effector polypeptide of the second cell-targeting molecule differs from the Shiga toxin effector polypeptide of the first cell-targeting molecule in that it comprises a wild-type, Shiga toxin effector polypeptide comprising a Shiga toxin A 1 fragment region having a carboxy terminus and/or a wild-type furin-cleavage site at the carboxy terminus of the A 1 fragment region of the wild-type, Shiga toxin effector polypeptide.

For certain embodiments, the cell-targeting molecule of the present invention is capable of exhibiting (i) a catalytic activity level comparable to a wild-type Shiga toxin A 1 fragment or wild-type Shiga toxin effector polypeptide, (ii) a ribosome inhibition activity with a half-maximal inhibitory concentration (ICso) value of 10,000 picomolar or less, and/or (iii) a significant level of Shiga toxin catalytic activity.

For certain embodiments of the cell-targeting molecule of the present invention, whereby administration of the cell-targeting molecule to a cell physically coupled with the extracellular target biomolecule of the cell-targeting molecule's binding region, the cell-targeting molecule is capable of causing death of the cell. In certain further embodiments, administration of the cell-targeting molecule of the invention to two different populations of cell types which differ with respect to the presence or level of the extracellular target biomolecule, the cell-targeting molecule is capable of causing cell death to the cell-types physically coupled with an extracellular target biomolecule of the cytotoxic cell-targeting molecule's binding region at a CD50 at least three times or less than the CD50 to cell types which are not physically coupled with an extracellular target biomolecule of the cell-targeting molecule's binding region. For certain embodiments, whereby administration of the cell-targeting molecule of the present invention to a first populations of cells whose members are physically coupled to extracellular target biomolecules of the cell-targeting molecule's binding region, and a second population of cells whose members are not physically coupled to any extracellular target biomolecule of the binding region, the cytotoxic effect of the cell-targeting molecule to members of said first population of cells relative to members of said second population of cells is at least 3-fold greater. For certain embodiments, whereby administration of the cell-targeting molecule of the present invention to a first populations of cells whose members are physically coupled to a significant amount of the extracellular target biomolecule of the cell-targeting molecule's binding region, and a second population of cells whose members are not physically coupled to a significant amount of any extracellular target biomolecule of the binding region, the cytotoxic effect of the cell-targeting molecule to members of said first population of cells relative to members of said second population of cells is at least 3-fold greater. For certain embodiments, whereby administration of the cell-targeting molecule of the present invention to a first population of target biomolecule positive cells, and a second population of cells whose members do not express a significant amount of a target biomolecule of the cell-targeting molecule's binding region at a cellular surface, the cytotoxic effect of the cell-targeting molecule to members of the first population of cells relative to members of the second population of cells is at least 3-fold greater.

For certain embodiments, the cell-targeting molecule of the present invention is capable when introduced to cells of exhibiting a cytotoxicity with a half-maximal inhibitory concentration (CD50) value of 300 nM or less and/or capable of exhibiting a significant level of Shiga toxin cytotoxicity.

For certain embodiments, the cell-targeting molecule of the present invention exhibits low cytotoxic potency (i.e. is not capable when introduced to certain positive target cell types of exhibiting a cytotoxicity greater than 1% cell death of a cell population at a cell-targeting molecule concentration of 1000 nM, 500 nM, 100 nM, 75 nM, or 50 nM).

In certain embodiments, the cell-targeting molecule of the present invention does not comprise a naturally occurring Shiga toxin B Subunit. In certain embodiments, the cell-targeting molecule of the invention does not comprise any polypeptide comprising or consisting essentially of a functional binding domain of a native, Shiga toxin B subunit. Rather, in certain embodiments of the cell-targeting molecules of the invention, the Shiga toxin A Subunit polypeptide(s) are functionally associated with heterologous, binding regions to effectuate cell targeting.

In certain embodiments of the cell-targeting molecules of the present invention, the heterologous, CD8+ T-cell epitope-peptide is not embedded in the Shiga toxin A1 fragment region. In certain embodiments, the heterologous, CD8+ T-cell epitope-peptide is not embedded in the Shiga toxin effector polypeptide.

In certain embodiments of the cell-targeting molecules of the present invention, the heterologous, CD8+ T-cell epitope-peptide is not inserted in the Shiga toxin A1 fragment region. In certain embodiments, the heterologous, CD8+ T-cell epitope-peptide is not inserted in the Shiga toxin effector polypeptide.

In certain embodiments of the cell-targeting molecule of the present invention, the heterologous, CD8+ T-cell epitope-peptide does not comprise or consist of the polypeptide shown in SEQ ID NO: 10. In certain embodiments, the cell-targeting molecule of the present invention does not comprise the Shiga toxin effector polypeptide comprising the CD8+ T-cell epitope-peptide GILGFVFTL (SEQ ID NO: 10) embedded at native position 53 in SLT-1A (SEQ ID NO:1). In certain embodiments, the cell-targeting molecule of the present invention does not comprise the polypeptide shown in SEQ ID NO: 10. In certain embodiments, the cell-targeting molecule of the present invention does not comprise any Shiga toxin effector polypeptide comprising any embedded or inserted, CD8+ T-cell epitope.

In certain embodiments, the cell-targeting molecule of the present invention does not comprise the linker shown in SEQ ID NO:71 wherein the linker is fused, either directly or indirectly, between a binding region and a Shiga toxin effector polypeptide and wherein the binding region is positioned amino-terminal to the Shiga toxin effector polypeptide. In certain embodiments, the cell-targeting molecule of the present invention does not comprise the linker shown in SEQ ID NO:71 wherein the linker is fused between a binding region and a Shiga toxin effector polypeptide.

In certain embodiments, the cell-targeting molecule of the present invention does not comprise any heterologous, CD8+ T-cell epitope-peptide fused between a binding region and a Shiga toxin effector polypeptide wherein the binding region is positioned amino-terminal to the Shiga toxin effector. In certain embodiments, the cell-targeting molecule of the present invention does not comprise any heterologous, CD8+ T-cell epitope-peptide fused between a binding region and a Shiga toxin effector polypeptide.

For certain embodiments of the cell-targeting molecule of the present invention, the target cell is not a professional antigen presenting cell, such as a dendritic cell type. For certain embodiments of the cell-targeting molecule of the present invention, the extracellular target biomolecule of the binding region is not expressed by a professional antigen presenting cell. For certain embodiments of the cell-targeting molecule of the present invention, the extracellular target biomolecule of the binding region is not physically associated in significant quantities with a professional antigen presenting cell. For certain embodiments of the cell-targeting molecule of the present invention, the extracellular target biomolecule of the binding region is not physically associated with a professional antigen presenting cell. For certain embodiments of the cell-targeting molecules of the present invention, the target biomolecule of the binding region is not expressed in significant amounts on the cellular surface of any professional antigen presenting cell within the chordate subject to be treated.

In certain embodiments of the cell-targeting molecule of the present invention, the heterologous, CD8+ T-cell epitope-peptide is not directly associated with any amino acid residue of the Shiga toxin A 1 fragment derived region of the Shiga toxin effector polypeptide. In certain embodiments of the cell-targeting molecule of the present invention, the heterologous, CD8+ T-cell epitope-peptide is not directly associated with any internal amino acid residue of the Shiga toxin effector polypeptide, meaning either the amino- or carboxy-terminal amino acid residue of the Shiga toxin effector polypeptide may be directly linked to a heterologous, CD8+ T-cell epitope-peptide.

In certain embodiments of the cell-targeting molecule of the present invention, the heterologous, CD8+ T-cell epitope-peptide is not embedded in the Shiga toxin effector polypeptide. In certain embodiments of the cell-targeting molecule of the present invention, the heterologous, CD8+ T-cell epitope-peptide is not inserted in the Shiga toxin effector polypeptide.

In certain embodiments of the cell-targeting molecule of the present invention, the binding region does not comprise a fragment of human CD4 corresponding to amino acid residues 19-183. In certain embodiments of the cell-targeting molecule of the present invention, the binding region does not comprise a fragment of human CD4, a type-I transmembrane glycoprotein. In certain embodiments of the cell-targeting molecule of the present invention, the binding region does not comprise a fragment of a human, immune cell surface co-receptor.

Among certain embodiments of the present invention is a method of delivering into a cell a CD8+ T-cell epitope capable of being presented by a MHC class I molecule of the cell, the method comprising the step of contacting the cell with the cell-targeting molecule of the present invention and/or a composition thereof (e.g., a pharmaceutical or diagnostic composition of the present invention).

Among certain embodiments of the present invention is a method of inducing a cell to present an exogenously administered CD8+ T-cell epitope complexed to a MHC class I molecule, the method comprising the step of contacting the cell, either in vitro or in vivo, with the cell-targeting molecule of the present invention, which comprises the CD8+ T-cell epitope, and/or a composition thereof (e.g., a pharmaceutical or diagnostic composition of the present invention comprising such a cell-targeting molecule of the present invention).

Among certain embodiments of the present invention is a method of inducing an immune cell-mediated response to target cell via a CD8+ T-cell epitope MHC class I molecule complex, the method comprising the step of contacting the target cell either in vitro or in vivo, with the cell-targeting molecule of the present invention, which comprises the CD8+ T-cell epitope, and/or a composition thereof (e.g., a pharmaceutical or diagnostic composition of the present invention comprising such a cell-targeting molecule of the present invention). For certain further embodiments, the immune response is selected from the group consisting: CD8+ immune cell secretion of a cytokine(s), cytotoxic T lymphocyte- (CTL) induced growth arrest in the target cell, CTL-induced necrosis of the target cell, CTL-induced apoptosis of the target cell, immune cell-mediated cell killing of a cell other than the target cell.

Among certain embodiments of the present invention is a method of causing intercellular engagement of a CD8+ immune cell with a target cell, the method comprises the step of contacting the target cell with the cell-targeting molecule of the present invention in the presence of a CD8+ immune cell or with the subsequent step of contacting the target cell with one or more CD8+ immune cells. For certain embodiments, the contacting step occurs in vitro. For certain other embodiments, the contacting step occurs in vivo, such as, e.g., by administering the cell-targeting molecule to a chordate, vertebrate, and/or mammal. For certain embodiments, the intercellular engagement occurs in vitro. For certain embodiments, the intercellular engagement occurs in vivo.

Among certain embodiments of the present invention is a composition comprising a cell-targeting molecule of the present invention for “seeding” a tissue locus within a chordate.

For certain embodiments, a method of the present invention is for “seeding” a tissue locus within a chordate, the method comprising the step of: administering to the chordate a cell-targeting molecule of the present invention, a pharmaceutical composition of the present invention, and/or a diagnostic composition of the present invention. For certain further embodiments, the method is for “seeding” a tissue locus within a chordate which comprises a malignant, diseased, and/or inflamed tissue. For certain further embodiments, the method is for “seeding” a tissue locus within a chordate which comprises the tissue selected from the group consisting of: diseased tissue, tumor mass, cancerous growth, tumor, infected tissue, or abnormal cellular mass. For certain embodiments, the method for “seeding” a tissue locus within a chordate comprises the step of: administering to the chordate a cell-targeting molecule of the present invention comprising the heterologous, CD8+ T-cell epitope-peptide selected from the group consisting of: peptides not natively presented by the target cells of the cell-targeting molecule in MHC class I complexes, peptides not natively present within any protein expressed by the target cell, peptides not natively present within the transcriptome and/or proteome of the target cell, peptides not natively present in the extracellular microenvironment of the site to be seeded, and peptides not natively present in the tumor mass or infected tissue site to be targeted.

The present invention also provides pharmaceutical compositions comprising a cell-targeting molecule of the present invention and at least one pharmaceutically acceptable excipient or carrier; and the use of such a cell-targeting molecule, or a composition comprising it, in methods of the invention as further described herein. Certain embodiments of the present invention are pharmaceutical compositions comprising any cell-targeting molecule of the present invention; and at least one pharmaceutically acceptable excipient or carrier.

Among certain embodiments of the present invention is a diagnostic composition comprising a cell-targeting molecule of the present invention, or a composition thereof, and a detection promoting agent for the collection of information, such as diagnostically useful information about a cell-type, tissue, organ, disease, disorder, condition, and/or patient.

Beyond the cell-targeting molecules and compositions of the present invention, polynucleotides capable of encoding a cell-targeting molecule of the present invention, or a protein component thereof, are within the scope of the present invention, as well as expression vectors which comprise a polynucleotide of the invention and host cells comprising an expression vector of the invention. Host cells comprising an expression vector may be used, e.g., in methods for producing a cell-targeting molecule of the present invention, or a protein component or fragment thereof, by recombinant expression.

The present invention also encompasses any composition of matter of the present invention which is immobilized on a solid substrate. Such arrangements of the compositions of matter of the present invention may be utilized, e.g., in methods of screening molecules as described herein.

Additionally, the present invention provides methods of killing a cell(s) comprising the step of contacting a cell(s) with a cell-targeting molecule of the present invention or a pharmaceutical composition comprising a cell-targeting molecule of the invention. For certain embodiments, the step of contacting the cell(s) occurs in vitro. For certain other embodiments, the step of contacting the cell(s) occurs in vivo. For further embodiments of the cell-killing methods, the method is capable of selectively killing cell(s) and/or cell-types preferentially over other cell(s) and/or cell-types when contacting a mixture of cells which differ with respect to the extracellular presence and/or expression level of an extracellular target biomolecule of the binding region of the cell-targeting molecule.

The present invention further provides methods of treating diseases, disorders, and/or conditions in patients in need thereof comprising the step of administering to a patient in need thereof a therapeutically effective amount of a composition comprising a cell-targeting molecule or pharmaceutical composition of the present invention. For certain embodiments, the disease, disorder, or condition to be treated using this method of the invention is selected from: a cancer, tumor, growth abnormality, immune disorder, or microbial infection. For certain embodiments of this method, the cancer to be treated is selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. For certain embodiments of this method, the immune disorder to be treated is an immune disorder associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

Among certain embodiments of the present invention is a composition comprising a cell-targeting molecule of the present invention for the treatment or prevention of a cancer, tumor, growth abnormality, immune disorder, or microbial infection. Among certain embodiments of the present invention is the use of a composition of matter of the present invention in the manufacture of a medicament for the treatment or prevention of a cancer, tumor, growth abnormality, immune disorder, or microbial infection.

Among certain embodiments of the present invention is a composition comprising a cell-targeting molecule of the present invention for the delivery of one or more additional exogenous materials into a cell physically coupled with an extracellular target biomolecule of the binding region of the cell-targeting molecule of the present invention. Certain embodiments of the cell-targeting molecules of the present invention may be used to deliver one or more additional exogenous materials into a cell physically coupled with an extracellular target biomolecule of the binding region of the cell-targeting molecule of the present invention. Additionally, the present invention provides a method for delivering exogenous material to the inside of a cell(s) comprising contacting the cell(s), either in vitro or in vivo, with a cell-targeting molecule, pharmaceutical composition, and/or diagnostic composition of the present invention. The present invention further provides a method for delivering exogenous material to the inside of a cell(s) in a patient in need thereof, the method comprising the step of administering to the patient a cell-targeting molecule of the present invention, wherein the target cell(s) is physically coupled with an extracellular target biomolecule of the binding region of the cell-targeting molecule of the present invention.

The use of any composition of the present invention (e.g. a cell-targeting molecule, a pharmaceutical composition, or diagnostic composition) for the diagnosis, prognosis, and/or characterization of a disease, disorder, and/or condition is within the scope of the present invention.

Among certain embodiments of the present invention is the method of detecting a cell using a cell-targeting molecule and/or diagnostic composition of the invention comprising the steps of contacting a cell with said cell-targeting molecule and/or diagnostic composition and detecting the presence of said cell-targeting molecule and/or diagnostic composition. For certain embodiments, the step of contacting the cell(s) occurs in vitro. For certain embodiments, the step of contacting the cell(s) occurs in vivo. For certain embodiments, the step of detecting the cell(s) occurs in vitro. For certain embodiments, the step of detecting the cell(s) occurs in vivo.

For example, a diagnostic composition of the invention may be used to detect a cell in vivo by administering to a chordate subject a composition comprising cell-targeting molecule of the present invention which comprises a detection promoting agent and then detecting the presence of the cell-targeting molecule of the present invention and/or the heterologous, CD8+ T-cell epitope-peptide either in vitro or in vivo.

The use of any composition of the present invention for the treatment or prevention of a cancer, tumor, growth abnormality, and/or immune disorder is within the scope of the present invention.

Certain embodiments of the present invention include a method of treating cancer in a patient using immunotherapy, the method comprising the step of administering to the patient in need thereof the cell-targeting molecule and/or pharmaceutical composition of the present invention.

Among certain embodiments of the present invention are kits comprising a composition of matter of the present invention, and optionally, instructions for use, additional reagent(s), and/or pharmaceutical delivery device(s).

These and other features, aspects and advantages of the present invention will become better understood with regard to the following description, appended claims, and accompanying figures. The aforementioned elements of the invention may be individually combined or removed freely in order to make other embodiments of the invention, without any statement to object to such combination or removal hereinafter.

### BRIEF DESCRIPTION OF THE FIGURES

FIG. 1(A-B) shows the general arrangement of exemplary cell-targeting molecules of the present invention, each comprising a cell-targeting binding region (binding region), a Shiga toxin A Subunit effector polypeptide (Shiga toxin effector domain), and a heterologous, CD8+ T-cell epitope-peptide (epitope). The “N” and “C” labels denote an amino terminus and carboxy terminus, respectively, of Shiga toxin effector polypeptides. The depictions of exemplary molecules in FIG. 1 are for illustrative purposes of certain, general arrangements of the structural features of a limited set of embodiments of the present invention. It is to be understood that these exemplary molecules do not intend, nor should any be construed, to be wholly definitive as to the arrangement of any structural features and/or components of a molecule of the present invention. The relative size, location, or number of features shown in the schematics of FIG. 1 have been simplified. For example, the total numbers of heterologous, CD8+ T-cell epitope-peptide cargos per cell-targeting molecule may be greater than 2, 3, 4, 5, 10, 20, or 30, and a heterologous, CD8+ T-cell epitope peptide may be comprised within a larger antigenic molecule. The schematics in FIG. 1 are not intended to accurately portray any information regarding the relative sizes of molecular structures in any embodiment of the present invention.

FIG. 1B shows the general arrangement of exemplary cell-targeting molecules of the present invention which comprise a protease-cleavage resistant, Shiga toxin effector polypeptide (see e.g. WO 2015/191764) and wherein a heterologous, CD8+ T-cell epitope-peptide is associated with the cell-targeting molecule carboxy-terminal to the Shiga toxin effector polypeptide component.

FIG. 2 graphically shows fusing a heterologous, CD8+ T-cell epitope-peptide to a Shiga toxin A Subunit derived, cell-targeting molecule did not significantly impair the cytotoxic activity of the cell-targeting molecule toward target positive cells. The percent viability of cells was plotted over the logarithm to base 10 of the protein concentration. FIG. 2 graphically shows the results of a cell-kill assay where SLT-1A::scFv1::C2 (SEQ ID NO:61) exhibited cytotoxicity similar to the cytotoxicity of the parental cell-targeting molecule SLT-1A::scFv1 (SEQ ID NO:63), which lacked any heterologous, CD8+ T-cell epitope-peptide.

FIG. 3 graphically shows the results of a cell-kill assay where the cytotoxic activity of the exemplary cell-targeting molecule SLT-1A::scFv1::C2 (SEQ ID NO:61) was specific to target positive cells over a certain concentration range. The percent viability of cells was plotted over the logarithm to base 10 of the protein concentration. Cells negative for cell-surface expression of a target biomolecule of the binding region scFv2 were not killed (approximately 100% cell viability) by SLT-1A::scFv1::C2 (SEQ ID NO:61) over the molecule concentration range used to accurately measure the CD50 value of SLT-1A::scFv1::C2 (SEQ ID NO:61) toward target positive cells and as shown in FIG. 2.

FIG. 4 graphically shows cell-surface presentation of a cell-targeting molecule delivered, heterologous, CD8+ T-cell epitope-peptide complexed with MHC class I molecule by a target positive cancer cell as compared to a negative control. FIG. 4 shows overlays of the results of a TCR-STAR™ assay, flow cytometric analysis of sets of cells treated either with a negative control, the cell-targeting molecule SLT-1A::scFv1::C2 (SEQ ID NO:61), or the cell-targeting molecule SLT-1A::scFv2 (SEQ ID NO:64). The fluorescence-activated cell sorting (FACS) flow cytometry cell count of target positive cells was plotted over the light signal from PE-STAR™ multimer reagent in relative light units (RLU) representing the presence of cell-surface, MHC class I molecule (human HLA-A2) displayed C2 epitope-peptide (SEQ ID NO: 6) complexes. Target positive cells treated with the exemplary cell-targeting molecule of the present invention SLT-1A::scFv1::C2 (SEQ ID NO:61) displayed the C2 epitope-peptide (SEQ ID NO:6) complexed to MHC class I molecules on their cell surfaces (upper graph), whereas target positive cells treated with the related cell-targeting molecule SLT-1A::scFv2 (SEQ ID NO:64) did not display the C2 epitope-peptide (SEQ ID NO:6) on a cell surface (lower graph).

FIG. 5 graphically shows cell-surface presentation of a cell-targeting molecule delivered, heterologous, CD8+ T-cell epitope-peptide complexed with MHC class I molecule by a target positive cancer cell as compared to negative controls. In FIG. 5, the indexed, mean, fluorescent intensity (“iMFI,” the fluorescence of the positive population multiplied by the percent positive) of the PE-STAR™ multimer reagent in RLU corresponding to the sets of cells receiving the different treatments was graphed. FIG. 5 shows the results of a TCR-STAR Assay™, flow cytometric analysis of cells treated with either an exogenous C2 peptide (SEQ ID NO:6) control, “inactive SLT-1A::scFv” (SEQ ID NO:65), or the cell-targeting molecule “inactive SLT-1A::scFv2::C2” (SEQ ID NO:53). Exogenously administered C2 peptide ((SEQ ID NO: 6), as above) combined with a Peptide Loading Enhancer (“PLE,” Altor Biosicence Corp., Miramar, Fla., U.S.). The C2 peptide (SEQ ID NO: 6) combined with Peptide Loading Enhancer (PLE) treatment provides a positive control where exogenously administered C2 peptide (SEQ ID NO:6) may be loaded onto cell-surface MHC class I molecules without ever entering a cell. Target positive cells treated with the exemplary cell-targeting molecule of the present invention “inactive SLT-1A::scFv2::C2” (SEQ ID NO:53) displayed the C2 epitope-peptide (SEQ ID NO: 6) complexed to MHC class I molecules on their cell surfaces, whereas the same cells treated with only exogenous C2 epitope-peptide (SEQ ID NO: 6) or the parental cell-targeting molecule “inactive SLT-1A::scFv2” (SEQ ID NO:65) did not display the C2 epitope-peptide (SEQ ID NO: 6) on a cell surface.

FIG. 6 graphically shows cell-surface presentation of a cell-targeting molecule delivered, heterologous, CD8+ T-cell epitope-peptide complexed with MHC class I molecule by a target positive cancer cell for different incubation times (4 hours or 16 hours) as compared to a negative control. FIG. 6 shows overlays of the results of a TCR-STAR™ assay, flow cytometric analysis of sets of cells treated either with the cell-targeting molecule SLT-1A::scFv1::C2 (SEQ ID NO:61) or a negative control. The FACS cell count of target positive cells was plotted over the light signal from PE-STAR™ multimer reagent in relative light units (RLU) representing the presence of cell-surface, MHC class I molecule (human HLA-A2) displayed C2 epitope-peptide (SEQ ID NO:6) complexes. Target positive cells treated with the exemplary cell-targeting molecule of the present invention SLT-1A::scFv1::C2 (SEQ ID NO:61) displayed the C2 epitope-peptide (SEQ ID NO:6) complexed to MHC class I molecules on their cell surfaces after either a 4-hour (4 hrs) (upper graph) or 16-hour (16 hrs) (lower graph) incubation duration.

FIG. 7 graphically shows cell-surface presentation of a cell-targeting molecule delivered, heterologous, CD8+ T-cell epitope-peptide complexed with MHC class I molecule by a target positive cancer cell as compared to a negative control. FIG. 7 shows overlays of the results of a TCR-STAR™ assay, flow cytometric analysis of sets of cells treated either with a negative control, the cell-targeting molecule SLT-1A::scFv5::C2 (SEQ ID NO:57), or the cell-targeting molecule SLT-1A::scFv5 (SEQ ID NO:66). The FACS cell count of target positive cells was plotted over the light signal from PE-STAR™ multimer reagent in relative light units (RLU) representing the presence of cell-surface, MHC class I molecule (human HLA-A2) displayed C2 epitope-peptide (SEQ ID NO:6) complexes. Target positive cells treated with the exemplary cell-targeting molecule of the present invention SLT-1A::scFv5::C2 (SEQ ID NO:57) displayed the C2 epitope-peptide (SEQ ID NO:6) complexed to MHC class I molecules on their cell surfaces (upper graph), whereas target positive cells treated with the parental cell-targeting molecule SLT-1A::scFv5 (SEQ ID NO:66) did not display the C2 epitope-peptide (SEQ ID NO: 6) on a cell surface (lower graph).

FIG. 8 graphically shows cell-surface presentation of a cell-targeting molecule delivered, heterologous, CD8+ T-cell epitope-peptide complexed with MHC class I molecule by a target positive cancer cell as compared to a negative control. FIG. 7 shows overlays of the results of a TCR-STAR™ assay, flow cytometric analysis of sets of cells treated either with a negative control, the cell-targeting molecule SLT-1A::scFv7::C2 (SEQ ID NO:60), or the cell-targeting molecule SLT-1A::scFv7 (SEQ ID NO:69). The FACS cell count of target positive cells was plotted over the light signal from PE-STAR™ multimer reagent in relative light units (RLU) representing the presence of cell-surface, MHC class I molecule (human HLA-A2) displayed C2 epitope-peptide (SEQ ID NO:6) complexes. Target positive cells treated with the exemplary cell-targeting molecule of the present invention SLT-1A::scFv7::C2 (SEQ ID NO:60) displayed the C2 epitope-peptide (SEQ ID NO:6) complexed to MHC class I molecules on their cell surfaces (upper graph), whereas target positive cells treated with the parental cell-targeting molecule SLT-1A::scFv7 (SEQ ID NO:69) did not display the C2 epitope-peptide (SEQ ID NO: 6) on a cell surface (lower graph).

FIG. 9 graphically shows the results from an Interferon Gamma ELIspot assay with the number of spots, or secreting cells, plotted for each condition tested. For each sample, target positive cancer cells were treated with a cell-targeting molecule or negative control and, then, incubated with human PBMCs before performing the ELISPOT assay. Target positive cells treated with the exemplary cell-targeting molecule of the present invention “inactive SLT-1A::scFv2::C2” (SEQ ID NO:53) stimulated an intercellular response in the form of cytokine secretion by immune cells, whereas the results from target positive cells treated with the parental cell-targeting molecule “inactive SLT-1A::scFv2” (SEQ ID NO:65) presumably showed the background level of intercellular engagement of the PBMCs resulting in interferon-y secretion, which was about the same as the negative control treatment of “buffer only.”

FIG. 10 graphically shows the results from an intercellular T lymphocyte (T-cell) activation assay with luciferase activity plotted in RLU for each condition tested. For each sample, target positive cancer cells were treated with a cell-targeting molecule or negative control and, then, incubated with human T-cells expressing a human T-cell receptor (TCR) that specifically recognizes cell-surface presented, human MHC class I molecule (HLA-A2) F2 epitope (SEQ ID NO: 10) complexes, and comprising an NFAT transcriptional response element driving luciferase expression. Target positive cells treated with the exemplary cell-targeting molecule of the present invention “inactive SLT-1A::scFv6::F2” (SEQ ID NO:59) stimulated an intermolecular response in the form of T-cell activation via TCR recognition and NFAT signaling; whereas, the results from target positive cells treated with the parental cell-targeting molecule “inactive SLT-1A::scFv6” (SEQ ID NO: 68) presumably showed the background level of intermolecular T-cell signaling activation by NFAT, which was about the same as the negative control treatment of “buffer only.”

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art. In order that the present invention may be more readily understood, certain terms are defined below. Additional definitions may be found within the detailed description of the invention.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout this specification, the term “including” is used to mean “including but not limited to.” “Including” and “including but not limited to” are used interchangeably.

The term “amino acid residue” or “amino acid” includes reference to an amino acid that is incorporated into a protein, polypeptide, and/or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide of sizes less than about a total of 15 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino terminus to a carboxy terminus.

The terms “amino acid,” “amino acid residue,” “amino acid sequence,” or polypeptide sequence include naturally occurring amino acids (including L and D isosteriomers) and, unless otherwise limited, also include known analogs of natural amino acids that can function in a similar manner as naturally occurring amino acids, such as selenocysteine, pyrrolysine, N-formylmethionine, gamma-carboxyglutamate, hydroxyprolinehypusine, pyroglutamic acid, and selenomethionine {see e.g. Nagata K et al., Bioinformatics 30: 1681-9 (2014)). The amino acids referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The phrase “conservative substitution” with regard to an amino acid residue of a peptide, peptide region, polypeptide region, protein, or molecule refers to a change in the amino acid composition of the peptide, peptide region, polypeptide region, protein, or molecule that does not substantially alter the function and structure of the overall peptide, peptide region, polypeptide region, protein, or molecule {see Creighton, Proteins: Structures and Molecular Properties (W. H. Freeman and Company, New York (2nd ed., 1992))).

For purposes of the present invention, the phrase “derived from” when referring to a polypeptide or polypeptide region means that the polypeptide or polypeptide region comprises amino acid sequences originally found in a “parental” protein and which may now comprise certain amino acid residue additions, deletions, truncations, rearrangements, or other alterations relative to the original polypeptide or polypeptide region as long as a certain function(s) and a structure(s) of the “parental” molecule are substantially conserved. The skilled worker will be able to identify a parental molecule from which a polypeptide or polypeptide region was derived using techniques known in the art, e.g., protein sequence alignment software.

For purposes of the claimed invention and with regard to a Shiga toxin polypeptide sequence or Shiga toxin derived polypeptide, the term “wild-type” generally refers to a naturally occurring, Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

The terms “associated,” “associating,” “linked,” or “linking” with regard to the claimed invention refers to the state of two or more components of a molecule being joined, attached, connected, or otherwise coupled to form a single molecule or the act of making two molecules associated with each other to form a single molecule by creating an association, linkage, attachment, and/or any other connection between the two molecules. For example, the term “linked” may refer to two or more components associated by one or more atomic interactions such that a single molecule is formed and wherein the atomic interactions may be covalent and/or non-covalent. Non-limiting examples of covalent associations between two components include peptide bonds and cysteine-cysteine disulfide bonds. Non-limiting examples of non-covalent associations between two molecular components include ionic bonds.

For purposes of the present invention, the term “linked” refer to two or more molecular components associated by one or more atomic interactions such that a single molecule is formed and wherein the atomic interactions include at least one covalent bond. For purposes of the present invention, the term “linking” refers to the act of creating a linked molecule as described above.

For purposes of the present invention, the term “fused” refers to two or more proteinaceous components associated by at least one covalent bond which is a peptide bond, regardless of whether the peptide bond involves the participation of a carbon atom of a carboxyl acid group or involves another carbon atom, such as, e.g., the α-carbon, β-carbon, γ-carbon, σ-carbon, etc. Non-limiting examples of two proteinaceous components fused together include, e.g., an amino acid, peptide, or polypeptide fused to a polypeptide via a peptide bond such that the resulting molecule is a single, continuous polypeptide. For purposes of the present invention, the term “fusing” refers to the act of creating a fused molecule as described above, such as, e.g., a fusion protein generated from the recombinant fusion of genetic regions which when translated produces a single proteinaceous molecule.

The symbol “::” means the polypeptide regions before and after it are physically linked together to form a continuous polypeptide.

As used herein, the terms “expressed,” “expressing,” or “expresses,” and grammatical variants thereof, refer to translation of a polynucleotide or nucleic acid into a protein. The expressed protein may remain intracellular, become a component of the cell surface membrane or be secreted into an extracellular space.

As used herein, cells which express a significant amount of an extracellular target biomolecule at least one cellular surface are “target positive cells” or “target+ cells” and are cells physically coupled to the specified, extracellular target biomolecule.

As used herein, the symbol “a” is shorthand for an immunoglobulin-type binding region capable of binding to the biomolecule following the symbol. The symbol “a” is used to refer to the functional characteristic of an immunoglobulin-type binding region based on its ability to bind to the biomolecule following the symbol with a binding affinity described by a dissociation constant (KID) of 10⁻⁵ or less.

As used herein, the term “heavy chain variable (VH) domain” or “light chain variable (VL) domain” respectively refer to any antibody VH or VL domain (e.g. a human VH or VL domain) as well as any derivative thereof retaining at least qualitative antigen binding ability of the corresponding native antibody (e.g. a humanized VH or VL domain derived from a native murine VH or VL domain). A VH or VL domain consists of a “framework” region interrupted by the three CDRs or ABRs. The framework regions serve to align the CDRs or ABRs for specific binding to an epitope of an antigen. From amino terminus to carboxy terminus, both VH and VL domains comprise the following framework (FR) and CDR regions or ABR regions: FR1, CDR1, FR2, CDR2, FR3, CDR3, and FR4; or, similarly, FR1, ABR1, FR2, ABR2, FR3, ABR3, and FR4. As used herein, the terms “HCDR1,” “HCDR2,” or “HCDR3” are used to refer to CDRs 1, 2, or 3, respectively, in a V H domain, and the terms “LCDR1,” “LCDR2,” and “LCDR3” are used to refer to CDRs 1, 2, or 3, respectively, in a VL domain. As used herein, the terms “HABR1,” “HABR2,” or “HABR3” are used to refer to ABRs 1, 2, or 3, respectively, in a V H domain, and the terms “LABR1,” “LABR2,” or “LABR3” are used to refer to CDRs 1, 2, or 3, respectively, in a VL domain. For camelid VHH fragments, IgNARs of cartilaginous fish, VNAR fragments, certain single domain antibodies, and derivatives thereof, there is a single, heavy chain variable domain comprising the same basic arrangement: FR1, CDR1, FR2, CDR2, FR3, CDR3, and FR4. As used herein, the terms “HCDR1,” “HCDR2,” or “HCDR3” may be used to refer to CDRs 1, 2, or 3, respectively, in a single heavy chain variable domain.

For purposes of the present invention, the term “effector” means providing a biological activity, such as cytotoxicity, biological signaling, enzymatic catalysis, subcellular routing, and/or intermolecular binding resulting in an allosteric effect(s) and/or the recruitment of one or more factors.

For purposes of the present invention, the phrases “Shiga toxin effector polypeptide,” “Shiga toxin effector polypeptide region,” and “Shiga toxin effector region” refer to a polypeptide or polypeptide region derived from at least one Shiga toxin A Subunit of a member of the Shiga toxin family wherein the polypeptide or polypeptide region is capable of exhibiting at least one Shiga toxin function.

For purposes of the present invention, the term “heterologous” as describing a binding region means the binding region is from a different source than a naturally occurring Shiga toxin, e.g. a heterologous binding region which is a polypeptide is polypeptide not naturally found as part of any native Shiga toxin.

For purposes of the present invention, the term “heterologous” as describing a CD8+ T-cell epitope means the CD8+ T-cell epitope is from a different source than (1) an A Subunit of a naturally occurring Shiga toxin, e.g. a heterologous polypeptide is not naturally found as part of any A Subunit of a native Shiga toxin and (2) a prior art Shiga toxin effector polypeptide. For example, in certain embodiments of the cell-targeting molecules of the present invention, the term “heterologous” with regard to a CD8+ T-cell epitope-peptide refers to a peptide sequence which did not initially occur in a cell-targeting molecule to be modified (parental molecule), but which was added to the molecule, whether added via the processes of embedding, fusion, insertion, and/or amino acid substitution as described herein, or by any other engineering means to create a modified cell-targeting molecule. The result is a modified cell-targeting molecule comprising a CD8+ T-cell epitope-peptide which is foreign to the original, unmodified cell-targeting molecule, i.e. the CD8+ T-cell epitope was not present in the unmodified cell-targeting molecule (parental molecule).

In certain embodiments of the cell-targeting molecule of the present invention, the heterologous, CD8+ T-cell epitope is also heterologous to the binding region component(s) of the cell-targeting molecule, e.g. a heterologous epitope is one that is not required for the binding activity of the binding region and is not part of the structure of the minimum binding region structure which provides the binding activity of the binding region. For example, a CD8+ T-cell epitope not natively present in an immunoglobulin is heterologous to an immunoglobulin-type binding region derived from that immunoglobulin if it is not required for the binding activity of the immunoglobulin-type binding region and is not part of the structure of the minimum binding region structure which provides the binding activity of the immunoglobulin-type binding region.

For purposes of the claimed invention, the phrase “intercellular engagement” by a CD8+ immune cell refers to a CD8+ immune cell responding to different cell (for example, by sensing the other is displaying one or more pMHC Is) in fashion indicative of the activation of an immune response by the CD8+ immune cell, such as, e.g., responses involved in killing the other cell, recruiting and activating other immune cells {e.g. cytokine secretion), maturation of the CD8+ immune cell, activation of the CD8+ immune cell, etc.

As used herein, the term “CD8+ T-cell epitope delivering” when describing a functional activity of a molecule means that a molecule provides the biological activity of localizing within a cell to a subcellular compartment that is competent to result in the proteasomal cleavage of a proteinaceous part of the molecule which comprises a CD8+ T-cell epitope-peptide. The “CD8+ T-cell epitope delivering” function of a molecule can be assayed by observing the MHC presentation of a CD8+ T-cell epitope-peptide cargo of the molecule on a cell surface of a cell exogenously administered the molecule or in which the assay was begun with the cell containing the molecule in one or more of its endosomal compartments.

Generally, the ability of a molecule to deliver a CD8+ T-cell epitope to a proteasome can be determined where the initial location of the “CD8+ T-cell epitope delivering” molecule is an early endosomal compartment of a cell, and then, the molecule is empirically shown to deliver the epitope-peptide to the proteasome of the cell. However, a “CD8+ T-cell epitope delivering” ability may also be determined where the molecule starts at an extracellular location and is empirically shown, either directly or indirectly, to deliver the epitope into a cell and to proteasomes of the cell. For example, certain “CD8+ T-cell epitope delivering” molecules pass through an endosomal compartment of the cell, such as, e.g. after endocytotic entry into that cell. Alternatively, “CD8+ T-cell epitope delivering” activity may be observed for a molecule starting at an extracellular location whereby the molecule does not enter any endosomal compartment of a cell—instead the “CD8+ T-cell epitope delivering” molecule enters a cell and delivers a T-cell epitope-peptide to proteasomes of the cell, presumably because the “CD8+ T-cell epitope delivering” molecule directed its own routing to a subcellular compartment competent to result in proteasomal cleavage of its CD8+ T-cell epitope-peptide component.

For purposes of the present invention, a Shiga toxin effector function is a biological activity conferred by a polypeptide region derived from a Shiga toxin A Subunit. Non-limiting examples of Shiga toxin effector functions include promoting cell entry; lipid membrane deformation; promoting cellular internalization; stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activation, activating effector caspases, effectuating cytostatic effects, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. Shiga toxins are ribosome inactivating proteins (RIPs). RIPs can depurinate nucleic acids, polynucleosides, polynucleotides, rRNA, ssDNA, dsDNA, mRNA (and polyA), and viral nucleic acids (see e.g., Barbieri L et al., Biochem J 286: 1-4 (1992); Barbieri L et al., Nature 372: 624 (1994); Ling J et al., FEBS Lett 345: 143-6 (1994); Barbieri L et al., Biochem J 319: 507-13 (1996); Roncuzzi L, Gasperi-Campani A, FEBS Lett 392: 16-20 (1996); Stirpe F et al., FEBS Lett 382: 309-12 (1996); Barbieri L et al., Nucleic Acids Res 25: 518-22 (1997); Wang P, Turner N, Nucleic Acids Res 27: 1900-5 (1999); Barbieri L et al., Biochim Biophys Acta 1480: 258-66 (2000); Barbieri L et al., J Biochem 128: 883-9 (2000); Brigotti M et al., Toxicon 39: 341-8 (2001); Brigotti M et al., FASEB J 16: 365-72 (2002); Bagga S et al., J Biol Chem 278: 4813-20 (2003); Picard D et al., J Biol Chem 280: 20069-75 (2005)). Some RIPs show antiviral activity and superoxide dismutase activity (Erice A et al., Antimicrob Agents Chemother 37: 835-8 (1993); Au T et al., FEBS Lett 471: 169-72 (2000); Parikh B, Turner N, Mini Rev Med Chem 4: 523-43 (2004); Sharma N et al., Plant Physiol 134: 171-81 (2004)). Shiga toxin catalytic activities have been observed both in vitro and in vivo. Non-limiting examples of assays for Shiga toxin effector activity measure various activities, such as, e.g., protein synthesis inhibitory activity, depurination activity, inhibition of cell growth, cytotoxicity, supercoiled DNA relaxation activity, and nuclease activity.

As used herein, the retention of Shiga toxin effector function refers to being capable of exhibiting a level of Shiga toxin functional activity, as measured by an appropriate quantitative assay with reproducibility, comparable to a wild-type, Shiga toxin effector polypeptide control {e.g. a Shiga toxin A1 fragment) or cell-targeting molecule comprising a wild-type Shiga toxin effector polypeptide {e.g. a Shiga toxin A1 fragment) under the same conditions. For the Shiga toxin effector function of ribosome inactivation or ribosome inhibition, retained Shiga toxin effector function is exhibiting an ICso of 10,000 picomolar (pM) or less in an in vitro setting, such as, e.g., by using an assay known to the skilled worker and/or described herein. For the Shiga toxin effector function of cytotoxicity in a target positive cell-kill assay, retained Shiga toxin effector function is exhibiting a CD50 of 1,000 nanomolar (nM) or less, depending on the cell-type and its expression of the appropriate extracellular target biomolecule, as shown, e.g., by using an assay known to the skilled worker and/or described herein.

For purposes of the claimed invention, the term “equivalent” with regard to ribosome inhibition means an empirically measured level of ribosome inhibitory activity, as measured by an appropriate quantitative assay with reproducibility, which is reproducibly within 10% or less of the activity of the reference molecule (e.g., the second cell-targeting molecule or third cell-targeting molecule) under the same conditions.

For purposes of the claimed invention, the term “equivalent” with regard to cytotoxicity means an empirically measured level of cytotoxicity, as measured by an appropriate quantitative assay with reproducibility, which is reproducibly within 10% or less of the activity of the reference molecule (e.g., the second cell-targeting molecule or third cell-targeting molecule) under the same conditions.

A s used herein, the term “attenuated” with regard to cytotoxicity means a molecule exhibits or exhibited a CD50 between 10-fold to 100-fold of a CD50 exhibited by a reference molecule under the same conditions.

Inaccurate IC₅₀ and CD50 values should not be considered when determining a level of Shiga toxin effector function activity. For some samples, accurate values for either ICso or CD50 might b e unobtainable due to the inability to collect the required data points for an accurate curve fit. For example, theoretically, neither an IC50 nor CD50 can b e determined if greater than 50% ribosome inhibition or cell death, respectively, does not occur in a concentration series for a given sample. Data insufficient to accurately fit a curve as described in the analysis of the data from exemplary Shiga toxin effector function assays, such as, e.g., assays described in the Examples below, should not be considered as representative of actual Shiga toxin effector function.

A failure to detect activity in Shiga toxin effector function may be due to improper expression, polypeptide folding, and/or protein stability rather than a lack of cell entry, subcellular routing, and/or enzymatic activity. Assays for Shiga toxin effector functions may not require much polypeptide of the invention to measure significant amounts of Shiga toxin effector function activity. To the extent that an underlying cause of low or no effector function is determined empirically to relate to protein expression or stability, one of skill in the art may be able to compensate for such factors using protein chemistry and molecular engineering techniques known in the art, such that a Shiga toxin functional effector activity may be restored and measured. As examples, improper cell-based expression may be compensated for by using different expression control sequences; and improper polypeptide folding and/or stability may benefit from stabilizing terminal sequences, or compensatory mutations in non-effector regions which stabilize the three-dimensional structure of the molecule.

Certain Shiga toxin effector functions are not easily measurable, e.g. subcellular routing functions. For example, there is no routine, quantitative assay to distinguish whether the failure of a Shiga toxin effector polypeptide to be cytotoxic and/or deliver a heterologous, CD8+ T-cell epitope is due to improper subcellular routing, but at a time when tests are available, then Shiga toxin effector polypeptides may be analyzed for any significant level of subcellular routing as compared to the appropriate wild-type Shiga toxin effector polypeptide. However, if a Shiga toxin effector polypeptide component of a cell-targeting molecule of the present invention exhibits cytotoxicity comparable or equivalent to a wild-type Shiga toxin A Subunit construct, then the subcellular routing activity level is inferred to be comparable or equivalent, respectively, to the subcellular routing activity level of a wild-type Shiga toxin A Subunit construct at least under the conditions tested.

When new assays for individual Shiga toxin functions become available, Shiga toxin effector polypeptides and/or cell-targeting molecules comprising Shiga toxin effector polypeptides may be analyzed for any level of those Shiga toxin effector functions, such as a being within 1000-fold or 100-fold or less the activity of a wild-type Shiga toxin effector polypeptide or exhibiting 3-fold to 30-fold or greater activity as compared to a functional knockout, Shiga toxin effector polypeptide.

Sufficient subcellular routing may be merely deduced by observing a cell-targeting molecule's Shiga toxin cytotoxic activity levels in cytotoxicity assays, such as, e.g., cytotoxicity assays based on T-cell epitope presentation or based on a Shiga toxin effector function involving a cytosolic and/or endoplasmic reticulum-localized, target substrate.

A s used herein, the retention of “significant” Shiga toxin effector function refers to a level of Shiga toxin functional activity, as measured by an appropriate quantitative assay with reproducibility comparable to a wild-type Shiga toxin effector polypeptide control (e.g. a Shiga toxin A1 fragment). For in vitro ribosome inhibition, significant Shiga toxin effector function is exhibiting an ICso of 300 pM or less depending on the source of the ribosomes used in the assay (e.g. a bacterial, archaeal, or eukaryotic (algal, fungal, plant, or animal) source). This is significantly greater inhibition as compared to the approximate IC₅₀ of 100,000 pM for the catalytically disrupted SLT-1A 1-251 double mutant (Y77S/E167D). For cytotoxicity in a target-positive cell-kill assay in laboratory cell culture, significant Shiga toxin effector function is exhibiting a CD50 of 100, 50, 30 nM, or less, depending on the target biomolecule(s) of the binding region and the cell-type, particularly that cell-type's expression and/or cell-surface representation of the appropriate extracellular target biomolecule(s) and/or the extracellular epitope(s) targeted by the molecule being evaluated. This is significantly greater cytotoxicity to the appropriate, target-positive cell population as compared to a Shiga toxin A Subunit alone (or a wild-type Shiga toxin A 1 fragment), without a cell targeting binding region, which has a CD50 of 100-10,000 nM, depending on the cell line.

For purposes of the present invention and with regard to the Shiga toxin effector function of a molecule of the present invention, the term “reasonable activity” refers to exhibiting at least a moderate level (e.g. within 11-fold to 1,000-fold) of Shiga toxin effector activity as defined herein in relation to a molecule comprising a naturally occurring Shiga toxin, wherein the Shiga toxin effector activity is selected from the group consisting of: internalization efficiency, subcellular routing efficiency to the cytosol, delivered epitope presentation by a target cell(s), ribosome inhibition, and cytotoxicity. For cytotoxicity, a reasonable level of Shiga toxin effector activity includes being within 1,000-fold of a wild-type, Shiga toxin construct, such as, e.g., exhibiting a CD50 of 500 nM or less when a wild-type Shiga toxin construct exhibits a CD50 of 0.5 nM (e.g. a cell-targeting molecule comprising a wild-type Shiga toxin A 1 fragment).

For purposes of the present invention and with regard to the cytotoxicity of a molecule of the present invention, the term “optimal” refers to a level of Shiga toxin catalytic domain mediated cytotoxicity that is within 2, 3, 4, 5, 6, 7, 8, 9, or 10-fold of the cytotoxicity of a molecule comprising wild-type Shiga toxin A 1 fragment (e.g. a Shiga toxin A Subunit or certain truncated variants thereof) and/or a naturally occurring Shiga toxin.

It should be noted that even if the cytotoxicity of a Shiga toxin effector polypeptide is reduced relative to a wild-type Shiga toxin A Subunit or fragment thereof, in practice, applications using biological activity-attenuated, Shiga toxin effector polypeptides may be equally or more effective than using wild-type Shiga toxin effector polypeptides because the highest potency variants might exhibit undesirable effects which are minimized or reduced in reduced cytotoxic-potency variants. Wild-type Shiga toxins are very potent, being able to kill an intoxicated cell after only one toxin molecule has reached the cytosol of the intoxicated cell or perhaps after only forty toxin molecules have been internalized into the intoxicated cell. Shiga toxin effector polypeptides with even considerably reduced Shiga toxin effector functions, such as, e.g., subcellular routing or cytotoxicity, as compared to wild-type Shiga toxin effector polypeptides may still be potent enough for practical applications, such as, e.g., applications involving targeted cell-killing, heterologous epitope delivery, and/or detection of specific cells and their subcellular compartments. In addition, certain reduced-activity Shiga toxin effector polypeptides may be particularly useful for delivering cargos (e.g. an additional exogenous material or T-cell epitope) to certain intracellular locations or subcellular compartments of target cells.

The term “selective cytotoxicity” with regard to the cytotoxic activity of a molecule refers to the relative level of cytotoxicity between a biomolecule target positive cell population (e.g. a targeted cell-type) and a non-targeted bystander cell population (e.g. a biomolecule target negative cell-type), which can be expressed as a ratio of the half-maximal cytotoxic concentration (CD50) for a targeted cell-type over the CD50 for an untargeted cell-type to provide a metric of cytotoxic selectivity or indication of the preferentiality of killing of a targeted cell versus an untargeted cell.

The cell surface representation and/or density of a given extracellular target biomolecule (or extracellular epitope of a given target biomolecule) may influence the applications for which certain cell-targeting molecules of the present invention may be most suitably used. Differences in cell surface representation and/or density of a given target biomolecule between cells may alter, both quantitatively and qualitatively, the efficiency of cellular internalization and/or cytotoxicity potency of a given cell-targeting molecule of the present invention. The cell surface representation and/or density of a given target biomolecule can vary greatly among target biomolecule positive cells or even on the same cell at different points in the cell cycle or cell differentiation. The total cell surface representation of a given target biomolecule and/or of certain extracellular epitopes of a given target biomolecule on a particular cell or population of cells may be determined using methods known to the skilled worker, such as methods involving fluorescence-activated cell sorting (FACS) flow cytometry.

As used herein, the terms “disrupted,” “disruption,” or “disrupting,” and grammatical variants thereof, with regard to a polypeptide region or feature within a polypeptide refers to an alteration of at least one amino acid within the region or composing the disrupted feature. Amino acid alterations include various mutations, such as, e.g., a deletion, inversion, insertion, or substitution which alter the amino acid sequence of the polypeptide. Amino acid alterations also include chemical changes, such as, e.g., the alteration one or more atoms in an amino acid functional group or the addition of one or more atoms to an amino acid functional group.

As used herein, “de-immunized” means reduced antigenic and/or immunogenic potential after administration to a chordate as compared to a reference molecule, such as, e.g., a wild-type peptide region, polypeptide region, or polypeptide. This includes a reduction in overall antigenic and/or immunogenic potential despite the introduction of one or more, de novo, antigenic and/or immunogenic epitopes as compared to a reference molecule. For certain embodiments, “de-immunized” means a molecule exhibited reduced antigenicity and/or immunogenicity after administration to a mammal as compared to a “parental” molecule from which it was derived, such as, e.g., a wild-type Shiga toxin A1 fragment. In certain embodiments, the de-immunized, Shiga toxin effector polypeptide of the present invention is capable of exhibiting a relative antigenicity compared to a reference molecule which is reduced by 10%, 20%, 30%>, 40%, 50%, 60%, 70%, 80%, 90%), or greater than the antigenicity of the reference molecule under the same conditions measured by the same assay, such as, e.g., an assay known to the skilled worker and/or described herein like a quantitative ELISA or Western blot analysis. In certain embodiments, the de-immunized, Shiga toxin effector polypeptide of the present invention is capable of exhibiting a relative immunogenicity compared to a reference molecule which is reduced by 10%>, 20%, 30%, 40%, 50%, 60%, 70%, 80%, 90%, 95%, 97%, 99%, or greater than the immunogenicity of the reference molecule under the same conditions measured by the same assay, such as, e.g., an assay known to the skilled worker and/or described herein like a quantitative measurement of anti-molecule antibodies produced in a mammal(s) after receiving parenteral administration of the molecule at a given time-point.

The relative immunogenicities of exemplary cell-targeting molecules were determined using an assay for in vivo antibody responses to the cell-targeting molecules after repeat, parenteral administrations over periods of many.

For purposes of the present invention, the phrase “CD8+ T-cell hyper-immunized” means that the cell-targeting molecule, when present inside a nucleated, chordate cell within a living chordate, has an increased antigenic and/or immunogenic potential regarding CD8+ T-cell antigenicity or immunogenicity when compared to the same molecule that lacks any heterologous, CD8+ T-cell epitope-peptide.

The term “embedded” and grammatical variants thereof with regard to a T-cell epitope or T-cell epitope-peptide component of a polypeptide refers to the internal replacement of one or more amino acids within a polypeptide region with different amino acids in order to generate a new polypeptide sequence sharing the same total number of amino acid residues with the starting polypeptide region. Thus, the term “embedded” does not include any external, terminal fusion of any additional amino acid, peptide, or polypeptide component to the starting polypeptide nor any additional internal insertion of any additional amino acid residues, but rather includes only substitutions for existing amino acids. The internal replacement may be accomplished merely by amino acid residue substitution or by a series of substitutions, deletions, insertions, and/or inversions. If an insertion of one or more amino acids is used, then the equivalent number of proximal amino acids must be deleted next to the insertion to result in an embedded T-cell epitope. This is in contrast to use of the term “inserted” with regard to a T-cell epitope contained within a polypeptide of the present invention to refer to the insertion of one or more amino acids internally within a polypeptide resulting in a new polypeptide having an increased number of amino acids residues compared to the starting polypeptide.

The term “inserted” and grammatical variants thereof with regard to a T-cell epitope contained within a polypeptide refers to the insertion of one or more amino acids within a polypeptide resulting in a new polypeptide sequence having an increased number of amino acids residues compared to the starting polypeptide.

For purposes of the present invention, the phrase “proximal to an amino terminus” with reference to the position of a Shiga toxin effector polypeptide region of a cell-targeting molecule of the present invention refers to a distance wherein at least one amino acid residue of the Shiga toxin effector polypeptide region is within 1, 2, 3, 4, 5, 6, 7, 8, 9, 10, 11, 12 or more, e.g., up to 18-20 amino acid residues, of an amino terminus of the cell-targeting molecule as long as the cell-targeting molecule is capable of exhibiting the appropriate level of Shiga toxin effector functional activity noted herein (e.g., a certain level of cytotoxic potency). Thus for certain embodiments of the present invention, any amino acid residue(s) fused amino-terminal to the Shiga toxin effector polypeptide should not reduce any Shiga toxin effector function (e.g., by sterically hindering a structure(s) near the amino terminus of the Shiga toxin effector polypeptide region) such that a functional activity of the Shiga toxin effector polypeptide is reduced below the appropriate activity level required herein.

For purposes of the present invention, the phrase “more proximal to an amino terminus” with reference to the position of a Shiga toxin effector polypeptide region within a cell-targeting molecule of the present invention as compared to another component (e.g., a cell-targeting, binding region, molecular moiety, and/or additional exogenous material) refers to a position wherein at least one amino acid residue of the amino terminus of the Shiga toxin effector polypeptide is closer to the amino terminus of a linear, polypeptide component of the cell-targeting molecule of the present invention as compared to the other referenced component.

For purposes of the present invention, the phrase “active enzymatic domain derived from one A Subunit of a member of the Shiga toxin family” refers to having the ability to inhibit protein synthesis via a catalytic ribosome inactivation mechanism. The enzymatic activities of naturally occurring Shiga toxins may be defined by the ability to inhibit protein translation using assays known to the skilled worker, such as, e.g., in vitro assays involving RNA translation in the absence of living cells or in vivo assays involving RNA translation in a living cell. Using assays known to the skilled worker and/or described herein, the potency of a Shiga toxin enzymatic activity may be assessed directly by observing N-glycosidase activity toward ribosomal RNA (rRNA), such as, e.g., a ribosome nicking assay, and/or indirectly by observing inhibition of ribosome function and/or protein synthesis.

For purposes of the present invention, the term “Shiga toxin A1 fragment region” refers to a polypeptide region consisting essentially of a Shiga toxin A1 fragment and/or derived from a Shiga toxin A1 fragment of a Shiga toxin.

For purposes of the present invention, the terms “terminus,” “amino terminus,” or “carboxy terminus” with regard to a cell-targeting molecule refers generally to the last amino acid residue of a polypeptide chain of the cell-targeting molecule (e.g., a single, continuous polypeptide chain). A cell-targeting molecule may comprise more than one polypeptides or proteins, and, thus, a cell-targeting molecule of the present invention may comprise multiple amino-terminals and carboxy-terminals. For example, the “amino terminus” of a cell-targeting molecule may be defined by the first amino acid residue of a polypeptide chain representing the amino-terminal end of the polypeptide, which is generally characterized by a starting, amino acid residue which does not have a peptide bond with any amino acid residue involving the primary amino group of the starting amino acid residue or involving the equivalent nitrogen for starting amino acid residues which are members of the class of N-alkylated alpha amino acid residues. Similarly, the “carboxy terminus” of a cell-targeting molecule may be defined by the last amino acid residue of a polypeptide chain representing the carboxyl-terminal end of the polypeptide, which is generally characterized by a final, amino acid residue which does not have any amino acid residue linked by a peptide bond to the alpha-carbon of its primary carboxyl group.

For purposes of the present invention, the terms “terminus,” “amino terminus,” or “carboxy terminus” with regard to a polypeptide region refers to the regional boundaries of that region, regardless of whether additional amino acid residues are linked by peptide bonds outside of that region. In other words, the terminals of the polypeptide region regardless of whether that region is fused to other peptides or polypeptides. For example, a fusion protein comprising two proteinaceous regions, e.g., a binding region comprising a peptide or polypeptide and a Shiga toxin effector polypeptide, may have a Shiga toxin effector polypeptide region with a carboxy terminus ending at amino acid residue 251 of the Shiga toxin effector polypeptide region despite a peptide bond involving residue 251 to an amino acid residue at position 252 representing the beginning of another proteinaceous region, e.g., the binding region. In this example, the carboxy terminus of the Shiga toxin effector polypeptide region refers to residue 251, which is not a terminus of the fusion protein but rather represents an internal, regional boundary. Thus, for polypeptide regions, the terms “terminus,” “amino terminus,” and “carboxy terminus” are used to refer to the boundaries of polypeptide regions, whether the boundary is a physically terminus or an internal, position embedded within a larger polypeptide chain.

For purposes of the present invention, the phrase “Shiga toxin A1 fragment derived region” refers to all or part of a Shiga toxin effector polypeptide wherein the region consists of a polypeptide homologous to a naturally occurring Shiga toxin A1 fragment or truncation thereof, such as, e.g., a polypeptide consisting of or comprising amino acids 75-239 of SLT-1A (SEQ ID NO: 1), 75-239 of StxA (SEQ ID NO:2), or 77-238 of (SEQ ID NO:3) or the equivalent region in another A Subunit of a member of the Shiga toxin family. The carboxy-terminus of a “Shiga toxin A1 fragment derived region” is defined, relative to a naturally occurring Shiga toxin A1 fragment, (1) as ending with the carboxy-terminal amino acid residue sharing homology with a naturally occurring, Shiga toxin A1 fragment; (2) as ending at the junction of the A 1 fragment and the A2 fragment; (3) as ending with a furin-cleavage site or disrupted furin-cleave site; and/or (4) as ending with a carboxy-terminal truncation of a Shiga toxin A1 fragment, i.e. the carboxy-terminal amino acid residue sharing homology with a naturally occurring, Shiga toxin A 1 fragment.

For purposes of the present invention, the phrase “carboxy terminus region of a Shiga toxin A1 fragment” refers to a polypeptide region derived from a naturally occurring Shiga toxin A1 fragment, the region beginning with a hydrophobic residue (e.g., V236 of StxA-A1 and SLT-1A1, and V235 of SLT-2A1) that is followed by a hydrophobic residue and the region ending with the furin-cleavage site conserved among Shiga toxin A1 fragment polypeptides and ending at the junction between the A1 fragment and the A2 fragment in native, Shiga toxin A Subunits. For purposes of the present invention, the carboxy-terminal region of a Shiga toxin A1 fragment includes a peptidic region derived from the carboxy terminus of a Shiga toxin A1 fragment polypeptide, such as, e.g., a peptidic region comprising or consisting essentially of the carboxy terminus of a Shiga toxin A1 fragment. Non-limiting examples of peptidic regions derived from the carboxy terminus of a Shiga toxin A1 fragment include the amino acid residue sequences natively positioned from position 236 to position 239, 240, 241, 242, 243, 244, 245, 246, 247, 248, 249, 250, or 251 in Stx1A (SEQ ID NO:2) or SLT-1A (SEQ ID NO:1); and from position 235 to position 239, 240, 241, 242, 243, 244, 245, 246, 247, 248, 249, or 250 in SLT-2A (SEQ ID NO:3).

For purposes of the present invention, the phrase “proximal to the carboxy terminus of an A1 fragment polypeptide” with regard to a linked molecular moiety and/or binding region refers to being within 1, 2, 3, 4, 5, 6, 7, 8, 9, 10, 11, or 12 amino acid residues from the amino acid residue defining the last residue of the Shiga toxin A1 fragment polypeptide.

For purposes of the present invention, the phrase “sterically covers the carboxy terminus of the A1 fragment-derived region” includes any molecular moiety of a size of 4.5 kDa or greater (e.g., an immunoglobulin-type binding region) linked and/or fused to an amino acid residue in the carboxy terminus of the A1 fragment-derived region, such as, e.g., the amino acid residue derived from the amino acid residue natively positioned at any one of positions 236 to 251 in Stx1A (SEQ ID NO:2) or SLT-1A (SEQ ID NO:1) or from 235 to 250 in SLT-2A (SEQ ID NO:3). For purposes of the present invention, the phrase “sterically covers the carboxy terminus of the A1 fragment-derived region” also includes any molecular moiety of a size of 4.5 kDa or greater (e.g., an immunoglobulin-type binding region) linked and/or fused to an amino acid residue in the carboxy terminus of the A1 fragment-derived region, such as, e.g., the amino acid residue carboxy-terminal to the last amino acid A1 fragment-derived region and/or the Shiga toxin effector polypeptide. For purposes of the present invention, the phrase “sterically covers the carboxy terminus of the A1 fragment-derived region” also includes any molecular moiety of a size of 4.5 kDa or greater (e.g., an immunoglobulin-type binding region) physically preventing cellular recognition of the carboxy terminus of the A1 fragment-derived region, such as, e.g. recognition by the ERAD machinery of a eukaryotic cell.

For purposes of the present invention, a binding region, such as, e.g., an immunoglobulin-type binding region, that comprises a polypeptide comprising at least forty amino acids and that is linked (e.g., fused) to the carboxy terminus of the Shiga toxin effector polypeptide region comprising an A1 fragment-derived region is a molecular moiety which is “sterically covering the carboxy terminus of the A1 fragment-derived region.”

For purposes of the present invention, a binding region, such as, e.g., an immunoglobulin-type binding region, that comprises a polypeptide comprising at least forty amino acids and that is linked (e.g., fused) to the carboxy terminus of the Shiga toxin effector polypeptide region comprising an A1 fragment-derived region is a molecular moiety “encumbering the carboxy terminus of the A1 fragment-derived region.”

For purposes of the present invention, the term “A1 fragment of a member of the Shiga toxin family” refers to the remaining amino-terminal fragment of a Shiga toxin A Subunit after proteolysis by furin at the furin-cleavage site conserved among Shiga toxin A Subunits and positioned between the A1 fragment and the A2 fragment in wild-type Shiga toxin A Subunits.

For purposes of the claimed invention, the phrase “furin-cleavage motif at the carboxy terminus of the A1 fragment region” refers to a specific, furin-cleavage motif conserved among Shiga toxin A Subunits and bridging the junction between the A1 fragment and the A2 fragment in naturally occurring, Shiga toxin A Subunits.

For purposes of the present invention, the phrase “furin-cleavage site proximal to the carboxy terminus of the A1 fragment region” refers to any identifiable, furin-cleavage site having an amino acid residue within a distance of less than 1, 2, 3, 4, 5, 6, 7, or more amino acid residues of the amino acid residue defining the last amino acid residue in the A1 fragment region or A1 fragment derived region, including a furin-cleavage motif located carboxy-terminal of an A1 fragment region or A1 fragment derived region, such as, e.g., at a position proximal to the linkage of the A1 fragment-derived region to another component of the molecule, such as, e.g., a molecular moiety of a cell-targeting molecule of the present invention.

For purposes of the present invention, the phrase “disrupted furin-cleavage motif refers to (i) a specific furin-cleavage motif as described herein and (ii) which comprises a mutation and/or truncation that can confer a molecule with a reduction in furin-cleavage as compared to a reference molecule, such as, e.g., a reduction in furin-cleavage reproducibly observed to be 30%, 40%, 50%, 60%, 70%, 80%, 90%, 95%, 97%, 98%, 99%, or less (including 100% for no cleavage) than the furin-cleavage of a reference molecule observed in the same assay under the same conditions. The percentage of furin-cleavage as compared to a reference molecule can be expressed as a ratio of cleaved:uncleaved material of the molecule of interest divided by the cleaved:uncleaved material of the reference molecule (see Examples, infra). Non-limiting examples of suitable reference molecules include certain molecules comprising a wild-type Shiga toxin furin-cleavage motif and/or furin-cleavage site as described herein in Section I-B, Section IV-B, and/or the Examples) and/or molecules used as reference molecules in the Examples below.

For purposes of the present invention, the phrase “furin-cleavage resistant” means a molecule or specific polypeptide region thereof exhibits reproducibly less furin cleavage than (i) the carboxy terminus of a Shiga toxin A1 fragment in a wild-type Shiga toxin A Subunit or (ii) the carboxy terminus of the Shiga toxin A1 fragment derived region of construct wherein the naturally occurring furin-cleavage site natively positioned at the junction between the A1 and A2 fragments is not disrupted; as assayed by any available means to the skilled worker, including by using a method described herein.

For purposes of the present invention, the phrase “active enzymatic domain derived form an A Subunit of a member of the Shiga toxin family” refers to a polypeptide structure having the ability to inhibit protein synthesis via catalytic inactivation of a ribosome based on a Shiga toxin enzymatic activity. The ability of a molecular structure to exhibit inhibitory activity of protein synthesis and/or catalytic inactivation of a ribosome may be observed using various assays known to the skilled worker, such as, e.g., in vitro assays involving RNA translation assays in the absence of living cells or in vivo assays involving the ribosomes of living cells. For example, using assays known to the skilled worker, the enzymatic activity of a molecule based on a Shiga toxin enzymatic activity may be assessed directly by observing N-glycosidase activity toward ribosomal RNA (rRNA), such as, e.g., a ribosome nicking assay, and/or indirectly by observing inhibition of ribosome function, RNA translation, and/or protein synthesis.

As used herein with respect to a Shiga toxin effector polypeptide, a “combination” describes a Shiga toxin effector polypeptide comprising two or more sub-regions wherein each sub-region comprises at least one of the following: (1) a disruption in an endogenous epitope or epitope region and (2) a disrupted furin-cleavage motif at the carboxy terminus of a Shiga toxin A1 fragment derived region.

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

### Introduction

The present invention provides various exemplary, Shiga toxin A Subunit derived constructs capable of delivering heterologous, CD8+ T-ceil epitopes to the MHC class I system of a target cell resulting in cell surface presentation of the delivered epitope. Shiga toxin A Subunit derived polypeptides can be engineered to have cell-targeting specificity by linking them to specific cell-targeting binding regions. The present invention exploits the abilities of Shiga toxin A Subunit derived polypeptides to drive their own subcellular routing in order to deliver highly immunogenic, CD8+ T-cell antigens, such as e.g. peptide-epitopes, to the MHC class I presentation system of a chordate cell. Shiga toxin A Subunit effector polypeptides can induce cellular internalization, direct subcellular routing to the cytosol, and deliver a heterologous, CD8+ T-cell epitope cargo to the MHC class I pathway for presentation on the surface of a cell. Certain peptide-epitopes presented in complexes with MHC class I molecules on a cellular surface can signal CD8+ effector T-cells to kill the presenting cell as well as stimulate other immune responses in the local area. Thus, the present invention provides Shiga toxin A Subunit derived, cell-targeting molecules which kill specific target cells, such as, e.g., via presentation of certain CD8+ T-cell epitope-peptides by the MHC class I pathway. The cell-targeting molecules of the present invention may be utilized, e.g., as cell-killing molecules, cytotoxic therapeutics, therapeutic delivery agents, and diagnostic molecules.

### I. The General Structure of the Cell-Targeting Molecules of the Present Invention

The cell-targeting molecules of the present invention each comprise 1) a cell-targeting binding region, 2) a Shiga toxin A Subunit effector polypeptide, and 3) a CD8+ T-cell epitope-peptide which is heterologous to Shiga toxin A Subunits and the binding region of the molecule. This system is modular, in that any number of diverse, CD8+ T-cell epitope-peptides may be used as cargos for delivery to the MHC class I presentation pathway of target cells of the cell-targeting molecules of the present invention.

### A. Shiga Toxin A Subunit Effector Polypeptides

A Shiga toxin effector polypeptide of the present invention is a polypeptide derived from a Shiga toxin A Subunit of at least one member of the Shiga toxin family wherein the Shiga toxin effector polypeptide is capable of exhibiting at least one Shiga toxin function. Shiga toxin functions include, e.g., promoting cell entry, deforming lipid membranes, stimulating clathrin-mediated endocytosis, directing retrograde transport, directing subcellular routing, avoiding intracellular degradation, catalytically inactivating ribosomes, effectuating cytotoxicity, and effectuating cytostatic effects.

There are numerous Shiga toxin effector polypeptides known to the skilled worker (see e.g., Cheung M et al., Mol Cancer 9: 28 (2010); WO 2014/164693; WO 2015/113005; WO 2015/113007; WO 2015/138452; WO 2015/191764) that are suitable for use in the present invention or to use as parental polypeptides to be modified into a Shiga toxin effector polypeptide of the present invention using techniques known the art.

Shiga toxin effector polypeptides of the present invention comprise or consist essentially of a polypeptide derived from a Shiga toxin A Subunit dissociated from any form of its native Shiga toxin B Subunit. The Shiga toxin effector polypeptides of the present invention do not comprise the cell-targeting domain of a Shiga toxin B Subunit. Archetypal Shiga toxins naturally target the human cell-surface receptors globotriaosylceramide (Gb3, Gb3Cer, or CD77) and globotetraosylceramide (Gb4 or Gb4Cer) via the Shiga toxin B Subunit, which severely limits potential applications by restricting targeting cell-types and potentially unwanted targeting of vascular endothelial cells, certain renal epithelial cells, and/or respiratory epithelial cells (Tesh V et al., Infect Immun 61: 3392-402 (1993); Ling H et al., Biochemistry 37: 1777-88 (1998); Bast D et al., Mol Microbiol 32: 953-60 (1999); Rutjes N et al., Kidney Int 62: 832-45 (2002); Shimizu T et al., Microb Pathog 43: 88-95 (2007); Pina D et al., Biochim Biophys Acta 1768: 628-36 (2007); Shin I et al., BMB Rep 42: 310-4 (2009); Zumbrun S et al., Infect Immun 4488-99 (2010); Engedal N et al., Microb Biotechnol 4: 32-46 (2011); Gallegos K et al., PLoS ONE 7: e30368 (2012); Stahl A et al., PLoS Pathog 11: e1004619 (2015)). Gb3 and Gb4 are a common, neutral sphingolipid present on the extracellular leaflet of cell membranes of various, healthy cell-types, such as polymorphonuclear leukocytes and human endothelial cells from various vascular beds. The cell-targeting molecules of the present invention do not comprise any polypeptide comprising or consisting essentially of a functional binding domain of a native Shiga toxin B subunit. Rather, the Shiga toxin effector polypeptides of the present invention may be functionally associated with heterologous binding regions to effectuate cell targeting.

In certain embodiments, a Shiga toxin effector polypeptide of the present invention may comprise or consist essentially of a full-length Shiga toxin A Subunit (e.g. SLT-1A (SEQ ID NO:1), StxA (SEQ ID NO:2), or SLT-2A (SEQ ID NO:3)), noting that naturally occurring Shiga toxin A Subunits may comprise precursor forms containing signal sequences of about 22 amino acids at their amino-terminals which are removed to produce mature Shiga toxin A Subunits and are recognizable to the skilled worker. In other embodiments, the Shiga toxin effector polypeptide of the invention comprises or consists essentially of a truncated Shiga toxin A Subunit which is shorter than a full-length Shiga toxin A Subunit, such as, e.g., a truncation known in the art (see e.g., WO 2014/164693; WO 2015/113005; WO 2015/113007; WO 2015/138452; WO 2015/191764).

While any Shiga toxin effector polypeptide known to the skilled worker may be suitable for use as a component of a cell-targeting molecule of the present invention, it is unknown if any Shiga toxin effector polypeptide described in WO 2015/113005 is capable of providing sufficient subcellular delivery of a heterologous, CD8+ T-cell epitope-peptide, which is not inserted or embedded in the Shiga toxin effector polypeptide, to the MHC class I presentation pathway of a target cell in order to induce detectable cell-surface presentation of the delivered, heterologous, CD8+ T-cell epitope-peptide complexed to MHC class I molecule. Furthermore, it is unknown and upredictable if any Shiga toxin effector polypeptide described in WO 2015/113005 is combinable with any structural feature of the Shiga toxin effector polypeptides described as an invention in WO 2015/191764 such that the resulting combination molecule would be stable and capable of providing sufficient subcellular delivery of a heterologous, CD8+ T-cell epitope-peptide to the MHC class I presentation pathway of a target cell in order to induce detectable cell-surface presentation of the delivered, heterologous, CD8+ T-cell epitope-peptide complexed to MHC class I molecule.

### B. Heterologous, CD8+ T-Cell Epitope-Peptide Cargos for Delivery

The cell-targeting molecules of the present invention each comprise one or more CD8+ T-cell epitope-peptides that are heterologous to their respective Shiga toxin effector polypeptide(s) and binding region(s). A CD8+ T-cell epitope is a molecular structure recognizable by an immune system of at least one individual, i.e. an antigenic peptide. The heterologous, CD8+ T-cell epitope-peptide of the cell-targeting molecule of the present invention can be chosen from virtually any CD8+ T-cell epitope.

For purposes of the claimed invention, a CD8+ T-cell epitope (also known as a MHC class I epitope or MHC class I peptide) is a molecular structure which is comprised by an antigenic peptide and can be represented by a linear, amino acid sequence. Commonly, CD8+ T-cell epitopes are peptides of sizes of eight to eleven amino acid residues (Townsend A, Bodmer H, Annu Rev Immunol 7: 601-24 (1989)); however, certain CD8+ T-cell epitopes have lengths that are smaller than eight or larger than eleven amino acids long {see e.g. Livingstone A, Fathman C, Annu Rev Immunol 5: 477-501 (1987); Green K et al., Eur J Immunol 34: 2510-9 (2004)).

For purposes of the claimed invention, the term “heterologous” means of a different source than (1) an A Subunit of a naturally occurring Shiga toxin and (2) the binding region of the cell-targeting molecule comprising the heterologous component. A heterologous, CD8+ T-cell epitope-peptide of the cell-targeting molecule of the present invention is an CD8+ T-cell epitope-peptide not already present in a wild-type Shiga toxin A1 fragment; a naturally occurring Shiga toxin A1 fragment; and/or a prior art Shiga toxin effector polypeptide used as a component of the cell-targeting molecule.

In certain embodiments of the present invention, the heterologous, CD8+ T-cell epitope-peptide is at least seven amino acid residues in length. In certain embodiments of the present invention, the CD8+ T-cell epitope-peptide is bound by a TCR with a binding affinity characterized by a K_{D} less than 10 millimolar (mM) {e.g. 1-100 μM) as calculated using the formula in Stone J et al., Immunology 126: 165-76 (2009). However, it should be noted that the binding affinity within a given range between the MHC-epitope and TCR may not correlate with antigenicity and/or immunogenicity {see e.g. Al-Ramadi B et al., J Immunol 155: 662-73 (1995)), such as due to factors like MHC I-peptide-TCR complex stability, MHC I-peptide density and MHC-independent functions of TCR cofactors such as CD8 (Baker B et al., Immunity 13: 475-84 (2000); Hornell T et al., J Immunol 170: 4506-14 (2003); Woolridge L et al., J Immunol 111: 6650-60 (2003)).

T-cell epitopes may be chosen or derived from a number of source molecules for use in the present invention. T-cell epitopes may be created or derived from various naturally occurring proteins. T-cell epitopes may be created or derived from various naturally occurring proteins foreign to mammals, such as, e.g., proteins of microorganisms. T-cell epitopes may be created or derived from mutated human proteins and/or human proteins aberrantly expressed by malignant human cells. T-cell epitopes may be synthetically created or derived from synthetic molecules {see e.g., Carbone F et al., J Exp Med 167: 1767-9 (1988); Del Val M et al., J Virol 65: 3641-6 (1991); Appella E et al., Biomed Pept Proteins Nucleic Acids 1: 177-84 (1995); Perez S et al., Cancer 116: 2071-80 (2010)).

The CD8+ T-cell epitope-peptide of the cell-targeting molecule of the present invention can be chosen from various known antigens, such as, e.g., well-characterized immunogenic epitopes from human pathogens, typically the most common pathogenic viruses and bacteria.

CD8+ T-cell epitopes can be identified by reverse immunology methods known to the skilled worker, such as, e.g., genetic approaches, library screening, and eluting peptides off of cells displaying MHC class I molecules and sequencing them by mass-spectrometry, {see e.g. Van Der Bruggen P et al., Immunol Rev 188: 51-64 (2002)).

Additionally, other MHC I-peptide binding assays based on a measure of the ability of a peptide to stabilize the ternary MHC-peptide complex for a given MHC class I allele, as a comparison to known controls, have been developed {e.g., MHC I-peptide binding assay from Prolmmune, Inc., Sarasota, Fla., U.S.). Such approaches can help predict the effectiveness of a putative CD8+ T-cell epitope-peptide or to corroborate empirical evidence regarding a known CD8+ T-cell epitope.

Although any CD8+ T-cell epitope is contemplated as being used as a heterologous, CD8+ T-cell epitope of the present invention, certain CD8+ T-cell epitopes may be selected based on desirable properties. One objective is to create CD8+ T-cell hyper-immunized cell-targeting molecules, meaning that the heterologous, CD8+ T-cell epitope-peptide is highly immunogenic because it can elicit robust immune responses in vivo when displayed complexed with a MHC class I molecule on the surface of a cell.

CD8+ T-cell epitopes may be derived from a number of source molecules already known to be capable of eliciting a vertebrate immune response. CD8+ T-cell epitopes may be derived from various naturally occurring proteins foreign to vertebrates, such as, e.g., proteins of pathogenic microorganisms and non-self, cancer antigens. In particular, infectious microorganisms may contain numerous proteins with known antigenic and/or immunogenic properties. Further, infectious microorganisms may contain numerous proteins with known antigenic and/or immunogenic sub-regions or epitopes. CD8+ T-cell epitopes may be derived from mutated human proteins and/or human proteins aberrantly expressed by malignant human cells, such as, e.g., mutated proteins expressed by cancer cells (see e.g. Sjoblom T et al., Science 314: 268-74 (2006); Wood L et al., Science 318: 1108-13 (2007); Jones S et al., Science 321: 1801-6 (2008); Parsons D et al., Science 321: 1807-12 (2008); Wei X et al., Nat Genet 43: 442-6 (2011); Govindan R et al., Cell 150: 1121-34 (2012); Vogel stein B et al., Science 339: 1546-58 (2013)).

CD8+ T-cell epitopes may be chosen or derived from a number of source molecules already known to be capable of eliciting a mammalian immune response, including peptides, peptide components of proteins, and peptides derived from proteins. For example, the proteins of intracellular pathogens with mammalian hosts are sources for CD8+ T-cell epitopes. There are numerous intracellular pathogens, such as viruses, bacteria, fungi, and single-cell eukaryotes, with well-studied antigenic proteins or peptides. CD8+ T-cell epitopes can be selected or identified from human viruses or other intracellular pathogens, such as, e.g., bacteria like mycobacterium, fungi like toxoplasmae, and protists like trypanosomes.

For example, there are many known immunogenic viral peptide components of viral proteins from viruses that infect humans. Numerous human CD8+ T-cell epitopes have been mapped to peptides within proteins from influenza A viruses, such as peptides in the proteins HA glycoproteins FE17, S139/1, CH65, C05, hemagglutinin 1 (HA1), hemagglutinin 2 (HA2), nonstructural protein 1 and 2 (NS1 and NS2), matrix protein 1 and 2 (M1 and M2), nucleoprotein (NP), neuraminidase (NA)), and many of these peptides have been shown to elicit human immune responses, such as by using ex vivo assay (see e.g. Assarsson E et al, J Virol 82: 12241-51 (2008); Alexander J et al., Hum Immunol 71: 468-74 (2010); Wang M et al., PLoS One 5: e10533 (2010); Wu J et al., Clin Infect Dis 51: 1184-91 (2010); Tan P et al., Human Vaccin 7: 402-9 (2011); Grant E et al., Immunol Cell Biol 91: 184-94 (2013); Terajima M et al., Virol J 10: 244 (2013)). Similarly, numerous human CD8+ T-cell epitopes have been mapped to peptide components of proteins from human cytomegaloviruses (HCMV), such as peptides in the proteins pp65 (UL83), UL128-131, immediate-early 1 (IE-1; UL123), glycoprotein B, tegument proteins, and many of these peptides have been shown to elicit human immune responses, such as by using ex vivo assays (Schoppel K et al., J Infect Dis 175: 533-44 (1997); Elkington R et al, J Virol 11: 5226-40 (2003); Gibson L et al., J Immunol 111: 2256-64 (2004); Ryckman B et al., J Virol 82: 60-70 (2008); Sacre K et al., J Virol 82: 10143-52 (2008)).

Another example is there are many immunogenic, cancer antigens in humans. The CD8+ T-cell epitopes of cancer and/or tumor cell antigens can be identified by the skilled worker using techniques known in the art, such as, e.g., differential genomics, differential proteomics, immunoproteomics, prediction then validation, and genetic approaches like reverse-genetic transfection (see e.g., Admon A et al., Mol Cell Proteomics 2: 388-98 (2003); Purcell A, Gorman J, Mol Cell Proteomics 3: 193-208 (2004); Comber J, Philip R, Ther Adv Vaccines 2: 77-89 (2014)). There are many antigenic and/or immunogenic T-cell epitopes already identified or predicted to occur in human cancer and/or tumor cells. For example, T-cell epitopes have been predicted in human proteins commonly mutated or overexpressed in neoplastic cells, such as, e.g., ALK, CEA, N-acetylglucosaminyl-transferase V (GnT-V), HCA587, HER-2/neu, MAGE, Melan-A/MART-1, MUC-1, p53, and TRAG-3 (see e.g., van der Bruggen P et al., Science 254: 1643-7 (1991); Kawakami Y et al., J Exp Med 180: 347-52 (1994); Fisk B et al., J Exp Med 181: 2109-17 (1995); Guilloux Y et al., J Exp Med 183: 1173 (1996); Skipper J et al., J ExpMed 183: 527 (1996); Brossart P et al., 93: 4309-17 (1999); Kawashima I et al., Cancer Res 59: 431-5 (1999); Papadopoulos K et al., Clin Cancer Res 5: 2089-93 (1999); Zhu B et al., Clin Cancer Res 9: 1850-7 (2003); Li B et al., Clin Exp Immunol 140: 310-9 (2005); Ait-Tahar K et al., Int J Cancer 118: 688-95 (2006); Akiyama Y et al., Cancer Immunol Immunother 61: 23 11-9 (2012)). In addition, synthetic variants of T-cell epitopes from human cancer cells have been created (see e.g., Lazoura E, Apostolopoulos V, CurrMed Chem 12: 629-39 (2005); Douat-Casassus C et al., J Med Chem 50: 1598-609 (2007)).

While any heterologous, CD8+ T-cell epitope may be used in the compositions and methods of the present invention, certain CD8+ T-cell epitopes may be preferred based on their known and/or empirically determined characteristics Immunogenic peptide-epitopes that elicit a human, CD8+ T-cell responses have been described and/or can be identified using techniques known to the skilled worker (see e.g. Kalish R, J Invest Dermatol 94: 108S-1 1IS (1990); Altman J et al., Science 274: 94-6 (1996); Callan M et al., J Exp Med 187: 1395-402 (1998); Dunbar P et al., Curr Biol 8: 413-6 (1998); Sourdive D et al., J Exp Med 188: 71-82 (1998); Collins E et al., J Immunol 162: 331-7 (1999); Yee C et al., J Immunol 162: 2227-34 (1999); Burrows S et al., J Immunol 165: 6229-34 (2000); Cheuk E et al., J Immunol 169: 5571-80 (2002); Elkington R et al, J Virol 77: 5226-40 (2003); Oh S et al., Cancer Res 64: 2610-8 (2004); Hopkins L et al., Hum Immunol 66: 874-83 (2005); Assarsson E et al, J Virol 12241-51 (2008); Semeniuk C et al., AIDS 23: 771-7 (2009); Wang X et al., J Vis Exp 61: 3657 (2012); Song H et al., Virology 447: 181-6 (2013); Chen L et al., J Virol 88: 11760-73 (2014)).

In many species, the MHC gene encodes multiple MHC-I molecular variants. Because MHC class I protein polymorphisms can affect antigen-MHC class I complex recognition by CD8+ T-cells, heterologous T-cell epitopes may be chosen based on knowledge about certain MHC class I polymorphisms and/or the ability of certain antigen-MHC class I complexes to be recognized by T-cells of different genotypes.

There are well-defined peptide-epitopes that are known to be immunogenic, MHC class I restricted, and/or matched with a specific human leukocyte antigen (HLA) variant(s). For applications in humans or involving human target cells, HLA-Class I-restricted epitopes can be selected or identified by the skilled worker using standard techniques known in the art. The ability of peptides to bind to human MHC Class I molecules can be used to predict the immunogenic potential of putative, CD8+ T-cell epitopes. The ability of peptides to bind to human MHC class I molecules can be scored using software tools. CD8+ T-cell epitopes may be chosen for use as a CD8+ heterologous, T-cell epitope component of the present invention based on the peptide selectivity of the HLA variants encoded by the alleles more prevalent in certain human populations. For example, the human population is polymorphic for the alpha chain of MHC class I molecules, and the variable alleles are encoded by the HLA genes. Certain T-cell epitopes may be more efficiently presented by a specific HLA molecule, such as, e.g., the commonly occurring HLA variants encoded by the HLA-A allele groups HLA-A2 and HLA-A3.

When choosing CD8+ T-cell epitopes for use as a heterologous, CD8+ T-cell epitope-peptide component of the cell-targeting molecule of the present invention, CD8+ epitopes may be selected which best match the MHC Class I molecules present in the cell-type or cell populations to be targeted. Different MHC class I molecules exhibit preferential binding to particular peptide sequences, and particular peptide-MHC class I variant complexes are specifically recognized by the TCRs of effector T-cells. The skilled worker can use knowledge about MHC class I molecule specificities and TCR specificities to optimize the selection of heterologous T-cell epitopes used in the present invention.

In certain embodiments of the cell-targeting molecule of the present invention, the heterologous, CD8+ T-cell epitope-peptide is comprised within a heterologous polypeptide, such as, e.g., an antigen or antigenic protein. In certain further embodiments, the heterologous polypeptide is no larger than 27 kDa, 28 kDa, 29 kDa, or 30 kDa.

In certain embodiments, the cell-targeting molecule of the present invention comprises two or more heterologous, CD8+ T-cell epitope-peptides. In certain further embodiments, the combined size of all the heterologous, CD8+ T-cell epitope-peptides is no larger than 27 kDa, 28 kDa, 29 kDa, or 30 kDa.

In certain embodiments of the cell-targeting molecule of the present invention, the heterologous, CD8+ T-cell epitope-peptide is processed better in cells with more immunoproteasomes, intermediate proteasomes, and/or thymoproteasomes as compared to standard proteasomes; however, in other embodiments the opposite is true.

When choosing CD8+ T-cell epitope-peptides for use as a heterologous, CD8+ T-cell epitope-peptide component of a cell-targeting molecule of the present invention, multiple factors in the MHC class I presentation system may be considered that can influence CD8+ T-cell epitope generation and transport to receptive MHC class I molecules, such as, e.g., the epitope specificity of the following factors in the target cell: proteasome, ERAAP/ERAP1, tapasin, and TAPs can (see e.g. Akram A, Inman R, Clin Immunol 143: 99-115 (2012)).

In certain embodiments of the cell-targeting molecule of the present invention, the heterologous, CD8+ T-cell epitope-peptide is only proteolytically processed in an intact form by an intermediate proteasome (see e.g. Guillaume B et al., Proc Natl Acad Sci USA 107: 18599-604 (2010); Guillaume B et al., J Immunol 189: 3538-47 (2012)).

In certain embodiments of the cell-targeting molecule of the present invention, the heterologous, CD8+ T-cell epitope-peptide is not destroyed by standard proteasomes, immunoproteasomes, intermediate proteasomes, and/or thymoproteasomes, which also may depend on the cell type, cytokine environment, tissue location, etc. (see e.g., Morel S et al., Immunity 12: 107-17 (2000); Chapiro J et al., J Immunol 176: 1053-61 (2006); Guillaume B et al., Proc Natl Acad Sci U.S.A. 107: 18599-604 (2010); Dalet A et al., Eur J Immunol 41: 39\hat{ }16 (2011); Basler M et al., J Immunol 189: 1868-77 (2012); Guillaume B et al., J Immunol 189: 3538-47 (2012)).

In certain embodiments of the cell-targeting molecule of the present invention, the heterologous, CD8+ T-cell epitope-peptide is considered a “weak” epitope, such as, e.g., “weak” in vivo at eliciting a CD8+ CTL response in a given subject or genotype group or cells derived from the aforementioned (see e.g. Cao W et al., J Immunol 157: 505-11 (1996)).

In certain embodiments of the cell-targeting molecule of the present invention, the heterologous, CD8+ T-cell epitope-peptide is a tumor cell epitope, such as, e.g., NY-ESO-1 157-165A (see e.g. Jager E et al. J Exp Med 187: 265-70 (1998)).

In certain embodiments of the cell-targeting molecule of the present invention, the heterologous, CD8+ T-cell epitope-peptide has been modified to have a bulky or a charged residue at its amino terminus in order to increase ubiquitination (see e.g., Grant E et al., J Immunol 155: 3750-8 (1995); Townsend A et al., J Exp Med 168: 1211-24 (1998); Kwon Y et al., Proc Natl Acad Sci U.S.A. 95: 7898-903 (1998)).

In certain embodiments of the cell-targeting molecule of the present invention, the heterologous, CD8+ T-cell epitope-peptide has been modified to have a hydrophobic amino acid residue at its carboxy terminus in order to increase proteolytic cleavage probability (see e.g., Driscoll J et al., Nature 365: 262-4 (1993); Gaczynska M et al., Nature 365: 264-7 (1993)).

In certain embodiments of the cell-targeting molecule of the present invention, the heterologous, CD8+ T-cell epitope-peptide is a Tregitope. Tregitopes are functionally defined as epitope-peptides capable of inducing an immuno-suppressive result. Examples of naturally occurring Tregitopes include sub-regions of human immunoglobulin G heavy chain constant regions (Fes) and Fabs (see e.g., Sumida T et al., Arthritis Rheum 40: 2271-3 (1997); Bluestone J, Abbas A, Nat Rev Immunol 3: 253-7 (2003); Hahn B et al., J Immunol 175: 7728-37 (2005); Durinovic-Bello I et al., Proc Natl Acad Sci USA 103: 11683-8 (2006); Sharabi A et al., Proc Natl Acad Sci USA 103: 8810-5 (2006); De Groot A et al., Blood 112: 3303-11 (2008); Sharabi A et al., J Clin Immunol 30: 34-4 (2010); Mozes E, Sharabi A, Autoimmun Rev 10: 22-6 (2010)).

While the position of the heterologous, CD8+ T-cell epitope-peptide of the cell-targeting molecule of the present invention is not generally restricted. In certain embodiments of the present invention, the heterologous, CD8+ T-cell epitope-peptide is linked to the cell-targeting molecule at a location carboxy-terminal to the Shiga toxin A 1 fragment derived region.

### C. Cell-Targeting Binding Regions

The cell-targeting molecules of the present invention comprise a cell-targeting binding region capable of specifically binding an extracellular target biomolecule.

In certain embodiments, a binding region of a cell-targeting molecule of the present invention is a cell-targeting component, such as, e.g., a domain, molecular moiety, or agent, capable of binding specifically to an extracellular part of a target biomolecule (e.g. an extracellular target biomolecule) with high affinity. There are numerous types of binding regions known to skilled worker or which may be discovered by the skilled worker using techniques known in the art. For example, any cell-targeting component that exhibits the requisite binding characteristics described herein may be used as the binding region in certain embodiments of the cell-targeting molecules of the present invention.

An extracellular part of a target biomolecule refers to a portion of its structure exposed to the extracellular environment when the molecule is physically coupled to a cell, such as, e.g., when the target biomolecule is expressed at a cellular surface by the cell. In this context, exposed to the extracellular environment means that part of the target biomolecule is accessible by, e.g., an antibody or at least a binding moiety smaller than an antibody such as a single-domain antibody domain, a nanobody, a heavy-chain antibody domain derived from camelids or cartilaginous fishes, a single-chain variable fragment, or any number of engineered alternative scaffolds to immunoglobulins (see below). The exposure to the extracellular environment of or accessibility to a part of target biomolecule physically coupled to a cell may be empirically determined by the skilled worker using methods well known in the art.

A binding region of a cell-targeting molecule of the present invention may be, e.g., a ligand, peptide, immunoglobulin-type binding region, monoclonal antibody, engineered antibody derivative, or engineered alternative to antibodies.

In certain embodiments, the binding region of a cell-targeting molecule of the present invention is a proteinaceous moiety capable of binding specifically to an extracellular part of target biomolecule with high affinity. A binding region of a cell-targeting molecule of the present invention may comprise one or more various peptidic or polypeptide moieties, such as randomly generated peptide sequences, naturally occurring ligands or derivatives thereof, immunoglobulin derived domains, synthetically engineered scaffolds as alternatives to immunoglobulin domains, and the like (see e.g., WO 2005/092917; WO 2007/033497; Cheung M et al., Mol Cancer 9: 28 (2010); US 2013/0196928; WO 2014/164693; WO 2015/113005; WO 2015/113007; WO 2015/138452; WO 2015/191764). In certain embodiments, a cell-targeting molecule of the present invention comprises a binding region comprising one or more polypeptides capable of selectively and specifically binding an extracellular target biomolecule.

There are numerous binding regions known in the art that are useful for targeting molecules to specific cell-types via their binding characteristics, such as certain ligands, monoclonal antibodies, engineered antibody derivatives, and engineered alternatives to antibodies.

According to one specific but non-limiting aspect, the binding region of a cell-targeting molecule of the present invention comprises a naturally occurring ligand or derivative thereof that retains binding functionality to an extracellular target biomolecule, commonly a cell surface receptor. For example, various cytokines, growth factors, and hormones known in the art may be used to target the cell-targeting molecule of the present invention to the cell-surface of specific cell-types expressing a cognate cytokine receptor, growth factor receptor, or hormone receptor. Certain non-limiting examples of ligands include (alternative names are indicated in parentheses) angiogenin, B-cell activating factors (BAFFs, APRIL), colony stimulating factors (CSFs), epidermal growth factors (EGFs), fibroblast growth factors (FGFs), vascular endothelial growth factors (VEGFs), insulin-like growth factors (IGFs), interferons, interleukins (such as IL-2, IL-6, and IL-23), nerve growth factors (NGFs), platelet derived growth factors, transforming growth factors (TGFs), and tumor necrosis factors (TNFs).

According to certain other embodiments of the cell-targeting molecules of the present invention, the binding region comprises a synthetic ligand capable of binding an extracellular target biomolecule (see e.g. Liang S et al., J Mol Med 84: 764-73 (2006); Ahmed S et al., Anal Chem 82: 7533-41 (2010); Kaur K et al., Methods Mol Biol 1248: 239-47 (2015)).

In certain embodiments, the binding region comprises a peptidomimetic, such as, e.g., an AApeptide, gamma-AApeptide, and/or sulfono-y-AApeptide {see e.g., Pilsl L, Reiser O, Amino Acids 41: 709-18 (2011); Akram O et al., Mol Cancer Res 12: 967-78 (2014); Wu H et al., Chemistry 21: 2501-7 (2015); Teng P et al., Chemistry 2016 Mar. 4)).

According to one specific, but non-limiting aspect, the binding region may comprise an immunoglobulin-type binding region. The term “immunoglobulin-type binding region” as used herein refers to a polypeptide region capable of binding one or more target biomolecules, such as an antigen or epitope. Binding regions may be functionally defined by their ability to bind to target molecules Immunoglobulin-type binding regions are commonly derived from antibody or antibody-like structures; however, alternative scaffolds from other sources are contemplated within the scope of the term.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (Igl or I-set). Some Ig domains may be associated with a complementarity determining region (CDR), also called a “complementary determining region,” which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

An immunoglobulin-type binding region may be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other improvements for in vivo and/or therapeutic applications. The possible variations are many and may range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions contemplated as components of the present invention. In certain embodiments, the immunoglobulin-type binding region is derived from an immunoglobulin binding region, such as an antibody paratope capable of binding an extracellular target biomolecule. In certain other embodiments, the immunoglobulin-type binding region comprises an engineered polypeptide not derived from any immunoglobulin domain but which functions like an immunoglobulin binding region by providing high-affinity binding to an extracellular target biomolecule. This engineered polypeptide may optionally include polypeptide scaffolds comprising or consisting essentially of complementary determining regions from immunoglobulins as described herein.

There are also numerous binding regions in the prior art that are useful for targeting polypeptides to specific cell-types via their high-affinity binding characteristics. In certain embodiments of the cell-targeting molecules of the present invention, the binding region comprises immunoglobulin domain selected from the group which includes autonomous VH domains, single-domain antibody domains (sdAbs), heavy-chain antibody domains derived from camelids (VHH fragments or VH domain fragments), heavy-chain antibody domains derived from camelid VHH fragments or VH domain fragments, heavy-chain antibody domains derived from cartilaginous fishes, immunoglobulin new antigen receptors (IgNARs), VNAR fragments, single-chain variable (scFv) fragments, nanobodies, Fd fragments consisting of the heavy chain and CHI domains, permutated Fvs (pFv), single chain FV-CH3 minibodies, dimeric CH2 domain fragments (CH2D), F C antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMTP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the VL, VH, CL and CHI domains, bivalent nanobodies, bivalent minibodies, bivalent F(ab′)₂ fragments (Fab dimers), bispecific tandem VHH fragments, bispecific tandem scFv fragments, bispecific nanobodies, bispecific minibodies, and any genetically manipulated counterparts of the foregoing that retains its binding functionality (Worn A, Pliickthun A, J Mol Biol 305: 989-1010 (2001); Xu L et al., Chem Biol 9: 933-42 (2002); Wikman M et al., Protein EngDes Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Byla P et al., J Biol Chem 285: 12096 (2010); Zoller F et al., Molecules 16: 2467-85 (2011); Alfarano P et al., Protein Sci 21: 1298-314 (2012); Madhurantakam C et al., Protein Sci 21: 1015-28 (2012); Varadamsetty G et al., J Mol Biol 424: 68-87 (2012); Reichen C et al., J Struct Biol 185: 147-62 (2014)).

In certain embodiments, the binding region of the cell-targeting molecule of the present invention is selected from the group which includes autonomous VH domains, single-domain antibody domains (sdAbs), heavy-chain antibody domains derived from camelids (vim fragments or VH domain fragments), heavy-chain antibody domains derived from camelid VHH fragments or VH domain fragments, heavy-chain antibody domains derived from cartilaginous fishes, immunoglobulin new antigen receptors (IgNARs), VNAR fragments, single-chain variable (scFv) fragments, nanobodies, Fd fragments consisting of the heavy chain and CHI domains, single chain FV-CH3 minibodies, dimeric CH2 domain fragments (CH2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the VL, VH, CL and CHI domains, bivalent nanobodies, bivalent minibodies, bivalent F(ab′)₂ fragments (Fab dimers), bispecific tandem VHH fragments, bispecific tandem scFv fragments, bispecific nanobodies, bispecific minibodies, and any genetically manipulated counterparts of the foregoing that retain its paratope and binding function (see Ward E et al., Nature 341: 544-6 (1989); Davies J, Riechmann L, Biotechnology (NY) 13: 475-9 (1995); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002); Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1: 26-8 (2009); Weiner L, Cell 148: 1081-4 (2012); Ahmad Z et al., Clin Dev Immunol 2012: 980250 (2012)).

There are a variety of binding regions comprising polypeptides derived from the constant regions of immunoglobulins, such as, e.g., engineered dimeric Fc domains, monomeric Fes (mFcs), scFv-Fcs, VHH-FCS, CH2 domains, monomeric CH3 S domains (mCH3s), synthetically reprogrammed immunoglobulin domains, and/or hybrid fusions of immunoglobulin domains with ligands (Hofer T et al., Proc Natl Acad Sci U.S.A. 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein EngDes Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014); Ying T et al., Biochimica Biophys Acta 1844: 1977-82 (2014)).

In accordance with certain other embodiments, the binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and may provide improved characteristics to certain immunoglobulin domains, such as, e.g., greater stability or reduced immunogenicity. Generally, alternative scaffolds to immunoglobulins are less than 20 kilodaltons (kDa), consist of a single polypeptide chain, lack cysteine residues, and exhibit relatively high thermodynamic stability.

In certain embodiments of the cell-targeting molecules of the present invention, the immunoglobulin-type binding region is selected from the group which includes engineered, Armadillo repeat polypeptides (ArmRPs); engineered, fibronectin-derived, 10^{th} fibronectin type III (10Fn3) domains (monobodies, AdNectins™, or AdNexins™); engineered, tenascin-derived, tenascin type III domains (Centryns™); engineered, ankyrin repeat motif containing polypeptides (DARPins™); engineered, low-density-lipoprotein-receptor-derived, A domains (LDLR-A) (Avimers™); lipocalins (anticalins); engineered, protease inhibitor-derived, Kunitz domains; engineered, Protein-A-derived, Z domains (Affibodies™); engineered, gamma-B crystalline-derived scaffold or engineered, ubiquitin-derived scaffolds (Affilins); Sac7d-derived polypeptides (Nanoffitins® or affitins); engineered, Fyn-derived, SH2 domains (Fynomers®); and engineered antibody mimics and any genetically manipulated counterparts of the foregoing that retains its binding functionality (Worn A, Pluckthun A, JMolBiol 305: 989-1010 (2001); Xu L et al., Chem Biol 9: 933-42 (2002); Wikman M et al., Protein EngDes Sel 17: 455-62 (2004); Binz H et al., Nat Biotechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Byla P et al., J Biol Chem 285: 12096 (2010); Zoller F et al., Molecules 16: 2467-85 (2011); Alfarano P et al., Protein Sci 21: 1298-314 (2012); Madhurantakam C et al., Protein Sci 21: 1015-28 (2012); Varadamsetty G et al., J Mol Biol 424: 68-87 (2012)).

For example, there is an engineered Fn3(CD20) binding region scaffold which exhibits high-affinity binding to CD20 expressing cells (Natarajan A et al., Clin Cancer Res 19: 6820-9 (2013)).

For example, numerous alternative scaffolds have been identified which bind to the extracellular receptor HER2 (see e.g. Wikman M et al., Protein EngDes Sel 17: 455-62 (2004); Orlova A et al. Cancer Res 66: 4339-8 (2006); Ahlgren S et al., Bioconjug Chem 19: 235-43 (2008); Feldwisch J et al., J Mol Biol 398: 232-47 (2010); U.S. Pat. Nos. 5,578,482; 5,856,110; 5,869,445; 5,985,553; 6,333,169; 6,987,088; 7,019,017; 7,282,365; 7,306,801; 7,435,797; 7,446,185; 7,449,480; 7,560,111; 7,674,460; 7,815,906; 7,879,325; 7,884,194; 7,993,650; 8,241,630; 8,349,585; 8,389,227; 8,501,909; 8,512,967; 8,652,474; and U.S. patent application 2011/0059090). In addition to alternative antibody formats, antibody-like binding abilities may be conferred by non-proteinaceous compounds, such as, e.g., oligomers, RNA molecules, DNA molecules, carbohydrates, and glycocalyxcalixarenes (see e.g. Sansone F, Casnati A, Chem Soc Rev 42: 4623-39 (2013)) or partially proteinaceous compounds, such as, e.g., phenol-formaldehyde cyclic oligomers coupled with peptides and calixarene-peptide compositions (see e.g. U.S. Pat. No. 5,770,380).

Any of the above binding region structures may be used as a component of a cell-targeting molecule of the present invention as long as the binding region component has a dissociation constant of 10⁻⁵ to 10⁻¹² moles per liter, preferably less than 200 nanomolar (nM), towards an extracellular target biomolecule.

In certain embodiments, the cell-targeting molecules of the present invention comprise a Shiga toxin effector polypeptide of the present invention linked and/or fused to a binding region capable of specifically binding an extracellular part of a target biomolecule or an extracellular target biomolecule. Extracellular target biomolecules may be selected based on numerous criteria, such as a criterion described herein.

### Extracellular Target Biomolecules Bound by the Binding Regions

In certain embodiments, the binding region of a cell-targeting molecules of the present invention comprises a proteinaceous region capable of binding specifically to an extracellular part of a target biomolecule or an extracellular target biomolecule, preferably which is physically coupled to the surface of a cell-type of interest, such as, e.g., a cancer cell, tumor cell, plasma cell, infected cell, or host cell harboring an intracellular pathogen. Preferably, the targeted cell-type will be expressing a MHC class I molecule(s). Target biomolecules bound by the binding region of a cell-targeting molecule of the present invention may include biomarkers over-proportionately or exclusively present on cancer cells, immune cells, and/or cells infected with intracellular pathogens, such as, e.g., viruses, bacteria, fungi, prions, or protozoans.

The term “target biomolecule” refers to a biological molecule, commonly a proteinaceous molecule or a protein modified by post-translational modifications, such as glycosylation, that is bound by a binding region of a cell-targeting molecule of the present invention resulting in the targeting of the cell-targeting molecule to a specific cell, cell-type, and/or location within a multicellular organism.

For purposes of the present invention, the term “extracellular” with regard to a target biomolecule refers to a biomolecule that has at least a portion of its structure exposed to the extracellular environment. The exposure to the extracellular environment of or accessibility to a part of target biomolecule coupled to a cell may be empirically determined by the skilled worker using methods well known in the art. Non-limiting examples of extracellular target biomolecules include cell membrane components, transmembrane spanning proteins, cell membrane-anchored biomolecules, cell-surface-bound biomolecules, and secreted biomolecules.

With regard to the present invention, the phrase “physically coupled” when used to describe a target biomolecule means covalent and/or non-covalent intermolecular interactions couple the target biomolecule, or a portion thereof, to the outside of a cell, such as a plurality of non-covalent interactions between the target biomolecule and the cell where the energy of each single interaction is on the order of at least about 1-5 kiloCalories (e.g., electrostatic bonds, hydrogen bonds, ionic bonds, Van der Walls interactions, hydrophobic forces, etc.). All integral membrane proteins can be found physically coupled to a cell membrane, as well as peripheral membrane proteins. For example, an extracellular target biomolecule might comprise a transmembrane spanning region, a lipid anchor, a glycolipid anchor, and/or be non-covalently associated (e.g. via non-specific hydrophobic interactions and/or lipid binding interactions) with a factor comprising any one of the foregoing.

Extracellular parts of target biomolecules may include various epitopes, including unmodified polypeptides, polypeptides modified by the addition of biochemical functional groups, and glycolipids (see e.g. U.S. Pat. No. 5,091,178; EP2431743).

The binding regions of the cell-targeting molecules of the present invention may be designed or selected based on numerous criteria, such as the cell-type specific expression of their target biomolecules, the physical localization of their target biomolecules with regard to specific cell-types, and/or the properties of their target biomolecules. For example, certain cell-targeting molecules of the present invention comprise binding regions capable of binding cell-surface target biomolecules that are expressed at a cellular surface exclusively by only one cell-type of a species or only one cell-type within a multicellular organism. It is desirable, but not necessary, that an extracellular target biomolecule be intrinsically internalized or be readily forced to internalize upon interacting with a cell-targeting molecule of the present invention.

It will be appreciated by the skilled worker that any desired target biomolecule may be used to design or select a suitable binding region to be associated and/or coupled with a Shiga toxin effector polypeptide to produce a cell-targeting molecule of the present invention.

The general structure of the cell-targeting molecules of the present invention is modular, in that various, diverse cell-targeting binding regions may be used with various Shiga toxin effector polypeptides and CD8+ T-cell epitope-peptides to provide for diverse targeting and delivery of various epitopes to the MHC class I system of diverse target cell-types. Optionally, a cell-targeting molecule of the invention (e.g. protein) may further comprise a carboxy-terminal endoplasmic retention/retrieval signal motif, such as, e.g., the amino acids KDEL at the carboxy terminus of a proteinaceous component of the cell-targeting molecule (see e.g. PCT/US2015/19684).

### D. Linkages Connecting Components of the Cell-Targeting Molecules of the Invention

Individual cell-targeting binding regions, Shiga toxin effector polypeptides, CD8+ T-cell epitopes, and/or other components of the cell-targeting molecules present invention may be suitably linked to each other via one or more linkers well known in the art and/or described herein (see e.g., WO 2014/164693; WO 2015/113005; WO 2015/113007; WO 2015/138452; WO 2015/191764). Individual polypeptide subcomponents of the binding regions, e.g. heavy chain variable regions (VH), light chain variable regions (VL), CDR, and/or ABR regions, may be suitably linked to each other via one or more linkers well known in the art and/or described herein. Proteinaceous components of the invention, e.g., multi-chain binding regions, may be suitably linked to each other or other polypeptide components of the invention via one or more linkers well known in the art. Peptide components of the invention, e.g., a heterologous, CD8+ T-cell epitope-peptide, may be suitably linked to another component of the invention via one or more linkers, such as a proteinaceous linker, which is well known in the art.

Suitable linkers are generally those which allow each polypeptide component of the present invention to fold with a three-dimensional structure very similar to the polypeptide components produced individually without any linker or another component associated with it. Suitable linkers include single amino acids, peptides, polypeptides, and linkers lacking any of the aforementioned, such as various non-proteinaceous carbon chains, whether branched or cyclic.

Suitable linkers may be proteinaceous and comprise one or more amino acids, peptides, and/or polypeptides. Proteinaceous linkers are suitable for both recombinant fusion proteins and chemically linked conjugates. A proteinaceous linker typically has from about 2 to about 50 amino acid residues, such as, e.g., from about 5 to about 30 or from about 6 to about 25 amino acid residues. The length of the linker selected will depend upon a variety of factors, such as, e.g., the desired property or properties for which the linker is being selected. In certain embodiments, the linker is proteinaceous and is linked near the terminus of a protein component of the present invention, typically within about 20 amino acids of the terminus.

Suitable linkers may be non-proteinaceous, such as, e.g. chemical linkers.

Suitable methods for linkage of the components of the cell-targeting molecules of the present invention may be by any method presently known in the art for accomplishing such, as long as the attachment does not substantially impede the binding capability of the cell-targeting binding region and/or when appropriate the desired Shiga toxin effector function(s) as measured by an appropriate assay, including assays described herein. For example, disulfide bonds and thioether bonds may be used to link two or more proteinaceous components of a cell-targeting molecule of the present invention.

For the purposes of the cell-targeting molecules of the present invention, the specific order or orientation is not fixed for the components unless stipulated. The arrangement of the Shiga toxin effector polypeptide(s), heterologous, CD8+ T-cell epitope(s), the binding region(s), and any optional linker(s), in relation to each other or the entire cell-targeting molecule is not fixed (see e.g. FIG. 1) unless specifically noted. In general, the components of the cell-targeting molecules of the present invention may be arranged in any order provided that the desired activity(ies) of the binding region, Shiga toxin effector polypeptide, and heterologous, CD8+ T-cell epitope are not eliminated.

### II. Examples of Specific Structural Variations of the Cell-Targeting Molecules of the Present Invention

The cell-targeting molecules of the present invention comprise a Shiga toxin A Subunit effector polypeptide, a cell-targeting binding region, and a heterologous, CD8+ T-cell epitope-peptide. A cell-targeting molecule with the ability to deliver a CD8+ T-cell epitope to the MHC class I presentation pathway of a target cell may be created, in principle, by linking any heterologous, CD8+ T-cell epitope-peptide to any combination of cell-targeting binding region and Shiga toxin A Subunit effector polypeptide as long as the resulting cell-targeting molecule has a cellular internalization capability (such as, e.g., via endocytosis) provided by at least the Shiga toxin effector, the cell-targeting moiety, or the structural combination of them together, and as long as the Shiga toxin effector polypeptide component or the cell-targeting molecule structure as a whole, provides, once inside a target cell, sufficient subcellular routing to a subcellular compartment competent for delivery of the T-cell epitope-peptide to the MHC class I presentation pathway of the target cell, such as, e.g., to the cytosol or the endoplasmic reticulum (ER).

The cell-targeting molecules of the present invention each comprise at least one Shiga toxin A Subunit effector polypeptide derived from at least one A Subunit of a member of the Shiga toxin family. In certain embodiments, the Shiga toxin effector polypeptide of the cell-targeting molecule of the present invention comprises or consists essentially of a truncated Shiga toxin A Subunit. Truncations of Shiga toxin A Subunits might result in the deletion of an entire epitope(s) and/or epitope region(s), B-cell epitopes, CD4+ T-cell epitopes, and/or furin-cleavage sites without affecting Shiga toxin effector functions, such as, e.g., catalytic activity and cytotoxicity. The smallest Shiga toxin A Subunit fragment shown to exhibit full enzymatic activity was a polypeptide composed of residues 1-239 of Sltl A (LaPointe P et al., J Biol Chem 280: 233 10-18 (2005)). The smallest Shiga toxin A Subunit fragment shown to exhibit significant enzymatic activity was a polypeptide composed of residues 75-247 of StxA (Al-Jaufy A et al., Infect Immun 62: 956-60 (1994)).

Although Shiga toxin effector polypeptides of the present invention may commonly be smaller than the full-length Shiga toxin A Subunit, the Shiga toxin effector polypeptide of a cell-targeting molecule of the present invention may need to maintain the polypeptide region from amino acid position 77 to 239 (SLT-1A (SEQ ID NO: 1) or StxA (SEQ ID NO:2)) or the equivalent in other A Subunits of members of the Shiga toxin family {e.g. 77 to 238 of (SEQ ID NO:3)). For example, in certain embodiments of the molecules of the present invention, the Shiga toxin effector polypeptides of the present invention derived from SLT-1A may comprise or consist essentially of amino acids 75 to 251 of SEQ ID NO:1, 1 to 241 of SEQ ID NO:1, 1 to 251 of SEQ ID NO: 1, or amino acids 1 to 261 of SEQ ID NO: 1. Similarly, Shiga toxin effector polypeptides derived from StxA may comprise or consist essentially of amino acids 75 to 251 of SEQ ID NO:2, 1 to 241 of SEQ ID NO:2, 1 to 251 of SEQ ID NO:2, or amino acids 1 to 261 of SEQ ID NO:2. Additionally, Shiga toxin effector polypeptides derived from SLT-2 may comprise or consist essentially of amino acids 75 to 251 of SEQ ID NO:3, 1 to 241 of SEQ ID NO:3, 1 to 251 of SEQ ID NO:3, or amino acids 1 to 261 of SEQ ID NO:3.

Although derived from a wild-type Shiga toxin A Subunit polypeptide, for certain embodiments of the molecules of the present invention, the Shiga toxin effector polypeptide differs from a naturally occurring Shiga toxin A Subunit by up to 1, 2, 3, 4, 5, 6, 7, 8, 9, 10, 15, 20, 25, 30, 35, 40 or more amino acid residues (but by no more than that which retains at least 85%, 90%, 95%, 99%, or more amino acid sequence identity).

The invention further provides variants of the cell-targeting molecules of the present invention, wherein the Shiga toxin effector polypeptide differs from a naturally occurring Shiga toxin A Subunit by only or up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a molecule of the present invention derived from an A Subunit of a member of the Shiga toxin family may comprise additions, deletions, truncations, or other alterations from the original sequence as long as at least 85%, 90%, 95%, 99% or more amino acid sequence identity is maintained to a naturally occurring Shiga toxin A Subunit, such as, e.g., wherein there is a disrupted, furin-cleavage motif at the carboxy terminus of a Shiga toxin A 1 fragment derived region.

Accordingly, in certain embodiments, the Shiga toxin effector polypeptide of a molecule of the present invention comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga toxin A Subunit, such as SLT-1A (SEQ ID NO:1), StxA (SEQ ID NO:2), and/or SLT-2A (SEQ ID NO:3), such as, e.g., wherein there is a disrupted, furin-cleavage motif at the carboxy terminus of a Shiga toxin A1 fragment derived region.

Optionally, either a full-length or a truncated version of the Shiga toxin effector polypeptide of a cell-targeting molecule of the present of invention, wherein the Shiga toxin derived polypeptide comprises one or more mutations (e.g. substitutions, deletions, insertions, or inversions) as compared to a naturally occurring Shiga toxin A Subunit. It is preferred in certain embodiments of the invention that the Shiga toxin effector polypeptides have sufficient sequence identity to a wild-type Shiga toxin A Subunit to retain cytotoxicity after entry into a cell, either by well-known methods of host cell transformation, transfection, infection or induction, or by internalization mediated by a cell-targeting binding region linked with the Shiga toxin effector polypeptide. The most critical residues for enzymatic activity and/or cytotoxicity in the Shiga toxin A Subunits have been mapped to the following residue-positions: asparagine-75, tyrosine-77, glutamate-167, arginine-170, and arginine-176 among others (Di R et al., Toxicon 57: 525-39 (2011)). In any one of the embodiments of the invention, the Shiga toxin effector polypeptides may preferably but not necessarily maintain one or more conserved amino acids at positions, such as those found at positions 77, 167, 170, and 176 in StxA, SLT-1A, or the equivalent conserved position in other members of the Shiga toxin family which are typically required for potent cytotoxic activity. The capacity of a cytotoxic cell-targeting molecule of the present invention to cause cell death, e.g. its cytotoxicity, may be measured using any one or more of a number of assays well known in the art.

It should be noted that cell-targeting molecules of the invention that comprise Shiga toxin effector polypeptides with even considerable reductions in the Shiga toxin effector function(s) of subcellular routing as compared to wild-type Shiga toxin effector polypeptides may still b e capable of delivering their heterologous, CD8+ T-cell epitope-peptide components to the MHC class I presentation pathway of a target cell, such as, e.g., in sufficient quantities to induce an immune response involving intercellular engagement of a CD8+ immune cell and/or to detect certain subcellular compartments of specific cell-types as even presentation of a single pMHC I complex is sufficient for intercellular engagement of a presenting cell by a CTL for cytolysis (Sykulev Y et al., Immunity 4: 565-71 (1996)).

In certain embodiments of the cell-targeting molecule of the present invention, the Shiga toxin effector polypeptide comprises (1) a Shiga toxin A1 fragment derived polypeptide having a carboxy-terminus and (2) a disrupted furin-cleavage motif at the carboxy-terminus of the Shiga toxin A1 fragment derived polypeptide. The carboxy-terminus of a Shiga toxin A1 fragment derived polypeptide may be identified by the skilled worker by using techniques known in the art, such as, e.g., by using protein sequence alignment software to identify (i) a furin-cleavage motif conserved with a naturally occurring Shiga toxin, (ii) a surface exposed, extended loop conserved with a naturally occurring Shiga toxin, and/or (iii) a stretch of amino acid residues which are predominantly hydrophobic (i.e. a hydrophobic “patch”) that may be recognized by the ERAD system.

The Shiga toxin effector polypeptide of the cell-targeting molecule of the present invention (1) may completely lack any furin-cleavage motif at a carboxy-terminus of its Shiga toxin A1 fragment region and/or (2) comprise a disrupted furin-cleavage motif at the carboxy-terminus of its Shiga toxin A1 fragment region and/or region derived from the carboxy-terminus of a Shiga toxin A1 fragment. A disruption of a furin-cleavage motif includes various alterations to an amino acid residue in the furin-cleavage motif, such as, e.g., a post-translation modification(s), an alteration of one or more atoms in an amino acid functional group, the addition of one or more atoms to an amino acid functional group, the association to a non-proteinaceous moiety(ies), and/or the linkage to an amino acid residue, peptide, polypeptide such as resulting in a branched proteinaceous structure. For example, the linkage of a heterologous, CD8+ T-cell epitope-peptide to the carboxy-terminus of the Shiga toxin A1 fragment region of a wild-type Shiga toxin effector polypeptide may result in reduced furin-cleavage of the Shiga toxin effector polypeptide as compared to a reference molecule lacking the linked epitope-peptide.

Protease-cleavage resistant, Shiga toxin effector polypeptides may be created from a Shiga toxin effector polypeptide and/or Shiga toxin A Subunit polypeptide, whether naturally occurring or not, using a method described herein, described in WO 2015/191764, and/or known to the skilled worker, wherein the resulting molecule still retains one or more Shiga toxin A Subunit functions.

For purposes of the present invention with regard to a furin-cleavage site or furin-cleavage motif, the term “disruption” or “disrupted” refers to an alteration from the naturally occurring furin-cleavage site and/or furin-cleavage motif, such as, e.g., a mutation, that results in a reduction in furin-cleavage proximal to the carboxy-terminus of a Shiga toxin A1 fragment region, or identifiable region derived thereof, as compared to the furin-cleavage of a wild-type Shiga toxin A Subunit or a polypeptide derived from a wild-type Shiga toxin A Subunit comprising only wild-type polypeptide sequences. An alteration to an amino acid residue in the furin-cleavage motif includes a mutation in the furin-cleavage motif, such as, e.g., a deletion, insertion, inversion, substitution, and/or carboxy-terminal truncation of the furin-cleavage motif, as well as a post-translation modification, such as, e.g., as a result of glycosylation, albumination, and the like which involve conjugating or linking a molecule to the functional group of an amino acid residue. Because the furin-cleavage motif is comprised of about twenty, amino acid residues, in theory, alterations, modifications, mutations, deletions, insertions, and/or truncations involving one or more amino acid residues of any one of these twenty positions might result in a reduction of furin-cleavage sensitivity (Tian S et al., Sci Rep 2:261 (2012)).

For purposes of the present invention, a “disrupted furin-cleavage motif is furin-cleavage motif comprising an alteration to one or more amino acid residues derived from the 20 amino acid residue region representing a conserved, furin-cleavage motif found in native, Shiga toxin A Subunits at the junction between the Shiga toxin A1 fragment and A2 fragment regions and positioned such that furin cleavage of a Shiga toxin A Subunit results in the production of the A1 and A2 fragments; wherein the disrupted furin-cleavage motif exhibits reduced furin cleavage in an experimentally reproducible way as compared to a reference molecule comprising a wild-type, Shiga toxin A1 fragment region fused to a carboxy-terminal polypeptide of a size large enough to monitor furin cleavage using the appropriate assay known to the skilled worker and/or described herein.

Examples of types of mutations which can disrupt a furin-cleavage site and furin-cleavage motif are amino acid residue deletions, insertions, truncations, inversions, and/or substitutions, including substitutions with non-standard amino acids and/or non-natural amino acids. In addition, furin-cleavage sites and furin-cleavage motifs can be disrupted by mutations comprising the modification of an amino acid by the addition of a covalently-linked structure which masks at least one amino acid in the site or motif, such as, e.g., as a result of PEGylation, the coupling of small molecule adjuvants, and/or site-specific albumination.

If a furin-cleavage motif has been disrupted by mutation and/or the presence of non-natural amino acid residues, certain disrupted furin-cleavage motifs may not be easily recognizable as being related to any furin-cleavage motif; however, the carboxy-terminus of the Shiga toxin A1 fragment derived region will be recognizable and will define where the furin-cleavage motif would be located were it not disrupted. For example, a disrupted furin-cleavage motif may comprise less than the twenty, amino acid residues of the furin-cleavage motif due to a carboxy-terminal truncation as compared to a Shiga toxin A Subunit and/or Shiga toxin A1 fragment.

In certain embodiments of the cell-targeting molecule of the present invention, the Shiga toxin effector polypeptide comprises (1) a Shiga toxin A1 fragment derived polypeptide having a carboxy-terminus and (2) a disrupted furin-cleavage motif at the carboxy-terminus of the Shiga toxin A1 fragment polypeptide region; wherein the cell-targeting molecule is more furin-cleavage resistant as compared to a reference molecule, such as, e.g., a related molecule comprising only a wild-type Shiga toxin polypeptide component(s) or only a Shiga toxin effector polypeptide component (s) having a conserved, furin-cleavage motif between A1 and A2 fragments. For example, a reduction in furin cleavage of one molecule compared to a reference molecule may be determined using an in vitro, furin-cleavage assay described in WO 2015/191764, conducted using the same conditions, and then performing a quantitation of the band density of any fragments resulting from cleavage to quantitatively measure in change in furin cleavage.

In general, the protease-cleavage sensitivity of a cell-targeting molecule of the present invention is tested by comparing it to the same molecule having its furin-cleavage resistant, Shiga toxin effector polypeptide component(s) replaced with a wild-type, Shiga toxin effector polypeptide component(s) comprising a Shiga toxin A1 fragment. In certain embodiments, the molecules of the present invention comprising a disrupted furin-cleavage motif exhibit a reduction in in vitro furin cleavage of 30%, 40%, 50%, 60%, 70%, 80%, 90%, 95%, 97%, 98% or greater compared to a reference molecule comprising a wild-type, Shiga toxin A1 fragment fused at its carboxy-terminus to a peptide or polypeptide.

In certain embodiments of the cell-targeting molecules of the present invention, the Shiga toxin effector polypeptide comprises a disruption in one or more amino acids derived from the conserved, highly accessible, protease-cleavage sensitive loop of Shiga toxin A Subunits. In certain further embodiments, the Shiga toxin effector polypeptide comprising a disrupted furin-cleavage motif comprising a mutation in the surface-exposed, protease sensitive loop conserved among Shiga toxin A Subunits. In certain further embodiments, the mutation reduces the surface accessibility of certain amino acid residues within the loop such that furin-cleavage sensitivity is reduced.

In certain embodiments, the disrupted furin-cleavage motif of a Shiga toxin effector polypeptide of a cell-targeting molecule of the present invention comprises a disruption in terms of existence, position, or functional group of one or both of the consensus amino acid residues PI and P4, such as, e.g., the amino acid residues in positions 1 and 4 of the minimal furin-cleavage motif R/Y-x-x-R. For example, mutating one or both of the two arginine residues in the minimal, furin consensus site R-x-x-R to alanine will disrupt a furin-cleavage motif by reducing or abolishing furin-cleavage at that site. For example, mutating one or both arginine residues to histidine will cause reduction in furin cleavage. Similarly, amino acid residue substitutions of one or both of the arginine residues in the minimal furin-cleavage motif R-x-x-R to any non-conservative amino acid residue known to the skilled worker will reduced the furin-cleavage sensitivity of the motif. In particular, amino acid residue substitutions of arginine to any non-basic amino acid residue which lacks a positive charge, such as, e.g., A, G, P, S, T, D, E, Q, N, C, I, L, M, V, F, W, and Y, will result in a disrupted furin-cleavage motif.

In certain embodiments, the disrupted furin-cleavage motif of a Shiga toxin effector polypeptide of the present invention comprises a disruption in the spacing between the consensus amino acid residues P4 and PI in terms of the number of intervening amino acid residues being other than two, and, thus, changing either P4 and/or PI into a different position and eliminating the P4 and/or PI designations. For example, deletions within the furin-cleavage motif of the minimal furin-cleavage site or the core, furin-cleavage motif will reduce the furin-cleavage sensitivity of the furin-cleavage motif.

In certain embodiments of the cell-targeting molecules of the present invention, the disrupted furin-cleavage motif comprises one or more amino acid residue substitutions, as compared to a wild-type, Shiga toxin A Subunit. In certain further embodiments, the disrupted furin-cleavage motif comprises one or more amino acid residue substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In certain embodiments of the cell-targeting molecules of the present invention, the disrupted furin-cleavage motif comprises an un-disrupted, minimal furin-cleavage site R/Y-x-x-R but instead comprises a disrupted flanking region, such as, e.g., amino acid residue substitutions in one or more amino acid residues in the furin-cleavage motif flanking regions natively position at, e.g., 241-247 and/or 252-259. In certain further embodiments, the disrupted furin cleavage motif comprises a substitution of one or more of the amino acid residues located in the Pi-p6 region of the furin-cleavage motif; mutating P1′ to a bulky amino acid, such as, e.g., R, W, Y, F, and H; and mutating P2′ to a polar and hydrophilic amino acid residue; and substituting one or more of the amino acid residues located in the P1′-P6′ region of the furin-cleavage motif with one or more bulky and hydrophobic amino acid residues

In certain embodiments of the cell-targeting molecules of the present invention, the disrupted furin-cleavage motif comprises a deletion, insertion, inversion, and/or substitution of at least one amino acid residue within the furin-cleavage motif relative to a wild-type Shiga toxin A Subunit. In certain further embodiments, the disrupted furin-cleavage motif comprises a disruption of the amino acid sequence natively positioned at 249-251 of the A Subunit of Shiga-like toxin 1 (SEQ ID NO: 1) or Shiga toxin (SEQ ID NO:2), or at 247-250 of the A Subunit of Shiga-like toxin 2 (SEQ ID NO:3) or the equivalent position in a conserved Shiga toxin effector polypeptide and/or non-native Shiga toxin effector polypeptide sequence. In certain further embodiments, the disrupted furin-cleavage motif comprises a disruption which comprises a mutation, such as, e.g., an amino acid substitution to a non-standard amino acid or an amino acid with a chemically modified side chain. In certain further embodiments, the disrupted furin-cleavage motif comprises comprise a disruption which comprises a deletion of at least one amino acid within the furin-cleavage motif. In certain further embodiments, the disrupted furin-cleavage motif comprises the deletion of nine, ten, eleven, or more of the carboxy-terminal amino acid residues within the furin-cleavage motif. In these embodiments, the disrupted furin-cleavage motif will not comprise a furin-cleavage site or a minimal furin-cleavage motif. In other words, certain embodiments lack a furin-cleavage site at the carboxy-terminus of the A1 fragment region.

In certain embodiments of the cell-targeting molecules of the present invention, the disrupted furin-cleavage motif comprises an amino acid residue deletion and an amino acid residue substitution as well as a carboxy-terminal truncation as compared to a wild-type, Shiga toxin A Subunit. In certain further embodiments, the disrupted furin-cleavage motif comprises one or more amino acid residue deletions and substitutions within the minimal furin-cleavage site R/Y-x-x-R.

In certain embodiments of the cell-targeting molecules of the present invention, the disrupted furin-cleavage motif comprises both an amino acid substitution within the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate.

In certain embodiments of the cell-targeting molecules of the present invention, the disrupted furin-cleavage motif comprises both an amino acid residue deletion and an amino acid residue substitution as compared to a wild-type, Shiga toxin A Subunit. In certain further embodiments, the disrupted furin-cleavage motif comprises one or more amino acid residue deletions and substitutions within the minimal furin-cleavage site R/Y-x-x-R.

In certain embodiments of the cell-targeting molecule of the present invention, the disrupted furin-cleavage motif comprises an amino acid residue deletion, an amino acid residue insertion, an amino acid residue substitution and/or a carboxy-terminal truncation as compared to a wild-type, Shiga toxin A Subunit.

The cell-targeting molecules of the present invention each comprise one or more, heterologous, CD8+ T-cell epitope-peptides. In certain embodiments, the CD8+ T-cell epitope-peptide is an antigenic and/or immunogenic epitope in a human. In certain embodiments, the CD8+ T-cell epitope-peptide component of the cell-targeting molecules of the present invention comprises or consists essentially of an 8-11 amino acid long peptide derived from a molecule of a microbial pathogen which infects humans, such as, e.g., an antigen from a virus that infects humans. In certain further embodiments, the CD8+ T-cell epitope-peptide component of the cell-targeting molecules of the invention comprises or consists essentially of any one of the peptides shown in SEQ ID NOs: 4-12.

In certain embodiments of the cell-targeting molecules of the present invention, the heterologous, CD8+ T-cell epitope-peptide is linked to the cell-targeting molecule via a disulfide bond. In certain further embodiments, the disulfide bond is a cysteine to cysteine disulfide bond.

In certain embodiments of the cell-targeting molecules of the present invention, the heterologous, CD8+ T-cell epitope-peptide is linked to the cell-targeting molecule via a disulfide bond involving the functional group of a cysteine residue of a Shiga toxin effector polypeptide component of the cell-targeting molecule, such as, e.g., C241 of SLT-2A (SEQ ID NO:3) or 242 of StxA (SEQ ID NO:2) or SLT-1A (SEQ ID NO: 1). In certain further embodiments, the cysteine residue is positioned carboxy-terminal to the carboxy terminus of the Shiga toxin A1 fragment region of the Shiga toxin effector polypeptide (e.g., the cysteine residue C260 of SLT-2A (SEQ ID NO:3) or C261 of StxA (SEQ ID NO:2) or SLT-1A (SEQ ID NO: 1)).

The cell-targeting molecules of the present invention comprise at least one cell-targeting binding region. Among certain embodiments of the cell-targeting molecules of the present invention, the binding region is derived from an immunoglobulin-type polypeptide selected for specific and high-affinity binding to a surface antigen on the cell surface of a cancer or tumor cell, where the antigen is restricted in expression to cancer or tumor cells (see Glokler J et al., Molecules 15: 2478-90 (2010); Liu Y et al., Lab Chip 9: 1033-6 (2009). In accordance with other embodiments, the binding region is selected for specific and high-affinity binding to a surface antigen on the cell surface of a cancer cell, where the antigen is over-expressed or preferentially expressed by cancer cells as compared to non-cancer cells. Some representative target biomolecules include, but are not limited to, the following enumerated targets associated with cancers and/or specific immune cell-types.

Many immunoglobulin-type binding regions that bind with high affinity to extracellular epitopes associated with cancer cells are known to the skilled worker, such as binding regions that bind any one of the following target biomolecules: annexin AI, B3 melanoma antigen, B4 melanoma antigen, CD2, CD3, CD4, CD 19, CD20 (B-lymphocyte antigen protein CD20), CD22, CD25 (interleukin-2 receptor IL2R), CD30 (TNFRSF8), CD37, CD38 (cyclic ADP ribose hydrolase), CD40, CD44 (hyaluronan receptor), ITGAV (CD51), CD56, CD66, CD70, CD71 (transferrin receptor), CD73, CD74 (ULA-DR antigens-associated invariant chain), CD79, CD98, endoglin (END, CD 105), CD 106 (VCAM-1), CD 138, chemokine receptor type 4 (CDCR-4, fusin, CD 184), CD200, insulin-like growth factor 1 receptor (CD221), mucin 1 (MUC1, CD227, CA6, CanAg), basal cell adhesion molecule (B-CAM, CD239), CD248 (endosialin, TEM1), tumor necrosis factor receptor 10b (TNFRSF10B, CD262), tumor necrosis factor receptor 13B (TNFRSF13B, TACI, CD276), vascular endothelial growth factor receptor 2 (KDR, CD309), epithelial cell adhesion molecule (EpCAM, CD326), human epidermal growth factor receptor 2 (HER2, Neu, ErbB2, CD340), cancer antigen 15-3 (CA15-3), cancer antigen 19-9 (CA 19-9), cancer antigen 125 (CA125, MUC16), CA242, carcinoembryonic antigen-related cell adhesion molecules (e.g. CEACAM3 (CD66d) and CEACAM5), carcinoembryonic antigen protein (CEA), choline transporter-like protein 4 (SLC44A4), chondroitin sulfate proteoglycan 4 (CSP4, MCSP, NG2), CTLA4, delta-like proteins (e.g. DLL3, DLL4), ectonucleotide pyrophosphatase/phosphodiesterase proteins (e.g. ENPP3), endothelin receptors (ETBRs), epidermal growth factor receptor (EGFR, ErbBl), folate receptors (FOLRs, e.g. FRa), G-28, ganglioside GD2, ganglioside GD3, HLA-DrlO, HLA-DRB, human epidermal growth factor receptor 1 (HER1), HER3/ErbB-3, Ephrin type-B receptor 2 (EphB2), epithelial cell adhesion molecule (EpCAM), fibroblast activation protein (FAP/seprase), guanylyl cyclase c (GCC), insulin-like growth factor 1 receptor (IGFIR), interleukin 2 receptor (IL-2R), interleukin 6 receptor (IL-6R), integrins alpha-V beta-3 (αvβ3), integrins alpha-V beta-5 (αvβ5), integrins alpha-5 beta-1 (α₅β_{ι}), L6, zinc transporter (LIV-1), MPG, melanoma-associated antigen 1 protein (MAGE-1), melanoma-associated antigen 3 (MAGE-3), mesothelin (MSLN), metalloreductase STEAP1, MPG, MS4A, NaPi2b, nectins (e.g. nectin-4), p21, p9′7, polio virus receptor-like 4 (PVRL4), protease-activated-receptors (such as PARI), prostate-specific membrane antigen proteins (PSMAs), SLIT and NTRK-like proteins (e.g. SLITRK6), Thomas-Friedenreich antigen, transmembrane glycoprotein (GPNMB), trophoblast glycoproteins (TPGB, 5T4, WAIF1), and tumor-associated calcium signal transducers (TACSTDs, e.g. Trop-2, EGP-1, etc.) (see e.g. Lui B et al., Cancer Res 64: 704-10 (2004); Novellino L et al., Cancer Immunol Immunother 54: 187-207 (2005); Bagley R et al., Int J Oncol 34: 619-27 (2009); Gerber H et al., mAbs 1: 247-53 (2009); Beck A et al., Nat Rev Immunol 10: 345-52 (2010); Andersen J et al., J Biol Chem 287: 22927-37 (2012); Nolan-Stevaux O et al., PLoS One 7: e50920 (2012); Rust S et al., Mol Cancer 12: 11 (2013)). This list of target biomolecules is intended to be non-limiting. It will be appreciated by the skilled worker that any desired target biomolecule associated with a cancer cell or other desired cell-type may be used to design or select a binding region which may be suitable for use as a component of a cell-targeting molecule of the present invention.

Examples of other target biomolecules which are strongly associated with cancer cells and are bound with high-affinity by a known immunoglobulin-type binding region include BAGE proteins (B melanoma antigens), basal cell adhesion molecules (BCAMs or Lutheran blood group glycoproteins), bladder tumor antigen (BTA), cancer-testis antigen NY-ESO-1, cancer-testis antigen LAGE proteins, CD 19 (B-lymphocyte antigen protein CD 19), CD21 (complement receptor-2 or complement 3d receptor), CD26 (dipeptidyl peptidase-4, DPP4, or adenosine deaminase complexing protein 2), CD33 (sialic acid-binding immunoglobulin-type lectin-3), CD52 (CAMPATH-1 antigen), CD56, CSl (SLAM family number 7 or SLAMF7), cell surface A33 antigen protein (gpA33), Epstein-Barr virus antigen proteins, GAGE/PAGE proteins (melanoma associated cancer/testis antigens), hepatocyte growth factor receptor (HGFR or c-Met), MAGE proteins, melanoma antigen recognized by T-cells 1 protein (MART-1/MelanA, MARTI), mucins, Preferentially Expressed Antigen of Melanoma (PRAME) proteins, prostate specific antigen protein (PSA), prostate stem cell antigen protein (PSCA), Receptor for Advanced Glycation Endroducts (RAGE), tumor-associated glycoprotein 72 (TAG-72), vascular endothelial growth factor receptors (VEGFRs), and Wilms' tumor antigen.

Examples of other target biomolecules which are strongly associated with cancer cells are carbonic anhydrase IX (CA9/CAIX), claudin proteins (CLDN3, CLDN4), ephrin type-A receptor 3 (EphA3), folate binding proteins (FBP), ganglioside GM2, insulin-like growth factor receptors, integrins (such as CD1 1a-c), receptor activator of nuclear factor kappa B (RANK), receptor tyrosine-protein kinase erB-3, tumor necrosis factor receptor 10A (TRAIL-R1/DR4), tumor necrosis factor receptor 10B (TRAIL-R2), tenascin C, and CD64 (FcyRI) (see Hough C et al., Cancer Res 60: 6281-7 (2000); Thepen T et al., Nat Biotechnol 18: 48-51 (2000); Pastan I et al., Nat Rev Cancer 6: 559-65 (2006); Pastan, Annu Rev Med 58: 221-37 (2007); Fitzgerald D et al., Cancer Res l 1: 6300-9 (2011); Scott A et al., Cancer Immun 12: 14-22 (2012)). This list of target biomolecules is intended to be non-limiting.

In addition, there are numerous other examples of contemplated, target biomolecules, such as, e.g., ADAM metalloproteinases (e.g. ADAM-9, ADAM-10, ADAM-12, ADAM-15, ADAM-17), ADP-ribosyltransferases (ART1, ART4), antigen F4/80, bone marrow stroma antigens (BST1, BST2), break point cluster region-c-abl oncogene (BCR-ABL) proteins, C3aR (complement component 3a receptors), CD7, CD 13, CD 14, CD 15 (Lewis X or stage-specific embryonic antigen 1), CD23 (FC epsilon RII), CD45 (protein tyrosine phosphatase receptor type C), CD49d, CD53, CD54 (intercellular adhesion molecule 1), CD63 (tetraspanin), CD69, CD80, CD86, CD88 (complement component 5a receptor 1), CD115 (colony stimulating factor 1 receptor), IL-1R (interleukin-1 receptor), CD123 (interleukin-3 receptor), CD129 (interleukin 9 receptor), CD183 (chemokine receptor CXCR3), CD191 (CCR1), CD193 (CCR3), CD195 (chemokine receptor CCR5), CD203c, CD225 (interferon-induced transmembrane protein 1), CD244 (Natural Killer Cell Receptor 2B4), CD282 (Toll-like receptor 2), CD284 (Toll-like receptor 4), CD294 (GPR44), CD305 (leukocyte-associated immunoglobulin-like receptor 1), ephrin type-A receptor 2 (EphA2), FceRIa, galectin-9, alpha-fetoprotein antigen 17-A1 protein, human aspartyl (asparaginyl) beta-hydroxylase (HAAH), immunoglobulin-like transcript ILT-3, lysophosphatidlglycerol acyltransferase 1 (LPGAT1/IAA0205), lysosome-associated membrane proteins (LAMPs, such as CD 107), melanocyte protein PMEL (gplOO), myeloid-related protein-14 (mrp-14), NKG2D ligands (e.g., MICA, MICB, ULBP1, ULBP2, UL-16-binding proteins, H-60s, Rae-ls, and homologs thereof), receptor tyrosine-protein kinase erbB-3, SART proteins, scavenger receptors (such as CD64 and CD68), Siglecs (sialic acid-binding immunoglobulin-type lectins), syndecans (such as SDC1 or CD 138), tyrosinase, tyrosinease-related protein 1 (TRP-1), tyrosinease-related protein 2 (TRP-2), tyrosinase associated antigen (TAA), APO-3, BCMA, CD2, CD3, CD4, CD8, CD 18, CD27, CD28, CD29, CD41, CD49, CD90, CD95 (Fas), CD 103, CD 104, CD134 (OX40), CD137 (4-1BB), CD152 (CTLA-4), chemokine receptors, complement proteins, cytokine receptors, histocompatibility proteins, ICOS, interferon-alpha, interferon-beta, c-myc, osteoprotegerin, PD-1, RANK, TACI, TNF receptor superfamily member (TNF-R1, TNFR-2), Apo2/TRAIL-R1, TRAIL-R2, TRAIL-R3, and TRAIL-R4 (see Scott A et al., Cancer Immunity 12: 14 (2012); Cheever M et al., Clin Cancer Res 15: 5323-37 (2009)), for target biomolecules and note the target biomolecules described therein are non-limiting examples).

In certain embodiments, the binding region comprises or consists essentially of an immunoglobulin-type binding region capable of specifically binding with high-affinity to the cellular surface of a cell-type of the immune system. For example, immunoglobulin-type binding domains are known which bind to immune cell surface factors, such as, e.g., CD1, CD2, CD3, CD4, CD5, CD6, CD7, CD8, CD9, CDIO, CD11, CD12, CD13, CD14, CD15, CD16, CD17, CD18, CD19, CD20, CD21, CD22, CD23, CD24, CD25, CD26, CD27, CD28, CD29, CD30, CD31, CD33, CD34, CD35, CD36, CD37, CD38, CD40, CD41, CD56, CD61, CD62, CD66, CD95, CD117, CD123, CD235, CD146, CD326, interleukin-1 receptor (IL-1R), interleukin-2 receptor (IL-2R), receptor activator of nuclear factor kappa B (RANKL), SLAM-associated protein (SAP), and TNFSF 18 (tumor necrosis factor ligand 18 or GITRL).

For further examples of target biomolecules and binding regions envisioned for use in the molecules of the present invention, see WO 2005/092917, WO 2007/033497, US2009/0156417, JP4339511, EP1727827, DE602004027168, EP1945660, JP4934761, EP2228383, US2013/0196928, WO 2014/164680, WO 2014/164693, WO 2015/138435, WO 2015/138452, WO 2015/113005, WO 2015/113007, WO 2015/191764, US20150259428, 62/168,758, 62/168,759, 62/168,760, 62/168,761, 62/168,762, 62/168,763, and PCT/US2016/016580.

Certain embodiments of the cell-targeting molecules of the present invention are cytotoxic, cell-targeting, fusion proteins. Certain further embodiments are the cell-targeting molecules which comprise or consist essentially of one of the polypeptides shown in SEQ ID NOs: 13-40, 42, 44-50, 52, 54-58, 60-61, and 72-115.

In certain embodiments, the cell-targeting molecule of the present invention is a fusion protein, such as, e.g. immunotoxins and ligand-toxin fusion. Certain embodiments of the cell-targeting molecules of the present invention are reduced-cytotoxicity or non-cytotoxic, cell-targeting, fusion proteins. Certain further embodiments are the cell-targeting molecules which comprise or consist essentially of one of the polypeptides shown in SEQ ID NOs: 41, 43, 51, 53, and 59. Other further embodiments are the cell-targeting molecules which comprise or consist essentially of one of the polypeptides shown in SEQ ID NOs: 13-40, 42, 44-50, 52, 54-58, 60-61, and 72-115 which further comprises one or more amino acid substitutions in the Shiga toxin effector polypeptide component(s) altering the natively positioned residue selected from the group consisting of: A23 IE, R75A, Y77S, Y 114S, E167D, R170A, R176K and/or W203A in SEQ ID NO: 1, SEQ ID NO:2, or SEQ ID NO:3. or the equivalent amino acid residue in a Shiga toxin A Subunit.

Cell-targeting molecules of the present invention each comprise a cell-targeting binding region which can bind specifically to at least one extracellular target biomolecule in physical association with a cell, such as a target biomolecule expressed on the surface of a cell. This general structure is modular in that any number of diverse cell-targeting moieties may be used as a binding region of a cell-targeting molecule of the present invention. It is within the scope of the present invention to use fragments, variants, and/or derivatives of the cell-targeting molecules of the present invention which contain a functional binding site to any extracellular part of a target biomolecule, and even more preferably capable of binding a target biomolecule with high affinity (e.g. as shown by a KD less than 10⁻⁹ moles/liter). For example, while the invention provides polypeptide sequences that can bind to human proteins, any binding region that binds an extracellular part of a target biomolecule with a dissociation constant (KD) of 10⁻⁵ to 10⁻¹² moles/liter, preferably less than 200 nM, may be substituted for use in making cell-targeting molecules of the invention and methods of the invention.

### III. General Functions of the Cell-Targeting Molecules of the Present Invention

The present invention provides cell-targeting molecules comprising (1) Shiga toxin A Subunit derived, toxin effector polypeptides capable of exhibiting at least one Shiga toxin function and (2) CD8+ T-cell epitope-peptide cargos unrelated to Shiga toxin A Subunits; whereby administration of the cell-targeting molecule to a cell can result in the cell-targeting molecule entering the cell and delivering its heterologous, CD8+ T-cell epitope-peptide cargo to the MHC class I pathway of the target cell. This system is modular, in that any number of diverse binding regions may be used to target diverse cell-types and any number of diverse CD8+ T-cell epitope-peptides may be delivered to target cells. The cell-targeting molecules of the present invention may be used as therapeutic molecules, cytotoxic molecules, cell-labeling molecules, and diagnostic molecules.

For certain embodiments, the cell-targeting molecule of the present invention provides, after administration to a chordate, one or more of the following: 1) potent and selective killing of targeted cells, e.g., infected and/or neoplastic cells, 2) linkage stability between the cell-targeting binding region and the Shiga toxin effector polypeptide while the cell-targeting molecule is present in extracellular spaces (see e.g. WO 2015/191764), 3) low levels of off-target cell deaths and/or unwanted tissue damage (see e.g. WO 2015/191764), and 4) cell-targeted delivery of heterologous, CD8+ T-cell epitopes for presentation by target cells in order to stimulate desirable immune responses, such as, e.g., the recruitment of CD8+ CTLs and the localized release of immuno-stimulatory cytokines at a tissue locus, e.g. a tumor mass. Furthermore, the presentation of delivered, heterologous, CD8+ T-cell epitope-peptides by target cells marks those presenting cells with pMHC Is that can be detected for the purposes of gathering information, such as, e.g., for diagnostic information.

The cell-targeting molecules of the present invention are useful in diverse applications involving, e.g., targeted delivery of a CD8+ T-cell epitope-cargo, immune response stimulation, targeted cell-killing, targeted cell growth inhibition, biological information gathering, and/or remediation of a health condition. The cell-targeting molecules of the present invention are useful as therapeutic and/or diagnostic molecules, such as, e.g., as cell-targeting, nontoxic, delivery vehicles; cell-targeting, cytotoxic, therapeutic molecules; and/or cell-targeting, diagnostic molecules; for examples in applications involving the in vivo targeting of specific cell-types for the diagnosis or treatment of a variety of diseases, including cancers, immune disorders, and microbial infections. Certain cell-targeting molecules of the present invention may be used to treat a chordate afflicted with a tumor or cancer by enhancing the effectiveness of that chordate's anti-tumor immunity, particularly involving CD8+ T-cell mediated mechanisms (see e.g. Ostrand-Rosenberg S, Curr Opin Immunol 6: 722-7 (1994); Pietersz G et al., Cell Mol Life Sci 57: 290-310 (2000); Lazoura E et al., Immunology 119: 306-16 (2006)).

Depending on the embodiment, a cell-targeting molecule of the present invention may have or provide one or more of the following characteristics or functionalities: (1) in vivo stimulation of CD8+ T-cell immune response(s), (2) de-immunization (see e.g. WO 2015/113007), (3) protease-cleavage resistance (see e.g. WO 2015/191764), (4) potent cytotoxicity at certain concentrations, (5) selective cytotoxicity, (6) low off-target toxicity in multicellular organisms at certain doses or dosages (see e.g. WO 2015/191764), and/or (7) intracellular delivery of a cargo consisting of an additional material (e.g. a nucleic acid or detection promoting agent). Certain embodiments of the cell-targeting molecules of the present invention are multi-functional because the molecules have two or more of the characteristics or functionalities described herein. Certain further embodiments of the cell-targeting molecules of the present invention provide all of the aforementioned characteristics and functionalities in a single molecule.

The mechanisms of action of the therapeutic, cell-targeting molecules of the present invention include direct target cell-killing via Shiga toxin effector functions, indirect cell-killing via intercellular immune-cell-mediated processes, and/or educating a recipient's immune system to reject certain cells and tissue loci, e.g. a tumor mass, as a result of “CD8+ T-cell epitope seeding.”

A. Delivery of the Heterologous. CD8+ T-Cell Epitope to the MHC Class I Presentation Pathway of a Target Cell

One of the primary functions of the cell-targeting molecules of the present invention is cell-targeted delivery of one or more heterologous, CD8+ T-cell epitope-peptides for MHC class I presentation by a chordate cell. The cell-targeting molecules of the present invention are modular scaffolds for use as general delivery vehicles of virtually any CD8+ T-cell epitope to virtually any chordate target cell. Targeted delivery requires the cell-targeting molecule to specifically bind to a certain target cell, enter the target cell, and deliver an intact heterologous, CD8+ T-cell epitope-peptide(s) to a subcellular compartment competent for entry into the MHC class I presentation pathway. Delivery of a CD8+ T-cell epitope-peptide to the MHC class I presentation pathway of a target cell using a cell-targeting molecule of the invention can be used to induce the target cell to present the epitope-peptide in association with MHC class I molecules on a cell surface.

By using immunogenic MHC class I epitopes, such as, e.g., from a known viral antigen, as heterologous, CD8+ T-cell epitope-peptide cargos of the cell-targeting molecules of the present invention, the targeted delivery and presentation of immuno-stimulatory antigens may be accomplished in order to stimulate a beneficial function(s) of a chordate immune cell, e.g. in vitro, and/or a chordate immune system in vivo.

In a chordate, the presentation of an immunogenic, CD8+ T-cell epitope by the MHC class I complex can target the presenting cell for killing by CTL-mediated cytolysis, promote immune cells into altering the microenvironment, and signal for the recruitment of more immune cells to the target cell site within the chordate. Certain cell-targeting molecules of the present invention are capable of delivering under physiological conditions its heterologous, CD8+ T-cell epitope-peptide cargo to the MHC class I pathway of a target chordate cell for presentation of the delivered T-cell epitope complexed with a MHC class I molecule. This may be accomplished by exogenous administration of the cell-targeting molecule into an extracellular space, such as, e.g., the lumen of a blood vessel, and then allowing for the cell-targeting molecule to find a target cell, enter the cell, and intracellularly deliver its CD8+ T-cell epitope cargo. The presentation of a CD8+ T-cell epitope by a target cell within a chordate can lead to an immune response(s), including responses directly to the target cell and/or general responses in the tissue locale of the target cell within the chordate.

The applications of these CD8+ T-cell epitope delivery and MHC class I presenting functions of the cell-targeting molecules of the present invention are vast. For example, the delivery of a CD8+ epitope to a cell and the MHC class I presentation of the delivered epitope by the cell in a chordate can cause the intercellular engagement of a CD8+ effector T-cell and may lead to a CTL(s) killing the target cell and/or secreting immuno-stimulatory cytokines.

The cell-targeting molecules of the present invention are capable, upon exogenous administration, of delivering one or more CD8+ T-cell epitopes for MHC class I presentation by a nucleated, chordate cell. For certain embodiments, the cell-targeting molecules of the present invention are capable of binding extracellular target biomolecules associated with the cell surface of particular cell-types and entering those cells. Once internalized within a targeted cell-type, certain embodiments of the cell-targeting molecules of the invention are capable of routing a Shiga toxin effector polypeptide component (whether catalytically active, reduced-cytotoxicity, or non-toxic) to the cytosol of the target cell.

For certain embodiments, the cell-targeting molecule of the present invention is capable, from an extracellular space, of delivering one or more heterologous, CD8+ T-cell epitope-peptides to the proteasome of a target cell. The delivered CD8+ T-cell epitope-peptide can then be proteolytic processed and presented by the MHC class I pathway on the surface of the target cell. For certain embodiments, the cell-targeting molecule of the present invention is capable of delivering the heterologous, CD8+ T-cell epitope-peptide, which is associated with the cell-targeting molecule, to a MHC class I molecule of a cell for presentation of the epitope-peptide by the MHC class I molecule on a surface of the cell. For certain embodiments, upon contacting a cell with the cell-targeting molecule of the present invention, the cell-targeting molecule is capable of delivering the heterologous, CD8+ T-cell epitope-peptide, which is associated with the cell-targeting molecule, to a MHC class I molecule of the cell for presentation of the epitope-peptide by the MHC class I molecule on a surface of the cell.

For certain embodiments, the cell-targeting molecule of the present invention is capable, upon administration to a chordate subject, of targeting delivery of one or more heterologous, CD8+ T-cell epitopes for MHC class I presentation by specific target cells within the subject.

In principle, any CD8+ T-cell epitope-peptide may be chosen for use in a cell-targeting molecule of the present invention. Thus, cell-targeting molecules of the invention are useful for labeling the surfaces of target cells with MHC class I molecules complexed with the epitope-peptide of your choice.

Every nucleated cell in a mammalian organism may be capable of MHC class I pathway presentation of immunogenic, CD8+ T-cell epitope peptides on their cell outer surfaces complexed to MHC class I molecules. In addition, the sensitivity of T-cell epitope recognition is so exquisite that only a few MHC-I peptide complexes are required to be presented to result in an immune response, e.g., even presentation of a single complex can be sufficient for the intercellular engagement of a CD8+ effector T-cell (Sykulev Y et al., Immunity 4: 565-71 (1996)). Target cells of a cell-targeting molecule of the present invention can be virtually any nucleated chordate cell-type and need not be immune cells and/or professional antigen presenting cells. Examples of professional antigen presenting cells include dendritic cells, macrophages, and specialized epithelial cells with functional MHC class II systems. In fact, preferred embodiments of the cell-targeting molecules of the present invention do not target professional antigen presenting cells. One reason is that an undesirable immune response as a result of the administration of the cell-targeting molecule of the present invention would be a humoral response directed to the cell-targeting molecule itself, such as, e.g., an anti-cell-targeting molecule antibody recognizing an epitope in the cell-targeting molecule. Thus, professional antigen presenting cells and certain immune cell-types are not to be targeted by certain embodiments of the cell-targeting molecules of the present invention because the uptake of the cell-targeting molecule of the present invention by these cells may lead to the recognition of CD4+ T-cell and B-cell epitopes present in the cell-targeting molecule, particularly in the Shiga toxin effector polypeptide component(s) and/or an antigenic cargo, but also including in the binding region.

The ability to deliver a CD8+ T-cell epitope by certain embodiments of the cell-targeting molecules of the present invention may be accomplished under varied conditions and in the presence of non-targeted bystander cells, such as, e.g., an ex vivo manipulated target cell, a target cell cultured in vitro, a target cell within a tissue sample cultured in vitro, or a target cell in an in vivo setting like within a multicellular organism.

In order for a cell-targeting molecule of the present invention to function as designed, the cell-targeting molecule must 1) enter a target cell and 2) localize its CD8+ T-cell epitope-peptide cargo to a subcellular location competent for entry into the MHC class I pathway. Commonly, cell-targeting molecules of the invention accomplish target cell internalization via endocytosis. Once the cell-targeting molecule of the invention is internalized, it will typical reside in an early endosomal compartment, such as, e.g., endocytotic vesicle and be destined for destruction in a lysosome or late endosome. A cell-targeting molecule must avoid complete sequestration and degradation such that at least a portion of the cell-target molecule comprising the T-cell epitope-peptide cargo escapes to another subcellular compartment. Furthermore, the target cell should either express a MHC class I molecule or be capable of being induced to express a MHC class I molecule.

The expression of the MHC class I molecule need not be native in order for cell-surface presentation of a heterologous, CD8+ T-cell epitope-peptide (delivered by a cell-targeting molecule of the present invention) complexed with a MHC class I molecule. For certain embodiments of the present invention, the target cell may be induced to express MHC class I molecule(s) using a method known to the skilled worker, such as, e.g., by treatment with IFN-γ.

Commonly, cell-targeting molecules of the invention accomplish MHC class I pathway delivery by localizing their CD8+ T-cell epitope-peptide cargos to proteasomes in cytosolic compartments of target cells. However, for certain embodiments, the cell-targeting molecule of the present invention may deliver a heterologous, CD8+ epitope-peptide to the MHC class I presentation pathway without the epitope-peptide ever entering a cytosolic compartment and/or without the epitope-peptide ever being proteolytically processed by the proteasome.

For certain embodiments of the present invention, the target cell may be induced to express different proteasome subunits and/or proteasome subtypes using a method known to the skilled worker, such as, e.g., by treatment with IFN-γ and/or TNF-α. This can alter the positioning and/or relative efficiency of proteolytic processing of CD8+ epitope peptides delivered into the cell, such as, e.g., by altering the relative levels of peptidase activities of proteasomes and proteasome subtypes.

The CD8+ T-cell epitope delivering functions of the cell-targeting molecules of the present invention can be detected and monitored by a variety of standard methods known in the art to the skilled worker and/or described herein. For example, the ability of cell-targeting molecules of the present invention to deliver a CD8+ T-cell epitope-peptide and drive presentation of the peptide by the MHC class I system of target cells may be investigated using various in vitro and in vivo assays, including, e.g., the direct detection/visualization of MHC class I/peptide complexes (pMHC Is), measurement of binding affinities for the T-cell peptide to MHC class I molecules, and/or measurement of functional consequences of pMHC I presentation on target cells, e.g., by monitoring cytotoxic T-lymphocyte (CTL) responses {see e.g. Examples, infra).

Certain assays to monitor and quantitate the CD8+ T-cell epitope delivering function of the cell-targeting molecules of the present invention involve the direct detection of a specific pMHC Is in vitro or ex vivo. Common methods for direct visualization and quantitation of pMHC Is involve various immuno-detection reagents known to the skilled worker. For example, specific monoclonal antibodies can be developed to recognize a particular pMHC I. Similarly, soluble, multimeric T cell receptors, such as the TCR-STAR reagents (Altor Bioscience Corp., Miramar, Fla., U.S.) can be used to directly visualize or quantitate specific pMHC Is (Zhu X et al., J Immunol 176: 3223-32 (2006); see e.g., Examples, infra). These specific mAbs or soluble, multimeric T-cell receptors may be used with various detection methods, including, e.g. immunohistochemistry, flow cytometry, and enzyme-linked immunosorbent assay (ELISA).

An alternative method for direct identification and quantification of pMHCs involves mass spectrometry analyses, such as, e.g., the ProPresent Antigen Presentation Assay (Prolmmune, Inc., Sarasota, Fla., U.S.) in which peptide-MHC class I complexes are extracted from the surfaces of cells, then the peptides are purified and identified by sequencing mass spectrometry (Falk K et al., Nature 351: 290-6 (1991)).

In certain assays to monitor the CD8+ T-cell epitope delivery and MHC class I presentation function of the cell-targeting molecules of the present invention involve computational and/or experimental methods to monitor MHC class I and peptide binding and stability. Several software programs are available for use by the skilled worker for predicting the binding responses of peptides to MHC class I alleles, such as, e.g., The Immune Epitope Database and Analysis Resource (IEDB) Analysis Resource MHC-I binding prediction Consensus tool (Kim Y et al., Nucleic Acid Res 40: W525-30 (2012)). Several experimental assays have been routinely applied, such as, e.g., cell surface binding assays and/or surface plasmon resonance assays to quantify and/or compare binding kinetics (Miles K et al., Mol Immunol 48: 728-32 (2011)).

Alternatively, measurements of the consequence of pMHC I presentation on the cell surface can be performed by monitoring the cytotoxic T lymphocyte (CTL) response to the specific complex. These measurements by include direct labeling of the CTLs with MHC class I tetramer or pentamer reagents. Tetramers or pentamers bind directly to T cell receptors of a particular specificity, determined by the Major Histocompatibility Complex (MHC) allele and peptide complex. Additionally, the quantification of released cytokines, such as interferon gamma or interleukins by ELISA or enzyme-linked immunospot (ELIspot) is commonly assayed to identify specific CTL responses. The cytotoxic capacity of CTL can be measured using a number of assays, including the classical 51 Chromium (Cr) release assay or alternative non-radioactive cytotoxicity assays (e.g., CytoTox96® non-radioactive kits and CellTox™ CellTiter-GLO® kits available from Promega Corp., Madison, Wis., U.S.), Granzyme B ELISpot, Caspase Activity Assays or LAMP-1 translocation flow cytometric assays. To specifically monitor the killing of target cells, carboxyfluorescein diacetate succinimidyl ester (CFSE) can be used to easily and quickly label a cell population of interest for in vitro or in vivo investigation to monitor killing of epitope specific CSFE labeled target cells (Durward M et al., J Vis Exp 45 pii 2250 (2010)).

In vivo responses to MHC class I presentation can be followed by administering a MHC class I/antigen promoting agent {e.g., a peptide, protein or inactivated/attenuated virus vaccine) followed by challenge with an active agent {e.g. a virus) and monitoring responses to that agent, typically in comparison with unvaccinated controls. Ex vivo samples can be monitored for CTL activity with methods similar to those described previously {e.g. CTL cytotoxicity assays and quantification of cytokine release).

MHC class I presentation in an organism can be followed by reverse immunology. For example, HLA-A, HLA-B, and/or HLA-C molecule complexes are isolated from cells intoxicated with a cell-targeting molecule of the present invention comprising antigen X after lysis using immune affinity {e.g., an anti-MHC I antibody “pulldown” purification) and associated peptides {i.e., the peptides that were bound by the isolated pMHC Is) are recovered from the purified complexes. The recovered peptides are analyzed by sequencing mass spectrometry. The mass spectrometry data is compared against a protein database library consisting of the sequence of the exogenous (non-self) peptide (antigen X) and the international protein index for humans (representing “self or non-immunogenic peptides). The peptides are ranked by significance according to a probability database. The detected antigenic (non-self) peptide sequences are listed. The data is verified by searching against a scrambled decoy database to reduce false hits {see e.g. Ma B, Johnson R, Mol Cell Proteomics 11:0111.014902 (2012)). The results can demonstrate which peptides from the CD8+ T-cell antigen X are presented in MHC I complexes on the surface of cell-targeting molecule intoxicated target cells.

B. Cell Kill: Directly Targeted Shiga Toxin Cytotoxicity and/or Indirectly Targeted Cell-Mediated Cytotoxicity Via the Recruitment of CTLs

Cell-targeting molecules of the present invention can provide cell-type specific delivery of: 1) CD8+ T-cell epitopes to the MHC class I presentation pathway for presentation and intercellular engagement of CTL(s) as well as 2) potent Shiga toxin cytotoxicity to the cytosol. These multiple cytotoxic mechanisms may complement each other, such as by providing both direct (e.g. Shiga toxin catalysis mediated) target-cell-killing and indirect (e.g. CTL-mediated) target-cell-killing.

For certain embodiments, the cell-targeting molecule of the present invention is cytotoxic at certain concentrations. The cell-targeting molecules of the present invention may be used in application involving indirect (e.g. via intercellular CD8+ immune cell engagement) and/or direct cell killing mechanisms (e.g. via intracellular toxin effector activity). Because Shiga toxins are adapted to killing eukaryotic cells, cytotoxic cell-targeting molecules designed using Shiga toxin A Subunit derived polypeptides can show potent cell-kill activity. Shiga toxin A Subunits and derivatives thereof which comprise active enzymatic domains can kill a eukaryotic cell once in the cell's cytosol. The fusion of a cell-targeting binding region and a heterologous, CD8+ T-cell epitope-peptide to a Shiga toxin A Subunit effector polypeptide can be accomplished without significantly reducing the Shiga toxin effector polypeptide's catalytic and cytotoxic activities (see Examples, infra). Thus, certain cell-targeting molecules of the present invention can provide at least two redundant, mechanisms of target cell killing—(1) indirect, immune cell-mediated killing as a result of heterologous, CD8+ epitope cargo delivery by the cell-targeting molecule of the present invention and (2) direct killing via the functional activity(ies) of a Shiga toxin effector polypeptide component of the cell-targeting molecule of the invention.

For certain embodiments of the cell-targeting molecules of the present invention, upon contacting a target cell physically coupled with an extracellular target biomolecule of the binding region of the molecule, the cell-targeting molecule is capable of causing death of the target cell. The mechanism of cell-kill may be direct, e.g. via the enzymatic activity of the Shiga toxin effector polypeptide, or indirect via immune cell-mediated mechanisms, e.g. CTL-mediated target cell cytolysis, and may be under varied conditions of target cells, such as an ex vivo manipulated target cell, a target cell cultured in vitro, a target cell within a tissue sample cultured in vitro, or a target cell in vivo.

The expression of the target biomolecule need not be native in order for targeted cell killing by a cell-targeting molecule of the invention. Cell-surface expression of the target biomolecule could be the result of an infection, the presence of a pathogen, and/or the presence of an intracellular microbial pathogen. Expression of a target biomolecule could be artificial such as, for example, by forced or induced expression after infection with a viral expression vector, see e.g. adenoviral, adeno-associated viral, and retroviral systems. An example of inducing expression of a target biomolecule is the upregulation of CD38 expression of cells exposed to retinoids, like all-tram-retinoic acid and various synthetic retinoids, or any retinoic acid receptor (RAR) agonist (Drach J et al., Cancer Res 54: 1746-52 (1994); Uruno A et al., J Leukoc Biol 90: 235-47 (2011)). In another example, CD20, HER2, and EGFR expression may be induced by exposing a cell to ionizing radiation (Wattenberg M et al., Br J Cancer 110: 1472-80 (2014)).

For certain embodiments of the cell-targeting molecules of the present invention, the cell targeting molecules are cytotoxic because delivery of the molecule's heterologous, CD8+ T-cell epitope(s) cargo results in MHC class I presentation of the delivered epitope(s) by the target cell and immune cell mediated killing of the target cell.

Certain cell-targeting molecules of the present invention may be used in applications involving indirect cell kill mechanisms, such as, e.g., stimulating CD8+ immune cell mediated, target cell killing. The presentation by targeted cells of immuno-stimulatory non-self antigens, such as, e.g., known viral epitope-peptides with high immunogenicity, can signal to other immune cells to destroy the target cells and recruit more immune cells to the target cell site within an organism. Under certain conditions, the cell-surface presentation of immunogenic CD8+ epitope-peptides by the MHC class I complex targets simulates the immune system to kill the presenting cell for killing by CD8+ CTL-mediated cytolysis.

For certain embodiments of the cell-targeting molecules of the present invention, upon contacting a cell physically coupled with an extracellular target biomolecule of the molecule's binding region, the cell-targeting molecule is capable of indirectly causing the death of the cell, such as, e.g., via the presentation of one or more T-cell epitopes by the target cell and the subsequent recruitment of a CTLs.

In addition, within a chordate, the presentation by target cells of a CD8+ T-cell epitope delivered by the cell-targeting molecule of the present invention may provide the additional functionality of immuno-stimulation to the local area and/or breaking immuno-tolerance to certain malignant cells in a local area and/or systemically throughout the chordate.

For certain embodiments of the cell-targeting molecules of the present invention, upon contacting a cell physically coupled with an extracellular target biomolecule of the binding region, the cell-targeting molecule of the invention is capable of directly causing the death of the cell, such as, e.g., via the enzymatic activity of a Shiga toxin effector polypeptide or a cytotoxic agent described herein. For certain further embodiments of the cell-targeting molecules of the present invention, the cell-targeting molecules are cytotoxic because they comprise a catalytically active, Shiga toxin effector polypeptide component regardless of any functional result of delivery of any heterologous, CD8+ T-cell epitope-peptide to the MHC class I presentation pathway by the cell-targeting molecule.

In addition, a cytotoxic cell-targeting molecule of the present invention that exhibits Shiga toxin effector polypeptide catalytic activity based cytotoxicity may be engineered by the skilled worker using routine methods into enzymatically inactive variants to reduce or eliminate Shiga toxin effector based cytotoxicity. The resulting “inactivated” cell-targeting molecule may or may not still be cytotoxic due to its ability to deliver a heterologous, CD8+ T-cell epitope to the MHC class I system of a target cell and subsequent presentation of the delivered CD8+ T-cell epitope-peptide by MHC class I molecules on the surface of the target cell.

### C. Selective Cytotoxicity Among Cell-Types

Certain cell-targeting molecules of the present invention have uses in the selective killing of specific target cells in the presence of untargeted, bystander cells. By targeting the delivery of immunogenic, CD8+ T-cell epitopes to the MHC class I pathway of target cells, the subsequent presentation of delivered CD8+ T-cell epitopes and the TCR specific regulation of CTL-mediated cytolysis of epitope-presenting target cells can be restricted to preferentially killing selected cell-types in the presence of untargeted cells. In addition, the killing of target cells by the potent cytotoxic activity of various Shiga toxin effector polypeptides can be restricted to preferentially killing target cells with the simultaneous delivery of an immunogenic T-cell epitope and a cytotoxic toxin effector polypeptide.

For certain embodiments, upon administration of the cell-targeting molecule of the present invention to a mixture of cell-types, the cell-targeting molecule is capable of selectively killing those cells which are physically coupled with an extracellular target biomolecule compared to cell-types not physically coupled with an extracellular target biomolecule.

For certain embodiments, upon administration of the cell-targeting molecule of the present invention to a mixture of cell-types, the cytotoxic cell-targeting molecule is capable of selectively killing those cells which are physically coupled with an extracellular target biomolecule compared to cell-types not physically coupled with an extracellular target biomolecule. For certain embodiments, the cytotoxic cell-targeting molecule of the present invention is capable of selectively or preferentially causing the death of a specific cell-type within a mixture of two or more different cell-types. This enables targeting cytotoxic activity to specific cell-types with a high preferentiality, such as a 3-fold cytotoxic effect, over “bystander” cell-types that do not express the target biomolecule. Alternatively, the expression of the target biomolecule of the binding region may be non-exclusive to one cell-type if the target biomolecule is expressed in low enough amounts and/or physically coupled in low amounts with cell-types that are not to be targeted. This enables the targeted cell-killing of specific cell-types with a high preferentiality, such as a 3-fold cytotoxic effect, over “bystander” cell-types that do not express significant amounts of the target biomolecule or are not physically coupled to significant amounts of the target biomolecule.

For certain further embodiments, upon administration of the cytotoxic cell-targeting molecule to two different populations of cell-types, the cytotoxic cell-targeting molecule is capable of causing cell death as defined by the half-maximal cytotoxic concentration (CD50) on a population of target cells, whose members express an extracellular target biomolecule of the binding region of the cytotoxic cell-targeting molecule, at a dose at least three-times lower than the CD50 dose of the same cytotoxic cell-targeting molecule to a population of cells whose members do not express an extracellular target biomolecule of the binding region of the cytotoxic cell-targeting molecule.

For certain embodiments, the cytotoxic activity of a cell-targeting molecule of the present invention toward populations of cell-types physically coupled with an extracellular target biomolecule is at least 3-fold higher than the cytotoxic activity toward populations of cell-types not physically coupled with any extracellular target biomolecule of the binding region. According to the present invention, selective cytotoxicity may be quantified in terms of the ratio (a/b) of (a) cytotoxicity towards a population of cells of a specific cell-type physically coupled with a target biomolecule of the binding region to (b) cytotoxicity towards a population of cells of a cell-type not physically coupled with a target biomolecule of the binding region. For certain embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of cells or cell-types physically coupled with a target biomolecule of the binding region compared to populations of cells or cell-types not physically coupled with a target biomolecule of the binding region.

For certain embodiments, the preferential cell-killing function or selective cytotoxicity of a cell-targeting molecule of the present invention is due to an additional exogenous material (e.g. a cytotoxic material) and/or heterologous, CD8+ T-cell epitope present in the cell-targeting molecule of the present invention and not necessarily a result of the catalytic activity of a Shiga toxin effector polypeptide component of the cell-targeting molecule.

It is important to note that for certain embodiments of the cell-targeting molecules of the present invention, upon administration of the cell-targeting molecule to a chordate, the cell-targeting molecule may cause the death of untargeted cells which are in the vicinity of a target cell and/or which are related to a target cell by sharing a common malignant condition. The presentation of certain T-cell epitopes by target cells within a chordate may result in CTL-mediated killing of the target cells as well as the killing of other cells not presenting the delivered epitope but in the vicinity of epitope-presenting cells. Additionally, the presentation of certain T-cell epitopes by targeted tumor cells within a chordate may result in intermolecular epitope spreading, re-programming of the tumor microenvironment to stimulatory conditions, release of existing immune cells from anergy or removal of de-sensitization to target cells or damaged tissues comprising them, and overcoming the physiological state of tolerance of the subject's immune system to non-self tumor antigens (see Section X. Methods of Using a Cell-Targeting Molecule, infra).

D. Delivery of Additional Exogenous Material into the Interior of a Target Cell

In addition to direct cell killing, cell-targeting molecules of the present invention optionally may be used for delivery of additional exogenous materials into the interiors of target cells. The delivery of additional exogenous materials may be used, e.g., for cytotoxic, cytostatic, immune system stimulation, immune cell targeting, information gathering, and/or diagnostic functions. Non-cytotoxic variants of the cytotoxic, cell-targeting molecules of the invention, or optionally toxic variants, may be used to deliver additional exogenous materials to and/or label the interiors of cells physically coupled with an extracellular target biomolecule of the cell-targeting molecule. Various types of cells and/or cell populations which express target biomolecules to at least one cellular surface may be targeted by the cell-targeting molecules of the invention for receiving exogenous materials.

Because the cell-targeting molecules of the present invention, including nontoxic forms thereof, are capable of entering cells physically coupled with an extracellular target biomolecule recognized by its binding region, certain embodiments of the cell-targeting molecules of the present invention may be used to deliver additional exogenous materials into the interior of targeted cell-types. In one sense, the entire cell-targeting molecule of the invention is an exogenous material which will enter the cell; thus, the “additional” exogenous materials are heterologous materials linked to but other than the core cell-targeting molecule itself. Non-toxic, cell-targeting molecules of the present invention which comprise a heterologous, CD8+ T-cell epitope-peptide(s) which does not stimulate CTL-mediated cell killing in certain situations may still be useful for delivering a “benign” CD8+ T-cell-epitope-peptide which does not result in cell-killing upon MHC class I presentation but allows for information gathering, such as, e.g., regarding immune system function in an individual, MHC class I variant expression, and operability of the MHC class I system in a certain cell.

“Additional exogenous material” as used herein refers to one or more molecules, often not generally present within a native target cell, where the proteins of the present invention can be used to specifically transport such material to the interior of a cell. Non-limiting examples of additional exogenous materials are cytotoxic agents, peptides, polypeptides, proteins, polynucleotides, detection promoting agents, and small molecule chemotherapeutic agents.

In certain embodiments of the proteins of the present invention for delivery of additional exogenous material, the additional exogenous material is a cytotoxic agent, such as, e.g., a small molecule chemotherapeutic agent, cytotoxic antibiotic, alkylating agent, antimetabolite, topoisomerase inhibitor, and/or tubulin inhibitor. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

In certain embodiments, the additional exogenous material comprises a protein or polypeptide comprising an enzyme. In certain other embodiments, the additional exogenous material is a nucleic acid, such as, e.g. a ribonucleic acid that functions as a small inhibiting RNA (siRNA) or microRNA (miRNA). In certain embodiments, the additional exogenous material is an antigen, such as antigens derived from bacterial proteins, viral proteins, proteins mutated in cancer, proteins aberrantly expressed in cancer, or T-cell complementary determining regions. For example, exogenous materials include antigens, such as those characteristic of antigen-presenting cells infected by bacteria, and T-cell complementary determining regions capable of functioning as exogenous antigens. Additional examples of exogenous materials include polypeptides and proteins larger than an antigenic peptide, such as enzymes.

In certain embodiments, the additional exogenous material comprises a proapoptotic peptide, polypeptide, or protein, such as, e.g., BCL-2, caspases (e.g. fragments of caspase-3 or caspase-6), cytochromes, granzyme B, apoptosis-inducing factor (AIF), BAX, tBid (truncated Bid), and proapoptotic fragments or derivatives thereof (see e.g., Ellerby H et al., NatMed 5: 1032-8 (1999); Mai J et al., Cancer Res 61: 7709-12 (2001); Jia L et al. Cancer Res 63: 3257-62 (2003); Liu Y et al., Mol Cancer Ther 2: 1341-50 (2003); Perea S et al., Cancer Res 64: 7127-9 (2004); Xu Y et al., J Immunol 173: 61-7 (2004); Dalken B et al., Cell Death Differ 13: 576-85 (2006); Wang T et al, Cancer Res 67: 11830-9 (2007); Kwon M et al., Mol Cancer Ther 7: 1514-22 (2008); Shan L et al., Cancer Biol Ther 1717-22 (2008); Qiu X et al, Mol Cancer Ther 7: 1890-9 (2008); Wang F et al., Clin Cancer Res 16: 2284-94 (2010); Kim J et al., J Virol 85: 1507-16 (2011)).

### E. Information Gathering for Diagnostic Functions

The cell-targeting molecules of the present invention may be used for information gathering functions. Certain embodiments of the cell-targeting molecules of the present invention may be used for imaging of specific pMHC I presenting cells using antibodies specific to pMHC Is that recognize a heterologous, CD8+ T-cell epitope-peptide (delivered by a cell-targeting molecule of the present invention) complexed with a MHC class I molecule on a cell surface. In addition, certain cell-targeting molecules of the present invention have uses in the in vitro and/or in vivo detection of specific cells, cell-types, and/or cell populations. In certain embodiments, the cell-targeting molecules described herein are used for both diagnosis and treatment, or for diagnosis alone.

The ability to conjugate detection promoting agents known in the art to various cell-targeting molecules of the present invention provides useful compositions for the detection of cancer, tumor, growth abnormality, immune, and infected cells. These diagnostic embodiments of the cell-targeting molecules of the invention may be used for information gathering via various imaging techniques and assays known in the art. For example, diagnostic embodiments of the cell-targeting molecules of the invention may be used for information gathering via imaging of intracellular organelles {e.g. endocytotic, Golgi, endoplasmic reticulum, and cytosolic compartments) of individual cancer cells, immune cells, or infected cells in a patient or biopsy sample.

Various types of information may be gathered using the diagnostic embodiments of the cell-targeting molecules of the invention whether for diagnostic uses or other uses. This information may be useful, for example, in diagnosing neoplastic cell subtypes, determining MHC class I pathway and/or TAP system functionality in specific cell-types, determining changes to MHC class I pathway and/or TAP system functionality in specific cell-types over time, determining therapeutic susceptibilities of a patient's disease, assaying the progression of antineoplastic therapies over time, assaying the progression of immuno-modulatory therapies over time, assaying the progression of antimicrobial therapies over time, evaluating the presence of infected cells in transplantation materials, evaluating the presence of unwanted cell-types in transplantation materials, and/or evaluating the presence of residual tumor cells after surgical excision of a tumor mass.

For example, subpopulations of patients might be ascertained using information gathered using the diagnostic variants of the cell-targeting molecules of the invention, and then individual patients could be categorized into subpopulations based on their unique characteristic(s) revealed using those diagnostic embodiments. For example, the effectiveness of specific pharmaceuticals or therapies might be one type of criterion used to define a patient subpopulation. For example, a nontoxic diagnostic variant of a particular cytotoxic, cell-targeting molecule of the invention may be used to differentiate which patients are in a class or subpopulation of patients predicted to respond positively to a cytotoxic variant of the same cell-targeting molecule of the invention. Accordingly, associated methods for patient identification, patient stratification, and diagnosis using cell-targeting molecules of the present invention, including non-toxic variants of cytotoxic, cell-targeting molecules of the present invention, are considered to be within the scope of the present invention.

### IV. Variations in the Polypeptide Sequence of the Protein Components of the Cell-Targeting Molecules of the Present Invention

The skilled worker will recognize that variations may be made to the cell-targeting molecules of the present invention described above, and polynucleotides encoding any of the former, without diminishing their biological activities, e.g., by maintaining the overall structure and function of the cell-targeting molecules in delivering their heterologous, CD8+ T-cell epitope-peptide cargos to the MHC class I presentation pathways of target cells after exogenous administration to the target cells. For example, some modifications may facilitate expression, facilitate purification, improve pharmacokinetic properties, and/or improve immunogenicity. Such modifications are well known to the skilled worker and include, for example, a methionine added at the amino terminus to provide an initiation site, additional amino acids placed on either terminus to create conveniently located restriction sites or termination codons, and biochemical affinity tags fused to either terminus to provide for convenient detection and/or purification. A common modification to improve the immunogenicity of a polypeptide is to remove, after the production of the polypeptide, the starting methionine residue, which may be formylated during production in a bacterial host system, because, e.g., the presence of N-formylmethionine (fMet) might induce undesirable immune responses in chordates.

In certain variations of embodiments of the cell-targeting molecules of the invention, certain cell-targeting functionality of the binding region must be maintained so that the specificity and selectivity of target biomolecule binding is significantly preserved. In certain variations of embodiments of the cell-targeting molecules of the invention, certain biological activities of the Shiga toxin effector polypeptide may need to be preserved, e.g., inducing cellular internalization, intracellular routing to certain subcellular compartments (like compartments competent for entry into the MHC class I pathway), and/or ability to deliver exogenous material(s) to certain subcellular compartments of target cells.

Also contemplated herein is the inclusion of additional amino acid residues at the amino and/or carboxy termini, such as sequences for biochemical tags or other moieties. The additional amino acid residues may be used for various purposes including, e.g., to facilitate cloning, expression, post-translational modification, synthesis, purification, detection, and/or administration. Non-limiting examples of biochemical tags and moieties are: chitin binding protein domains, enteropeptidase cleavage sites, Factor Xa cleavage sites, FIAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and V5 epitope tags.

In certain of the above embodiments, the protein sequence of the cell-targeting molecules of the present invention, or polypeptide components thereof, are varied by one or more conservative amino acid substitutions introduced into the protein or polypeptide component(s) as long as the cell-targeting molecule retains the ability to deliver its heterologous, CD8+ T-cell epitope-peptide cargo to a MHC class I presentation system of a target cell after exogenous administration to the target cells such that the delivery and/or cell-surface MHC class I presentation of the delivered CD8+ T-cell epitope is detectable using an assay known to the skilled worker and/or described herein.

As used herein, the term “conservative substitution” denotes that one or more amino acids are replaced by another, biologically similar amino acid residue. Examples include substitution of amino acid residues with similar characteristics, e.g. small amino acids, acidic amino acids, polar amino acids, basic amino acids, hydrophobic amino acids, and aromatic amino acids (see, for example, Table B, infra). An example of a conservative substitution with a residue normally not found in endogenous, mammalian peptides and proteins is the conservative substitution of an arginine or lysine residue with, for example, ornithine, canavanine, aminoethylcysteine, or another basic amino acid. For further information concerning phenotypically silent substitutions in peptides and proteins see, e.g., Bowie J et al., Science 247: 1306-10 (1990).

TABLE B
Examples of Conservative Amino Acid Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table B above, exemplary conservative substitutions of amino acids are grouped by physicochemical properties—1: neutral, hydrophilic; II: acids and amides; III: basic; IV: hydrophobic; V: aromatic, bulky amino acids, VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

In certain embodiments, the cell-targeting molecules of the present invention (e.g. cell-targeting fusion proteins) may comprise functional fragments or variants of a polypeptide region of the invention that have, at most, 20, 15, 10, 9, 8, 7, 6, 5, 4, 3, 2, or 1 amino acid residue substitutions compared to a polypeptide sequence recited herein, as long as the cell-targeting molecule comprising it is capable of delivering its heterologous, CD8+ T-cell epitope-peptide cargo to a MHC class I presentation pathway of a target cell. Variants of the cell-targeting molecules of the invention are within the scope of the present invention as a result of changing a polypeptide component of the cell-targeting protein of the invention by altering one or more amino acids or deleting or inserting one or more amino acids, such as within the binding region or the Shiga toxin effector polypeptide region, in order to achieve desired properties, such as changed cytotoxicity, changed cytostatic effects, changed immunogenicity, and/or changed serum half-life. A cell-targeting molecule of the invention, or polypeptide component thereof, may further be with or without a signal sequence.

Accordingly, in certain embodiments, the binding region of cell-targeting molecules of the present invention comprises or consists essentially of amino acid sequences having at least 80%, 85%, 90%, 95%, 97%, 98%, 99%, 99.5% or 99.7% overall sequence identity to a binding region recited herein or otherwise already known when compared to an aligned sequence in which the alignment is done by a computer homology program known in the art, as long as the binding region exhibits, as a component of the cell-targeting molecule, a reasonable amount of extracellular target biomolecule binding specificity and affinity, such as, e.g. by exhibiting ′AK′O to the target biomolecule of 10⁻⁵ to 10⁻¹² moles/liter.

In certain embodiments, the Shiga toxin effector polypeptide region of cell-targeting molecules of the present invention comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring toxin, such as, e.g., Shiga toxin A Subunit, such as SLT-1A (SEQ ID NO: 1), StxA (SEQ ID NO:2), and/or SLT-2A (SEQ ID NO:3) when compared to an aligned sequence in which the alignment is done by a computer homology program known in the art, as long as the Shiga toxin effector polypeptide exhibits, as a component of the cell-targeting molecule, the required level of the Shiga toxin effector function(s) related to intracellular delivery of a the cell-targeting molecule's heterologous, CD8+ T-cell epitope-peptide cargo to the MHC class I presentation pathway of at least one target cell-type.

In certain embodiments, the Shiga toxin effector polypeptide components of the cell-targeting molecules of the present invention may be altered to change the enzymatic activity and/or cytotoxicity of the Shiga toxin effector polypeptide, as long as the Shiga toxin effector polypeptide exhibits, as a component of the cell-targeting molecule, the required level of the Shiga toxin effector function(s) related to intracellular delivery of a the cell-targeting molecule's CD8+ T-cell epitope-peptide cargo to the MHC class I presentation pathway of at least one target cell-type. This change may or may not result in a change in the cytotoxicity of the Shiga toxin effector polypeptide or cell-targeting molecule of which the altered Shiga toxin effector polypeptide is a component. Both Shiga toxin enzymatic activity and cytotoxicity may be altered, reduced, or eliminated by mutation or truncation. Possible alterations include mutations to the Shiga toxin effector polypeptide selected from the group consisting of: a truncation, deletion, inversion, insertion, rearrangement, and substitution as long as the Shiga toxin effector polypeptide retains, as a component of the cell-targeting molecule, the required level of the Shiga toxin effector function(s) related to intracellular delivery of a the cell-targeting molecule's heterologous, CD8+ T-cell epitope-peptide cargo to the MHC class I presentation pathway of at least one target cell-type.

The cytotoxicity of the A Subunits of members of the Shiga toxin family may be altered, reduced, or eliminated by mutation or truncation. The cell-targeting molecules of the present invention each comprise a Shiga toxin A Subunit effector polypeptide region which provide each cell-targeting molecule the ability to deliver the cell-targeting molecule's heterologous, CD8+ T-cell epitope-peptide cargo to the MHC class I presentation pathway of at least one target cell-type regardless of Shiga toxin effector polypeptide catalytic activity. As shown in the Examples below, the catalytic activity and cytotoxicity of Shiga toxin effector polypeptides may be uncoupled from other Shiga toxin effector functions required to provide a cell-targeting molecule of the present invention with the ability to deliver a fused, heterologous, CD8+ T-cell epitope to the MHC class I presentation pathway of a target cell-type. Thus in certain embodiments of the cell-targeting molecules of the present invention, the Shiga toxin effector polypeptide component is engineered to exhibit diminished or abolished Shiga toxin cytotoxicity, such as, e.g., due to the presence of amino acid residue mutations relative to a wild-type Shiga toxin A Subunit in one or more key residues involved in enzymatic activity. This provides cell-targeting molecules of the invention which do not kill target cells directly via the Shiga toxin function of cytotoxicity. Such cell-targeting molecules of the invention, which lack cytotoxic Shiga toxin effector polypeptide regions, are useful for effectuating 1) cell-killing via the delivery of a heterologous, CD8+ T-cell epitope-peptide for MHC class I presentation by a target cell, 2) the stimulation of desirable, intercellular immune cell response(s) to a target cells as a result of the delivery of a heterologous, CD8+ T-cell epitope-peptide to the MHC class I system of target cells, and/or 3) the labeling of target cells with specific CD8+ T-cell epitope-peptide/MHC class I molecule complexes when the target cell is not defective in the machinery required to do so.

The catalytic and/or cytotoxic activity of the A Subunits of members of the Shiga toxin family may be diminished or eliminated by mutation or truncation. The most critical residues for enzymatic activity and/or cytotoxicity in the Shiga toxin A Subunits have been mapped to the following residue-positions: aspargine-75, tyrosine-77, glutamate-167, arginine-170, arginine-176, and tryptophan-203 among others (Di R et al., Toxicon 57: 525-39 (2011)). In particular, a double-mutant construct of Stx2A containing glutamate-E167-to-lysine and arginine-176-to-lysine mutations was completely inactivated; whereas, many single mutations in Stx1 and Stx2 showed a 10-fold reduction in cytotoxicity. The positions labeled tyrosine-77, glutamate-167, arginine-170, tyrosine-114, and tryptophan-203 have been shown to be important for the catalytic activity of Stx, Stx1, and Stx2 (Hovde C et al., Proc Natl Acad Sci USA 85: 2568-72 (1988); Deresiewicz R et al., Biochemistry 31: 3272-80 (1992); Deresiewicz R et al., Mol Gen Genet 241: 467-73 (1993); Ohmura M et al., Microb Pathog 15: 169-76 (1993); Cao C et al., Microbiol Immunol 38: 441-7 (1994); Suhan M, Hovde C, Infect 66: 5252-9 (1998)). Mutating both glutamate-167 and arginine-170 eliminated the enzymatic activity of Slt-I A1 in a cell-free ribosome inactivation assay (LaPointe P et al., J Biol Chem 280: 23310-18 (2005)). In another approach using de novo expression of Slt-I A1 in the endoplasmic reticulum, mutating both glutamate-167 and arginine-170 eliminated Slt-I A1 fragment cytotoxicity at that expression level (LaPointe P et al., J Biol Chem 280: 23310-18 (2005)).

Further, truncation of Stx1A to 1-239 or 1-240 reduced its cytotoxicity, and similarly, truncation of Stx2A to a conserved hydrophobic residue reduced its cytotoxicity. The most critical residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others (McCluskey A et al., PLoS One 7: e31 191 (2012)).

In certain embodiments of the cell-targeting molecules of the invention, the Shiga toxin A Subunit effector polypeptide derived from or comprising a component derived from SLT-1A (SEQ ID NO: 1) or StxA (SEQ ID NO:2) comprises an alteration from a wild-type Shiga toxin, polypeptide sequence, such as, e.g., one or more of the following amino acid residue substitution(s): asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to alanine, substitution of the glutamate at position 167 to aspartate, substitution of the arginine at position 170 to alanine, substitution of the arginine at position 176 to lysine, and/or substitution of the tryptophan at position 203 to alanine. Other mutations which either enhance or reduce Shiga toxin A Subunit effector polypeptide enzymatic activity and/or cytotoxicity are within the scope of the present invention and may be determined using well known techniques and assays disclosed herein.

In certain embodiments, the cell-targeting molecule of the present invention, or a proteinaceous component thereof, comprises one or more post-translational modifications, such as, e.g., phosphorylation, acetylation, glycosylation, amidation, hydroxylation, and/or methylation (see e.g. Nagata K et al., Bioinformatics 30: 1681-9 (2014)).

In certain embodiments of the cell-targeting molecules of the present invention, one or more amino acid residues may be mutated, inserted, or deleted in order to increase the enzymatic activity of the Shiga toxin effector polypeptide region as long as the cell-targeting molecule is capable of delivering its heterologous, CD8+ T-cell epitope-peptide cargo to the MHC class I presentation pathway of a target cell. For example, mutating residue-position alanine-231 in Stx1A to glutamate increased its enzymatic activity in vitro (Suhan M, Hovde C, Infect Immun 66: 5252-9 (1998)).

The cell-targeting molecules of the present invention may optionally be conjugated to one or more additional agents, which may include therapeutic and/or diagnostic agents known in the art, including such agents as described herein.

### V. Production, Manufacture, and Purification of Cell-Targeting Molecules of the Present Invention

The cell-targeting molecules of the present invention may be produced using biochemical engineering techniques well known to those of skill in the art. For example, cell-targeting molecules of the invention and/or protein components thereof may be manufactured by standard synthetic methods, by use of recombinant expression systems, or by any other suitable method. Thus, certain cell-targeting molecules of the present invention, and protein components thereof, may be synthesized in a number of ways, including, e.g. methods comprising: (1) synthesizing a polypeptide or polypeptide component of a protein using standard solid-phase or liquid-phase methodology, either stepwise or by fragment assembly, and isolating and purifying the final polypeptide or protein compound product; (2) expressing a polynucleotide that encodes a polypeptide or polypeptide component of a cell-targeting molecule of the invention in a host cell and recovering the expression product from the host cell or host cell culture; or (3) cell-free in vitro expression of a polynucleotide encoding a polypeptide or polypeptide component of a cell-targeting molecule of the invention, and recovering the expression product; or by any combination of the methods of (1), (2) or (3) to obtain fragments of the peptide component, subsequently joining {e.g. ligating) the fragments to obtain the peptide component, and recovering the peptide component. For example, polypeptide and/or peptide components may be ligated together using coupling reagents, such as, e.g., N,N′-dicyclohexycarbodiimide and N-ethyl-5-phenyl-isoxazolium-3′-sulfonate (Woodward's reagent K).

It may be preferable to synthesize a cell-targeting molecule or a proteinaceous component of a cell-targeting molecule of the invention by means of solid-phase or liquid-phase peptide synthesis. Cell-targeting molecules of the invention and components thereof may suitably be manufactured by standard synthetic methods. Thus, peptides may be synthesized by, e.g. methods comprising synthesizing the peptide by standard solid-phase or liquid-phase methodology, either stepwise or by fragment assembly, and isolating and purifying the final peptide product. In this context, reference may be made to WO 1998/11125 or, inter alia, Fields G et al., Principles and Practice of Solid-Phase Peptide Synthesis (Synthetic Peptides, Grant G, ed., Oxford University Press, U.K., 2nd ed., 2002) and the synthesis examples therein.

Cell-targeting molecules of the present invention which are fusion proteins may be prepared (produced and purified) using recombinant techniques well known in the art. In general, methods for preparing proteins by culturing host cells transformed or transfected with a vector comprising the encoding polynucleotide and recovering the protein from cell culture are described in, e.g. Sambrook J et al., Molecular Cloning: A Laboratory Manual (Cold Spring Harbor Laboratory Press, NY, U.S., 1989); Dieffenbach C et al., PCR Primer: A Laboratory Manual (Cold Spring Harbor Laboratory Press, N.Y., U.S., 1995). Any suitable host cell may be used to produce a cell-targeting protein of the present invention or a proteinaceous component of a cell-targeting molecule of the present invention. Host cells may be cells stably or transiently transfected, transformed, transduced or infected with one or more expression vectors which drive expression of a cell-targeting molecule of the present invention and/or protein component thereof. In addition, a cell-targeting molecule of the present invention may be produced by modifying the polynucleotide encoding the cell-targeting protein of the present invention or a proteinaceous component of a cell-targeting molecule of the present invention that result in altering one or more amino acids or deleting or inserting one or more amino acids in order to achieve desired properties, such as changed cytotoxicity, changed cytostatic effects, and/or changed serum half-life.

There are a wide variety of expression systems which may be chosen to produce a cell-targeting molecule of the present invention. For example, host organisms for expression of cell-targeting proteins of the invention include prokaryotes, such as E. coli and B. subtilis, eukaryotic cells, such as yeast and filamentous fungi (like S. cerevisiae, P. pastoris, A. awamori, and K. lactis), algae (like C. reinhardtii), insect cell lines, mammalian cells (like CHO cells), plant cell lines, and eukaryotic organisms such as transgenic plants (like A. thaliana and N. benthamiand).

Accordingly, the present invention also provides methods for producing a cell-targeting molecule of the present invention according to above recited methods and using (i) a polynucleotide encoding part or all of a molecule of the invention or a polypeptide component of a cell-targeting molecule of the present invention, (ii) an expression vector comprising at least one polynucleotide of the invention capable of encoding part or all of a molecule of the invention or a polypeptide component thereof when introduced into a suitable host cell or cell-free expression system, and/or (iii) a host cell comprising a polynucleotide or expression vector of the invention.

When a protein is expressed using recombinant techniques in a host cell or cell-free system, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g. ethanol precipitation or ammonium sulfate precipitation). Any number of biochemical purification techniques may be used to increase the purity of a cell-targeting molecule of the present invention. In certain embodiments, the cell-targeting molecules of the invention may optionally be purified in homo-multimeric forms (e.g. a stable complex of two or more identical cell-targeting molecules of the invention) or in hetero-multimeric forms (e.g. a stable complex of two or more non-identical cell-targeting molecules of the invention).

In the Examples below are descriptions of non-limiting examples of methods for producing a cell-targeting molecule of the present invention or polypeptide component thereof, as well as specific but non-limiting aspects of production for exemplary cell-targeting molecules of the present invention.

### VI. Pharmaceutical and Diagnostic Compositions Comprising a Cell-Targeting Molecule of the Present Invention

The present invention provides cell-targeting molecules for use, alone or in combination with one or more additional therapeutic agents, in a pharmaceutical composition, for treatment or prophylaxis of conditions, diseases, disorders, or symptoms described in further detail below (e.g. cancers, malignant tumors, non-malignant tumors, growth abnormalities, immune disorders, and microbial infections). The present invention further provides pharmaceutical compositions comprising a cell-targeting molecule of the invention, or a pharmaceutically acceptable salt or solvate thereof, according to the invention, together with at least one pharmaceutically acceptable carrier, excipient, or vehicle. In certain embodiments, the pharmaceutical composition of the present invention may comprise homo-multimeric and/or hetero-multimeric forms of the cell-targeting molecules of the invention. The pharmaceutical compositions will be useful in methods of treating, ameliorating, or preventing a disease, condition, disorder, or symptom described in further detail below. Each such disease, condition, disorder, or symptom is envisioned to be a separate embodiment with respect to uses of a pharmaceutical composition according to the invention. The invention further provides pharmaceutical compositions for use in at least one method of treatment according to the invention, as described in more detail below.

As used herein, the terms “patient” and “subject” are used interchangeably to refer to any organism, commonly vertebrates such as humans and animals, which presents symptoms, signs, and/or indications of at least one disease, disorder, or condition. These terms include mammals such as the non-limiting examples of primates, livestock animals (e.g. cattle, horses, pigs, sheep, goats, etc.), companion animals (e.g. cats, dogs, etc.) and laboratory animals (e.g. mice, rabbits, rats, etc.).

As used herein, “treat,” “treating,” or “treatment” and grammatical variants thereof refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease, disorder, or condition. With regard to tumors and/or cancers, treatment includes reductions in overall tumor burden and/or individual tumor size.

As used herein, the terms “prevent,” “preventing,” “prevention” and grammatical variants thereof refer to an approach for preventing the development of, or altering the pathology of, a condition, disease, or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g. a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount” or “therapeutically effective amount” is an amount or dose of a composition (e.g. a therapeutic composition or agent) that produces at least one desired therapeutic effect in a subject, such as preventing or treating a target condition or beneficially alleviating a symptom associated with the condition. The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic compound (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly (see e.g. Remington: The Science and Practice of Pharmacy (Gennaro A, ed., Mack Publishing Co., Easton, Pa., U.S., 19th ed., 1995)).

Diagnostic compositions of the present invention comprise a cell-targeting molecule of the invention and one or more detection promoting agents. Various detection promoting agents are known in the art, such as isotopes, dyes, colorimetric agents, contrast enhancing agents, fluorescent agents, bioluminescent agents, and magnetic agents. These agents may be incorporated into the cell-targeting molecule of the invention at any position. The incorporation of the agent may be via an amino acid residue(s) of the protein or via some type of linkage known in the art, including via linkers and/or chelators. The incorporation of the agent is in such a way to enable the detection of the presence of the diagnostic composition in a screen, assay, diagnostic procedure, and/or imaging technique.

When producing or manufacturing a diagnostic composition of the present invention, a cell-targeting molecule of the invention may be directly or indirectly linked to one or more detection promoting agents. There are numerous detection promoting agents known to the skilled worker which can be operably linked to the polypeptides or cell-targeting molecules of the invention for information gathering methods, such as for diagnostic and/or prognostic applications to diseases, disorders, or conditions of an organism (see e.g. Cai W et al., J Nucl Med 48: 304-10 (2007); Nayak T, Brechbiel M, Bioconjug Chem 20: 825-41 (2009); Paudyal P et al., Oncol Rep 22: 115-9 (2009); Qiao J et al., PLoS ONE 6: e18103 (2011); Sano K et al., Breast Cancer Res 14: R61 (2012)). For example, detection promoting agents include image enhancing contrast agents, such as fluorescent dyes (e.g. Alexa680, indocyanine green, and Cy5.5), isotopes and radionuclides, such as ¹¹C, ¹³N, ¹⁵O, ¹⁸F, ³²P, ⁵¹Mn, ⁵²mMn, ⁵²Fe, ⁵⁵Co, ⁶²Cu, ⁶⁴Cu, ⁶⁷Cu, ⁶⁷Ga, ⁶⁸Ga, ⁷²As, ⁷³Se, ⁷⁵Br, ⁷⁶Br, ⁸²mRb, ⁸³Sr, ⁸⁶Y, ⁹⁰Y, ⁸⁹Zr, ⁹⁴mTc, ⁹⁴Tc, ″mTc, ¹¹⁰In, ¹¹¹In, ¹²⁰I, ¹²³I, ¹²⁴I, ¹²⁵I, ¹³¹I, ¹⁵⁴Gd, ¹⁵⁵Gd, ¹⁵⁶Gd, ¹⁵⁷Gd, ¹⁵⁸Gd, ¹⁷⁷Lu, ¹⁸⁶Re, ¹⁸⁸Re, and ²²³R; paramagnetic ions, such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III); metals, such as lanthanum (III), gold (III), lead (II), and bismuth (III); ultrasound-contrast enhancing agents, such as liposomes; radiopaque agents, such as barium, gallium, and thallium compounds. Detection promoting agents may be incorporated directly or indirectly by using an intermediary functional group, such as chelators like 2-benzyl DTPA, PAMAM, NOTA, DOTA, TETA, analogs thereof, and functional equivalents of any of the foregoing {see Leyton J et al., Clin Cancer Res 14: 7488-96 (2008)).

There are numerous standard techniques known to the skilled worker for incorporating, affixing, and/or conjugating various detection promoting agents to proteins, especially to immunoglobulins and immunoglobulin-derived domains (Wu A, Methods 65: 139-47 (2014)). Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MRI), positron emission tomography (PET), single-photon emission computed tomography (SPECT), ultrasound, and x-ray computed tomography imaging {see Kaur S et al., Cancer Lett 315: 97-111 (2012), for review).

VII. Production or Manufacture of a Pharmaceutical and/or Diagnostic Composition Comprising a Cell-Targeting Molecule of the Present Invention

Pharmaceutically acceptable salts or solvates of any of the cell-targeting molecules of the invention are likewise within the scope of the present invention.

The term “solvate” in the context of the present invention refers to a complex of defined stoichiometry formed between a solute {in casu, a cell-targeting molecule or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

Cell-targeting molecules of the present invention, or salts thereof, may be formulated as pharmaceutical compositions prepared for storage or administration, which typically comprise a therapeutically effective amount of a compound of the present invention, or a salt thereof, in a pharmaceutically acceptable carrier. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences (Mack Publishing Co. (A. Gennaro, ed., 1985)). As used herein, “pharmaceutically acceptable carrier” includes any and all physiologically acceptable, i.e. compatible, solvents, dispersion media, coatings, antimicrobial agents, isotonic, and absorption delaying agents, and the like. Pharmaceutically acceptable carriers or diluents include those used in formulations suitable for oral, rectal, nasal or parenteral (including subcutaneous, intramuscular, intravenous, intradermal, and transdermal) administration. Exemplary pharmaceutically acceptable carriers include sterile aqueous solutions or dispersions and sterile powders for the extemporaneous preparation of sterile injectable solutions or dispersions. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants. In certain embodiments, the carrier is suitable for intravenous, intramuscular, subcutaneous, parenteral, spinal or epidermal administration {e.g. by injection or infusion). Depending on selected route of administration, the protein or other pharmaceutical component may be coated in a material intended to protect the compound from the action of low pH and other natural inactivating conditions to which the active protein may encounter when administered to a patient by a particular route of administration.

The formulations of the pharmaceutical compositions of the invention may conveniently be presented in unit dosage form and may be prepared by any of the methods well known in the art of pharmacy. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Subcutaneous or transdermal modes of administration may be particularly suitable for pharmaceutical compositions and therapeutic molecules described herein.

The pharmaceutical compositions of the present invention may also contain adjuvants such as preservatives, wetting agents, emulsifying agents and dispersing agents. Preventing the presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

A pharmaceutical composition of the present invention also optionally includes a pharmaceutically acceptable antioxidant. Exemplary pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

In another aspect, the present invention provides pharmaceutical compositions comprising one or a combination of different cell-targeting molecules of the invention, or an ester, salt or amide of any of the foregoing, and at least one pharmaceutically acceptable carrier.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g. glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g. sugars, polyalcohols such as mannitol, sorbitol, or sodium chloride may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a cell-targeting molecule of the invention in the required amount in an appropriate solvent with one or a combination of ingredients described above, as required, followed by sterilization microfiltration. Dispersions may be prepared by incorporating the active compound into a sterile vehicle that contains a dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a cell-targeting molecule of the invention is designed to be administered by, e.g. intravenous, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or another vehicle as known in the art. A pharmaceutical composition of the present invention may also contain stabilizers, preservatives, buffers, antioxidants, or other additives well known to those of skill in the art.

As described elsewhere herein, a cell-targeting molecule, or composition of the present invention (e.g. pharmaceutical or diagnostic composition) may be prepared with carriers that will protect the cell-targeting molecule against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see e.g. Sustained and Controlled Release Drug Delivery Systems (Robinson J, ed., Marcel Dekker, Inc., NY, U.S., 1978)).

In certain embodiments, the composition of the present invention (e.g. pharmaceutical or diagnostic composition) may be formulated to ensure a desired distribution in vivo. For example, the blood-brain barrier excludes many large and/or hydrophilic compounds. To target a therapeutic cell-targeting molecule or composition of the invention to a particular in vivo location, it can be formulated, for example, in liposomes which may comprise one or more moieties that are selectively transported into specific cells or organs, thus enhancing targeted drug delivery. Exemplary targeting moieties include folate or biotin; mannosides; antibodies; surfactant protein A receptor; pi20 catenin and the like.

Pharmaceutical compositions include parenteral formulations designed to be used as implants or particulate systems. Examples of implants are depot formulations composed of polymeric or hydrophobic components such as emulsions, ion exchange resins, and soluble salt solutions. Examples of particulate systems are microspheres, microparticles, nanocapsules, nanospheres, and nanoparticles (see e.g. Honda M et al., Int J Nanomedicine 8: 495-503 (2013); Sharma A et al., Biomed Res Int 2013: 960821 (2013); Ramishetti S, Huang L, TherDeliv 3: 1429-45 (2012)). Controlled release formulations may be prepared using polymers sensitive to ions, such as, e.g. liposomes, polaxamer 407, and hydroxyapatite.

### VIII. Polynucleotides, Expression Vectors, and Host Cells of the Invention

Beyond the cell-targeting molecules of the present invention and their polypeptide components, the polynucleotides that encode the polypeptides and cell-targeting molecules of the invention, or functional portions thereof, are also encompassed within the scope of the present invention. The term “polynucleotide” is equivalent to the term “nucleic acid,” each of which includes one or more of: polymers of deoxyribonucleic acids (DNAs), polymers of ribonucleic acids (RNAs), analogs of these DNAs or RNAs generated using nucleotide analogs, and derivatives, fragments and homologs thereof. The polynucleotide of the present invention may be single-, double-, or triple-stranded. Such polynucleotides are specifically disclosed to include all polynucleotides capable of encoding an exemplary protein, for example, taking into account the wobble known to be tolerated in the third position of RNA codons, yet encoding for the same amino acid as a different RNA codon (see Stothard P, Biotechniques 28: 1102-4 (2000)).

In one aspect, the invention provides polynucleotides which encode a cell-targeting molecule of the invention (e.g. a fusion protein), or a polypeptide fragment or derivative thereof. The polynucleotides may include, e.g., nucleic acid sequence encoding a polypeptide of at least 50%, 55%, 60%, 65%, 70%, 75%, 80%, 85%, 90%, 95%, 99% or more, identity to a polypeptide comprising one of the amino acid sequences of the protein. The invention also includes polynucleotides comprising nucleotide sequences that hybridize under stringent conditions to a polynucleotide which encodes a cell-targeting molecule of the invention, or a polypeptide fragment or derivative thereof, or the antisense or complement of any such sequence.

Derivatives or analogs of the cell-targeting molecules of the present invention include, inter alia, polynucleotide (or polypeptide) molecules having regions that are substantially homologous to the polynucleotides, cell-targeting molecules, or polypeptide components of the cell-targeting molecules of the present invention, e.g. by at least about 45%, 50%, 70%, 80%, 95%, 98%, or even 99% identity (with a preferred identity of 80-99%) over a polynucleotide or polypeptide sequence of the same size or when compared to an aligned sequence in which the alignment is done by a computer homology program known in the art. An exemplary program is the GAP program (Wisconsin Sequence Analysis Package, Version 8 for UNIX, Genetics Computer Group, University Research Park, Madison, Wis., U.S.) using the default settings, which uses the algorithm of Smith T, Waterman M, AdvApplMath 2: 482-9 (1981). Also included are polynucleotides capable of hybridizing to the complement of a sequence encoding the cell-targeting molecule of the invention under stringent conditions (see e.g. Ausubel F et al., Current Protocols in Molecular Biology (John Wiley & Sons, New York, N.Y., U.S., 1993)), and below. Stringent conditions are known to those skilled in the art and may be found, e.g., in Current Protocols in Molecular Biology (John Wiley & Sons, NY, U.S., Ch. Sec. 6.3.1-6.3.6 (1989)).

The present invention further provides expression vectors that comprise the polynucleotides within the scope of the present invention. The polynucleotides capable of encoding the cell-targeting molecules of the invention, or polypeptide components thereof, may be inserted into known vectors, including bacterial plasmids, viral vectors and phage vectors, using material and methods well known in the art to produce expression vectors. Such expression vectors will include the polynucleotides necessary to support production of contemplated cell-targeting molecules of the invention within any host cell of choice or cell-free expression systems {e.g. pTxb1 and pIVEX2.3). The specific polynucleotides comprising expression vectors for use with specific types of host cells or cell-free expression systems are well known to one of ordinary skill in the art, can be determined using routine experimentation, or may be purchased.

The term “expression vector,” as used herein, refers to a polynucleotide, linear or circular, comprising one or more expression units. The term “expression unit” denotes a polynucleotide segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. An expression vector contains one or more expression units. Thus, in the context of the present invention, an expression vector encoding a cell-targeting molecule of the invention {e.g. a scFv genetically recombined with a Shiga toxin effector polypeptide fused to a T-cell epitope-peptide) includes at least an expression unit for the single polypeptide chain, whereas a protein comprising, e.g. two or more polypeptide chains {e.g. one chain comprising a VL domain and a second chain comprising a VH domain linked to a toxin effector region) includes at least two expression units, one for each of the two polypeptide chains of the protein. For expression of multi-chain cell-targeting proteins of the invention, an expression unit for each polypeptide chain may also be separately contained on different expression vectors {e.g. expression may be achieved with a single host cell into which expression vectors for each polypeptide chain has been introduced).

Expression vectors capable of directing transient or stable expression of polypeptides and proteins are well known in the art. The expression vectors generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

The term “host cell” refers to a cell which can support the replication or expression of the expression vector. Host cells may be prokaryotic cells, such as E. coli or eukaryotic cells {e.g. yeast, insect, amphibian, bird, or mammalian cells). Creation and isolation of host cell lines comprising a polynucleotide of the invention or capable of producing a cell-targeting molecule of the invention, or polypeptide component thereof, can be accomplished using standard techniques known in the art.

Cell-targeting molecules within the scope of the present invention may be variants or derivatives of the polypeptides and proteins described herein that are produced by modifying the polynucleotide encoding a polypeptide and/or protein by altering one or more amino acids or deleting or inserting one or more amino acids that may render it more suitable to achieve desired properties, such as more optimal expression by a host cell.

### IX. Delivery Devices and Kits

In certain embodiments, the invention relates to a device comprising one or more compositions of matter of the invention, such as a pharmaceutical composition, for delivery to a subject in need thereof. Thus, a delivery device comprising one or more compounds of the invention may be used to administer to a patient a composition of matter of the invention by various delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

Also within the scope of the present invention are kits comprising at least one composition of matter of the invention, and optionally, packaging and instructions for use. Kits may be useful for drug administration and/or diagnostic information gathering. A kit of the invention may optionally comprise at least one additional reagent (e.g., standards, markers and the like). Kits typically include a label indicating the intended use of the contents of the kit. The kit may further comprise reagents and other tools for detecting a cell-type (e.g. a tumor cell) in a sample or in a subject, or for diagnosing whether a patient belongs to a group that responds to a therapeutic strategy which makes use of a cell-targeting molecule of the present invention, or composition thereof, or related method of the present invention as described herein.

X. Methods for Using a Cell-Targeting Molecule of the Present Invention and Pharmaceutical Composition and/or Diagnostic Composition Thereof

Generally, it is an object of the present invention to provide pharmacologically active agents, as well as compositions comprising the same, that can be used in the prevention and/or treatment of diseases, disorders, and conditions, such as certain cancers, tumors, growth abnormalities, immune disorders, or further pathological conditions mentioned herein. Accordingly, the present invention provides methods of using the cell-targeting molecules, pharmaceutical compositions, and diagnostic compositions of the present invention for the delivery of a CD8+ T-cell epitope-peptide to the MHC class I presentation pathways of target cells, targeted killing of specific cells, labeling of the cell-surfaces of target cells with specific pMHC Is and/or specific interior compartments of target cells, for collecting diagnostic information, and for treating diseases, disorders, and conditions as described herein. For example, the methods of the present invention may be used as an immunotherapy to prevent or treat cancers, cancer initiation, tumor initiation, metastasis, and/or cancer disease reoccurrence.

In particular, it is an object of the present invention to provide such pharmacologically active agents, compositions, and/or methods that have certain advantages compared to the agents, compositions, and/or methods that are currently known in the art. Accordingly, the present invention provides methods of using cell-targeting molecules characterized by specified protein sequences and pharmaceutical compositions thereof. For example, any of the polypeptide sequences in SEQ ID NOs: 1-62 and 71-115 may be specifically utilized as a component of the cell-targeting molecules used in the following methods or any method for using a cell-targeting molecule known to the skilled worker, such as, e.g., various methods described in WO 2014/164680, WO 2014/164693, WO 2015/138435, WO 2015/138452, WO 2015/113005, WO 2015/113007, WO 2015/191764, US2015/0259428, and US2014/965882.

The present invention provides methods of delivering a CD8+ T-cell epitope-peptide to a cell, the method comprising the step of contacting the cell, either in vitro or in vivo, with a cell-targeting molecule or pharmaceutical composition of the present invention. In certain further embodiments, the cell-targeting molecule of the present invention causes, after the contacting step, an intercellular engagement of the cell by an immune cell, such as, e.g., a CD8+ T-cell and/or CTL, either in vitro cell culture or in vivo within a living chordate. The presentation of a CD8+ T-cell epitope by a target cell within an organism can lead to the activation of robust immune responses to a target cell and/or its general locale within an organism. Thus, the targeted delivery of a CD8+ T-cell epitope for presentation may be utilized for as a mechanism for activating CD8+ T-cell responses during a therapeutic regime and/or vaccination strategy.

The present invention provides methods of delivering to a MHC class I presentation pathway of a chordate cell a CD 8+ T-cell epitope-peptide, the method comprising the step of contacting the cell, either in vitro or in vivo, with a cell-targeting molecule, pharmaceutical composition, and/or diagnostic composition of the present invention. In certain further embodiments, the cell-targeting molecule of the present invention causes, after the contacting step, an intercellular engagement of the cell by an immune cell, such as, e.g., a CD8+ T-cell and/or CTL, either in vitro cell culture or in vivo within a chordate.

The delivery of the CD8+ T-cell epitope-peptide to the MHC class I presentation pathway of a target cell using a cell-targeting molecule of the invention can be used to induce the target cell to present the epitope-peptide in association with MHC class I molecules on a cell surface. In a chordate, the presentation of an immunogenic, CD8+ T-cell epitope by the MHC class I complex can sensitize the presenting cell for killing by CTL-mediated cytolysis, induce immune cells into altering the microenvironment, and signal for the recruitment of more immune cells to the target cell site within the chordate. Thus, the cell-targeting molecules of the present invention, and compositions thereof, can be used to kill a specific cell-type upon contacting a cell or cells with a cell-targeting molecule of the present invention and/or can be used to stimulate an immune response in a chordate.

By engineering MHC class I epitopes, such as, e.g., from a known viral antigen, into cell-targeting molecules, the targeted delivery and presentation of immuno-stimulatory antigens may be used to harness and direct beneficial function(s) of a chordate immune cell, e.g. in vitro, and/or a chordate immune system in vivo. This may be accomplished by exogenous administration of the cell-targeting molecule into an extracellular space, such as, e.g., the lumen of a blood vessel, and then allowing for the cell-targeting molecule to find a target cell, enter the cell, and intracellularly deliver its CD8+ T-cell epitope cargo. The applications of these CD8+ T-cell epitope delivery and MHC class I presenting functions of the cell-targeting molecules of the present invention are vast. For example, the delivery of a CD8+ epitope to a cell and the MHC class I presentation of the delivered epitope by the cell in a chordate can cause the intercellular engagement of a CD8+ effector T-cell and may lead to a CTL(s) killing the target cell and/or secreting immuno-stimulatory cytokines.

Certain embodiments of the present invention is an immunotherapeutic method, the method comprising the step of administering to a patient, in need thereof, a cell-targeting molecule and/or pharmaceutical composition of the present invention. In certain further embodiments, the immunotherapeutic method is a method of treating a disease, disorder, and/or condition (such as, e.g., a cancer, tumor, growth abnormality, immune disorder, and/or microbial infection), by stimulating a beneficial immune response in the patient.

Certain embodiments of the present invention is an immunotherapeutic method of treating cancer, the method comprising the step of administering to a patient, in need thereof, a cell-targeting molecule and/or pharmaceutical composition of the present invention.

The present invention provides immunotherapy methods involving delivering a CD8+ T-cell epitope-peptide to a target cell in a chordate and causing an immune response, the method comprising the step of administering to the chordate a cell-targeting molecule or pharmaceutical composition of the present invention. For certain further embodiments, the immune response is an intercellular immune cell response selected from the group consisting of: CD8+ immune cell secretion of a cytokine(s), CTL induced growth arrest in the target cell, CTL induced necrosis of the target cell, CTL induced apoptosis of the target cell, non-specific cell death in a tissue locus, intermolecular epitope spreading, breaking immunological tolerance to a malignant cell type, and the chordate acquiring persistent immunity to a malignant cell-type {see e.g. Matsushita H et al., Cancer Immunol Res 3: 26-36 (2015)). These immune responses can be detected and/or quantified using techniques known to the skilled worker. For example, CD8+ immune cells can release immuno-stimulatory cytokines, such as, e.g., IFN-γ, tumor necrosis factor alpha (TNFa), macrophage inflammatory protein-1 beta (MIP-Iβ), and interleukins such as IL-17, IL-4, IL-22, and IL-2 {see e.g. Examples, infra; Seder R et al., Nat Rev Immunol 8: 247-58 (2008)). IFN-γ can increase MHC class I molecule expression and sensitize neoplastic cells to CTL-mediated cell killing (Vlkova V et al., Oncotarget 5: 6923-35 (2014)). Inflammatory cytokines can stimulate bystander T-cells that harbor unrelated TCR specificities to the cytokine releasing cell {see e.g. Tough D et al., Science 272: 1947-50 (1996)). Activated CTLs can indiscriminately kill cells proximal to epitope-MHC class I complex presenting cell regardless of the proximal cell's present peptide-MHC class I complex repertoire (Wiedemann A et al., Proc Natl Acad Sci USA 103: 10985-90 (2006)). Thus, for certain further embodiments, the immune response is an intercellular immune cell response selected from the group consisting of: proximal cell killing mediated by immune cells where the proximal cell is not displaying any CD8+ T-cell epitope-peptide delivered by the cell-targeting molecule of the present invention and regardless of the presence of any extracellular target biomolecule of the binding region of the cell-targeting molecule physically coupled to the proximal cell(s) that is killed.

The presence of non-self epitopes in CTL-lysed cells, whether target cells or cells merely proximal to target cells, can be recognized and targeted as foreign by the immune system, including recognition of non-self epitopes in target cells via the mechanism of intermolecular epitope spreading {see McCluskey J et al., Immunol Rev 164: 209-29 (1998); Vanderlugt C et al., Immunol Rev 164: 63-72 (1998); Vanderlugt C, Miller S, Nat Rev Immunol 2: 85-95 (2002)). Proximal cells may include non-neoplastic cells, such as, e.g., cancer associated fibroblasts, mesenchymal stem cells, tumor-associated endothelial cells, and immature myeloid-derived suppressor cells. For example, a cancer cell may harbor on average 25 to 500 nonsynonymous mutations in coding sequences {see e.g. Fritsch E et al., Cancer Immunol Res 2: 522-9 (2014)). Both cancer driver mutations and non-driver mutations are part of the mutational landscape of a cancer cell which may provide numerous non-self epitopes per cell and the average tumor may possess ten or more non-self epitopes {see e.g. Segal N et al., Cancer Res 68: 889-92 (2008)). For example, mutant forms of the tumor protein p53 can contain non-self epitopes {see e.g. Vigneron N et al., Cancer Immun 13: 15 (2013)). In addition, the presence of non-self epitopes, such as mutated self-proteins, can result in the production of memory cells specific to those new epitope(s). Because certain embodiments of the cell-targeting molecules of the present invention may increase dendritic cell sampling at a targeted tissue locus, the probability of cross-priming the immune system with intracellular antigens may be increased {see e.g. Chiang C et al., Expert OpinBiol Ther 15: 569-82 (2015)). Thus, as a result of cell-targeting molecule delivery of a heterologous, CD8+ T-cell epitope and MHC class I presentation of that epitope, target cells and other proximal cells containing non-self epitopes can be rejected by the immune system, including via non-self epitopes other than epitopes delivered by a cell-targeting molecule of the invention. Such mechanisms could, e.g., induce antitumor immunity against tumor cells which do not express the extracellular target biomolecule of the binding region of the cell-targeting molecule.

Immune responses which involve cytokine secretion and/or T-cell activation may result in modulation of the immuno-microenvironment of a locus within a chordate. A method of the present invention may be used to alter the microenvironment of a tissue locus within a chordate in order to change the regulatory homeostasis on immune cells, such as, e.g. tumor-associated macrophages, T-cells, T helper cells, antigen presenting cells, and natural killer cells.

For certain embodiments, a method of the present invention may be used to enhance anti-tumor cell immunity in a chordate subject and/or to create a persistent anti-tumor immunity in a chordate, such as, e.g., due to the development of memory T-cells and/or alterations to the tumor microenvironment.

Certain embodiments of the cell-targeting molecules of the present invention, or pharmaceutical compositions thereof, can b e used to “seed” a locus within a chordate with non-self, CD8+ T-cell epitope-peptide presenting cells in order to stimulate the immune system to police the locus with greater strength and/or to alleviate immuno-inhibitory signals, e.g., anergy inducing signals. In certain further embodiments of this “seeding” method of the present invention, the locus is a tumor mass or infected tissue site. In certain embodiments of this “seeding” method of the present invention, the non-self, CD8+ T-cell epitope-peptide is selected from the group consisting of: peptides not already presented by the target cells of the cell-targeting molecule, peptides not present within any protein expressed by the target cell, peptides not present within the proteome or transcriptome of the target cell, peptides not present in the extracellular microenvironment of the site to be seeded, and peptides not present in the tumor mass or infect tissue site to be targeting.

This “seeding” method functions to label one or more target cells within a chordate with one or more MHC class I presented CD8+ T-cell epitopes (pMHC Is) for intercellular recognition by immune cells and activation of downstream immune responses. By exploiting the cell-internalizing, intracellularly routing, and/or MHC class I epitope delivering functions of the cell-targeting molecules of the present invention, the target cells that display the delivered CD8+ T-cell epitope can be recognized by immunosurveillance mechanisms of the chordate's immune cells and result in intercellular engagement of the presenting target cell by CD8+ T-cells, such as, e.g., CTLs. This “seeding” method of using a cell-targeting molecule of the present invention may stimulate immune cell mediated killing of target cells regardless of whether they are presenting a cell-targeting molecule-delivered T-cell epitope(s), such as, e.g., as a result of intermolecular epitope spreading and/or breaking of immuno-tolerance to the target cell based on presentation of endogenous antigens as opposed to artificially delivered epitopes. This “seeding” method of using a cell-targeting molecule of the present invention may provide a vaccination effect (new epitope(s) exposure) and/or vaccination-booster-dose effect (epitope re-exposure) by inducing adaptive immune responses to cells within the seeded microenvironment, such as, e.g. a tumor mass or infected tissue site, based on the detection of epitopes which are either recognized as foreign by naive T-cells and/or already recognizable as non-self (i.e. recall antigens) by memory T-cells. This “seeding” method may also induce the breaking of immuno-tolerance to a target cell population, a tumor mass, diseased tissue site, and/or infected tissue site within a chordate, either peripherally or systemically.

Certain methods of the present invention involving the seeding of a locus within a chordate with one or more antigenic and/or immunogenic CD8+ T-cell epitopes may be combined with the administration of immunologic adjuvants, whether administered locally or systemically, to stimulate the immune response to certain antigens, such as, e.g., the co-administration of a composition of the present invention with one or more immunologic adjuvants like a cytokine, bacterial product, or plant saponin. Other examples of immunologic adjuvants which may be suitable for use in the methods of the present invention include aluminum salts and oils, such as, e.g., alums, aluminum hydroxide, mineral oils, squalene, paraffin oils, peanut oils, and thimerosal.

Certain methods of the present invention involve promoting immunogenic cross-presentation and/or cross-priming of naive CD8+ T-cells in a chordate. For certain methods of the present invention, cross-priming occurs as a result of the death, and/or the manner of death, of a target cell caused by a cell-targeting molecule of the present invention such that the exposure of intracellular antigens in the dying or dead target cell to immunosurveillance mechanisms is promoted.

Because multiple, heterologous, CD8+ T-cell epitopes may be delivered by a single cell-targeting molecule of the present invention, a single embodiment of the cell-targeting molecule of the present invention may be therapeutically effective in different individual chordates of the same species with different MHC I class molecule variants, such as, e.g., in humans with different HLA alleles. This ability of certain embodiments of the present invention may allow for the combining within a single cell-targeting molecule of different CD8+ T-cell epitope-peptides with different therapeutic effectiveness in different sub-populations of subjects based on MHC class I molecule diversity and polymorphisms. For example, human MHC class I molecules, the HLA proteins, vary among humans based on genetic ancestry, e.g. African (sub-Saharan), Amerindian, Caucasiod, Mongoloid, New Guinean and Australian, or Pacific islander.

Cell-targeting molecules of the present invention which comprise heterologous, CD8+ T-cell epitopes from CMV antigens may be particularly effective because a majority of the human population has specific sets of CD8+ T-cells primed to react to CMV antigens and are constantly repressing chronic CMV infections to remain asymptomatic for their entire life. In addition, elderly humans may reactive even more quickly and strongly to CMV CD8+ T-cell epitopes due to age-related changes in the adaptive immune system regarding CMV, such as, e.g., a potentially more focused immune surveillance toward CMV and as shown by the composition of the T-cell antigen receptor repertoire and relative CTL levels in more elderly humans (see e.g. Koch S et al., Ann N Y Acad Sci 1114: 23-35 (2007); Vescovini R et al., J Immunol 184: 3242-9 (2010); Cicin-Sain L et al., J Immunol 187: 1722-32 (2011); Fulop T et al., Front Immunol 4: 271 (2013); Pawelec G, Exp Gerontol 54: 1-5 (2014)).

The present invention provides methods of killing a cell comprising the step of contacting the cell, either in vitro or in vivo, with a cell-targeting molecule or pharmaceutical composition of the present invention. The cell-targeting molecules and pharmaceutical compositions of the present invention can be used to kill a specific cell-type upon contacting a cell or cells with one of the claimed compositions of matter. In certain embodiments, a cell-targeting molecule or pharmaceutical composition of the present invention can be used to kill specific cell-types in a mixture of different cell-types, such as mixtures comprising cancer cells, infected cells, and/or hematological cells. In certain embodiments, a cell-targeting molecule or pharmaceutical composition of the present invention can be used to kill cancer cells in a mixture of different cell-types. In certain embodiments, a cell-targeting molecule or pharmaceutical composition of the present invention can be used to kill specific cell-types in a mixture of different cell-types, such as pre-transplantation tissues. In certain embodiments, a cell-targeting molecule or pharmaceutical composition of the present invention can be used to kill specific cell-types in a mixture of cell-types, such as pre-administration tissue material for therapeutic purposes. In certain embodiments, a cell-targeting molecule or pharmaceutical composition of the present invention can be used to selectively kill cells infected by viruses or microorganisms, or otherwise selectively kill cells expressing a particular extracellular target biomolecule, such as a cell surface biomolecule. The cell-targeting molecules and pharmaceutical compositions of the present invention have varied applications, including, e.g., uses in depleting unwanted cell-types from tissues either in vitro or in vivo, uses as antiviral agents, uses as anti-parasitic agents, and uses in purging transplantation tissues of unwanted cell-types. In certain embodiments, a cell-targeting molecule and/or pharmaceutical composition of the present invention can be used to kill specific cell-types in a mixture of different cell-types, such as pre-administration tissue material for therapeutic purposes, e.g., pre-transplantation tissues. In certain embodiments, a cell-targeting molecule or pharmaceutical composition of the present invention can be used to selectively kill cells infected by viruses or microorganisms, or otherwise selectively kill cells expressing a particular extracellular target biomolecule, such as a cell surface biomolecule.

The present invention provides a method of killing a cell in a patient in need thereof, the method comprising the step of administering to the patient at least one cell-targeting molecule of the present invention or a pharmaceutical composition thereof. In certain embodiments of the Shiga toxin effector polypeptide or cell-targeting molecule of the present invention, or pharmaceutical compositions thereof, can be used to kill an infected cell in a patient by targeting an extracellular biomolecule found physically coupled with an infected cell.

In certain embodiments, the cell-targeting molecule of the present invention or pharmaceutical compositions thereof can be used to kill a cancer cell in a patient by targeting an extracellular biomolecule found physically coupled with a cancer or tumor cell. The terms “cancer cell” or “cancerous cell” refers to various neoplastic cells which grow and divide in an abnormally accelerated and/or unregulated fashion and will be clear to the skilled person. The term “tumor cell” includes both malignant and non-malignant cells. Generally, cancers and/or tumors can be defined as diseases, disorders, or conditions that are amenable to treatment and/or prevention. The cancers and tumors (either malignant or non-malignant) which are comprised of cancer cells and/or tumor cells which may benefit from methods and compositions of the invention will be clear to the skilled person. Neoplastic cells are often associated with one or more of the following: unregulated growth, lack of differentiation, local tissue invasion, angiogenesis, and metastasis. The diseases, disorders, and conditions resulting from cancers and/or tumors (either malignant or non-malignant) which may benefit from the methods and compositions of the present invention targeting certain cancer cells and/or tumor cells will be clear to the skilled person.

Certain embodiments of the cell-targeting molecules and compositions of the present invention may be used to kill cancer stem cells, tumor stem cells, pre-malignant cancer-initiating cells, and tumor-initiating cells, which commonly are slow dividing and resistant to cancer therapies like chemotherapy and radiation. For example, acute myeloid leukemias (AMLs) may be treated with the present invention by killing AML stem cells and/or dormant AML progenitor cells {see e.g. Shlush L et al., Blood 120: 603-12 (2012)). Cancer stem cells often overexpress cell surface targets, such as, e.g., CD44, CD200, and others listed herein, which can be targets of certain binding regions of certain embodiments of the cell-targeting molecules of the present invention {see e.g. Kawasaki B et al., Biochem Biophys Res Commun 364:778-82 (2007); Reim F et al., Cancer Res 69: 8058-66 (2009)).

Because of the Shiga toxin A Subunit based mechanism of action, compositions of matter of the present invention may be more effectively used in methods involving their combination with, or in complementary fashion with other therapies, such as, e.g., chemotherapies, immunotherapies, radiation, stem cell transplantation, and immune checkpoint inhibitors, and/or effective against chemoresistant/radiation-resistant and/or resting tumor cells/tumor initiating cells/stem cells. Similarly, compositions of matter of the present invention may be more effectively used in methods involving in combination with other cell-targeted therapies targeting other than the same epitope on, non-overlapping, or different targets for the same disease disorder or condition.

Certain embodiments of the cell-targeting molecules of the present invention, or pharmaceutical compositions thereof, can be used to kill an immune cell (whether healthy or malignant) in a patient by targeting an extracellular biomolecule found physically coupled with an immune cell.

It is within the scope of the present invention to utilize a cell-targeting molecule of the present invention, or pharmaceutical composition thereof, for the purposes of purging cell populations {e.g. bone marrow) of malignant and/or neoplastic cells and then reinfusing the target-cell-depleted material into a patient in need thereof.

Additionally, the present invention provides a method of treating a disease, disorder, or condition in a patient comprising the step of administering to a patient in need thereof a therapeutically effective amount of at least one of the cell-targeting molecules of the present invention, or a pharmaceutical composition thereof. Contemplated diseases, disorders, and conditions that can be treated using this method include cancers, malignant tumors, non-malignant tumors, growth abnormalities, immune disorders, and microbial infections. Administration of a “therapeutically effective dosage” of a composition of the present invention can result in a decrease in severity of disease symptoms, an increase in frequency and duration of disease symptom-free periods, or a prevention of impairment or disability due to the disease affliction.

The therapeutically effective amount of a composition of the present invention will depend on the route of administration, the type of subject being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

An acceptable route of administration may refer to any administration pathway known in the art, including but not limited to aerosol, enteral, nasal, ophthalmic, oral, parenteral, rectal, vaginal, or transdermal (e.g. topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

For administration of a pharmaceutical composition of the present invention, the dosage range will generally be from about 0.001 to 10 milligrams per kilogram (mg/kg), and more, usually 0.001 to 0.5 mg/kg, of the subject's body weight. Exemplary dosages may be 0.01 mg/kg body weight, 0.03 mg/kg body weight, 0.07 mg/kg body weight, 0.09 mg/kg body weight or 0.1 mg/kg body weight or within the range of 1-10 mg/kg. An exemplary treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular patient.

Pharmaceutical compositions of the present invention will typically be administered to the same patient on multiple occasions. Intervals between single dosages can be, for example, 2-5 days, weekly, monthly, every two or three months, every six months, or yearly. Intervals between administrations can also be irregular, based on regulating blood levels or other markers in the subject or patient. Dosage regimens for a composition of the present invention include intravenous administration of 1 mg/kg body weight or 3 mg/kg body weight with the composition administered every two to four weeks for six dosages, then every three months at 3 mg/kg body weight or 1 mg/kg body weight.

A pharmaceutical composition of the present invention may be administered via one or more routes of administration, using one or more of a variety of methods known in the art. As will be appreciated by the skilled worker, the route and/or mode of administration will vary depending upon the desired results. Routes of administration for cell-targeting molecules, pharmaceutical compositions, and diagnostic compositions of the present invention include, e.g. intravenous, intramuscular, intradermal, intraperitoneal, subcutaneous, spinal, or other parenteral routes of administration, for example by injection or infusion. For other embodiments, a cell-targeting molecules, pharmaceutical composition, and diagnostic composition of the present invention may be administered by a non-parenteral route, such as a topical, epidermal or mucosal route of administration, for example, intranasally, orally, vaginally, rectally, sublingually, or topically.

Therapeutic cell-targeting molecules of the present invention, or pharmaceutical compositions thereof, may be administered with one or more of a variety of medical devices known in the art. For example, in one embodiment, a pharmaceutical composition of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

In certain embodiments, a cell-targeting molecule or pharmaceutical composition of the present invention, alone or in combination with other compounds or pharmaceutical compositions, can show potent cell-kill activity when administered to a population of cells, in vitro or in vivo in a subject such as in a patient in need of treatment. By targeting the delivery of the Shiga toxin effector polypeptide associated with a heterologous CD8+ T-cell epitope cargo using high-affinity binding regions to specific cell-types, Shiga toxin effector and/or CD8+ T-cell epitope presentation mediated cell-killing activities can be restricted to specifically and selectively kill certain cell-types within an organism, such as certain cancer cells, neoplastic cells, malignant cells, non-malignant tumor cells, or infected cells.

The cell-targeting molecule of the present invention, or pharmaceutical composition thereof, may be administered alone or in combination with one or more other therapeutic or diagnostic agents. A combination therapy may include a cell-targeting molecule of the present invention, or pharmaceutical composition thereof, combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutic molecules which may complement or otherwise be beneficial in a therapeutic or prophylactic treatment regimen.

Treatment of a patient with a cell-targeting molecule or pharmaceutical composition of the present invention preferably leads to cell death of targeted cells and/or the inhibition of growth of targeted cells. As such, cell-targeting molecules of the present invention, and pharmaceutical compositions comprising them, will be useful in methods for treating a variety of pathological disorders in which killing or depleting target cells may be beneficial, such as, inter alia, cancers, tumors, growth abnormalities, immune disorders, and infected cells. The present invention provides methods for suppressing cell proliferation and treating cell disorders, including neoplasia and/or unwanted proliferation of certain cell-types.

In certain embodiments, the cell-targeting molecules and pharmaceutical compositions of the present invention can be used to treat or prevent cancers, tumors (malignant and non-malignant), growth abnormalities, immune disorders, and microbial infections. In a further aspect, the above ex vivo method can be combined with the above in vivo method to provide methods of treating or preventing rejection in bone marrow transplant recipients, and for achieving immunological tolerance.

The cell-targeting molecules and pharmaceutical compositions of the present invention are commonly anti-neoplastic agents—meaning they are capable of treating and/or preventing the development, maturation, or spread of neoplastic or malignant cells by inhibiting the growth and/or causing the death of cancer or tumor cells. In certain embodiments, the present invention provides methods for treating malignancies or neoplasms and other blood cell associated cancers in a mammalian subject, such as a human, the method comprising the step of administering to a subject in need thereof a therapeutically effective amount of a cell-targeting molecule or pharmaceutical composition of the invention.

In another aspect, certain embodiments of the cell-targeting molecules and pharmaceutical compositions of the present invention are antimicrobial agents-meaning they are capable of treating and/or preventing the acquisition, development, or consequences of microbiological pathogenic infections, such as caused by viruses, bacteria, fungi, prions, or protozoans.

The cell-targeting molecules and/or pharmaceutical compositions of the present invention may be utilized in a method of treating cancer comprising administering to a patient, in need thereof, a therapeutically effective amount of a cell-targeting molecule or pharmaceutical composition of the present invention. In certain embodiments of the methods of the present invention, the cancer being treated is selected from the group consisting of: bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), and uterine cancer.

The cell-targeting molecules and pharmaceutical compositions of the present invention may be utilized in a method of treating an immune disorder comprising administering to a patient, in need thereof, a therapeutically effective amount of the cell-targeting molecule or pharmaceutical composition of the present invention. In certain embodiments of the methods of the present invention, the immune disorder is related to an inflammation associated with a disease selected from the group consisting of: amyloidosis, ankylosing spondylitis, asthma, autism, cardiogenesis, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders, multiple sclerosis, myasthenia gravis, neuroinflammation, polyarteritis nodosa, polyarthritis, psoriasis, psoriatic arthritis, rheumatoid arthritis, scleroderma, septic shock, Sjorgren's syndrome, systemic lupus erythematosus, ulcerative colitis, vasculitis.

Among certain embodiments of the present invention is using the cell-targeting molecule of the present invention as a component of a pharmaceutical composition or medicament for the treatment or prevention of a cancer, tumor, other growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Among certain embodiments of the present invention is a method of using a cell-targeting molecule, pharmaceutical composition, and/or diagnostic composition of the present invention for the purpose of information gathering regarding diseases, conditions and/or disorders. For example, the cell-targeting molecule of the present invention may be used for imaging of pMHC I presentation by tumor cells using antibodies specific to certain pMHC Is. The detection of such labeled target cells after being treated with a cell-targeting molecule of the present invention may provide a readout regarding a targeted cell-type's competency at antigen processing and MHC class I presentation as well as the percentage of such competent target cells within a population of target cells when combined with readouts from diagnostic variants of the cell-targeting molecules of the invention.

Among certain embodiments of the present invention is a method of using a cell-targeting molecule, pharmaceutical composition, and/or diagnostic composition of the present invention to detect the presence of a cell-type for the purpose of information gathering regarding diseases, conditions and/or disorders. The method comprises contacting a cell with a diagnostically sufficient amount of a cell-targeting molecule of the present invention in order to detect the molecule by an assay or diagnostic technique. The phrase “diagnostically sufficient amount” refers to an amount that provides adequate detection and accurate measurement for information gathering purposes by the particular assay or diagnostic technique utilized. Generally, the diagnostically sufficient amount for whole organism in vivo diagnostic use will be a non-cumulative dose of between 0.01 mg to 10 mg of the detection promoting agent linked cell-targeting molecule of the invention per kg of subject per subject. Typically, the amount of cell-targeting molecule of the invention used in these information gathering methods will be as low as possible provided that it is still a diagnostically sufficient amount. For example, for in vivo detection in an organism, the amount of cell-targeting molecule or diagnostic composition of the invention administered to a subject will be as low as feasibly possible.

The cell-type specific targeting of cell-targeting molecules of the present invention combined with detection promoting agents provides a way to detect and image cells physically coupled with an extracellular target biomolecule of a binding region of the molecule of the invention. Alternatively, the display of a cell-targeting molecule delivered heterologous, CD8+ T-cell epitope can provide a way to detect and image cells which internalized a cell-targeting molecule of the present invention. Imaging of cells using the cell-targeting molecules and diagnostic compositions of the present invention may be performed in vitro or in vivo by any suitable technique known in the art. Diagnostic information may be collected using various methods known in the art, including whole body imaging of an organism or using ex vivo samples taken from an organism. The term “sample” used herein refers to any number of things, but not limited to, fluids such as blood, urine, serum, lymph, saliva, anal secretions, vaginal secretions, and semen, and tissues obtained by biopsy procedures. For example, various detection promoting agents may be utilized for non-invasive in vivo tumor imaging by techniques such as magnetic resonance imaging (MRI), optical methods (such as direct, fluorescent, and bioluminescent imaging), positron emission tomography (PET), single-photon emission computed tomography (SPECT), ultrasound, x-ray computed tomography, and combinations of the aforementioned {see, Kaur S et al., Cancer Lett 315: 97-111 (2012), for review).

Among certain embodiment of the present invention is a method of using a cell-targeting molecule, pharmaceutical composition, and/or diagnostic composition of the present invention to label or detect the interiors of neoplastic cells and/or immune cell-types {see e.g., Koyama Y et al., Clin Cancer Res 13: 2936-45 (2007); Ogawa M et al., Cancer Res 69: 1268-72 (2009); Yang L et al., Small 5: 235-43 (2009)). This may be based on the ability of certain cell-targeting molecules of the present invention to enter specific cell-types and route within cells via retrograde intracellular transport to specific subcellular compartments such that interior compartments of specific cell-types are labeled for detection. This can be performed on cells in situ within a patient or in vitro on cells and tissues removed from an organism, e.g. biopsy materials.

Diagnostic compositions of the present invention may be used to characterize a disease, disorder, or condition as potentially treatable by a related pharmaceutical composition of the present invention. Certain compositions of matter of the present invention may be used to determine whether a patient belongs to a group that responds to a therapeutic strategy which makes use of a cell-targeting molecule of the invention, or composition thereof, or related method of the present invention as described herein or is well suited for using a delivery device of the invention.

Diagnostic compositions of the present invention may be used after a disease, e.g. a cancer, is detected in order to better characterize it, such as to monitor distant metastases, heterogeneity, and stage of cancer progression. The phenotypic assessment of disease disorder or infection can help prognostic and prediction during therapeutic decision making. In disease reoccurrence, certain methods of the invention may be used to determine if a localized or systemic problem.

Diagnostic compositions of the present invention may be used to assess responses to therapeutic(s) regardless of the type of therapeutic, e.g. small molecule drug, biological drug, or cell-based therapy. For example, certain embodiments of the diagnostic compositions of the invention may be used to measure changes in tumor size, changes in antigen positive cell populations including number and distribution, or monitoring a different marker than the antigen targeted by a therapy already being administered to a patient (see Smith-Jones P et al., Nat. Biotechnol 22: 701-6 (2004); Evans M et al., Proc. Natl. Acad. Sci. U.S.A. 108: 9578-82 (2011)).

Diagnostic compositions of the present invention may be used to assess the MHC class I system functionality in target cell-types. For example, certain malignant cells, such as infected, tumor, or cancer cells, can exhibit alterations, defects, and perturbations to their MHC class I presentation pathways. This can be studied in vitro or in vivo. Diagnostic compositions of the invention may be used to monitor changes in MHC class I presentation among individual cells within a population of target cells within an organism or to count or determine percentages of MHC class I presentation defective target cells within an organism, tumor biopsy, etc.

In certain embodiments of the method used to detect the presence of a cell-type may be used to gather information regarding diseases, disorders, and conditions, such as, for example bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), uterine cancer, AIDS, amyloidosis, ankylosing spondylitis, asthma, autism, cardiogenesis, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders, multiple sclerosis, myasthenia gravis, neuroinflammation, polyarteritis nodosa, polyarthritis, psoriasis, psoriatic arthritis, rheumatoid arthritis, scleroderma, septic shock, Sjorgren's syndrome, systemic lupus erythematosus, ulcerative colitis, vasculitis, cell proliferation, inflammation, leukocyte activation, leukocyte adhesion, leukocyte chemotaxis, leukocyte maturation, leukocyte migration, neuronal differentiation, acute lymphoblastic leukemia (ALL), T acute lymphocytic leukemia/lymphoma (ALL), acute myelogenous leukemia, acute myeloid leukemia (AML), B-cell chronic lymphocytic leukemia (B-CLL), B-cell prolymphocytic lymphoma, Burkitt's lymphoma (BL), chronic lymphocytic leukemia (CLL), chronic myelogenous leukemia (CML-BP), chronic myeloid leukemia (CML), diffuse large B-cell lymphoma, follicular lymphoma, hairy cell leukemia (HCL), Hodgkin's Lymphoma (HL), intravascular large B-cell lymphoma, lymphomatoid granulomatosis, lymphoplasmacytic lymphoma, MALT lymphoma, mantle cell lymphoma, multiple myeloma (MM), natural killer cell leukemia, nodal marginal B-cell lymphoma, Non-Hodgkin's lymphoma (NHL), plasma cell leukemia, plasmacytoma, primary effusion lymphoma, pro-lymphocytic leukemia, promyelocytic leukemia, small lymphocytic lymphoma, splenic marginal zone lymphoma, T-cell lymphoma (TCL), heavy chain disease, monoclonal gammopathy, monoclonal immunoglobulin deposition disease, myelodusplastic syndromes (MDS), smoldering multiple myeloma, and Waldenstrom macroglobulinemia.

In certain embodiments, the cell-targeting molecules of the present invention, or pharmaceutical compositions thereof, are used for both diagnosis and treatment, or for diagnosis alone. In some situations, it would be desirable to determine or verify the HLA variant(s) and/or HLA alleles expressed in the subject and/or diseased tissue from the subject, such as, e.g., a patient in need of treatment, before selecting a cell-targeting molecule of the invention for use in treatment(s). In some situations, it would be desirable to determine, for an individual subject, the immunogenicity of certain CD8+ T-cell epitopes before selecting which cell-targeting molecule, or composition thereof, to use in a method of the present invention.

The present invention is further illustrated by the following non-limiting examples of cell-targeting molecules comprising the aforementioned structures and functions, in particular the function of extracellular targeting the delivery of CD8+ T-cell epitope to specific cells and then intracellular delivery of the CD8+ T-cell epitope to the MHC class I pathway for presentation on a cell surface.

### EXAMPLES

The following examples demonstrate certain embodiments of the present invention. However, it is to be understood that these examples are for illustration purposes only and do not intend, nor should any be construed, to be wholly definitive as to conditions and scope of this invention. The experiments in the following examples were carried out using standard techniques, which are well known and routine to those of skill in the art, except where otherwise described in detail.

Cell-targeting, Shiga toxin effector polypeptides can be engineered to deliver immunogenic epitope-peptides for presentation by target cells. These cell-targeting polypeptides provide targeted delivery of epitopes and may be used in applications involving cell-type specific presentation of immuno-stimulatory epitopes within a chordate. The presentation of a T-cell immunogenic epitope by the MHC class I system within a chordate targets the epitope presenting cell for killing by CD8+ CTL-mediated lysis and may also stimulate other immune responses in the vicinity.

In the examples, T-cell antigens were fused to cell-targeting molecules comprising Shiga toxin A Subunit effector polypeptides. All these fusion polypeptides involve the addition of at least one peptide to the starting polypeptide scaffold and do not require the embedding or inserting of any heterologous, CD8+ T-cell epitope internally within a Shiga toxin effector polypeptide region. Thus, in certain exemplary cell-targeting molecules of the present invention, the Shiga toxin effector polypeptide region consists of a completely wild-type, Shiga toxin polypeptide.

The examples below describe exemplary, cell-targeting proteins of the present invention, each comprising an immunoglobulin-type binding region, a Shiga toxin effector polypeptide, and a fused, heterologous, CD8+ T-cell epitope-peptide. The exemplary cell-targeting molecules of the invention bound to target biomolecules expressed by targeted cell-types and entered the targeted cells. The internalized exemplary cell-targeting proteins of the invention effectively routed their Shiga toxin effector polypeptide regions to the cytosol and killed target cells. An exemplary cell-targeting protein delivered, within target cells, its fused, T-cell epitope-peptide to the MHC class I pathway resulting in presentation of the T-cell epitope-peptide on the surface of target cells. The display of delivered T-cell epitopes by a target may signal to CD8+ effector T-cells to kill the epitope-displaying target cells as well as stimulate other immune responses in the vicinity of epitope-display target cells.

### Example 1. Cell-Targeting Molecules Comprising Shiga Toxin A Subunit Derived Polypeptide Regions and Fused, T-Cell Epitope-Peptides

Cell-targeting molecules were created and tested—the cell-targeting molecules each comprising 1) a cell-targeting binding region, 2) a Shiga toxin effector polypeptide region, and 3) a T-cell epitope-peptide region. Previously, Shiga toxin A Subunit derived, cell-targeting molecules have been constructed and shown to promote cellular internalization and direct their own intracellular routing to the cytosol (see e.g. WO 2014/164680, WO 2014/164693, WO 2015/138435, WO 2015/138452, WO 2015/113005, WO 2015/113007, and WO 2015/191764). T-cell epitope-peptides were fused to modular polypeptide components of these Shiga toxin A Subunit derived, cell-targeting molecules in order to create novel cell-targeting molecules.

As demonstrated below in this Example, several cell-targeting proteins of the present invention were capable, upon exogenous administration, of delivering a heterologous, T-cell epitope-peptide to the MHC class I pathway for presentation by targeted, human, cancer cells. Also demonstrated below in this Example, certain cell-targeting proteins of the present invention were capable of specifically killing targeted, human, cancer cells via their Shiga toxin effector polypeptide regions. The cell-targeting binding regions of the exemplary cell-targeting proteins of the invention of this Example were each capable of exhibiting high-affinity binding to an extracellular target biomolecule physically-coupled to the surface of a specific cell-type(s). The exemplary cell-targeting proteins of the invention of this Example are capable of selectively targeting cells expressing a target biomolecule of their cell-targeting binding region and internalizing into these target cells.

### I. Human CD8+ T-Cell Epitope Components for Cell-Targeting Molecules

In this Example, epitope-peptides which are known to be immunogenic to human, CD8+ T-cells were selected for fusing to Shiga toxin derived, cell-targeting proteins. In particular, immunogenic epitope-peptides were selected from viral proteins of viruses which infect humans, and these T-cell epitope-peptides were fused to cell-targeting proteins comprising Shiga toxin effector polypeptides which have the intrinsic ability to intracellularly route to the cytosol via the endoplasmic reticulum.

The viral, immunogenic, T-cell epitope-peptides of this Example were chosen based on their ability to bind to human MHC class I molecules and thus provoke human, CTL-mediated immune response(s). There are many known immunogenic viral proteins and peptide components of viral proteins from human viruses, such as human influenza A viruses and human CMV viruses. Seven, viral epitope-peptides (SEQ ID NOs: 4-12) were scored for the ability to bind to common human MHC class I human leukocyte antigen (HLA) variants encoded by the more prevalent alleles in human populations using the Immune Epitope Database (IEDB) Analysis Resource MHC-I binding prediction's consensus tool and recommended parameters (Kim Y et al., Nucleic Acids Res 40: W252-30 (2012)). The IEDB MHC-I binding prediction analysis consensus tool predicted the “ANN affinity”—an estimated binding affinity between the input peptide and the selected human HLA variant where IC₅₀ values less than 50 nanomolar (nM) are considered high affinity, IC50 values between 50 and 500 nM are considered intermediate affinity, and IC50 values between 500 and 5000 nM are considered low affinity. The IEDB MHC-I binding prediction analysis indicated higher-affinity binders with lower percentile rankings. Table 1 shows the IEDB MHC-I binding prediction analysis percentile rank and predicted binding affinity of the seven, in silico tested, T cell epitope-peptides (SEQ ID NOs: 4-12) binding to certain human HLA variants.

TABLE 1
Predicted Binding Affinities of Epitope-
Peptides to Human, MHC Class I Molecules

| T-Cell | | MHC Class I Molecule | | |
|---|---|---|---|---|
| Epitope- | | Binding Prediction | | |
| Peptide | | | percentile | predicted |
| name | sequence | HLA allele | rank | affinity |
| C1 | VTEHDTLLY | HLA-A*01:01 | 0.20 | high |
| C1- | GLDRNSGNY | HLA-A*01:01 | 0.80 | inter- |
| 2 | | | | mediate |
| C2 | NLVPMVATV | HLA-A*02:01 | 1.00 | high |
| C3 | GVMTRGRLK | HLA-A*03:01 | 0.35 | high |
| C24 | VYALPLKML | HLA-A*24:02 | 0.85 | inter- |
| | | | | mediate |
| C24- | QYDPVAALF | HLA-A*24-02 | 0.50 | inter- |
| 2 | | | | mediate |
| F2 | GILGFVFTL | HLA-A*02:01 | 0.80 | high |
| F3 | ILRGSVAHK | HLA-A*03:01 | 0.25 | high |
| E2 | CLGGLLTMV | HLA-A*02:01 | 2.00 | inter- |
| | | | | mediate |

The results of the IEDB MHC-I binding prediction analysis show that some peptides were predicted to binding with high affinity to at least one human MHC class I molecule, whereas other peptides were predicted to bind with more moderate affinities to the analyzed, human, MHC class I molecules.

### II. Creating Cell-Targeting, Fusion Proteins Comprising Shiga Toxin A Subunit Effector Polypeptide Regions and Fused, T-Cell Epitope-Peptide Regions

The exemplary, cell-targeting, fusion proteins of this Example each comprised a cell-targeting binding region polypeptide, a Shiga toxin A Subunit effector polypeptide, a proteinaceous linker, and a human CD8+ T-cell epitope from Table 1.

Using techniques known in the art, exemplary cell-targeting fusion proteins were created by genetically fusing a human CD8+ T-cell epitope-peptide to the amino terminus (N-terminus) or carboxy terminus (C-terminus) of a polypeptide component of a parental, cell-targeting protein comprising 1) a Shiga toxin A Subunit effector polypeptide and 2) a cell-targeting binding region polypeptide separated by a proteinaceous linker. The fused, CD 8+ T-cell epitopes were chosen from among several T-cell epitope-peptides originating in viruses that commonly infect humans (see Table 1). The resulting cell-targeting, fusion proteins of this Example were constructed such that each comprised a single, continuous polypeptide comprising a cell-targeting, binding region polypeptide, a Shiga toxin A Subunit effector polypeptide, and a fused, heterologous, CD8+ T-cell epitope.

The cell-targeting molecules of the present invention that were produced and tested in this Example included: C2::SLT-1A::scFv2 (SEQ ID NO:50), “inactive C2::SLT-1A::scFv2” (SEQ ID NO: 51), SLT-1A::scFv1::C2 (SEQ ID NO:61), SLT-1A::scFv2::C2 (SEQ ID NO:52), “inactive SLT-1A::scFv2::C2” (SEQ ID NO:53), F2::SLT-1A::scFv2 (SEQ ID NO:54), scFv3::F2::SLT-1A (SEQ ID NO:55), scFv4::F2::SLT-1A (SEQ ID NO:56), SLT-1A::scFv5::C2 (SEQ ID NO:57), SLT-1A::scFv6::F2 (SEQ ID NO:58), “inactive SLT-1A::scFv6::F2” (SEQ ID NO:59), and SLT-1A::scFv7::C2 (SEQ ID NO:60). Other cell-targeting molecules of the present invention that were tested in this Example included: C1::SLT-1A::scFv1 (similar to SEQ ID NO:13), C1-2::SLT-1A::scFv1 (similar to SEQ ID NO:14), C3::SLT-1A::scFv1 (similar to SEQ ID NO: 15), C24::SLT-1A::scFv1 (similar to SEQ ID NO: 16), SLT-1A::scFv1::C1 (similar to SEQ ID NO:21), SLT-1A::scFv1::C24-2 (similar to SEQ ID NO:23), SLT-1A::scFv1::E2 (similar to SEQ ID NO:24), and SLT-1A::scFv1::F3 (similar to SEQ ID NO:25). These exemplary, cell-targeting, fusion proteins of the present invention each comprised a cell-targeting binding region comprising a single-chain variable fragment (scFv), a Shiga toxin A Subunit effector polypeptide derived from the A Subunit of Shiga-like toxin 1 (SLT-1A), and a human CD8+ T-cell epitope-peptide fused to either the binding region or the Shiga toxin effector polypeptide.

All the Shiga toxin effector polypeptide regions of the cell-targeting molecules of this Example consisted of or were derived from amino acids 1-251 of SLT-1A (SEQ ID NO: 1), and some of them contained two or more amino acid residue substitutions relative to a wild-type Shiga toxin A Subunit, such as, e.g., the catalytic domain inactivating substitution E167D, C242S, and/or substitutions resulting in furin-cleavage resistance R248A/R251A (see e.g. WO 2015/191764). As used in this Example, the cell-targeting molecule nomenclature “inactive” refers to a molecule comprising only the Shiga toxin effector polypeptide component(s) that has the E167D substitution.

The immunoglobulin-type binding regions scFv1, scFv2, scFv3, scFv4, scFv5, scFv6, and scFv7 are each single-chain variable fragments that bound with high-affinity to a certain cell-surface, target biomolecule physically coupled to the surface of certain human cancer cells. Both scFv 1 and scFv2 bind with high affinity and specificity to the same extracellular target biomolecule.

All of the cell-targeting molecules tested in the experiments of this Example, including reference cell-targeting molecules (e.g. SEQ ID NOs: 63-70), were produced in a bacterial system and purified by column chromatography using techniques known to the skilled worker.

III. Testing the Shiga Toxin A Subunit Effector Polypeptide Components of Cell-Targeting Molecules for Retention of Shiga Toxin Functions after the Fusion of Binding Regions and T-Cell Epitope-Peptides

Exemplary cell-targeting proteins were tested for retention of Shiga toxin A Subunit effector functions after the fusion of heterologous, CD8+ T-cell epitope-peptides. The Shiga toxin A Subunit effector functions analyzed were: catalytic inactivation of eukaryotic ribosomes, cytotoxicity, and by inference self-directing subcellular routing to the cytosol. At least seven, exemplary, cell-targeting proteins of the present invention exhibited catalytic activity comparable to a wild-type, Shiga toxin effector polypeptide not fused to any heterologous, T-cell epitope-peptide or additional polypeptide moiety.

### A. Testing the Ribosome Inhibition Ability of Exemplary Cell-Targeting Molecules of the Invention

The catalytic activities of Shiga toxin A Subunit derived Shiga toxin effector polypeptide regions of cell-targeting molecules of the invention was tested using a ribosome inhibition assay.

The ribosome inactivation capabilities of exemplary cell-targeting proteins of this Example were determined using a cell-free, in vitro protein translation assay using the TNT® Quick Coupled Transcription/Translation Kit (LI 170 Promega Madison, Wis., U.S.). The kit includes Luciferase T7 Control DNA (L4821 Promega Madison, Wis., U.S.) and TNT® Quick Master Mix. The ribosome activity reaction was prepared according to manufacturer's instructions. A series of 10-fold dilutions of the Shiga toxin derived, cell-targeting protein to be tested was prepared in an appropriate buffer and a series of identical TNT reaction mixture components were created for each dilution. Each sample in the dilution series was combined with each of the TNT reaction mixtures along with the Luciferase T7 Control DNA. The test samples were incubated for 1.5 hours at 30 degrees Celsius (° C.). After the incubation, Luciferase Assay Reagent (E1483 Promega, Madison, Wis., U.S.) was added to all test samples and the amount of luciferase protein translation was measured by luminescence according to manufacturer's instructions.

The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (ICso) value was calculated for each sample using the Prism software function of log(inhibitor) vs. response (three parameters) [Y=Bottom+((Top−Bottom)/(1+10\hat{ }(X−Log ICso)))] under the heading dose-response-inhibition. The ICso values for each Shiga toxin derived, cell-targeting protein from one or more experiments was calculated and is shown in Table 2 in picomolar (pM). Any exemplary cell-targeting molecule of the invention which exhibited an ICso within 10-fold of a positive control molecule comprising a wild-type, Shiga toxin effector polypeptide (e.g. SLT-1A-WT (SEQ ID NO:62)) is considered herein to exhibit ribosome inhibition activity comparable to wild-type.

TABLE 2
Ribosomal Inhibition by Shiga Toxin Derived, Cell-Targeting
Proteins Fused to Heterologous Epitope-Peptides

| | | | |
|---|---|---|---|
| | | | ribosomal |
| | fused | | inhibition |
| Protein | epitope | fusion location | IC₅₀ (pM) |

Experiment 1

| | | | |
|---|---|---|---|
| C1::SLT-1A::scFv1 | C1 | N-terminal fusion | 3.2 |
| C1-2::SLT-1A::scFv1 | C1-2 | N-terminal fusion | 1.2 |
| C3::SLT-1A::scFv1 | C3 | N-terminal fusion | 5.6 |
| C24::SLT-1A::scFv1 | C24 | N-terminal fusion | 1.4 |

| | | |
|---|---|---|
| SLT-1A::scFv1 | none; control molecule having no fused epitope | 1.2 |

Experiment 2

| | | | |
|---|---|---|---|
| C2::SLT-1A::scFv2 | C2 | N-terminal fusion | 12.6 |
| SLT-1A::scFv2::C2 | C2 | C-terminal fusion | 13.1 |

| | | |
|---|---|---|
| SLT-1A::scFv2 | none; control molecule having no fused epitope | 8.3 |

Experiment 3

| | | | |
|---|---|---|---|
| F2::SLT-1A::scFv2 | F2 | N-terminal fusion | 2.2 |

| | | |
|---|---|---|
| SLT-1A::scFv2 | none; control molecule having no fused epitope | 8.2 |

Experiment 4

| | | | |
|---|---|---|---|
| scFv3::F2::SLT-1A | F2 | between binding region and Shiga toxin | 6.0 |
| | | effector (N-terminal of Shiga toxin | |
| | | effector) | |
| scFv4::F2::SLT-1A | F2 | between binding region and Shiga toxin | 5.0 |
| | | effector (N-terminal of Shiga toxin | |
| | | effector) | |

| | | |
|---|---|---|
| SLT-1A-WT only | none; control molecule having no fused epitope | 9.8 |

Experiment 5

| | | | |
|---|---|---|---|
| SLT-1A::scFv5::C2 | C2 | C-terminal fusion | 1.0 |

| | | |
|---|---|---|
| SLT-1A::scFv5 | none; control molecule having no fused epitope | 2.1 |

Experiment 6

| | | | |
|---|---|---|---|
| SLT-1A::scFv6::F2 | F2 | C-terminal fusion | 5.6 |

| | | |
|---|---|---|
| SLT-1A::scFv6 | none; control molecule having no fused epitope | 3.2 |
| SLT-1A-WT only | none; control molecule having no fused epitope | 6.1 |

As shown in Table 2, exemplary cell-targeting proteins exhibited potent ribosome inhibition comparable to the positive controls: 1) a “SLT-1A-WT only” polypeptide (SEQ ID NO:62) comprising only a wild-type Shiga toxin A Subunit polypeptide sequence and 2) a cell-targeting protein comprising a SLT-IA derived Shiga toxin effector polypeptide fused to a scFv binding region but lacking any fused, heterologous, CD8+ T-cell epitope-peptide, e.g., SLT-1A::scFv1 (SEQ ID NO:63), SLT-1A::scFv2 (SEQ ID NO: 64), SLT-1A::scFv5 (SEQ ID NO:66), or SLT-1A::scFv6 (SEQ ID NO:67).

### B. Testing the Cytotoxic Activities of Exemplary Cell-Targeting Molecules of the Invention

The cytotoxic activities of exemplary cell-targeting molecules of the invention were measured using a tissue culture cell-based toxicity assay. The concentration of exogenously administered cell-targeting molecule which kills half the cells in a homogenous cell population (half-maximal cytotoxic concentration) was determined for certain cell-targeting molecules of the invention. The cytotoxicities of exemplary cell-targeting molecules were tested using cell-kill assays involving either target biomolecule positive or target biomolecule negative cells with respect to the target biomolecule of each cell-targeting molecule's binding region.

The cell-kill assays were performed as follows. Human tumor cell line cells were plated (typically at 2×10³ cells per well for adherent cells, plated the day prior to protein addition, or 7.5×10³ cells per well for suspension cells, plated the same day as protein addition) in 20 μL cell culture medium in 384-well plates. A series of 10-fold dilutions of the proteins to be tested was prepared in an appropriate buffer, and 5 μL of the dilutions or only buffer as a negative control were added to the cells. Control wells containing only cell culture medium were used for baseline correction. The cell samples were incubated with the proteins or just buffer for 3 or 5 days at 37° C. and in an atmosphere of 5% carbon dioxide (CO2). The total cell survival or percent viability was determined using a luminescent readout using the CellTiter-Glo® Luminescent Cell Viability Assay (G7573 Promega Madison, Wis., U.S.) according to the manufacturer's instructions as measured in relative light units (RLU).

The Percent Viability of experimental wells was calculated using the following equation: (Test RLU−Average Media RLU)/(Average Cells RLU−Average Media RLU)*100. Log protein concentration versus Percent Viability was plotted in Prism (GraphPad Prism, San Diego, Calif., U.S.) and log (inhibitor) versus response (3 parameter) analysis were used to determine the half-maximal cytotoxic concentration (CD50) value for the tested proteins. The CD50 values for each exemplary cell-targeting protein tested was calculated when possible.

The specificity of the cytotoxic activity of a given cell-targeting molecule was determined by comparing cell kill activities toward cells expressing a significant amount of a target biomolecule of the binding region of the cell-targeting molecule (target positive cells) with cell-kill activities toward cells which do not exhibit any significant amount of any target biomolecule of the binding region of the cell-targeting molecule physically coupled to any cellular surface (target negative cells). This was accomplished by determining the half-maximal cytotoxic concentrations of a given cell-targeting molecule of the invention toward cell populations which were positive for cell surface expression of the target biomolecule of the cell-targeting molecule being analyzed, and, then, using the same cell-targeting molecule concentration range to attempt to determine the half-maximal cytotoxic concentrations toward cell populations which were negative for cell surface expression of the target biomolecule of the cell-targeting molecule. In some experiments, the target negative cells treated with the maximum amount of the Shia-toxin containing molecule did not show any change in viability as compared to a “buffer only” negative control.

The cytotoxic activity levels of various molecules tested using the cell-kill assay described above are reported in Table 3. As reported in Table 3, exemplary cell targeting proteins of the invention which were tested in this assay exhibited potent cytotoxicity. While the fusion of a heterologous, CD8+ T-cell epitope-peptide to a Shiga toxin derived, cell-targeting protein can result in no change in cytotoxicity, some exemplary cell-targeting proteins exhibited reduced cytotoxicity as compared to the parental protein from which it was derived, which did not comprise any fused, heterologous epitope-peptide (Table 3). As reported in the Examples, a molecule exhibiting a CD50 value within 10-fold of a CD50 value measured for a reference molecule is considered to exhibit cytotoxic activity comparable to that reference molecule. In particular, any exemplary cell-targeting molecule of the present invention that exhibited a CD50 value to a target positive cell population within 10-fold of the CD50 value of a reference cell-targeting molecule comprising the same binding region and a wild-type, Shiga toxin effector polypeptide (e.g. SLT-1A-WT (SEQ ID NO:62)) but not comprising any fused, heterologous, T-cell epitope-peptide, toward the same cell-type is referred to herein as “comparable to wild-type.” Cell-targeting molecules that exhibited a CD50 value to a target positive cell population within 100-fold to 10-fold of a reference molecule comprising the same binding region and the same Shiga toxin effector polypeptide but not comprising any fused, heterologous, T-cell epitope-peptide is referred to herein as active but “attenuated.”

TABLE 3
Cytotoxic Activities of Shiga Toxin Derived, Cell-Targeting
Proteins Comprising Fused, Heterologous Epitope-Peptides

| | | | | |
|---|---|---|---|---|
| | fused | | cell-type in | Cytotoxicity |
| Protein | epitope | fusion location | assay | CD₅₀ (nM) |

Experiment 1

| | | | | |
|---|---|---|---|---|
| C1::SLT-1A::scFv1 | C1 | N-terminus | Cell Line A | 0.025 |
| | | | (target positive) | |
| C1-2::SLT-1A::scFv1 | C1-2 | N-terminus | Cell Line A | 0.067 |
| | | | (target positive) | |
| C3::SLT-1A::scFv1 | C3 | N-terminus | Cell Line A | 0.059 |
| | | | (target positive) | |
| C24::SLT-1A::scFv1 | C24 | N-terminus | Cell Line A | 0.240 |
| | | | (target positive) | |

| | | | |
|---|---|---|---|
| SLT-1A::scFv1 | none; control molecule having | Cell Line A | 0.010 |
| | no fused epitope | (target positive) | |
| SLT-1A-WT only | none; control molecule having | Cell Line A | >100 nM |
| | no fused epitope | (target positive) | |

Experiment 2

| | | | | |
|---|---|---|---|---|
| SLT-1A::scFv1::C1 | C1 | C-terminus | Cell Line A | 0.009 |
| | | | (target positive) | |
| SLT-1A::scFv1::C24-2 | C24-2 | C-terminus | Cell Line A | 0.263 |
| | | | (target positive) | |
| SLT-1A::scFv1::F3 | F3 | C-terminus | Cell Line A | 0.041 |
| | | | (target positive) | |
| SLT-1A::scFv1::E2 | E2 | C-terminus | Cell Line A | 0.213 |
| | | | (target positive) | |

| | | | |
|---|---|---|---|
| SLT-1A::scFv1 | none; control molecule having | Cell Line A | 0.004 |
| | no fused epitope | (target positive) | |

Experiment 3

| SLT-1A::scFv1::C2 | C2 | C-terminus | Cell Line B | 0.041 |
|---|---|---|---|---|
| | | | (target positive) | |
| SLT-1A::scFv1 | none; control molecule having | | Cell Line B | 0.097 |
| | no fused epitope | | (target positive) | |
| SLT-1A-WT only | none; control molecule having | | Cell Line B | >100 nM |
| | no fused epitope | | (target positive) | |
| SLT-1A::scFv1::C2 | C2 | C-terminus | Cell Line C | >100 nM |
| | | | (target negative) | |

| | | | |
|---|---|---|---|
| SLT-1A::scFv1 | none; control molecule having | Cell Line C | >100 nM |
| | no fused epitope | (target negative) | |
| SLT-1A-WT only | none; control molecule having | Cell Line C | >100 nM |
| | no fused epitope | (target negative) | |

Experiment 4

| F2::SLT-1A::scFv2 | F2 | N-terminus | Cell Line A | 0.016 |
|---|---|---|---|---|
| | | | (target positive) | |
| SLT-1A::scFv2 | none; control molecule having | | Cell Line A | 0.016 |
| | no fused epitope | | (target positive) | |
| SLT-1A-WT only | none; control molecule having | | Cell Line A | 33.000 |
| | no fused epitope | | (target positive) | |
| F2::SLT-1A::scFv2 | F2 | N-terminus | Cell Line B | 0.0140 |
| | | | (target positive) | |

| | | | |
|---|---|---|---|
| SLT-1A::scFv2 | none; control molecule having | Cell Line B | 0.0250 |
| | no fused epitope | (target positive) | |
| SLT-1A-WT only | none; control molecule having | Cell Line B | 310.000 |
| | no fused epitope | (target positive) | |

Experiment 5

| | | | | |
|---|---|---|---|---|
| C2::SLT-1A::scFv2 | C2 | N-terminus | Cell Line B | 0.35 |
| | | | (target positive) | |
| SLT-1A::scFv2::C2 | C2 | C-terminus | Cell Line B | 0.31 |
| | | | (target positive) | |
| inactive C2::SLT-1A::scFv2 | C2 | N-terminus | Cell Line B | >100 nM |
| | | | (target positive) | |

| | | | |
|---|---|---|---|
| SLT-1A::scFv2::C2 | none; control molecule having | Cell Line B | 0.11 |
| | no fused epitope | (target positive) | |
| SLT-1A-WT only | none; control molecule having | Cell Line B | >100 nM |
| | no fused epitope | (target positive) | |

Experiment 6

| | | | | |
|---|---|---|---|---|
| scFv3::F2::SLT-1A | F2 | between binding region | Cell Line D | 1.42 |
| | | and Shiga toxin effector | (target positive) | |
| | | (N-terminal of Shiga | | |
| | | toxin effector) | | |

| | | | |
|---|---|---|---|
| scFv3::SLT-1A | none; control molecule having | Cell Line D | 1.35 |
| | no fused epitope | (target positive) | |
| SLT-1A-WT only | none; control molecule having | Cell Line D | >100 nM |
| | no fused epitope | (target positive) | |

Experiment 7

| | | | | |
|---|---|---|---|---|
| SLT-1A::scFv5::C2 | C2 | C-terminus | Cell Line E | 0.33 |
| | | | (target positive) | |

| | | | |
|---|---|---|---|
| SLT-1A::scFv5 | none; control molecule having | Cell Line E | 0.25 |
| | no fused epitope | (target positive) | |
| SLT-1A-WT | none; control molecule having | Cell Line E | >100 nM |
| | no fused epitope | (target positive) | |

Experiment 8

| SLT-1A::scFv6::F2 | F2 | C-terminus | Cell Line F | 0.061 |
|---|---|---|---|---|
| | | | (target positive) | |
| SLT-1A::scFv6 | none; control molecule having | | Cell Line F | 0.142 |
| | no fused epitope | | (target positive) | |
| SLT-1A::scFv7::C2 | C2 | C-terminus | Cell Line F | 0.011 |
| | | | (target positive) | |

| | | | |
|---|---|---|---|
| SLT-1A::scFv7 | none; control molecule having | Cell Line F | 0.018 |
| | no fused epitope | (target positive) | |

All the tested, exemplary cell-targeting proteins potently killed target positive cells (Table 3) but did not kill comparable percentages of target negative cells at the same dosages (see e.g. FIGS. 2 and 3). FIGS. 2 and 3 graphically show the specific cytotoxicity of the exemplary cell-targeting protein SLT-1A::scFv1::C2 (SEQ ID NO:61) was only specific to target expressing cells (FIG. 2) but not target negative cells over the concentration range tested (FIG. 3). The CD50 values of cell-targeting proteins toward target negative cells could not be calculated from the concentration range of cell-targeting protein tested because an accurate curve could not be generated when there was not a sizeable decrease in cell viability at the highest tested concentrations (see e.g. FIG. 3).

### IV. Testing Epitope-Peptide Delivery and Target Cell Surface Presentation of Delivered Epitope-Peptides

The successful delivery of a T-cell epitope can be determined by detecting specific cell surface, MHC class I molecule/epitope complexes (pMHC Is). In order to test whether a cell-targeting protein can deliver a fused T-cell epitope to the MHC class I presentation pathway of target cells, an assay was employed which detects human, MHC Class I molecules complexed with specific epitopes. A flow cytometry method was used to demonstrate delivery of a T-cell epitope (fused to a Shiga toxin A Subunit derived cell-targeting protein) and extracellular display of the delivered T-cell epitope-peptide in complex with MHC Class I molecules on the surfaces of target cells. This flow cytometry method utilizes soluble human T-cell receptor (TCR) multimer reagents (Soluble T-Cell Antigen Receptor STAR™ Multimer, Altor Bioscience Corp., Miramar, Fla., U.S.), each with high-affinity binding to a different epitope-human HLA complex.

Each STAR™ TCR multimer reagent is derived from a specific T-cell receptor and allows detection of a specific peptide-MHC complex based on the ability of the chosen TCR to recognize a specific peptide presented in the context of a particular MHC class I molecule. These TCR multimers are composed of recombinant human TCRs which have been biotinylated and multimerized with streptavidin. The TCR multimers are labeled with phycoerythrin (PE). These TCR multimer reagents allow the detection of specific peptide-MHC Class I complexes presented on the surfaces of human cells because each soluble TCR multimer type recognizes and stably binds to a specific peptide-MHC complex under varied conditions (Zhu X et al., J Immunol 176: 3223-32 (2006)). These TCR multimer reagents allow the identification and quantitation by flow cytometry of peptide-MHC class I complexes present on the surfaces of cells.

The TCR CMV-pp65-PE STAR™ multimer reagent (Altor Bioscience Corp., Miramar, Fla., U.S.) was used in this Example. MHC class I pathway presentation of the human CMV C2 peptide (NLVPMVATV (SEQ ID NO:6)) by human cells expressing the HLA-A2 can be detected with the TCR CMV-pp65-PE STAR™ multimer reagent which exhibits high affinity recognition of the CMV-pp65 epitope-peptide (residues 495-503, NLVPMVATV) complexed to human HLA-A2 and is labeled with PE.

The target cells used in this Example (target positive cell lines B, E, F, G, and H) were immortalized human cancer cells available from the ATCC (Manassas Va., U.S.) or the DSMZ (The Leibniz Deutsche Sammlung von Mikroorganismen and Zellkulture) (Braunschweig, DE)). Using standard flow cytometry methods known in the art, the target cells were confirmed to express on their cell surfaces both the HLA-A2 MHC-Class I molecule and the extracellular target biomolecules of the cell-targeting proteins used in this Example. In some experiments, the human cancer cells were pretreated with human interferon gamma (IFN-γ) to enhance expression of human HLA-A2.

Sets of target cells were treated by exogenous administration of cell-targeting molecules comprising a carboxy-terminal fused, viral, CD8+ T-cell epitope: SLT-1A::scFv1::C2 (SEQ ID NO:61), “inactive SLT-1A::scFv2::C2” (SEQ ID NO:53), SLT-1A::scFv5::C2 (SEQ ID NO: 57), and SLT-1A::scFv7::C2 (SEQ ID NO: 60); or were treated by exogenous administration of a negative-control cell-targeting fusion protein which did not comprise any fused, heterologous, viral epitope-peptide (SLT-1A::scFv1 (SEQ ID NO:63), SLT-1A::scFv2 (SEQ ID NO:65), “inactive SLT-1A::scFv2” (SEQ ID NO: 64), SLT-1A::scFv5 (SEQ ID NO: 66), or SLT-1A::scFv7 (SEQ ID NO:69)). The cell-targeting molecules and reference molecules used in these experiments include both catalytically active, cytotoxic cell-targeting molecules and “inactived” cell-targeting molecules—meaning all their Shiga toxin effector polypeptide components comprised the mutation E167D which severly reduces the catalytic activity of Shiga toxin A Subunits and Shiga toxins. These treatments were at cell-targeting molecule concentrations similar to those used by others taking into account cell-type specific sensitivities to Shiga toxins (see e.g. WO 2015/113005). The treated cells were then incubated for 4-16 hours in standard conditions, including at 37° C. and an atmosphere with 5% carbon dioxide, to allow for intoxication mediated by a Shiga toxin effector polypeptide. Then the cells were washed and incubated with the TCR CMV-pp65-PE STAR™ multimer reagent to “stain” C2 peptide-HLA-A2 complex-presenting cells.

As controls, sets of target cells were treated in three conditions: 1) without any treatment (“untreated”) meaning there was addition of only buffer to the cells and no addition of any exogenous molecules, 2) with exogenously administered CMV C2 peptide (CMV-pp65, aa495-503: sequence NLVPMVATV, synthesized by BioSynthesis, Lewisville, Tex., U.S.) (SEQ ID NO:6), and/or 3) with exogenously administered CMV C2 peptide ((SEQ ID NO:6), as above) combined with a Peptide Loading Enhancer (“PLE,” Altor Biosicence Corp., Miramar, Fla., U.S.). The C2 peptide (SEQ ID NO: 6) combined with PLE treatment allowed for exogenous peptide loading and served as a positive control. Cells displaying the appropriate MHC class I haplotype can be forced to load the appropriate exogenously applied peptide from an extracellular space (i.e. in the absence of cellular internalization of the applied peptide) or in the presence of PLE, which is a mixture of B2-microglobulin and other components.

After the treatments, all the sets of cells were washed and incubated with the TCR CMV-pp65-PE STAR™ multimer reagent for one hour on ice. The cells were washed and the fluorescence of the samples was measured by flow cytometry using an Accuri™ C6 flow cytometer (BD Biosciences, San Jose, Calif., U.S.) to detect the presence of and quantify any TCR CMV-pp65-PE STAR™ multimer bound to cells in the population (sometimes referred to herein as “staining”) in relative light units (RLU).

Table 4 and FIGS. 4-8 show results from experiments using the TCR STAR™ assay detecting cell-surface complexes of C2 epitope/HLA-A2 MHC class I molecule. For each experiment, the untreated control sample was used to identify the positive and negative cell populations by employing a gate which results in less than 1% of cells from the untreated control in the “positive” gate (representing background signal). The same gate was then applied to the other samples to characterize the positive population for each sample. Positive cells in this assay were cells which were bound by the TCR-CMV-pp65-PE STAR™ reagent and counted in the positive gate described above. In FIG. 4 and FIGS. 6-8, the flow cytometry histograms are given with the counts (number of cells) on the Y-axis and the relative fluorescent units (RFU) representing TCR CMV-pp65 STAR™ multimer, PE staining signal on the X-axis (log scale). The black line shows the results for the untreated-cells-only sample, and the gray line shows the results for the negative controls (treatment with only a parental, cell-targeting protein lacking any viral epitope-peptide), or the treatment with a specific, exemplary, cell-targeting protein of the invention. In FIGS. 4, 7, and 8, the top panels show the results for the untreated cell samples using black lines and the results for the cell-targeting molecule treated samples using gray lines. In FIGS. 4, 7, and 8, the bottom panels show the results for untreated cell samples using black lines and the results for the control proteins, which did not comprise any fused epitope-peptide, using gray lines. In FIG. 6, the top panel shows the results from a 4-hour incubation and the bottom panel shows the results for a 16-hour incubation. In Table 4, the percentage of cells in a treatment set which stained positive for the C2-epitope-peptide-HLA-A2 MHC class I molecule complex is given. Table 4 also shows the corresponding indexed, mean, fluorescent intensity (“iMFI,” the fluorescence of the positive population multiplied by the percent positive) in RFU for each treatment set.

TABLE 4
Detection of Cell Surface, MHC Class I/C2 Epitope Complexes after
Delivery of C2 Epitope-Peptides by Exemplary Cell-Targeting
Proteins of the invention: Peptide-epitope C2/MHC class I complexes
detected on the surfaces of intoxicated, target cells

| | | | | |
|---|---|---|---|---|
| | target | incubation | percentage of | |
| | positive | duration | pMHC I complex | iMFI |
| Protein | cell-type | (hours) | presenting cells | (RFU) |

Experiment 1

| | | | | |
|---|---|---|---|---|
| SLT-1A::scFv1::C2 | Cell Line B | 4 hours | 33.0% | 440 |
| SLT-1A::scFv2 | Cell Line B | 4 hours | 5.0% | 80 |

Experiment 2

| | | | | |
|---|---|---|---|---|
| SLT-1A::scFv1::C2 | Cell Line B | 16 hours | 95.4% | 28,800 |
| SLT-1A::scFv1 | Cell Line B | 16 hours | 5.0% | 154 |

Experiment 3

| | | | | |
|---|---|---|---|---|
| inactive SLT-1A::scFv2::C2 | Cell Line G | 24 hours | 43.3% | 4,034 |
| inactive SLT-1A::scFv2 | Cell Line G | 24 hours | 0.2% | 17 |
| C2 peptide | Cell Line G | 24 hours | 57.8% | 5,114 |
| C2 peptide + PLE | Cell Line G | 24 hours | 0.5% | 79 |

Experiment 4

| | | | | |
|---|---|---|---|---|
| inactive SLT-1A::scFv2::C2 | Cell Line B | 16 hours | 80.5% | 3,170 |
| inactive SLT-1A::scFv2 | Cell Line B | 16 hours | 4.1% | 63 |
| inactive SLT-1A::scFv2::C2 | Cell Line H | 16 hours | 67.9% | 2,550 |
| inactive SLT-1A::scFv2 | Cell Line H | 16 hours | 3.5% | 47 |

Experiment 5

| | | | | |
|---|---|---|---|---|
| SLT-1A::scFv5::C2 | Cell Line E | 24 hours | 41.9% | 17,846 |
| SLT-1A::scFv5 | Cell Line E | 24 hours | 0.5% | 64 |
| C2 peptide | Cell Line E | 24 hours | 2.4% | 357 |
| C2 peptide + PLE | Cell Line E | 24 hours | 93.2% | 42,429 |

Experiment 6

| | | | | |
|---|---|---|---|---|
| SLT-1A::scFv7::C2 | Cell Line F | 16 hours | 27.6% | 6,132 |
| SLT-1A::scFv7 | Cell Line F | 16 hours | 1.7% | 365 |

As seen in Table 4 and FIGS. 4-8, cell samples treated with exemplary cell-targeting proteins of the present invention displayed expression of the C2-epitope/F£LA-A2 MHC class I molecule complex on the surfaces of a majority of the treated cells depending on the incubation duration. Cells treated with the exogenous cell-targeting proteins SLT-1A::scFv1::C2 (SEQ ID NO:61) or “inactive SLT-1A::scFv2::C2” (SEQ ID NO:53), SLT-1A::scFv5::C2 (SEQ ID NO:57), and SLT-1A::scFv7::C2 (SEQ ID NO: 60) showed a positive signal for cell-surface, C2-epitope/HLA-A2 complexes on 33-95% of the cells in the samples analyzed (Table 4). In contrast, cells that were treated with parental cell-targeting proteins, which did not contain any fused T-cell epitope-peptide as a negative control, exhibited positive cell staining of five percent or less of the cells in the treated cell population (Table 4; FIGS. 4-8).

While the majority of cells treated with exemplary cell-targeting proteins of the present invention displayed on a cell surface the C2-epitope/HLA-A2 complex, five percent or less of the cells in “untreated” cell populations displayed TCR STAR™ staining for C2-epitope/HLA-A2 complexes (Table 4; FIG. 4; FIG. 6). The positive control treatment showed robust staining of 99% of the cells in the population due exclusively to loading of only exogenous C2 epitope-peptide (SEQ ID NO: 6) in the presence of the peptide loading enhancer (FIG. 5). Due to processing efficiency and kinetics, which were not measured, it is possible that the percentage of presented C2-epitope/HLA-A2 complexes detected at a single time-point in a “cell-targeting protein” treatment sample may not accurately reflect the maximum quantity of C2-epitope/HLA-A2 presentation possible after delivery by a given, exemplary, cell-targeting protein of the present invention.

The detection of the T-cell epitope C2 (SEQ ID NO: 6) complexed with human MHC Class I molecules (C2 epitope-peptide/HLA-A2) on the cell surface of cell-targeting molecule treated target cells demonstrated that exemplary cell-targeting proteins (SLT-1A::scFv1::C2 (SEQ ID NO:61), “inactive SLT-1A::scFv2::C2” (SEQ ID NO:53), SLT-1A::scFv5::C2 (SEQ ID NO:57), and SLT-1A::scFv7::C2 (SEQ ID NO:60)) comprising this fused epitope-peptide (C2 (SEQ ID NO:6)) were capable of entering target cells, performing sufficient sub-cellular routing, and delivering sufficient C2 (SEQ ID NO:6) epitope to the MHC class I pathway for surface presentation by target cell surface.

### V. Testing Cytotoxic T-Cell Mediated Cytolysis of Intoxicated Target Cells and Other Immune Responses Triggered by MHC Class I Presentation of T-Cell Epitopes Delivered by Cell-Targeting Molecules of the Present Invention

In this Example, standard assays known in the art are used to investigate the functional consequences of target cells' MHC class I presentation of T-cell epitopes delivered by exemplary cell-targeting molecules of the invention. The functional consequences to investigate include CTL activation (e.g. signal cascade induction), CTL mediated target cell killing, and CTL cytokine release by CTLs.

A CTL-based cytotoxicity assay is used to assess the consequences of epitope presentation. The assay involves tissue-cultured target cells and T-cells. Target cells are intoxicated with exemplary cell-targeting molecules of the invention as described above in Section IV. Testing Epitope-Peptide Delivery and Target Cell Surface Presentation etc. Briefly, target positive cells are incubated for twenty hours in standard conditions with different exogenously administered molecules, including a cell-targeting molecule of the invention. Next, CTLs are added to the treated target cells and incubated to allow for the CTLs to recognize and bind any target cells displaying epitope-peptide/MHC class I complexes (pMHC Is). Then certain functional consequences of pMHC I recognition are investigated using standard methods known to the skilled worker, including CTL binding to target cells, epitope-presenting target cell killing by CTL-mediated cytolysis, and the release of cytokines, such as IFN-γ or interleukins by ELISA or ELISPOT.

Assays were performed to assess functional consequences of intercellular engagement of T-cells in response to cell-surface epitope presentation by targeted cancer cells displaying epitopes delivered by exemplary cell-targeting molecules of the present invention.

FIG. 9 and Table 5 show the results of an Interferon Gamma ELIspot assay (Mabtech, Inc., Cincinnati, Ohio, U.S.) used according to manufacturer's instructions. This ELISPOT assay can be used to quantify IFN-γ secretion as each spot indicates a IFN-γ secreting cell. Briefly, samples of cells of target positive cell line G were incubated for 20 hours with either just phosphate buffered saline (PBS) buffer (“buffer only”), “inactive SLT-1A::scFv2::C2” (SEQ ID NO:53), or the reference molecule “inactive SLT-1A::scFv2” (SEQ ID NO:65). The samples were washed with PBS and added to ELISPOT plates already loaded with human PBMCs (HLA-A2 serotype) from Cellular Technology Limited (Shaker Heights, Ohio, U.S.). The plates were incubated for an additional 24 hours, and then spots were detected according to the Interferon Gamma ELIspot assay MabTech kit instructions and quantified using an ELISPOT plate reader (Zellnet Consulting, Inc., Fort Lee, N.J., U.S.).

TABLE 5
Interferon Gamma Secretion by PBMCs after Recognizing
Epitope Presentation by Target Cells Incubated with
“inactive SLTA-1A::scFv2::C2”

| | target | Average | |
|---|---|---|---|
| | positive | number of | Average Area |
| Protein | cell-type | spots | per spot |
| inactive SLT-1A::scFv2::C2 | Cell Line G | 490 | 2,636,291 |
| inactive SLT-1A::scFv2 | Cell Line G | 280 | 1,511,726 |
| buffer only | Cell Line G | 334 | 2,144,217 |

The results in Table 5 and FIG. 9 show that the incubation of cell line G cells with the exemplary cell-targeting molecule of the present invention “inactive SLT-1A::scFv2::C2” (SEQ ID NO:53) resulted in a PBMC luciferase activity signal greater than the background signal determined using the buffer only treated cell sample or the luciferase signal from the sample cells treated with the reference molecule “inactive SLT-1A::scFv2” (SEQ ID NO:65). The results from this in vitro intercellular immune cell engagement assay showed that Shiga toxin effector polypeptide-mediated delivery of a fused epitope-peptide to target positive cancer cells and subsequent cell-surface presentation of the epitope by the targeted cancer cells can result in intercellular engagement of immune cells with functional consequences, specifically IFN-γ secretion by PBMCs.

When an effector T-cell recognizes a specific epitope-MHC-I complex, the T-cell may initiate an intracellular signaling cascade that drives the translocation of nuclear factor of activated T-cells (NFAT) transcription factors from the cytosol into the nucleus and can result in the stimulation of the expression of genes that contain a NFAT response element(s) (RE) {see e.g. Macian F, Nat Rev Immunol 5: 472-84 (2005)). A J76 T-cell line engineered to express a human T-cell receptor that specifically recognizes the F2 peptide/human ULA A2 MHC class I molecule complex (Berdien B et al., Hum Vaccin Immunother 9: 1205-16 (2013)) was transfected with a luciferase expression vector (pGL4.30[luc2P/NFAT-RE/Hygro], CAT #E8481, Promega Corp., Madison, Wis., U.S.) that is regulated by an NFAT-RE. When the luciferase-reporter-transfected J76 TCR specific cell recognizes a cell displaying the HLA-A2/F2 epitope-peptide (SEQ ID NO: 10) complex, then expression of luciferase can be stimulated by NFAT transcription factors binding to the NFAT-RE of the expression vector. Luciferase activity levels in the transfected J76 cells can be quantified by the addition of a standard luciferase substrate and then reading luminescence levels using a photodetector.

An assay was performed to assess intercellular T-cell activation after recognition of cell-surface epitope presentation by targeted cancer cells displaying an epitope delivered by an exemplary cell-targeting molecule of the present invention. Briefly, cells samples of cell line F were incubated with “inactive SLT-1A::scFv6::F2” (SEQ ID NO:59), the reference molecule “inactive SLT-1A::scFv6” (SEQ ID NO:68), or just buffer alone for 6 hours, and then washed. Then, luciferase-reporter-transfected J76 T-cells were mixed with each sample, and the mixtures of cells were incubated for 18 hours. Next, luciferase activity was measured using the One-Glo™ Luciferase Assay System reagent (Promega Corp., Madison, Wis., U.S.). FIG. 10 and Table 6 shows the results from this intercellular T-cell engagement assay.

TABLE 6
Luciferase Signal Driven by the NFAT Response Element in Reporter
Cells after Recognition of Epitope Presentation by Target
Cells Incubated with “inactive SLTA-1A::scFv6::F2”

| | target positive | Average Luciferase Signal |
|---|---|---|
| Protein | cell-type | (RLU) |
| inactive SLT-1A::scFv6::F2 | Cell Line F | 565 |
| inactive SLT-1A::scFv6 | Cell Line F | 259 |
| buffer only | Cell Line F | 242 |

The results in Table 6 and FIG. 10 show that incubation with the exemplary cell-targeting molecule of the present invention “inactive SLT-1A::scFv6::F2” (SEQ ID NO:59) resulted in luciferase activity level greater than the background luciferase activity signal determined using “buffer only” treated cells or the luciferase activity from cell samples treated with the negative control molecule “inactive SLT-1A::scFv6” (SEQ ID NO:68). This in vitro T-cell engagement assay showed that Shiga toxin effector polypeptide-mediated delivery of a fused epitope-peptide to target positive cancer cells and subsequent cell-surface presentation of the epitope by the targeted cancer cells can result in intercellular engagement of T-cells and intracellular cell signaling characteristic of T-cell activation.

In addition, the activation of CTLs by target cells displaying epitope-peptide/MHC class I complexes (pMHC Is) is quantified using commercially available CTL response assays, e.g. CytoTox96® non-radioactive assays (Promega, Madison, Wis., U.S.), Granzyme B ELISpot assays (Mabtech, Inc., Cincinnati, Ohio, U.S.), caspase activity assays, and LAMP-1 translocation flow cytometric assays. To specifically monitor CTL-mediated killing of target cells, carboxyfluorescein succinimidyl ester (CFSE) is used to target-cells for in vitro and in vivo investigation as described in the art {see e.g. Durward M et al., J Vis Exp 45 pii 2250 (2010)).

In summary, multiple cell-targeting molecules, each comprising 1) a cell-targeting binding region, 2) a Shiga toxin effector polypeptide, and 3) a fused, heterologous, human CD8+ T-cell epitope cargo, exhibited a level of cytotoxicity that demonstrated they each exhibited a sufficient level of intracellular routing of a Shiga toxin effector polypeptide component to the cytosol (Table 7). Taken together, these results show that Shiga toxin effector functions, particularly subcellular routing, can be retained at high levels despite the presence of a fused epitope-peptide and regardless of the position of the epitope cargo within the molecule (Table 7). Furthermore, several cell-targeting molecules exhibited a level of epitope cargo delivery sufficient to produce a level of epitope-MHC class I presentation to stimulate intercellular, T-cell engagement with epitope-cargo-presenting cells.

TABLE 7
Summary of Experimental Results for the Exemplary, Cell-
Targeting Molecules of the Present Invention Tested Above

| | fusion | ribosome | cytotoxic |
|---|---|---|---|
| Structure | location | inhibition | activity |
| Epitope::Shiga toxin effector::binding region | N- | comparable | comparable |
| | terminus | to WT | to WT |
| Shiga toxin effector::binding region::Epitope | C- | comparable | comparable |
| | terminus | to WT | to WT |
| Shiga toxin effector::Epitope::binding region | internal | comparable | comparable |
| | | to WT | to WT |

### Example 2. IL-2R-targeting, Cell-Targeting Molecules Comprising Shiga Toxin A Subunit Effector Polypeptides and CD8+ T-Cell Epitope-Peptides

In this Example, the Shiga toxin effector polypeptide is derived from the A subunit of Shiga-like Toxin 1 (SLT-1A) as described above, optionally with amino acid residue substitutions conferring furin-cleavage resistance, such as, e.g., R248A/R251A (WO 2015/191764). A human, CD8+ T-cell epitope-peptide is selected based on MHC I molecule binding predictions, ULA types, already characterized immunogenicities, and readily available reagents as described above, such as the Cl-2 epitope-peptide GLDRNSGNY (SEQ ID NO:5). A proteinaceous binding region is derived from a ligand (the cytokine interleukin 2 or IL-2) for the human interleukin 2 receptor (IL-2R), which is capable of specifically binding an extracellular part of the human IL-2R. IL-2R is a cell-surface receptor expressed by various immune cell types, such as T-cells and natural killer cells.

### Construction and Production of IL-2R-Targeting, Cell-Targeting Fusion Proteins

The ligand-type binding region aIL-2R, the Shiga toxin effector polypeptide, and the CD8+ T-cell epitope are fused together to form a single, continuous polypeptide, such as “Cl-2::SLT-1A::IL-2” or “IL-2::C1-2::SLT-1A,” and, optionally, a KDEL is added to the carboxy terminus of the resulting polypeptide.

### Determining the In Vitro Characteristics of IL-2R-targeting, Cell-Targeting Molecules

The binding characteristics of cell-targeting molecules of this Example for IL-2R positive cells and IL-2R negative cells is determined by fluorescence-based, flow-cytometry. The Bmax for certain IL-2R-targeting, cell-targeting fusion proteins of this Example to positive cells is measured to be approximately 50,000-200,000 MFI with a K_{D} within the range of 0.01-100 nM, whereas there is no significant binding to IL-2R negative cells in this assay.

The ribosome inactivation abilities of IL-2R-targeting, cell-targeting fusion proteins of this Example are determined in a cell-free, in vitro protein translation as described above in the previous Examples. The inhibitory effect of the cell-targeting molecules of this Example on cell-free protein synthesis is significant. For certain IL-2R-targeting, cell-targeting fusion proteins, the ICso for protein synthesis in this cell-free assay is approximately 0.1-100 pM.

### Determining the Cytotoxicity of IL-2R-Targeting, Cell-Targeting Molecules Using a Cell-Kill Assay

The cytotoxicity characteristics of IL-2R-targeting, cell-targeting fusion proteins of this Example are determined by the general cell-kill assay as described above in the previous Examples using IL-2R positive cells. In addition, the selective cytotoxicity characteristics of the same IL-2R-targeting, cell-targeting fusion proteins of this Example are determined by the same general cell-kill assay using IL-2R negative cells as a comparison to the IL-2R positive cells. The CD50 values of the cell-targeting molecules of this Example are approximately 0.01-100 nM for IL-2R positive cells depending on the cell line. The CD50 values of IL-2R-targeting, cell-targeting fusion proteins of this Example are approximately 10-10,000 fold greater (less cytotoxic) for cells not expressing IL-2R on a cellular surface as compared to cells which do express IL-2R on a cellular surface. In addition, the induction of intermolecular CD8+ T-cell engagement of Cl-2-presenting target cells and cytotoxicity of IL-2R-targeting, cell-targeting fusion proteins of this Example is investigated for indirect cytotoxicity by heterologous, CD8+ T-cell epitope delivery and presentation leading to CTL-mediated cytotoxicity using assays known to the skilled worker and/or described herein.

### Determining the In Vivo Effects of the IL-2R-targeting, Cell-Targeting Molecules Using Animal Models

Animal models are used to determine the in vivo effects of certain IL-2R-targeting, cell-targeting fusion proteins of this Example on neoplastic cells. Various mice strains are used to test the effect of intravenous administration of IL-2R-targeting, cell-targeting fusion proteins of this Example on IL-2R positive cells in mice. Cell killing effects are investigated for both direct cytotoxicity and indirect cytotoxicity by CD8+ T-cell epitope delivery and presentation leading to CTL-mediated cytotoxicity using assays known to the skilled worker and/or described herein. Optionally, “inactive” variants of the cell-targeting molecules of this Example {e.g. E167D) are used to investigate indirect cytotoxicity by CD8+ T-cell epitope delivery in the absence of the catalytic activity of any Shiga toxin effector polypeptide component of the cell-targeting molecule.

### Example 3. CEA-Targeting, Cell-Targeting Molecules Comprising a Shiga Toxin Effector Polypeptide and a Heterologous, CD8+ T-Cell Epitope

Carcinoembryonic antigens (CEAs) expression in adult humans is associated with cancer cells, such as, e.g., adenocarcinomas of the breast, colon, lung, pancreas, and stomach. In this example, the Shiga toxin effector polypeptide is derived from the A subunit of Shiga Toxin (StxA), optionally with amino acid residue substitutions R248A/R25 1A conferring furin-cleavage resistance (WO 2015/191764). A human, CD8+ T-cell epitope-peptide is selected based on MHC I molecule binding predictions, HLA types, already characterized immunogenicities, and readily available reagents as described above, such as the F3-epitope ILRGSVAHK (SEQ ID NO: 11) described in Example 1 and Table 1. The immunoglobulin-type, binding region aCEA, which binds specifically and with high-affinity to an extracellular antigen on human carcinoembryonic antigen (CEA), such as the tenth human fibronectin type III domain derived binding region C743 as described in Pine C et al., J Biol Chem 286: 4165-72 (2011).

### Construction, Production, and Purification of CEA-Targeting, Cell-Targeting Molecules

The Shiga toxin effector polypeptide, aCEA binding region polypeptide, and heterologous, CD8+ T-cell epitope-peptide are operably linked together using standard methods known to the skilled worker to form cell-targeting molecules of the present invention. For example, fusion proteins are produced by expressing a polynucleotide encoding one or more of StxA::aCEA::F3, StxA::F3::aCEA, aCEA::StxA::F3, F3::aCEA::StxA, aCEA::F3::StxA, and F3::StxA::aCEA, which each optionally have one or more proteinaceous linkers described herein between the fused proteinaceous components. Expression of these exemplary CEA-targeting fusion proteins is accomplished using either bacterial and/or cell-free, protein translation systems as described in the previous Examples.

### Determining the In Vitro Characteristics of Exemplary CEA-Targeting, Cell-Targeting Fusion Proteins

The binding characteristics of cell-targeting molecule of this Example for CEA positive cells and CEA negative cells is determined by fluorescence-based, flow-cytometry. The Bmax for StxA::aCEA::F3, StxA::F3::aCEA, aCEA::StxA::F3, F3::aCEA::StxA, aCEA::F3::StxA, and F3::StxA::aCEA to CEA positive cells are each measured to be approximately 50,000-200,000 MFI with a KD within the range of 0.01-100 nM, whereas there is no significant binding to CEA negative cells in this assay.

The ribosome inactivation abilities of the fusion proteins of this Example are determined in a cell-free, in vitro protein translation as described above in the previous Examples. The inhibitory effect of the cytotoxic fusion proteins of this

Example on cell-free protein synthesis are significant. The ICso values on protein synthesis in this cell-free assay measured for StxA::aCEA::F3, StxA::F3::aCEA, aCEA::StxA::F3, F3::aCEA::StxA, aCEA::F3::StxA, and F3::StxA::aCEA are each approximately 0.1-100 pM.

### Determining the Cytotoxicity of Exemplary CEA-Targeting, Cell-Targeting Fusion Proteins Using a Cell-Kill Assay

The cytotoxicity characteristics of cell-targeting molecule of this Example are determined by the general cell-kill assay as described above in the previous Examples using CEA positive cells. In addition, the selective cytotoxicity characteristics of the exemplary CEA-targeting, cell-targeting fusion proteins are determined by the same general cell-kill assay using CEA negative cells as a comparison to the CEA antigen positive cells. The CD50 values measured for StxA::aCEA::F3, StxA::F3::aCEA, aCEA::StxA::F3, F3::aCEA::StxA, aCEA::F3::StxA, and F3::StxA::aCEA are approximately 0.01-100 nM for CEA positive cells depending on the cell line. The CD50 values of the CEA-targeting, cell-targeting fusion proteins of this Example are approximately 10-10,000 fold greater (less cytotoxic) for cells not expressing CEA on a cellular surface as compared to cells which do express CEA on a cellular surface. In addition, the induction of intermolecular CD8+ T-cell engagement of F3-presenting target cells and cytotoxicity of StxA::aCEA::F3, StxA::F3::aCEA, aCEA::StxA::F3, F3::aCEA::StxA, aCEA::F3::StxA, and F3::StxA::aCEA is investigated for indirect cytotoxicity by heterologous, CD8+ T-cell epitope delivery and presentation leading to CTL-mediated cytotoxicity using assays known to the skilled worker and/or described herein.

### Determining the In Vivo Effects of an Exemplary CEA-Targeting, Cell-Targeting Fusion Protein Using Animal Models

Animal models are used to determine the in vivo effects exemplary CEA-targeting fusion proteins on neoplastic cells. Various mice strains are used to test the effects on xenograft tumors of the cell-targeting fusion proteins StxA::aCEA::F3, StxA::F3::aCEA, aCEA::StxA::F3, F3::aCEA::StxA, aCEA::F3::StxA, and F3::StxA::aCEA after intravenous administration to mice injected with human neoplastic cells which express CEA(s) on their cell surfaces. Cell killing is investigated for both direct cytotoxicity and indirect cytotoxicity by CD8+ T-cell epitope cargo delivery and presentation leading to CTL-mediated cytotoxicity using assays known to the skilled worker and/or described herein. Optionally, “inactive” variants of the cell-targeting molecules of this Example {e.g. E167D) are used to investigate indirect cytotoxicity caused by CD8+ T-cell epitope delivery in the absence of the catalytic activity of any Shiga toxin effector polypeptide component of the cell-targeting molecule.

### Example 4. HER2-Targeting, Cell-Targeting Molecules Comprising a Shiga Toxin Effector Polypeptide and a Heterologous, CD8+ T-Cell Epitope

HER2 overexpression has been observed in breast, colorectal, endometrial, esophageal, gastric, head and neck, lung, ovarian, prostate, pancreatic, and testicular germ cell tumor cells. In this example, the Shiga toxin effector polypeptide is derived from the A subunit of Shiga Toxin (StxA), optionally with amino acid residue substitutions R248A/R25 1A conferring furin-cleavage resistance (WO 2015/191764). A human, CD8+ T-cell epitope-peptide is selected based on MHC I molecule binding predictions, HLA types, already characterized immunogenicities, and readily available reagents as described above, such as the C3-epitope GVMTRGRLK (SEQ ID NO:7) described in Example 1 and Table 1. The binding region aHER2, which binds an extracellular part of human HER2, is generated by screening or selected from available immunoglobulin-type polypeptides known to the skilled worker {see e.g. the anyrin repeat DARPin™ G3 which binds with high affinity to an extracellular epitope of HER2 (Goldstein R et al., Eur J Nucl Med Mol Imaging 42: 288-301 (2015))).

### Construction, Production, and Purification of HER2-Targeting, Cell-Targeting Molecules

The Shiga toxin effector polypeptide, aHER2 binding region polypeptide, and heterologous, CD8+ T-cell epitope-peptide are operably linked together using standard methods known to the skilled worker to form cell-targeting molecules of the present invention. For example, fusion proteins are produced by expressing a polynucleotide encoding one or more of StxA::aHER2::C3, StxA::C3::aHER2, aHER2::StxA::C3, C3::aHER2::StxA, aHER2::C3::StxA, and C3::StxA::aHER2, which each optionally have one or more proteinaceous linkers described herein between the fused proteinaceous components. Expression of these exemplary HER2-targeting fusion proteins is accomplished using either bacterial and/or cell-free, protein translation systems as described in the previous Examples.

### Determining the In Vitro Characteristics of Exemplary HER2-Targeting, Cell-Targeting Fusion Proteins

The binding characteristics of cell-targeting molecule of this Example for FIER2 positive cells and FIER2 negative cells is determined by fluorescence-based, flow-cytometry. The Bmax for StxA::aHER2::C3, StxA::C3::aHER2, aHER2::StxA::C3, C3::aHER2::StxA, aHER2::C3::StxA, and C3::StxA::aHER2 to FIER2 positive cells are each measured to be approximately 50,000-200,000 MFI with a K_{D} within the range of 0.01-100 nM, whereas there is no significant binding to FIER2 negative cells in this assay.

The ribosome inactivation abilities of the fusion proteins of this Example are determined in a cell-free, in vitro protein translation as described above in the previous Examples. The inhibitory effect of the cytotoxic fusion proteins of this Example on cell-free protein synthesis are significant. The ICso values on protein synthesis in this cell-free assay measured for StxA::aF£ER2::C3, StxA::C3::aHER2, αBER2::StxA::C3, C3::aHER2::StxA, aHER2::C3::StxA, and C3::StxA::aHER2 are each approximately 0.1-100 pM.

### Determining the Cytotoxicity of Exemplary HER2-Targeting, Cell-Targeting Fusion Proteins Using a Cell-Kill Assay

The cytotoxicity characteristics of cell-targeting molecule of this Example are determined by the general cell-kill assay as described above in the previous Examples using FIER2 positive cells. In addition, the selective cytotoxicity characteristics of the exemplary FIER2-targeting, cell-targeting fusion proteins are determined by the same general cell-kill assay using FIER2 negative cells as a comparison to the FIER2 antigen positive cells. The CD50 values measured for StxA::aHER2::C3, StxA::C3::aHER2, aHER2::StxA::C3, C3::aHER2::StxA, aHER2::C3::StxA, and C3::StxA::aHER2 are approximately 0.01-100 nM for FIER2 positive cells depending on the cell line. The CD50 values of the HER2-targeting, cell-targeting fusion proteins of this Example are approximately 10-10,000 fold greater (less cytotoxic) for cells not expressing FIER2 on a cellular surface as compared to cells which do express FIER2 on a cellular surface. In addition, the induction of intermolecular CD8+ T-cell engagement of C3-presenting target cells and cytotoxicity of StxA::aHER2::C3, StxA::C3::aHER2, aHER2::StxA::C3, C3::aHER2::StxA, aHER2::C3::StxA, and C3::StxA::aHER2 is investigated for indirect cytotoxicity by heterologous, CD8+ T-cell epitope delivery and presentation leading to CTL-mediated cytotoxicity using assays known to the skilled worker and/or described herein.

### Determining the In Vivo Effects of an Exemplary HER2-Targeting, Cell-Targeting Fusion Protein Using Animal Models

Animal models are used to determine the in vivo effects exemplary HER2-targeting fusion proteins on neoplastic cells. Various mice strains are used to test the effects on xenograft tumors of the cell-targeting fusion proteins StxA::aHER2::C3, StxA::C3::aHER2, aHER2::StxA::C3, C3::aHER2::StxA, aHER2::C3::StxA, and C3::StxA::aHER2 after intravenous administration to mice injected with human neoplastic cells which express HER2(s) on their cell surfaces. Cell killing is investigated for both direct cytotoxicity and indirect cytotoxicity by CD8+ T-cell epitope cargo delivery and presentation leading to CTL-mediated cytotoxicity using assays known to the skilled worker and/or described herein. Optionally, “inactive” variants of the cell-targeting molecules of this Example {e.g. E167D) are used to investigate indirect cytotoxicity caused by CD8+ T-cell epitope delivery in the absence of the catalytic activity of any Shiga toxin effector polypeptide component of the cell-targeting molecule.

### Example 5. EGFR-Targeting, Cell-Targeting Molecules Comprising a Shiga Toxin Effector Polypeptide and a Heterologous, CD8+ T-Cell Epitope

The expression of epidermal growth factor receptors is associated with human cancer cells, such as, e.g., lung cancer cells, breast cancer cells, and colon cancer cells. In this example, the Shiga toxin effector polypeptide is derived from the A subunit of Shiga Toxin (StxA), optionally with amino acid residue substitutions R248A/R251A conferring furin-cleavage resistance (WO 2015/191764). A human, CD8+ T-cell epitope-peptide is selected based on MHC I molecule binding predictions, HLA types, already characterized immunogenicities, and readily available reagents as described above, such as the CI-epitope VTEHDTLLY (SEQ ID NO:4) described in Example 1 and Table 1. The binding region aEGFR is derived from the AdNectin™ (GenBank Accession: 3QWQ_B), the Affibody™ (GenBank Accession: 2KZI_A; U.S. Pat. No. 8,598,113), or an antibody, all of which bind an extracellular part of one or more human epidermal growth factor receptors.

### Construction, Production, and Purification of EGFR-Targeting, Cell-Targeting Molecules

The Shiga toxin effector polypeptide, aEGFR binding region polypeptide, and heterologous, CD8+ T-cell epitope-peptide are operably linked together using standard methods known to the skilled worker to form cell-targeting molecules of the present invention. For example, fusion proteins are produced by expressing a polynucleotide encoding one or more of StxA::aEGFR::C1, StxA::C1::aEGFR, aEGFR::StxA::C1, C1::aEGFR::StxA, aEGFR::C1::StxA, and C1::StxA::aEGFR, which each optionally have one or more proteinaceous linkers described herein between the fused proteinaceous components. Expression of these exemplary EGFR-targeting fusion proteins is accomplished using either bacterial and/or cell-free, protein translation systems as described in the previous Examples.

### Determining the In Vitro Characteristics of Exemplary EGFR-Targeting, Cell-Targeting Fusion Proteins

The binding characteristics of cell-targeting molecule of this Example for EGFR+ cells and EGFR− cells is determined by fluorescence-based, flow-cytometry. The Bmax for StxA::aEGFR::C1, StxA::C1::aEGFR, aEGFR::StxA::C1, C1::aEGFR::StxA, aEGFR::C1::StxA, and CI::StxA::aEGFR to EGFR positive cells are each measured to be approximately 50,000-200,000 MFI with a KD within the range of 0.01-100 nM, whereas there is no significant binding to EGFR negative cells in this assay.

The ribosome inactivation abilities of the fusion proteins of this Example are determined in a cell-free, in vitro protein translation as described above in the previous Examples. The inhibitory effect of the cytotoxic fusion proteins of this Example on cell-free protein synthesis are significant. The ICso values on protein synthesis in this cell-free assay measured for StxA::aEGFR::C1, StxA::C1::aEGFR, aEGFR::StxA::C1, C1::aEGFR::StxA, aEGFR::C1::StxA, and C1::StxA::aEGFR are each approximately 0.1-100 pM.

### Determining the Cytotoxicity of Exemplary EGFR-Targeting, Cell-Targeting Fusion Proteins Using a Cell-Kill Assay

The cytotoxicity characteristics of cell-targeting molecule of this Example are determined by the general cell-kill assay as described above in the previous Examples using EGFR+ cells. In addition, the selective cytotoxicity characteristics of the exemplary EGFR-targeting, cell-targeting fusion proteins are determined by the same general cell-kill assay using EGFR-cells as a comparison to the EGFR antigen positive cells. The CD50 values measured for StxA::aEGFR::C1, StxA::C1::aEGFR, aEGFR::StxA::C1, C1::aEGFR::StxA, aEGFR::C1::StxA, and C1::StxA::aEGFR are approximately 0.01-100 nM for EGFR positive cells depending on the cell line. The CD50 values of the EGFR-targeting, cell-targeting fusion proteins of this Example are approximately 10-10,000 fold greater (less cytotoxic) for cells not expressing EGFR on a cellular surface as compared to cells which do express EGFR on a cellular surface. In addition, the induction of intermolecular CD8+ T-cell engagement of CI-presenting target cells and cytotoxicity of StxA::aEGFR::C1, StxA::C1::aEGFR, aEGFR::StxA::C1, C1::aEGFR::StxA, aEGFR::C1::StxA, and C1::StxA::aEGFR is investigated for indirect cytotoxicity by heterologous, CD8+ T-cell epitope delivery and presentation leading to CTL-mediated cytotoxicity using assays known to the skilled worker and/or described herein.

### Determining the In Vivo Effects of an Exemplary EGFR-Targeting, Cell-Targeting Fusion Protein Using Animal Models

Animal models are used to determine the in vivo effects exemplary EGFR-targeting fusion proteins on neoplastic cells. Various mice strains are used to test the effects on xenograft tumors of the cell-targeting fusion proteins StxA::aEGFR::C1, StxA::C1::aEGFR, aEGFR::StxA::C1, C1::aEGFR::StxA, aEGFR::C1::StxA, and C1::StxA::aEGFR after intravenous administration to mice injected with human neoplastic cells which express EGFR(s) on their cell surfaces. Cell killing is investigated for both direct cytotoxicity and indirect cytotoxicity by CD8+ T-cell epitope cargo delivery and presentation leading to CTL-mediated cytotoxicity using assays known to the skilled worker and/or described herein. Optionally, “inactive” variants of the cell-targeting molecules of this Example {e.g. E167D) are used to investigate indirect cytotoxicity caused by CD8+ T-cell epitope delivery in the absence of the catalytic activity of any Shiga toxin effector polypeptide component of the cell-targeting molecule.

### Example 6. Cell-Targeting Molecules Targeting Various Cell-Types, Each Comprising a Shiga Toxin A Subunit Effector Polypeptide and One or More, Heterologous, CD8+ T-Cell Epitope-Peptides Located Carboxy-Terminal to the Shiga Toxin A Subunit Effector Polypeptide Component

In this Example, three proteinaceous structures are associated with each other to form exemplary, cell-targeting molecules of the present invention. The Shiga toxin A Subunit effector polypeptide component having a Shiga toxin A 1 fragment region is derived from the A subunit of Shiga-like Toxin 1 (SLT-1A), Shiga toxin (StxA), and/or Shiga-like Toxin 2 (SLT-2A), optionally with amino acid residue substitutions conferring furin-cleavage resistance (WO 2015/191764). One or more CD8+ T-cell epitope-peptides are selected, such as, e.g., based on MHC I molecule binding predictions, HLA types, already characterized immunogenicities, and readily available reagents as described herein. A binding region component is derived from the immunoglobulin domain from the molecule chosen from column 1 of Table 8 and which binds the extracellular target biomolecule indicated in column 2 of Table 8.

Using reagents and techniques known in the art, the three components: 1) the immunoglobulin-derived binding region, 2) the Shiga toxin effector polypeptide, and 3) the CD8+ T-cell epitope-peptide(s) or a larger polypeptide comprising at least one heterologous CD8+ T-cell epitope-peptide, are associated with each other to form a cell-targeting molecule of the present invention wherein a CD8+ T-cell epitope-peptide is located carboxy-terminal to the carboxy terminus of the Shiga toxin A1 fragment region of the Shiga toxin effector polypeptide. The exemplary cell-targeting molecules of this Example are tested as described in the previous Examples using cells expressing the appropriate extracellular target biomolecules. The exemplary cell-targeting molecules of this Example may be used, e.g., to diagnose and treat diseases, conditions, and/or disorders indicated in column 3 of Table 8.

TABLE 8
Various Binding Regions for Cell Targeting

| Source of | Extracellular | |
|---|---|---|
| binding region | target | Application(s) |
| alemtuzumab | CD52 | B-cell cancers, such as lymphoma and leukemia, |
| | | and B-cell related immune disorders, such as |
| | | autoimmune disorders |
| basiliximab | CD25 | T-cell disorders, such as prevention of organ |
| | | transplant rejections, and some B-cell lineage |
| | | cancers |
| brentuximab | CD30 | hematological cancers, B-cell related immune |
| | | disorders, and T-cell related immune disorders |
| catumaxomab | EpCAM | various cancers, such as ovarian cancer, |
| | | malignant ascites, gastric cancer |
| cetuximab | EGFR | various cancers, such as colorectal cancer and |
| | | head and neck cancer |
| daclizumab | CD25 | B-cell lineage cancers and T-cell disorders, such |
| | | as rejection of organ transplants |
| daratumumab | CD38 | hematological cancers, B-cell related immune |
| | | disorders, and T-cell related immune disorders |
| dinutuximab | ganglioside | Various cancers, such as breast cancer, myeloid |
| | GD2 | cancers, and neuroblastoma |
| efalizumab | LFA-1 | autoimmune disorders, such as psoriasis |
| | (CD 11a) | |
| ertumaxomab | HER2/neu | various cancers and tumors, such as breast cancer |
| | | and colorectal cancer |
| gemtuzumab | CD33 | myeloid cancer or immune disorder |
| ibritumomab | CD20 | B-cell cancers, such as lymphoma and leukemia, |
| | | and B-cell related immune disorders, such as |
| | | autoimmune disorders |
| inotuzumab | CD22 | B-cell cancers, such as lymphoma and leukemia, |
| | | and B-cell related immune disorders, such as |
| | | autoimmune disorders |
| ipilimumab | CD 152 | T-cell related disorders and various cancers, such |
| | | as leukemia, melanoma |
| muromonab | CD3 | prevention of organ transplant rejections |
| natalizumab | initegrin α4 | autoimmune disorders, such as multiple sclerosis |
| | | and Crohn's disease |
| obinutuzumab | CD20 | B-cell cancers, such as lymphoma and leukemia, |
| | | and B-cell related immune disorders, such as |
| | | autoimmune disorders |
| ocaratuzumab | CD20 | B-cell cancers, such as lymphoma and leukemia, |
| | | and B-cell related immune disorders, such as |
| | | autoimmune disorders |
| ocrelizumab | CD20 | B-cell cancers, such as lymphoma and leukemia, |
| | | and B-cell related immune disorders, such as |
| | | autoimmune disorders |
| ofatumumab | CD20 | B-cell cancers, such as lymphoma and leukemia, |
| | | and B-cell related immune disorders, such as |
| | | autoimmune disorders |
| palivizumab | F protein of | treat respiratory syncytial virus |
| | respiratory | |
| | syncytial | |
| | virus | |
| panitumumab | EGFR | various cancers, such as colorectal cancer and |
| | | head and neck cancer |
| pertuzumab | HER2/neu | various cancers and tumors, such as breast cancer |
| | | and colorectal cancer |
| pro 140 | CCR5 | HIV infection and T-cell disorders |
| ramucirumab | VEGFR2 | various cancers and cancer related disorders, |
| | | such as solid tumors |
| rituximab | CD20 | B-cell cancers, such as lymphoma and leukemia, |
| | | and B-cell related immune disorders, such as |
| | | autoimmune disorders |
| tocilizumab or | IL-6 receptor | autoimmune disorders, such as rheumatoid |
| atlizumab | | arthritis |
| tositumomab | CD20 | B-cell cancers, such as lymphoma and leukemia, |
| | | and B-cell related immune disorders, such as |
| | | autoimmune disorders |
| trastuzumab | HER2/neu | various cancers and tumors, such as breast cancer |
| | | and colorectal cancer |
| ublituximab | CD20 | B-cell cancers, such as lymphoma and leukemia, |
| | | and B-cell related immune disorders, such as |
| | | autoimmune disorders |
| vedolizumab | integrin α4β7 | autoimmune disorders, such as Crohn's disease |
| | | and ulcerative colitis |
| CD20 binding scFv(s) | CD20 | B-cell cancers, such as lymphoma and leukemia, |
| Geng S et al., Cell | | and B-cell related immune disorders, such as |
| Mol Immunol 3: 439- | | autoimmune disorders |
| 43 (2006); Olafesn T | | |
| et al., Protein Eng Des | | |
| Sel 23: 243-9 (2010) | | |
| CD22 binding scFv(s) | CD22 | B-cell cancers or B-cell related immune disorders |
| Kawas S et al., MAbs | | |
| 3: 479-86 (2011) | | |
| CD25 binding scFv(s) | CD25 | various cancers of the B-cell lineage and immune |
| Muramatsu H et al., | | disorders related to T-cells |
| Cancer Lett 225: 225- | | |
| 36 (2005) | | |
| CD30 binding | CD30 | B-cell cancers or B-cell/T-cell related immune |
| monoclonal antibody(s) | | disorders |
| Klimka A et al., Br J | | |
| Cancer 83: 252-60 | | |
| (2000) | | |
| CD33 binding | CD33 | myeloid cancer or immune disorder |
| monoclonal antibody(s) | | |
| Benedict C et al., J | | |
| Immunol Methods | | |
| 201: 223-31 (1997) | | |
| CD38 binding | CD38 | hematological cancers, B-cell related immune |
| immunoglobulin | | disorders, and T-cell related immune disorders |
| domains U.S. Pat. No. | | |
| 8,153,765 | | |
| CD40 binding scFv(s) | CD40 | various cancers and immune disorders |
| Ellmark P et al., | | |
| Immunology 106: 456- | | |
| 63 (2002) | | |
| CD52 binding | CD52 | B-cell cancers, such as lymphoma and leukemia, |
| monoclonal | | and B-cell related immune disorders, such as |
| antibody(s) | | autoimmune disorders |
| U.S. Pat. No. 7,910,104 | | |
| B2 | | |
| CD56 binding | CD56 | immune disorders and various cancers, such as |
| monoclonal antibody(s) | | lung cancer, Merkel cell carcinoma, myeloma |
| Shin J et al., | | |
| Hybridoma 18: 521-7 | | |
| (1999) | | |
| CD79 binding | CD79 | B-cell cancers or B-cell related immune disorders |
| monoclonal antibody(s) | | |
| Zhang L et al., Ther | | |
| Immunol 2: 191-202 | | |
| (1995) | | |
| CD248 binding | CD248 | various cancers, such as inhibiting angiogenesis |
| scFv(s) | | |
| Zhao A et al., J | | |
| Immunol Methods | | |
| 363: 221-32 (2011) | | |
| EpCAM binding | EpCAM | various cancers, such as ovarian cancer, |
| monoclonal antibody(s) | | malignant ascites, gastric cancer |
| Schanzer J et al., J | | |
| Immunother 29: 477- | | |
| 88 (2006) | | |
| PSMA binding | PSMA | prostate cancer |
| monoclonal antibody(s) | | |
| Frigerio B et al., Eur J | | |
| Cancer 49: 2223-32 | | |
| (2013) | | |
| Eph-B2 binding | Eph-B2 | for various cancers such as colorectal cancer and |
| monoclonal antibody(s) | | prostate cancer |
| Abéngozar M et al., | | |
| Blood 119: 4565-76 | | |
| (2012) | | |
| Endoglin binding | Endoglin | various cancers, such as breast cancer and |
| monoclonal antibody(s) | | colorectal cancers |
| Völkel T et al., | | |
| Biochim Biophys Res | | |
| Acta 1663: 158-66 | | |
| (2004) | | |
| FAP binding | FAP | various cancers, such as sarcomas and bone |
| monoclonal antibody(s) | | cancers |
| Zhang J et al., FASEB | | |
| J 27: 581-9 (2013) | | |
| CEA binding | CEA | various cancers, such as gastrointestinal cancer, |
| antibody(s) and | | pancreatic cancer, lung cancer, and breast cancer |
| scFv(s) Neumaier | | |
| M et al., Cancer | | |
| Res 50: 2128-34 | | |
| (1990); Pavoni E et | | |
| al., BMC Cancer 6: 4 | | |
| (2006); Yazaki P et | | |
| al., Nucl Med Biol 35: | | |
| 151-8 (2008); Zhao J | | |
| et al., Oncol Res 17: | | |
| 217-22 (2008) | | |
| CD24 binding | CD24 | various cancers, such as bladder cancer |
| monoclonal antibody(s) | | |
| Kristiansen G et al., | | |
| Lab Invest 90: 1102- | | |
| 16 (2010) | | |
| LewisY antigen | LewisY | various cancers, such as cervical cancer and |
| binding scFv(s) | antigens | uterine cancer |
| Power B et al., | | |
| Protein Sci 12: 734-47 | | |
| (2003); monoclonal | | |
| antibody BR96 | | |
| Feridani A et al., | | |
| Cytometry 71: 361-70 | | |
| (2007) | | |
| adalimumab | TNF-α | various cancers and immune disorders, such as |
| | | Rheumatoid arthritis, Crohn's Disease, Plaque |
| | | Psoriasis, Psoriatic Arthritis, Ankylosing |
| | | Spondylitis, Juvenile Idiopathic Arthritis, |
| | | Hemolytic disease of the newborn |
| afelimomab | TNF-α | various cancers and immune disorders |
| ald518 | IL-6 | various cancers and immune disorders, such as |
| | | rheumatoid arthritis |
| anrukinzumab or ima- | IL-13 | various cancers and immune disorders |
| 638 | | |
| briakinumab | IL-12, IL-23 | various cancers and immune disorders, such as |
| | | psoriasis, rheumatoid arthritis, inflammatory |
| | | bowel diseases, multiple sclerosis |
| brodalumab | IL-17 | various cancers and immune disorders, such as |
| | | inflammatory diseases |
| canakinumab | IL-1 | various cancers and immune disorders, such as |
| | | rheumatoid arthritis |
| certolizumab | TNF-α | various cancers and immune disorders, such as |
| | | Crohn's disease |
| fezakinumab | IL-22 | various cancers and immune disorders, such as |
| | | rheumatoid arthritis, psoriasis |
| ganitumab | IGF-I | various cancers |
| golimumab | TNF-α | various cancers and immune disorders, such as |
| | | rheumatoid arthritis, psoriatic arthritis, |
| | | ankylosing spondylitis |
| infliximab | TNF-α | various cancers and immune disorders, such as |
| | | rheumatoid arthritis, ankylosing spondylitis, |
| | | psoriatic arthritis, psoriasis, Crohn's disease, |
| | | ulcerative colitis |
| ixekizumab | TL-17A | various cancers and immune disorders, such as |
| | | autoimmune diseases |
| mepolizumab | IL-5 | various immune disorders and cancers, such as |
| | | B-cell cancers |
| nerelimomab | TNF-α | various cancers and immune disorders |
| olokizumab | IL6 | various cancers and immune disorders |
| ozoralizumab | TNF-α | inflammation |
| perakizumab | IL17A | various cancers and immune disorders, such as |
| | | arthritis |
| placulumab | human TNF | various immune disorders and cancers |
| sarilumab | IL6 | various cancers and immune disorders, such as |
| | | rheumatoid arthritis, ankylosing spondylitis |
| siltuximab | IL-6 | various cancers and immune disorders |
| sirukumab | IL-6 | various cancers and immune disorders, such as |
| | | rheumatoid arthritis |
| tabalumab | BAFF | B-cell cancers |
| ticilimumab or | CTLA-4 | various cancers |
| tremelimumab | | |
| tildrakizumab | IL23 | immunologically mediated inflammatory |
| | | disorders |
| tnx-650 | IL-13 | various cancers and immune disorders, such as |
| | | B-cell cancers |
| tocilizumab or | IL-6 receptor | various cancers and immune disorders, such as |
| atlizumab | | rheumatoid arthritis |
| ustekinumab | IL-12, IL-23 | various cancers and immune disorders, such as |
| | | multiple sclerosis, psoriasis, psoriatic arthritis |
| Various growth | VEGFR, | various cancer, such as breast cancer and colon |
| factors: VEGF, EGF1, | EGFR, FGFR | cancer, and to inhibit vascularization |
| EGF2, FGF | | |
| Various cytokines: IL-2, | IL-2R, IL-6R, | various immune disorders and cancers |
| IL-6, IL-23, CCL2, | IL-23R, | |
| BAFFs, TNFs, | CD80/CD86, | |
| RANKL | TNFRSF13/ | |
| | TNFRSF17, | |
| | TNFR | |
| Broadly neutralizing | Influenza | viral infections |
| antibodies identified | surface | |
| from patient samples | antigens, e.g. | |
| Prabakaran et al., | hemagglutinins | |
| Front Microbiol 3: | and influenza | |
| 277 (2012) | matrix | |
| | protein 2 | |
| Broadly neutralizing | Coronavirus | viral infections |
| antibodies identified | surface | |
| from patient samples | antigens | |
| Prabakaran et al., | | |
| Front Microbiol 3: | | |
| 277 (2012) | | |
| Broadly neutralizing | Henipaviruses | viral infections |
| antibodies identified | surface | |
| from patient samples | antigens | |
| Prabakaran et al., | | |
| Front Microbiol 3: | | |
| 277 (2012) | | |

While some embodiments of the invention have been described by way of illustration, it will be apparent that the invention may be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety. The disclosures of U.S. provisional patent application Ser. Nos. 61/777,130, 61/932,000, 61/951,110, 61/951,121, 62/010,918, and 62/049,325 are each incorporated herein by reference in their entirety. The international patent application publications WO 2014/164680, WO 2014/164693, WO 2015/138435, WO 2015/138452, WO 2015/113005, WO 2015/113007, and WO 2015/191764, are each incorporated herein by reference in its entirety. The disclosures of U.S. patent application publications US 2007/0298434 A1, US 2009/0156417 A1, US 2013/0196928 A1, and US 2016/0177284 A1 are each incorporated here by reference in their entirety. The disclosure of international PCT patent application serial number PCT/US2016/016580 is incorporated herein by reference in its entirety. The complete disclosures of all electronically available biological sequence information from GenBank (National Center for Biotechnology Information, U.S.) for amino acid and nucleotide sequences cited herein are each incorporated herein by reference in their entirety.

Sequence_Listing

| | Text | |
|---|---|---|
| ID Number | Description | Biological_Sequence |
| SEQ ID NO: 1 | Shiga-like | KEFTLDF STAKTYVDSLNVIRS AIGTPLQTIS S |
| | toxin 1 Subunit | GGTSLLMIDSGSGDNLF AVDVRGIDPEEGRF |
| | A (SLT-1A) | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASRVA |
| | | RMASDEFPSMCPADGRVRGITHNKILWDSST |
| | | LGAILMRRTISS |
| SEQ ID NO: 2 | Shiga toxin | KEFTLDF STAKTYVDSLNVIRS AIGTPLQTIS S |
| | Subunit A | GGTSLLMIDSGSGDNLF AVDVRGIDPEEGRF |
| | (StxA) | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASRVA |
| | | RMASDEFPSMCPADGRVRGITHNKILWDSST |
| | | LGAILMRRTISS |
| SEQ ID NO: 3 | Shiga-like | DEFTVDFSSQKSYVDSLNSIRSAISTPLGNISQ |
| | toxin 2 Subunit | GGVSVSVFNHVLGGNYISLNVRGLDPYSERF |
| | A (SLT-2A) | NHLRLIMERNNL YVAGFFNTETNIFYRFSDF S |
| | | HISVPDVITVSMTTD SSYSSLQRIADLERTGM |
| | | QIGRHSLVGSYLDLMEFRGRSMTRASSRAM |
| | | LRFVTVIAEALRFRQIQRGFRPALSEASPLYT |
| | | MTAQDVDLTLNWGRISNVLPEYRGEEGVRI |
| | | GRISFNSLSAILGSVAVILNCHSTGSYSVRSVS |
| | | QKQKTECQIVGDRAAIKVNNVLWEANTIAA |
| | | LLNRKPQDLTEPNQ |
| SEQ ID NO: 4 | T-cell epitope- | VTEHDTLLY |
| | petide CI | |
| SEQ ID NO: 5 | T-cell epitope- | GLDRNSGNY |
| | petide CI-2 | |
| SEQ ID NO: 6 | T-cell epitope- | NLVPMVATV |
| | peptide C2 | |
| SEQ ID NO: 7 | T-cell epitope- | GVMTRGRLK |
| | peptide C3 | |
| SEQ ID NO: 8 | T-cell epitope- | VYALPLKML |
| | peptide C24 | |
| SEQ ID NO: 9 | T-cell epitope- | QYDPVAALF |
| | peptide C24-2 | |
| SEQ ID NO: 10 | T-cell epitope- | GILGFVFTL |
| | peptide F2 | |
| SEQ ID NO: 11 | T-cell epitope- | ILRGSVAHK |
| | peptide F3 | |
| SEQ ID NO: 12 | T-cell epitope- | CLGGLLTMV |
| | peptide E2 | |
| SEQ ID NO: 13 | cell-targeting | VTEHDTLLYKEFTLDFSTAKTYVDSLNVIRS |
| | molecule 1 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAEFPKPSTPPGSSGGAPDIQM |
| | | TQSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGGGGSQVQLQESGPGLVRPSQTLS |
| | | LTCTVSGFSLTSYGVHWVRQPPGRGLEWIG |
| | | VMWRGGSTDYNAAFMSRLNITKDNSKNQV |
| | | SLRLSSVTAADTAVYYCAKSMITTGFVMDS |
| | | WGQGSLVTVSS |
| SEQ ID NO: 14 | cell-targeting | GLDRNSGNYKEFTLDFSTAKTYVDSLNVIRS |
| | molecule 2 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAEFPKPSTPPGSSGGAPDIQM |
| | | TQSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGGGGSQVQLQESGPGLVRPSQTLS |
| | | LTCTVSGFSLTSYGVHWVRQPPGRGLEWIG |
| | | VMWRGGSTDYNAAFMSRLNITKDNSKNQV |
| | | SLRLSSVTAADTAVYYCAKSMITTGFVMDS |
| | | WGQGSLVTVSS |
| SEQ ID NO: 15 | cell-targeting | GVMTRGRLKEFTLDFSTAKTYVDSLNVIRSA |
| | molecule 3 | IGTPLQTISSGGTSLLMIDSGSGDNLFAVDVR |
| | | GIDPEEGRFNNLRLIVERNNLYVTGFVNRTN |
| | | NVFYRFADFSHVTFPGTTAVTLSGDSSYTTL |
| | | QRVAGISRTGMQINRHSLTTSYLDLMSHSGT |
| | | SLTQSVARAMLRFVTVTAEALRFRQIQRGFR |
| | | TTLDDLSGRSYVMTAEDVDLTLNWGRLSSV |
| | | LPDYHGQDSVRVGRISFGSINAILGSVALILN |
| | | SHHHASAVAAEFPKPSTPPGSSGGAPDIQMT |
| | | QSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGGGGSQVQLQESGPGLVRPSQTLS |
| | | LTCTVSGFSLTSYGVHWVRQPPGRGLEWIG |
| | | VMWRGGSTDYNAAFMSRLNITKDNSKNQV |
| | | SLRLSSVTAADTAVYYCAKSMITTGFVMDS |
| | | WGQGSLVTVSS |
| SEQ ID NO: 16 | cell-targeting | VYALPLKMLKEFTLDFSTAKTYVDSLNVIRS |
| | molecule 4 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAEFPKPSTPPGSSGGAPDIQM |
| | | TQSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGGGGSQVQLQESGPGLVRPSQTLS |
| | | LTCTVSGFSLTSYGVHWVRQPPGRGLEWIG |
| | | VMWRGGSTDYNAAFMSRLNITKDNSKNQV |
| | | SLRLSSVTAADTAVYYCAKSMITTGFVMDS |
| | | WGQGSLVTVSS |
| SEQ ID NO: 17 | cell-targeting | NLVPMVATVKEFTLDFSTAKTYVDSLNVIRS |
| | molecule 5 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NCHHHASAVAAEFPKPSTPPGSSGGAPDIQM |
| | | TQSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGSTSGSGKPGSGEGSTKGQVQLQE |
| | | SGPGLVRPSQTLSLTCTVSGFSLTSYGVHWV |
| | | RQPPGRGLEWIGVMWRGGSTDYNAAFMSR |
| | | LNITKDNSKNQVSLRLSSVTAADTAVYYCA |
| | | KSMITTGFVMDSWGQGSLVTVSS |
| SEQ ID NO: 18 | cell-targeting | GILGFVFTLKEFTLDFSTAKTYVDSLNVIRSAI |
| | molecule 6 | GTPLQTISSGGTSLLMIDSGSGDNLFAVDVR |
| | | GIDPEEGRFNNLRLIVERNNLYVTGFVNRTN |
| | | NVFYRFADFSHVTFPGTTAVTLSGDSSYTTL |
| | | QRVAGISRTGMQINRHSLTTSYLDLMSHSGT |
| | | SLTQSVARAMLRFVTVTAEALRFRQIQRGFR |
| | | TTLDDLSGRSYVMTAEDVDLTLNWGRLSSV |
| | | LPDYHGQDSVRVGRISFGSINAILGSVALILN |
| | | CHHHASAVAAEFPKPSTPPGSSGGAPDIQMT |
| | | QSPSSLSASVGDRVTITCKASEDIYNRLTWY |
| | | QQKPGKAPKLLISGATSLETGVPSRFSGSGSG |
| | | TDFTFTISSLQPEDIATYYCQQYWSNPYTFGQ |
| | | GTKVEIKGSTSGSGKPGSGEGSTKGQVQLQE |
| | | SGPGLVRPSQTLSLTCTVSGFSLTSYGVHWV |
| | | RQPPGRGLEWIGVMWRGGSTDYNAAFMSR |
| | | LNITKDNSKNQVSLRLSSVTAADTAVYYCA |
| | | KSMITTGFVMDSWGQGSLVTVS |
| SEQ ID NO: 19 | cell-targeting | DIQMTQSP SSL SASVGDRVTITCRASQD VNT |
| | molecule 7 | AVAWYQQKPGKAPKLLIYSASFLYSGVPSRF |
| | | SGSRS GTDF TLTISSLQPEDFATYYCQQHYTT |
| | | PPTFGQGTKVEIKRTGSTSGSGKPGSGEGSEV |
| | | QLVESGGGLVQPGGSLRLSCAASGFNIKDTY |
| | | IHWVRQAPGKGLEWVARIYPTNGYTRYAD S |
| | | VKGRFTISADTSKNTAYLQMNSLRAEDTAV |
| | | YYCSRWGGDGFYAMDVWGQGTLVTVSSEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRFADFHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVAR |
| SEQ ID NO: 20 | cell-targeting | QVQLQQPGAELVKPGASVKMSCKTSGYTFT |
| | molecule 8 | SYNVHWVKQTPGQGLEWIGAIYPGNGDTSF |
| | | NQKFKGKATLTADK SSSTVYMQLSSLTSED S |
| | | AVYYCARSNYYGS SYVWFFDVWGAGTTVT |
| | | VSSGSTSGSGKPGSGEGSQIVLSQSPTILSASP |
| | | GEKVTMTCRAS SSVSYMDWYQQKPGS SPKP |
| | | WIYATSNLASGVPARFSGSGSGTSYSLTISRV |
| | | EAEDAATYYCQQWISNPPTFGAGTKLELKEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRFADFSHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVAR |
| SEQ ID NO: 21 | cell-targeting | KEFTLDF STAKTYVDSLNVIRS AIGTPLQTIS S |
| | molecule 9 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNSHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSVTEHDTLLY |
| SEQ ID NO: 22 | cell-targeting | KEFTLDF STAKTYVDSLNVIRS AIGTPLQTIS S |
| | molecule 10 | GGTSLLMID SGSGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSNLVPMVATV |
| SEQ ID NO: 23 | cell-targeting | KEFTLDF STAKTYVDSLNVIRSAIGTPLQTIS S |
| | molecule 11 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNSHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSQYDPVAALF |
| SEQ ID NO: 24 | cell-targeting | KEFTLDF STAKTYVDSLNVIRSAIGTPLQTIS S |
| | molecule 12 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNSHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSCLGGLLTMV |
| SEQ ID NO: 25 | cell-targeting | KEFTLDF STAKTYVDSLNVIRSAIGTPLQTIS S |
| | molecule 13 | GGTSLLMID SGSGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNSHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSILRGSVAHK |
| SEQ ID NO: 26 | cell-targeting | KEFTLDF STAKTYVDSLNVIRSAIGTPLQTIS S |
| | molecule 14 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKGS |
| | | TSGSGKPGSGEGSTKGQVQLQESGPGLVRPS |
| | | QTLSLTCTVSGFSLTSYGVHWVRQPPGRGLE |
| | | WIGVMWRGGSTDYNAAFMSRLNITKDNSK |
| | | NQVSLRLSSVTAADTAVYYCAKSMITTGFV |
| | | MD SWGQGSLVTVSSNLVPMVATV |
| SEQ ID NO: 27 | cell-targeting | KEFTLDF STAKTYVDSLNVIRSAIGTPLQTIS S |
| | molecule 15 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNCHHHASAVA |
| | | AEFPKPSTPPGSSGGAPDIQMTQSPSSLSASV |
| | | GDRVTITCRASQDVNTAVAWYQQKPGKAP |
| | | KLLIYSASFLYSGVPSRFSGSRSGTDFTLTISS |
| | | LQPEDFATYYCQQHYTTPPTFGQGTKVEIKG |
| | | GGGSEVQLVESGGGLVQPGGSLRLSCAASGF |
| | | NIKDTYIHWVRQAPGKGLEWVARIYPTNGY |
| | | TRYADSVKGRFTISADTSKNTAYLQMNSLR |
| | | AEDTAVYYCSRWGGDGFYAMDYWGQGTL |
| | | VTVSSNLVPMVATV |
| SEQ ID NO: 28 | cell-targeting | KEFTLDF STAKTYVDSLNVIRSAIGTPLQTIS S |
| | molecule 16 | GGTSLLMID SGSGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKP STPPGSSGGAPEVQLVESGGGLVQP |
| | | GGSLRL SCAA SGFTF SD SWIHWVRQAPGKG |
| | | LEWVAWISPYGGSTYYADSVKGRFTISADTS |
| | | KNTAYLQMNSLRAEDTAVYYCARRHWPGG |
| | | FDYWGQGTLVTVSSGGGGSGGGGSGGGGS |
| | | GGGGSGGGGSDIQMTQSPSSLSASVGDRVTI |
| | | TCRASQDVSTAVAWYQQKPGKAPKLLIYSA |
| | | SFLYSGVPSRFSGSGSGTDFTLTISSLQPEDFA |
| | | TYYCQQYLYHPATFGQGTKVEIKGILGFVFT |
| | | L |
| SEQ ID NO: 29 | cell-targeting | KEFTLDF STAKTYVDSLNVIRSAIGTPLQTIS S |
| | molecule 17 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPDIQMTQSPSSLSASV |
| | | GDRVTITCRASQGIS SWLAWYQQKPEKAPKS |
| | | LIYAASSLQSGVPSRFSGSGSGTDFTLTISSLQ |
| | | PEDFATYYCQQYNSYPYTFGQGTKLEIKGGG |
| | | GSQVQLVQSGAEVKKPGASVKVSCKASGYT |
| | | FTSYDVHWVRQAPGQRLEWMGWLHADTGI |
| | | TKFSQKFQGRVTITRDTSASTAYMELSSLRSE |
| | | DTAVYYCARERIQLWFDYWGQGTLVTVSSN |
| | | LVPMVATV |
| SEQ ID NO: 30 | cell-targeting | KEFTLDF STAKTYVDSLNVIRSAIGTPLQTIS S |
| | molecule 18 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNCHHHASAVA |
| | | AEFPKPSTPPGSSGGAPDIQMTQSPSSLSASV |
| | | GDRVTITCKASEDIYNRLTWYQQKPGKAPK |
| | | LLISGATSLETGVPSRFSGSGSGTDFTFTISSL |
| | | QPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSNLVPMVATV |
| SEQ ID NO: 31 | cell-targeting | KEFTLDF STAKTYVDSLNVIRSAIGTPLQTIS S |
| | molecule 19 | GGTSLLMID SGSGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPQVQLQQPGAELVKP |
| | | GASVKMSCKTSGYTFTSYNVHWVKQTPGQ |
| | | GLEWIGAIYPGNGD TSFNQKFKGKATLTAD |
| | | K SSSTVYMQL SSLTSED SAVYYCARSNYYGS |
| | | SYVWFFDVWGAGTTVTVSSGSTSGSGKPGS |
| | | GEGSQIVLSQSPTILSASPGEKVTMTCRASSS |
| | | VSYMDWYQQKPGSSPKPWIYATSNLASGVP |
| | | ARFSGSGSGTSYSLTISRVEAEDAATYYCQQ |
| | | WISNPPTFGAGTKLELKNLVPMVATV |
| SEQ ID NO: 32 | cell-targeting | KEFTLDF STAKTYVDSLNVIRSAIGTPLQTIS S |
| | molecule 20 | GGTSLLMIDSGSGDNLF AVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKP STPPGS SGGAPQVQLVQSGAELVKP |
| | | GASVKMSCKASGYTFTSYNMHWVKQTPGQ |
| | | GLEWIGAIYPGNGDTSYNQKFKGKATLTAD |
| | | K SSSTAYMQL SSLTSED SAVYYCARAQLRPN |
| | | YWYFDVWGAGTTVTVSSGGGGSDIVLSQSP |
| | | AIL SASPGEKVTMTCRAS SSVSYMHWYQQK |
| | | PGSSPKPWIYATSNLASGVPARFSGSGSGTSY |
| | | SLTISRVEAEDAATYYCQQWISNPPTFGAGT |
| | | KLELKNLVPMVATV |
| SEQ ID NO: 33 | cell-targeting | KEFTLDF STAKTYVDSLNVIRSAIGTPLQTIS S |
| | molecule 21 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNCHHHASAVA |
| | | AEFPKPSTPPGSSGGAPQVQLQQPGAELVKP |
| | | GASVKMSCKASGYTFTSYNMHWVKQTPGR |
| | | GLEWIGAIYPGNGDTSYNQKFKGKATLTAD |
| | | K SSSTAYMQL SSLTSED SAVYYCARSTYYGG |
| | | DWYFNVWGAGTTVTVSAGSTSGSGKPGSGE |
| | | GSTKGQIVLSQSPAILSASPGEKVTMTCRASS |
| | | SVSYIHWFQQKPGSSPKPWIYATSNLASGVP |
| | | VRFSGSGSGTSYSLTISRVEAEDAATYYCQQ |
| | | WTSNPPTFGGGTKLEIKNLVPMVATV |
| SEQ ID NO: 34 | cell-targeting | KEFTLDF STAKTYVDSLNVIRSAIGTPLQTIS S |
| | molecule 22 | GGTSLLMID SG SGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPEVQLVESGGGLVQA |
| | | GGSLRLSCAASGITFSINTMGWYRQAPGKQR |
| | | ELVALISSIGDTYYADSVKGRFTISRDNAKNT |
| | | VYLQMNSLKPEDTAVYYCKRFRTAAQGTD |
| | | YWGQGTQVTVSSAHHSEDNLVPMVATV |
| SEQ ID NO: 35 | cell-targeting | KEFTLDF STAKTYVDSLNVIRSAIGTPLQTIS S |
| | molecule 23 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASAVA |
| | | AEFPKPSTPPGSSGGAPDIELTQSPSSFSVSLG |
| | | DRVTITCKASEDIYNRLAWYQQKPGNAPRLL |
| | | ISGATSLETGVPSRFSGSGSGKDYTLSITSLQT |
| | | EDVATYYCQQYWSTPTFGGGTKLEIKGS TSG |
| | | SGKPGSGEGSKVQLQESGPSLVQPSQRLSITC |
| | | TVSGFSLISYGVHWVRQSPGKGLEWLGVIW |
| | | RGGSTDYNAAFMSRLSITKDNSK SQVFFKM |
| | | NSLQADDTAIYFCAKTLITTGYAMDYWGQG |
| | | TTVTV SSNLVPMVATV |
| SEQ ID NO: 36 | cell-targeting | KEFTLDF STAKTYVDSLNVIRSAIGTPLQTIS S |
| | molecule 24 | GGTSLLMIDSGSGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNCHHHASAVA |
| | | AAHHSEDPSSKAPKAPEVQLVESGGGLVQA |
| | | GGSLRLSCAASGITFSINTMGWYRQAPGKQR |
| | | ELVALISSIGDTYYADSVKGRFTISRDNAKNT |
| | | VYLQMNSLKPEDTAVYYCKRFRTAAQGTD |
| | | YWGQGTQVTVSSNL VPMVATV |
| SEQ ID NO: 37 | cell-targeting | KEFTLDF STAKTYVDSLNVIRSAIGTPLQTIS S |
| | molecule 25 | GGTSLLMIDSGTGDNLFAVDVRGIDPEEGRF |
| | | NNLRLIVERNNLYVTGFVNRTNNVFYRFADF |
| | | SFTVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLSSVLPD YHGQD S |
| | | VRVGRISFGSrNAILGSVALILNSHHHASAVA |
| | | AEFPKPSTPPGSSGGAPASVSDVPRDLEVVA |
| | | ATPTSLLISWCRQRCADSYRITYGETGGNSP |
| | | VQEFTVPGSWKTATISGLKPGVDYTITVYVV |
| | | THYYGWDRYSFIPISrNYRTGSNLVPMVATV |
| SEQ ID NO: 38 | cell-targeting | ASVSDVPRDLEVVAATPTSLLISWCRQRCAD |
| | molecule 26 | SYRITYGETGGNSPVQEFTVPGSWKTATISGL |
| | | KPGVDYTITVYVVTHYYGWDRYSHPISrNYR |
| | | TGSEFPKPSTPPGS SGGAPKEFTLDF STAKTY |
| | | VDSLNVIRSAIGTPLQTIS SGGTSLLMIDSGTG |
| | | DNLFAVDVRGIDPEEGRFNNLRLIVERNNLY |
| | | VTGFVNRTNNVFYRFADF SHVTFPGTTAVTL |
| | | SGDSSYTTLQRVAGISRTGMQrNRHSLTTSYL |
| | | DLMSHSGTSLTQSVARAMLRFVTVTAEALR |
| | | FRQIQRGFRTTLDDLSGRSYVMTAEDVDLTL |
| | | NWGRLSSVLPDYHGQDSVRVGRISFGSrNAI |
| | | LGSVALILNCHHHASAVAANLVPMVATV |
| SEQ ID NO: 39 | cell-targeting | KEFTLDF STAKTYVDSLNVIRSAIGTPLQTIS S |
| | molecule 27 | GGTSLLMIDSGTGDNLFAVDVRGIDPEEGRF |
| | | NNLRLIVERNNLY VTGF VNRTNNVF YRF ADF |
| | | SHVTFPGTTAVTLSGDSSYTTLQRVAGISRTG |
| | | MQrNRHSLTTSYLDLMSHSGTSLTQSVARA |
| | | MLRFVTVTAEALRFRQIQRGFRTTLDDLSGR |
| | | SYVMTAEDVDLTLNWGRLSSVLPDYHGQD S |
| | | VRVGRISFGSrNAILGSVALILNSHHHASAVA |
| | | AEFPKPSTPPGS SGGAPAPTS SSTKKTQLQLE |
| | | HLLLDLQMILNGFNNYKNPKLTRMLTFKFY |
| | | MPKKATELKHLQCLEEELKPLEEVLNLAQSK |
| | | NFHLRPRDLISNrNVIVLELKGSETTFMCEYA |
| | | DETATIVEFLNRWITFCQSIISTLTNLVPMVA |
| | | TV |
| SEQ ID NO: 40 | cell-targeting | NLVPMVATVKEF TLDF STAKTYVD SLNVIRS |
| | molecule 28 | AIGTPLQTISSGGTSLLMID SGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQFNRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSrNAILGSVALIL |
| | | NCHHHASAVAAGGGGSGGDIQMTQSP SSLS |
| | | ASVGDRVTITCKASEDIYNRLTWYQQKPGK |
| | | APKLLISGATSLETGVPSRFSGSGSGTDFTFTI |
| | | SSLQPEDIATYYCQQYWSNPYTFGQGTKVEI |
| | | KGGGGSQVQLQESGPGLVRPSQTLSLTCTVS |
| | | GFSLTSYGVHWVRQPPGRGLEWIGVMWRG |
| | | GSTDYNAAFM SRLNITKDNSKNQVSLRLSSV |
| | | TAADTAVYYCAKSMITTGFVMDSWGQGSL |
| | | VTVSS |
| SEQ ID NO: 41 | cell-targeting | NLVPMVATVKEFTLDF STAKTYVD SLNVIRS |
| | molecule 29 | AIGTPLQTISSGGTSLLMID SGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTADALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAEFPKPSTPPGSSGGAPDIQM |
| | | TQSP SSL SA SVGDRVTITCKASEDIYNRLTWY |
| | | QQKPGKAPKLLISGATSLETGVPSRFSGSGSG |
| | | TDFTFTIS SLQPEDIATYYCQQYWSNPYTFGQ |
| | | GTKVEIKGSTSGSGKPGSGEGSTKGQVQLQE |
| | | SGPGLVRPSQTLSLTCTVSGFSLTSYGVHWV |
| | | RQPPGRGLEWIGVMWRGGSTDYNAAFMSR |
| | | LNITKDN SKNQVSLRLSSVTAADTAVYYCA |
| | | KSMITTGFVMDSWGQGSL VTVSS |
| SEQ ID NO: 42 | cell-targeting | NLVPMVATVKEFTLDF STAKTYVDSLNVIRS |
| | molecule 30 | AIGTPLQTI SSGGTSLLMID SGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NCHHHASAVAAEFPKPSTPPGS SGGAPDIQM |
| | | TQSP SSL SA SVGDRVTITCRASQDVNTAVAW |
| | | YQQKPGKAPKLLIYSA SFLYSGVPSRF SGSRS |
| | | GTDFTLTISSLQPEDFATYYCQQHYTTPPTFG |
| | | QGTKVEIKGGGGSEVQLVESGGGLVQPGGS |
| | | LRLSCAASGFNIKDTYIHWVRQAPGKGLEW |
| | | VARIYPTNGYTRYADSVKGRFTISADTSKNT |
| | | AYLQMNSLRAEDTAVYYCSRWGGDGFYAM |
| | | DYWGQGTL VTVSS |
| SEQ ID NO: 43 | cell-targeting | GILGFVFTLKEFTLDFSTAKTYVDSLNVIRSAI |
| | molecule 31 | GTPLQTISSGGTSLLMID SGSGDNLFAVDVR |
| | | GIDPEEGRFNNLRLIVERNNLYVTGFVNRTN |
| | | NVFYRFADF SHVTFPGTTAVTLSGDS SYTTL |
| | | QRVAGISRTGMQINRHSLTTSYLDLMSHSGT |
| | | SLTQSVARAMLRFVTVTADALRFRQIQRGFR |
| | | TTLDDLSGRSYVMTAEDVDLTLNWGRLSSV |
| | | LPDYHGQD SVRVGRISFGSINAILGS VALILN |
| | | SHHHASAVAAEFPKPSTPPGSSGGAPEVQLV |
| | | ESGGGLVQPGGSLRLSCAASGFTFSDSWIHW |
| | | VRQAPGKGLEWVAWISPYGGSTYYADSVK |
| | | GRFTISADTSKNTAYLQMNSLRAEDTAVYY |
| | | CARRHWPGGFDYWGQGTLVTVSSGGGGSG |
| | | GGGSGGGGSGGGGSGGGGSDIQMTQ SP SSL S |
| | | ASVGDRVTITCRASQDVSTAVAWYQQKPGK |
| | | APKLLIYSASFLYSGVPSRFSGSGSGTDFTLTI |
| | | SSLQPEDFATYYCQQYLYHPATFGQGTKVEI |
| | | K |
| SEQ ID NO: 44 | cell-targeting | NLVPMVATVKEFTLDF STAKTYVD SLNVIRS |
| | molecule 32 | AIGTPLQTISSGGTSLLMIDSGTGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NCHHHASAVAAGGGGSGGDIQMTQ SP SSLS |
| | | A SVGDRVTITCRASQGIS SWLAWYQQKPEK |
| | | APKSLIYAASSLQSGVPSRFSGSGSGTDFTLTI |
| | | SSLQPEDFATYYCQQYNSYPYTFGQGTKLEI |
| | | KGGGGSQVQLVQSGAEVKKPGA SVKVSCK |
| | | ASGYTFTSYDVHWVRQAPGQRLEWMGWLH |
| | | ADTGITKFSQKFQGRVTITRDTSASTAYMEL |
| | | SSLRSEDTAVYYCARERIQLWFDYWGQGTL |
| | | VTVSS |
| SEQ ID NO: 45 | cell-targeting | NLVPMVATVKEFTLDF STAKTYVD SLNVIRS |
| | molecule 33 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAEFPKPSTPPGSSGGAPDIQM |
| | | TQ SP SSL SASVGDRVTITCKASEDIYNRLTWY |
| | | QQKPGKAPKLLISGATSLETGVPSRFSGSGSG |
| | | TDFTFTIS SLQPEDIATYYCQQYWSNPYTFGQ |
| | | GTKVEIKGGGGSQVQLQESGPGLVRPSQTLS |
| | | LTCTVSGF SLTSYGVHWVRQPPGRGLEWIG |
| | | VMWRGGSTDYNAAFMSRLNITKDNSKNQV |
| | | SLRLSSVTAADTAVYYCAKSMITTGFVMDS |
| | | WGQGSLVTVSS |
| SEQ ID NO: 46 | cell-targeting | NLVPMVATVKEFTLDF STAKTYVD SLNVIRS |
| | molecule 34 | AIGTPLQTISSGGTSLLMIDSGSGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NCHHHASAVAAEFPKPSTPPGS SGGAPQVQL |
| | | QQPGAELVKPGASVKMSCKTSGYTFTSYNV |
| | | HWVKQTPGQGLEWIGAIYPGNGDTSFNQKF |
| | | KGKATLTADK SSSTVYMQLSSLTSED SAVY |
| | | YCARSNYYGS SYVWFFDVWGAGTTVTVSS |
| | | GSTSGSGKPGSGEGSQIVLSQSPTILSASPGEK |
| | | VTMTCRAS SSVSYMDWYQQKPGS SPKPWIY |
| | | ATSNLASGVPARFSGSGSGTSYSLTISRVEAE |
| | | DAATYYCQQWISNPPTFGAGTKLELK |
| SEQ ID NO: 47 | cell-targeting | NLVPMVATVKEFTLDF STAKTYVD SLNVIRS |
| | molecule 35 | AIGTPLQTISSGGTSLLMIDSGTGDNLFAVDV |
| | | RGIDPEEGRFNNLRLIVERNNLYVTGFVNRT |
| | | NNVFYRFADFSHVTFPGTTAVTLSGDSSYTT |
| | | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NSHHHASAVAAGGGGSGGQVQLVQSGAEL |
| | | VKPGASVKMSCKASGYTFTSYNMHWVKQT |
| | | PGQGLEWIGAIYPGNGDTSYNQKFKGKATL |
| | | TADKSSSTAYMQLSSLTSEDSAVYYCARAQ |
| | | LRPNYWYFDVWGAGTTVTVSSGGGGSGGG |
| | | GSGGGGSGGGGSGGGGSDIVLSQSPAILSASP |
| | | GEKVTMTCRAS SSVSYMHWYQQKPGS SPKP |
| | | WIYATSNLASGVPARFSGSGSGTSYSLTISRV |
| | | EAEDAATYYCQQWISNPPTFGAGTKLELK |
| SEQ ID NO: 48 | cell-targeting | APTSSSTKKTQLQLEHLLLDLQMILNGINNY |
| | molecule 36 | KNPKLTRMLTFKFYMPKKATELKHLQCLEE |
| | | ELKPLEEVLNLAQSKNFULRPRDLISNINVIV |
| | | LELKGSETTFMCEYADETATIVEFLNRWITF C |
| | | QSIISTLTEFPKPSTPPGS SGGAPNLVPMVATV |
| | | KEFTLDF STAKTYVDSLNVIRSAIGTPLQTIS S |
| | | GGTSLLMIDSGTGDNLFAVDVRGIDPEEGRF |
| | | NNLRLIVERNNLYVTGFVNRTNNVFYRFADF |
| | | SHVTFPGTTAVTLSGDSSYTTLQRVAGISRTG |
| | | MQINRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSINAILGSVALILNCHHHASAVA |
| | | A |
| SEQ ID NO: 49 | cell-targeting | GILGFVFTLKEFTLDFSTAKTYVDSLNVIRSAI |
| | molecule 37 | GTPLQTISSGGTSLLMIDSGTGDNLFAVDVR |
| | | GIDPEEGRFNNLRLIVERNNLYVTGFVNRTN |
| | | NVFYRFADF SHVTFPGTTAVTLSGDS SYTTL |
| | | QRVAGISRTGMQINRHSLTTSYLDLMSHSGT |
| | | SLTQSVARAMLRFVTVTAEALRFRQIQRGFR |
| | | TTLDDLSGRSYVMTAEDVDLTLNWGRLSSV |
| | | LPDYHGQD SVRVGRISFGSINAILGS VALILN |
| | | SHHHASAVAAEFPKPSTPPGSSGGAPAPTSSS |
| | | TKKTQLQLEHLLLDLQMILNGINNYKNPKLT |
| | | RMLTFKFYMPKKATELKHLQCLEEELKPLEE |
| | | VLNLAQSKNFHLRPRDLISNINVIVLELKGSE |
| | | TTFMCEYADETATIVEFLNRWITFCQSIISTLT |
| SEQ ID NO: 50 | exemplary | MNLVPMVATVKEF TLDF STAKTYVD SLNVI |
| | cell-targeting | R SAIGTPLQ TISSGGT SLLMID SGSGDNLFAV |
| | molecule 1 | DVRGIDPEEGRFNNLRLIVERNNLYVTGFVN |
| | (C2::SLT- | RTNNVFYRFADFSHVTFPGTTAVTLSGDSSY |
| | 1A::SCFV2) | TTLQRVAGISRTGMQINRHSLTTSYLDLMSH |
| | | SGTSLTQSVARAMLRFVTVTAEALRFRQIQR |
| | | GFRTTLDDLSGRSYVMTAEDVDLTLNWGRL |
| | | SSVLPDYHGQDSVRVGRISFGSINAILGSVAL |
| | | ILNCHHH ASAVAAEFPKP STPPGS SGGAPDIQ |
| | | MTQ SPSSLSASVGDRVTITCK ASEDIYNRLT |
| | | WYQQKPGK APKLLI SGATSLETGVP SRFSGS |
| | | GSGTDF TFTIS SLQPEDI ATYYCQQYWSNPYT |
| | | FGQGTKVEIKGS TSGSGKPGSGEGS TKGQVQ |
| | | LQESGPGLVRPSQTLSLTCTVSGFSLTSYGVH |
| | | WVRQPPGRGLEWIGVMWRGGSTDYNAAFM |
| | | SRLNITKDNSKNQVSLRLSSVTAADTAVYYC |
| | | AKSMITTGFVMDSWGQGSLVTVSS |
| SEQ ID NO: 51 | exemplary | MNLVPMVATVKEF TLDF STAKTYVD SLNVI |
| | cell-targeting | R SAIGTPLQ TISSGGT SLLMID SGSGDNLFAV |
| | molecule 2 | DVRGIDPEEGRFNNLRLIVERNNLYVTGFVN |
| | (inactive | RTNNVFYRFADFSHVTFPGTTAVTLSGDSSY |
| | C2::SLT- | TTLQRVAGISRTGMQINRHSLTTSYLDLMSH |
| | 1A::SCFV2) | SGTSLTQSVARAMLRFVTVTADALRFRQIQR |
| | | GFRTTLDDLSGRSYVMTAEDVDLTLNWGRL |
| | | SSVLPDYHGQDSVRVGRISFGSINAILGSVAL |
| | | ILNCHHH ASAVAAEFPKP STPPGS SGGAPDIQ |
| | | MTQ SP SSLSA SVGDRVTITCK ASEDIYNRLT |
| | | WYQQKPGK APKLLI SGATSLETGVP SRFSGS |
| | | GSGTDF TFTIS SLQPEDI ATYYCQQYWSNPYT |
| | | FGQGTKVEIKGS TSGSGKPGSGEGS TKGQVQ |
| | | LQESGPGLVRPSQTLSLTCTVSGFSLTSYGVH |
| | | WVRQPPGRGLEWIGVMWRGGSTDYNAAFM |
| | | SRLNITKDNSKNQVSLRLSSVTAADTAVYYC |
| | | AKSMITTGFVMDSWGQGSLVTVSS |
| SEQ ID NO: 52 | exemplary | MKEFTLDFSTAKTYVDSLNVIRSAIGTPLQTI |
| | cell-targeting | SSGGT SLLMID SGSGDNLF AVDVRGIDPEEG |
| | molecule 3 | RFNNLRLIVERNNLYVTGFVNRTNNVFYRFA |
| | (SLT- | DF SHVTFPGTT AVTL SGDSSYTTLQRVAGISR |
| | 1A::SCFV2::C2) | TGMQINRHSLTTSYLDLMSHSGTSLTQSVAR |
| | | AMLRFVTVTAEALRFRQIQRGFRTTLDDLSG |
| | | RSYVMT AEDVDLTLNWGRLS SVLPDYHGQ |
| | | DSVRVGRISFGSrNAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRFSGSGSGTDFTFTIS S |
| | | LQPEDIATYYCQQYWSNPYTFGQGTKVEIKG |
| | | STSGSGKPGSGEGSTKGQVQLQESGPGLVRP |
| | | SQTLSLTCTVSGFSLTSYGVHWVRQPPGRGL |
| | | EWIGVMWRGGSTDYNAAFMSRLNITKDNSK |
| | | NQVSLRLSSVTAADTAVYYCAKSMITTGFV |
| | | MD SWGQGSL VTVSSNLVPMV ATV |
| SEQ ID NO: 53 | exemplary | MKEFTLDFSTAKTYVDSLNVIRSAIGTPLQTI |
| | cell-targeting | SSGGT SLLMID SGSGDNLF AVDVRGIDPEEG |
| | molecule 4 | RFNNLRLIVERNNLYVTGFVNRTNNVFYRFA |
| | (inactive SLT- | DFSHVTFPGTT AVTL SGD SSYTTLQRVAGISR |
| | 1A::SCFV2::C2) | TGMQINRHSLTTSYLDLMSHSGTSLTQSVAR |
| | | AMLRFVTVTADALRFRQIQRGFRTTLDDLSG |
| | | RSYVMT AED VDLTLNWGRLS SVLPD YHGQ |
| | | DSVRVGRISFGSINAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | STSGSGKPGSGEGSTKGQVQLQESGPGLVRP |
| | | SQTLSLTCTVSGFSLTSYGVHWVRQPPGRGL |
| | | EWIGVMWRGGSTDYNAAFMSRLNITKDNSK |
| | | NQVSLRLSSVTAADTAVYYCAKSMITTGFV |
| | | MD SWGQGSL VTVS SNLVPMV ATV |
| SEQ ID NO: 54 | exemplary | MGILGF VF TLKEF TLDF STAKT YVD SLN VIRS |
| | cell-targeting | AIGTPLQTI SSGGT SLLMID SGSGDNLF AVDV |
| | molecule 5 | RGIDPEEGRFNNLRLIVERNNLYVTGFVNRT |
| | (F2::SLT- | NNVFYRFADFSHVTFPGTTAVTLSGDSSYTT |
| | 1A::SCFV2) | LQRVAGISRTGMQINRHSLTTSYLDLMSHSG |
| | | TSLTQSVARAMLRFVTVTAEALRFRQIQRGF |
| | | RTTLDDLSGRSYVMTAEDVDLTLNWGRLSS |
| | | VLPDYHGQDSVRVGRISFGSINAILGSVALIL |
| | | NCHHHAS AVAAEFPKPSTPPGS SGGAPDIQM |
| | | TQSP SSL SA SVGDRVTITCK ASEDIYNRLTW Y |
| | | QQKPGKAPKLLISGATSLETGVPSRFSGSGSG |
| | | TDF TF TIS SLQPEDI ATYYCQQYWSNPYTF GQ |
| | | GTK VEIKGS TSGSGKPGSGEGS TKGQVQLQE |
| | | SGPGLVRPSQTLSLTCTVSGFSLTSYGVHWV |
| | | RQPPGRGLEWIGVMWRGGSTDYNAAFMSR |
| | | LNITKDNSKNQVSLRLSSVTAADTAVYYCA |
| | | KSMITTGFVMDSWGQGSLVTVSS |
| SEQ ID NO: 55 | exemplary | MDIQMTQSPSSLSASVGDRVTITCRASQDVN |
| | cell-targeting | TAVAWYQQKPGKAPKLLIYSASFLYSGVPSR |
| | molecule 6 | FSGSRSGTDFTLTISSLQPEDFATYYCQQHYT |
| | (scFv3::F2::SL | TPPTFGQGTKVEIKRTGSTSGSGKPGSGEGSE |
| | T-1A) | VQLVESGGGLVQPGGSLRLSCAASGFNIKDT |
| | | YUrWVRQAPGKGLEWVARIYPTNGYTRYAD |
| | | SVKGRFTISADTSKNTAYLQMNSLRAEDTAV |
| | | YYC SRWGGDGF YAMD VWGQGTL VTVSSEF |
| | | PKPSTPPGS SGGAPGILGF VFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGF VNRTNNVF YRFADF SHVTFPGTT A |
| | | VTLSGDSSYTTLQRVAGISRTGMQrNRHSLT |
| | | TSYLDLMSHSGTSLTQSVARAMLRFVTVTA |
| | | EALRFRQIQRGFRTTLDDL SGRSYVMT AED V |
| | | DLTLNWGRLSSVLPDYHGQDSVRVGRISFGS |
| | | INAILGSVALILNCHHHASRVAR |
| SEQ ID NO: 56 | exemplary | MQVQLQQPGAELVKPGASVKMSCKTSGYTF |
| | cell-targeting | TSYNVHWVKQTPGQGLEWIGAIYPGNGDTS |
| | molecule 7 | FNQKFKGK ATLT ADK SSSTVYMQL SSLTSED |
| | (scFv4::F2::SL | SAVYYCARSNYYGSSYVWFFDVWGAGTTV |
| | T-1A) | TVSSGSTSGSGKPGSGEGSQIVLSQSPTILSAS |
| | | PGEK VTMTCRAS SSVSYMDW YQQKPGS SPK |
| | | PWIYATSNLASGVPARFSGSGSGTSYSLTISR |
| | | VEAEDAATYYCQQWISNPPTFGAGTKLELK |
| | | EFPKP STPP GSSGGAP GILGFVFTLKEF TLDF S |
| | | TAKTYVDSLNVIRSAIGTPLQTISSGGTSLLMI |
| | | DSGSGDNLFAVDVRGIDPEEGRFNNLRLIVE |
| | | RNNL YVTGF VNRTNNVF YRFADF SHVTFPG |
| | | TTAVTLSGDSSYTTLQRVAGISRTGMQrNRH |
| | | SLTTSYLDLMSHSGTSLTQSVARAMLRFVTV |
| | | TAEALRFRQIQRGFRTTLDDL SGRSY VMT AE |
| | | DVDLTLNWGRLSSVLPDYHGQDSVRVGRIS |
| | | FGSrNAILGSVALILNCHHHASRVAR |
| SEQ ID NO: 57 | exemplary | MKEFTLDFSTAKTYVDSLNVIRSAIGTPLQTI |
| | cell-targeting | SSGGT SLLMID SGSGDNLF AVDVRGIDPEEG |
| | molecule 8 | RFNNLRLIVERNNL YVTGF VNRTNNVF YRF A |
| | (SLT- | D F SHVTFPGTT AVTL SGD SSYTTLQRVAGISR |
| | 1A::SCFV5::C2) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPDYHGQ |
| | | DSVRVGRISFGSrNAILGSVALILNCHHHASA |
| | | VAAEFPKPSTPPGSSGGAPDIQMTQSPSSLSA |
| | | SVGDRVTITCRASQDVNTAVAWYQQKPGK |
| | | APKLLIYSASFLYSGVPSRFSGSRSGTDFTLTI |
| | | SSLQPEDFATYYCQQHYTTPPTFGQGTKVEI |
| | | KGGGGSE VQL VE SGGGL VQPGGSLRL SCAA |
| | | SGFNIKDTYIHWVRQAPGKGLEWVARIYPTN |
| | | GYTRYADSVKGRFTISADTSKNTAYLQMNS |
| | | LRAEDT AVYYC SRWGGDGF Y AMD YWGQG |
| | | TLVTVSSNLVPM VAT V |
| SEQ ID NO: 58 | exemplary | MKEFTLDFSTAKTYVDSLNVIRSAIGTPLQTI |
| | cell-targeting | SSGGTSLLMIDSGSGDNLFAVD VRGIDPEEG |
| | molecule 9 | RFNNLRLIVERNNL YVTGF VNRTNNVF YRF A |
| | (SLT- | D F SHVTFPGTT AVTL SGD SSYTTLQRVAGISR |
| | 1A::SCFV6::F2) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPDYHGQ |
| | | DSVRVGRISFGSrNAILGSVALILNCHHHASA |
| | | VAAEFPKP STPPG SSGGAPE VQLVESGGGLV |
| | | QPGGSLRLSCAASGF TF SD SWIHWVRQAPGK |
| | | GLEWVAWISPYGGSTYYADSVKGRFTISADT |
| | | SKNTAYLQMNSLRAEDTAVYYCARRHWPG |
| | | GFDYWGQGTLVTVSSGGGGSGGGGSGGGG |
| | | SGGGGSGGGGSDIQMTQ SP SSL SA SVGDRVT |
| | | ITCRASQDVSTAVAWYQQKPGKAPKLLIYSA |
| | | SFLYSGVPSRFSGSGSGTDFTLTISSLQPEDFA |
| | | TYYCQQYLYHPATFGQGTKVEIKGILGFVFT |
| | | L |
| SEQ ID NO: 59 | exemplary | MKEFTLDFSTAKTYVDSLNVIRSAIGTPLQTI |
| | cell-targeting | SSGGTSLLMIDSGSGDNLFAVDVRGIDPEEG |
| | molecule 10 | RFNNLRLIVERNNLYVTGFVNRTNNVFYRFA |
| | (inactive SLT- | DF SHVTFPGTTAVTLSGD SSYTTLQRVAGISR |
| | 1A::SCFV6::F2) | TGMQINRHSLTTSYLDLMSHSGTSLTQSVAR |
| | | AMLRFVTVTADALRFRQIQRGFRTTLDDLSG |
| | | RSYVMTAEDVDLTLNWGRLSSVLPDYHGQ |
| | | DSVRVGRISFGSrNAILGSVALILNCHHHASA |
| | | VAAEFPKPSTPPGSSGGAPEVQLVESGGGLV |
| | | QPGGSLRLSCAASGFTFSDSWffiWVRQAPGK |
| | | GLEWVAWISPYGGSTYYADSVKGRFTISADT |
| | | SKNTAYLQMNSLRAEDTAVYYCARRHWPG |
| | | GFDYWGQGTLVTVSSGGGGSGGGGSGGGG |
| | | SGGGGSGGGGSDIQMTQ SP SSL SA SVGDRVT |
| | | ITCRASQDVSTAVAWYQQKPGKAPKLLIYSA |
| | | SFLYSGVPSRFSGSGSGTDFTLTISSLQPEDFA |
| | | TYYCQQYLYHPATFGQGTKVEIKGILGFVFT |
| | | L |
| SEQ ID NO: 60 | exemplary | MKEFTLDFSTAKTYVDSLNVIRSAIGTPLQTI |
| | cell-targeting | SSGGTSLLMIDSGSGDNLFAVDVRGIDPEEG |
| | molecule 11 | RFNNLRLIVERNNLYVTGFVNRTNNVFYRFA |
| | (SLT- | DF SHVTFPGTTAVTLSGD SSYTTLQRVAGISR |
| | 1A::SCFV7::C2) | TGMQINRHSLTTSYLDLMSHSGTSLTQSVAR |
| | | AMLRFVTVTAEALRFRQIQRGFRTTLDDLSG |
| | | RSYVMTAEDVDLTLNWGRLSSVLPDYHGQ |
| | | DSVRVGRISFGSrNAILGSVALILNCHHHASA |
| | | VAAEFPKPSTPPGSSGGAPDIQMTQSPSSLSA |
| | | SVGDRVTITCRASQGIS SWLAWYQQKPEKAP |
| | | KSLIYAASSLQSGVPSRFSGSGSGTDFTLTISS |
| | | LQPEDFATYYCQQYNSYPYTFGQGTKLEIKG |
| | | GGGSQVQLVQSGAEVKKPGASVKVSCKASG |
| | | YTFTSYDVHWVRQAPGQRLEWMGWLHAD |
| | | TGITKFSQKFQGRVTITRDTSASTAYMELSSL |
| | | RSEDTAVYYCARERIQLWFDYWGQGTLVTV |
| | | SSNLVPMVATV |
| SEQ ID NO: 61 | exemplary | MKEFTLDFSTAKTYVDSLNVIRSAIGTPLQTI |
| | cell-targeting | SSGGTSLLMIDSGSGDNLFAVDVRGIDPEEG |
| | molecule 12 | RFNNLRLIVERNNLYVTGFVNRTNNVFYRFA |
| | (SLT- | DF SHVTFPGTTAVTLSGD SSYTTLQRVAGISR |
| | 1A::scFv1::C2) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSINAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSSNLVPMVATV |
| SEQ ID NO: 62 | wild-type | KEFTLDF STAKT YVDSLNVIRS AIGTPLQTIS S |
| | Shiga toxin | GGT SLLMID SG SGDNLFAVD VRGIDPEEGRF |
| | effector | NNLRLIVERNNLYVTGFVNRTNNVFYRFADF |
| | polypeptide | SFTVTFPGTTAVTLSGDSSYTTLQRVAGISRTG |
| | (SLT-1A-WT) | MQrNRHSLTTSYLDLMSHSGTSLTQSVARA |
| | | MLRFVTVTAEALRFRQIQRGFRTTLDDLSGR |
| | | SYVMT AED VDLTLNWGRLS SVLPD YHGQD S |
| | | VRVGRISFGSrNAILGSVALILNCHHHASRVA |
| | | R |
| SEQ ID NO: 63 | reference cell- | MKEFTLDFSTAKTYVDSLNVIRSAIGTPLQTI |
| | targeting | SSGGT SLLMID SGSGDNLF AVDVRGIDPEEG |
| | molecule 1 | RFNNLRLIVERNNLYVTGFVNRTNNVFYRFA |
| | (SLT- | DF SHVTFPGTT AVTL SGD SSYTTLQRVAGISR |
| | 1A::scFv1) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSrNAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | GGGSQVQLQESGPGLVRPSQTLSLTCTVSGF |
| | | SLTSYGVHWVRQPPGRGLEWIGVMWRGGS |
| | | TDYNAAFMSRLNITKDNSKNQVSLRLSSVTA |
| | | ADTAVYYCAKSMITTGFVMDSWGQGSLVT |
| | | VSS |
| SEQ ID NO: 64 | reference cell- | MKEFTLDFSTAKTYVDSLNVIRSAIGTPLQTI |
| | targeting | SSGGTSLLMIDSGSGDNLF AVD VRGIDPEEG |
| | molecule 2 | RFNNLRLIVERNNLYVTGFVNRTNNVFYRFA |
| | (SLT- | DF SHVTFPGTT AVTL SGD SSYTTLQRVAGISR |
| | 1A::SCFV2) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSrNAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | STSGSGKPGSGEGSTKGQVQLQESGPGLVRP |
| | | SQTLSLTCTVSGFSLTSYGVHWVRQPPGRGL |
| | | EWIGVMWRGGSTDYNAAFMSRLNITKDNSK |
| | | NQVSLRLSSVTAADTAVYYCAKSMITTGFV |
| | | MDSWGQGSLVTVSS |
| SEQ ID NO: 65 | reference cell- | MKEFTLDFSTAKTYVDSLNVIRSAIGTPLQTI |
| | targeting | SSGGT SLLMID SGSGDNLF AVD VRGIDPEEG |
| | molecule 3 | RFNNLRLIVERNNLYVTGFVNRTNNVFYRFA |
| | (inactive SLT- | DF SHVTFPGTT AVTL SGD SSYTTLQRVAGISR |
| | 1A::SCFV2) | TGMQINRHSLTTSYLDLMSHSGTSLTQSVAR |
| | | AMLRFVTVTADALRFRQIQRGFRTTLDDLSG |
| | | R SYVMT AED VDLTLNWGRLS SVLPD YHGQ |
| | | DSVRVGRISFGSINAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | STSGSGKPGSGEGSTKGQVQLQESGPGLVRP |
| | | SQTLSLTCTVSGFSLTSYGVHWVRQPPGRGL |
| | | EWIGVMWRGGSTDYNAAFMSRLNITKDNSK |
| | | NQVSLRLSSVTAADTAVYYCAKSMITTGFV |
| | | MDSWGQGSLVTVSS |
| SEQ ID NO: 66 | reference cell- | MKEFTLDFSTAKTYVDSLNVIRSAIGTPLQTI |
| | targeting | SSGGTSLLMIDSGSGDNLFAVD VRGIDPEEG |
| | molecule 4 | RFNNLRLIVERNNLYVTGFVNRTNNVFYRFA |
| | (SLT- | DF SHVTFPGTT AVTL SGD SSYTTLQRVAGISR |
| | 1A::SCFV5) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSINAILGSVALILNCHHHASA |
| | | VAAEFPKPSTPPGSSGGAPDIQMTQSPSSLSA |
| | | SVGDRVTITCRASQDVNTAVAWYQQKPGK |
| | | APKLLIYSASFLYSGVPSRFSGSRSGTDFTLTI |
| | | SSLQPEDFATYYCQQHYTTPPTFGQGTKVEI |
| | | KGGGGSE VQL VE SGGGL VQPGGSLRL SCAA |
| | | SGFNIKDTYIHWVRQAPGKGLEWVARIYPTN |
| | | GYTRYADSVKGRFTISADTSKNTAYLQMNS |
| | | LRAEDT AVYYC SRWGGDGF YAMD YWGQG |
| | | TLVTVSS |
| SEQ ID NO: 67 | reference cell- | MKEFTLDFSTAKTYVDSLNVIRSAIGTPLQTI |
| | targeting | SSGGTSLLMIDSGSGDNLFAVD VRGIDPEEG |
| | molecule 5 | RFNNLRLIVERNNLYVTGFVNRTNNVFYRFA |
| | (SLT- | DF SHVTFPGTT AVTL SGD SSYTTLQRVAGISR |
| | 1A::SCFV6) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSINAILGSVALILNCHHHASA |
| | | VAAEFPKP STPPG SSGGAPE VQL VESGGGL V |
| | | QPGGSLRL SCAA SGF TF SD SWIHW VRQ APGK |
| | | GLEWVAWISPYGGSTYYADSVKGRFTISADT |
| | | SKNTAYLQMNSLRAEDTAVYYCARRHWPG |
| | | GFDYWGQGTLVTVSSGGGGSGGGGSGGGG |
| | | SGGGGS GGGGSDIQMTQ SP SSL SA SVGDRVT |
| | | ITCRASQDVSTAVAWYQQKPGKAPKLLIYSA |
| | | SFLYSGVPSRFSGSGSGTDFTLTISSLQPEDFA |
| | | TYYCQQYLYHPATFGQGTKVEIK |
| SEQ ID NO: 68 | reference cell- | MKEFTLDFSTAKTYVDSLNVIRSAIGTPLQTI |
| | targeting | SSGGT SLLMID SG SGDNLF AVD VRGIDPEEG |
| | molecule 6 | RFNNLRLIVERNNLYVTGFVNRTNNVFYRFA |
| | (inactive SLT- | D F SHVTFPGTT AVTL SGD SSYTTLQRVAGISR |
| | 1A::SCFV6) | TGMQINRHSLTTSYLDLMSHSGTSLTQSVAR |
| | | AMLRFVTVTADALRFRQIQRGFRTTLDDLSG |
| | | R SYVMT AED VDLTLNWGRLS SVLPD YHGQ |
| | | DSVRVGRISFGSrNAILGSVALILNCHHHASA |
| | | VAAEFPKP STPPG SSGGAPE VQL VESGGGL V |
| | | QPGGSLRL SCAA SGF TF SD SWIHW VRQ APGK |
| | | GLEWVAWISPYGGSTYYADSVKGRFTISADT |
| | | SKNTAYLQMNSLRAEDTAVYYCARRHWPG |
| | | GFDYWGQGTLVTVSSGGGGSGGGGSGGGG |
| | | SGGGGS GGGGSDIQMTQ SP SSL SA SVGDRVT |
| | | ITCRASQDVSTAVAWYQQKPGKAPKLLIYSA |
| | | SFLYSGVPSRFSGSGSGTDFTLTISSLQPEDFA |
| | | TYYCQQYLYHPATFGQGTKVEIK |
| SEQ ID NO: 69 | reference cell- | MKEFTLDFSTAKTYVDSLNVIRSAIGTPLQTI |
| | targeting | SSGGTSLLMIDSGSGDNLFAVD VRGIDPEEG |
| | molecule 7 | RFNNLRLIVERNNLYVTGFVNRTNNVFYRFA |
| | (SLT- | D F SHVTFPGTT AVTL SGD SSYTTLQRVAGISR |
| | 1A::SCFV7) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSrNAILGSVALILNCHHHASA |
| | | VAAEFPKPSTPPGSSGGAPDIQMTQSPSSLSA |
| | | SVGDRVTITCRASQGIS SWLAWYQQKPEKAP |
| | | K SLIY AA SSLQ SGVP SRF SGSGSGTDF TLTI SS |
| | | LQPEDFATYYCQQYNSYPYTFGQGTKLEIKG |
| | | GGGSQVQLVQSGAEVKKPGASVKVSCKASG |
| | | YTFTSYDVHWVRQAPGQRLEWMGWLHAD |
| | | TGITKFSQKFQGRVTITRDTSASTAYMELSSL |
| | | RSEDTAVYYCARERIQLWFDYWGQGTLVTV |
| | | SS |
| SEQ ID NO: 70 | reference cell- | MKEFTLDFSTAKTYVDSLNVIRSAIGTPLQTI |
| | targeting | SSGGTSLLMIDSGSGDNLFAVD VRGIDPEEG |
| | molecule 8 | RFNNLRLIVERNNLYVTGFVNRTNNVFYRFA |
| | (SLT- | D F SHVTFPGTT AVTL SGD SSYTTLQRVAGISR |
| | 1A::SCFV2) | TGMQINRHSLTTSYLDLMSHSGTSLTQSVAR |
| | | AMLRFVTVTAEALRFRQIQRGFRTTLDDLSG |
| | | R SYVMT AED VDLTLNWGRLS SVLPD YHGQ |
| | | DSVRVGRISFGSrNAILGSVALILNCHHHASA |
| | | VAAEFPKPSTPPGSSGGAPDIQMTQSPSSLSA |
| | | SVGDRVTITCKASEDIYNRLTWYQQKPGKAP |
| | | KLLIS GAT SLETGVP SRF SGSGSGTDF TF TIS S |
| | | LQPEDIATYYCQQYWSNPYTFGQGTKVEIKG |
| | | STSGSGKPGSGEGSTKGQVQLQESGPGLVRP |
| | | SQTLSLTCTVSGFSLTSYGVHWVRQPPGRGL |
| | | EWIGVMWRGGSTDYNAAFMSRLNITKDNSK |
| | | NQVSLRLSSVTAADTAVYYCAKSMITTGFV |
| | | MDSWGQGSLVTVSS |
| SEQ ID NO: 71 | Linker with | EFPKPSTPPGSSGGAPGILGFVFTL |
| | extension | |
| SEQ ID NO: 72 | Protein 1 | QVQLQQPGAELVKPGASVKMSCKTSGYTFT |
| | | SYNVHWVKQTPGQGLEWIGAIYPGNGDTSF |
| | | NQKFKGK ATLTADK SSSTVYMQL SSLTSED S |
| | | AVYYCARSNYYGS SYVWFFDVWGAGTTVT |
| | | VSSGSTSGSGKPGSGEGSQIVLSQSPTILSASP |
| | | GEKVTMTCRAS SSV SYMDWYQQKPGS SPKP |
| | | WIYATSNLASGVPARFSGSGSGTSYSLTISRV |
| | | EAEDAATYYCQQWISNPPTFGAGTKLELKEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRFADF SHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVAR |
| SEQ ID NO: 73 | Protein 2 | MQVQLQQPGAELVKPGASVKMSCKTSGYTF |
| | | TSYNVHWVKQTPGQGLEWIGAIYPGNGDTS |
| | | FNQKFKGKATLTADK SSSTVYMQL SSLTSED |
| | | SAVYYCARSNYYGS SYVWFFDVWGAGTTV |
| | | TVSSGSTSGSGKPGSGEGSQIVLSQSPTILSAS |
| | | PGEKVTMTCRAS SSVSYMDWYQQKPGS SPK |
| | | PWIYATSNLASGVPARFSGSGSGTSYSLTISR |
| | | VEAEDAATYYCQQWISNPPTFGAGTKLELK |
| | | EFPKP STPP GSSGGAP GILGFVFTLKEFTLDF S |
| | | TAKTYVDSLNVIRSAIGTPLQTISIGGTSLLMI |
| | | D SGIGDNLFAVDVRGIAPEEGRFNNLRLIVER |
| | | NNLYVTGFVNRTNNVFYRFADF SHVTFPGTT |
| | | AVTLSADSSYTTLQRVAGISRTGMQINRHSL |
| | | TTSYLDLMSHSATSLTQSVARAMLRFVTVT |
| | | AEALRFRQIQRGFRTTLDDL SGRSYVMTAED |
| | | VDLTLNWGRL SSVLPDYHGQD SVRVGRISF |
| | | GSINAI LGSVALILNCHHHASAVAR |
| SEQ ID NO: 74 | Protein 3 | MDIQMTQSPSSLSASVGDRVTITCRASQDVN |
| | | TAVAWYQQKPGKAPKLLIYSASFLYSGVPSR |
| | | FSGSRSGTDFTLTISSLQPEDFATYYCQQHYT |
| | | TPPTFGQGTKVEIKRTGSTSGSGKPGSGEGSE |
| | | VQLVESGGGLVQPGGSLRLSCAASGFNIKDT |
| | | YIHWVRQAPGKGLEWVARIYPTNGYTRYAD |
| | | SVKGRFTISADTSKNTAYLQMNSLRAEDTAV |
| | | YYCSRWGGDGFYAMDVWGQGTLVTVSSEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRFADF SHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVARKDEL |
| SEQ ID NO: 75 | Protein 4 | MDIQMTQSPSSLSASVGDRVTITCRASQDVN |
| | | TAVAWYQQKPGKAPKLLIYSASFLYSGVPSR |
| | | FSGSRSGTDFTLTISSLQPEDFATYYCQQHYT |
| | | TPPTFGQGTKVEIKRTGSTSGSGKPGSGEGSE |
| | | VQLVESGGGLVQPGGSLRLSCAASGFNIKDT |
| | | YIHWVRQAPGKGLEWVARIYPTNGYTRYAD |
| | | SVKGRFTISADTSKNTAYLQMNSLRAEDTAV |
| | | YYCSRWGGDGFYAMDVWGQGTLVTVSSEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRFADF SHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVARKDEL |
| SEQ ID NO: 76 | Protein 5 | MDIELTQSPSSFSVSLGDRVTITCKASEDIYN |
| | | RLAWYQQKPGNAPRLLISGATSLETGVPSRF |
| | | SGSGSGKDYTLSITSLQTEDVATYYCQQYWS |
| | | TPTFGGGTKLEIKGS TSGSGKPGS GEGSKVQ |
| | | LQESGPSLVQPSQRLSITCTVSGFSLISYGVH |
| | | WVRQSPGKGLEWLGVIWRGGSTDYNAAFM |
| | | SRLSITKDNSKSQVFFKMNSLQADDTAIYFC |
| | | AKTLITTGYAMDYWGQGTTVTVSSEFPKPST |
| | | PPGS SGGAPGILGFVFTLKEFTLDF STAKTYV |
| | | D SLNVIRSAIGTPLQTISSGGTSLLMID SGSGD |
| | | NLFAVDVRGIDPEEGRFNNLRLIVERNNLYV |
| | | TGFVNRTNNVFYRFADF SHVTFPGTTAVTLS |
| | | GDSSYTTLQRVAGISRTGMQINRHSLTTSYL |
| | | DLMSHSGTSLTQSVARAMLRFVTVTAEALR |
| | | FRQIQRGFRTTLDDLSGRSYVMTAEDVDLTL |
| | | NWGRLSSVLPDYHGQDSVRVGRISFGSINAI |
| | | LGSVALILNCHHHASRVARKDEL |
| SEQ ID NO: 77 | Protein 6 | MDIELTQSPSSFSVSLGDRVTITCKASEDIYN |
| | | RLAWYQQKPGNAPRLLISGATSLETGVPSRF |
| | | SGSGSGKDYTLSITSLQTEDVATYYCQQYW S |
| | | TPTFGGGTKLEIKGS TSGSGKPGSGEGSKVQ |
| | | LQESGPSLVQPSQRLSITCTVSGFSLISYGVH |
| | | WVRQSPGKGLEWLGVIWRGGSTDYNAAFM |
| | | SRLSITKDNSKSQVFFKMNSLQADDTAIYFC |
| | | AKTLITTGYAMDYWGQGTTVTVSSEFPKPST |
| | | PPGS SGGAPGILGFVFTLKEFTLDF STAKTYV |
| | | D SLNVIRSAIGTPLQTISSGGTSLLMID SGSGD |
| | | NLFAVDVRGIDPEEGRFNNLRLIVERNNLYV |
| | | TGFVNRTNNVFYRF ADF SHVTFPGTTAVTLS |
| | | GDSSYTTLQRVAGISRTGMQINRHSLTTSYL |
| | | DLMSHSGTSLTQSVARAMLRFVTVTAEALR |
| | | FRQIQRGFRTTLDDLSGRSYVMTAEDVDLTL |
| | | NWGRLSSVLPDYHGQDSVRVGRISFGSrNAI |
| | | LGSVALILNCHHHASRVARKDEL |
| SEQ ID NO: 78 | Protein 7 | MDIVMTQAAPSIPVTPGESVSISCRSSKSLLN |
| | | SNGNTYLYWFLQRPGQSPQLLIYRMSNLASG |
| | | VPDRF SGSGSGTAFTLRISRVEAEDVGVYYC |
| | | MQHLEYPFTFGAGTKLELKGSTSGSGKPGSG |
| | | EGSEVQLQQSGPELIKPGASVKMSCKASGYT |
| | | FTSYVMHWVKQKPGQGLEWIGYINPYNDGT |
| | | KYNEKFKGKATLTSDKSSSTAYMELSSLTSE |
| | | DSAVYYCARGTYYYGSRVFDYWGQGTTLT |
| | | VSSAEFPKPSTPPGSSGGAPGILGFVFTLKEFT |
| | | LDF STAKTYVDSLNVIRSAIGTPLQTIS SGGTS |
| | | LLMIDSGSGDNLFAVDVRGIDPEEGRFNNLR |
| | | LIVERNNLYVTGFVNRTNNVFYRF ADF SHVT |
| | | FPGTTAVTLSGDSSYTTLQRVAGISRTGMQI |
| | | NRHSLTTSYLDLMSHSGTSLTQSVARAMLRF |
| | | VTVTAEALRFRQIQRGFRTTLDDLSGRSYVM |
| | | TAEDVDLTLNWGRL SSVLPDYHGQD SVRVG |
| | | RISFGSrNAILGSVALILNCHHHASRVARKDE |
| | | L |
| SEQ ID NO: 79 | Protein 8 | MDIVMTQAAPSIPVTPGESVSISCRSSKSLLN |
| | | SNGNTYLYWFLQRPGQSPQLLIYRMSNLASG |
| | | VPDRF SGSGSGTAFTLRISRVEAEDVGVYYC |
| | | MQHLEYPFTFGAGTKLELKGSTSGSGKPGSG |
| | | EGSEVQLQQSGPELIKPGASVKMSCKASGYT |
| | | FTSYVMHWVKQKPGQGLEWIGYINPYNDGT |
| | | KYNEKFKGKATLTSDKSSSTAYMELSSLTSE |
| | | DSAVYYCARGTYYYGSRVFDYWGQGTTLT |
| | | VSSAEFPKPSTPPGSSGGAPGILGFVFTLKEFT |
| | | LDF STAKTYVDSLNVIRSAIGTPLQTIS SGGTS |
| | | LLMIDSGSGDNLFAVDVRGIDPEEGRFNNLR |
| | | LIVERNNLYVTGF VNRTNNVF YRF ADF SHVT |
| | | FPGTTAVTLSGDSSYTTLQRVAGISRTGMQI |
| | | NRHSLTTSYLDLMSHSGTSLTQSVARAMLRF |
| | | VTVTAEALRFRQIQRGFRTTLDDLSGRSYVM |
| | | TAEDVDLTLNWRL SSVLPDYHGQD SVRVG |
| | | RISFGSrNAILGSVALILNCHHHASRVARKDE |
| | | L |
| SEQ ID NO: 80 | Protein 9 | MDIQLTQSPLSLPVTLGQPASISCRSSQSLVH |
| | | RNGNTYLHWFQQRPGQSPRLLIYTVSNRFSG |
| | | VPDRF SGSGSGTDFTLKISRVEAEDVGVYFC |
| | | SQSSHVPPTF GAGTRLEIKGSTSGSGKPGSGE |
| | | GSTKGQVQLQQSGSELKKPGASVKVSCKAS |
| | | GYTFTNYGVNWIKQAPGQGLQWMGWINPN |
| | | TGEPTFDDDFKGRFA FSLDTSVSTAYLQIS SL |
| | | KADDTAVYFCSRSRGKNEAWFAYWGQGTL |
| | | VTVSSEFPKPSTPPGSSGGAPGILGFVFTLKEF |
| | | TLDF STAKTYVD SLNVIRS AIGTPLQTISSGGT |
| | | SLLMID SGSGDNLFAVDVRGIDPEEGRFNNL |
| | | RLIVERNNLYVTGFVNRTNNVFYRFADF SHV |
| | | TFPGTTAVTLSGDSSYTTLQRVAGISRTGMQI |
| | | NRHSLTTSYLDLMSHSGTSLTQSVARAMLRF |
| | | VTVTAEALRFRQIQRGFRTTLDDLSGRSYVM |
| | | TAEDVDLTLNWGRL SSVLPDYHGQD SVRVG |
| | | RISFGSINAILGSVALILNCHHHASRVARKDE |
| | | L |
| SEQ ID NO: 81 | Protein 10 | MDIQLTQSPLSLPVTLGQPASISCRSSQSLVH |
| | | RNGNTYLHWFQQRPGQSPRLLIYTVSNRFSG |
| | | VPDRF SGSGSGTDFTLKISRVEAED VGVYFC |
| | | SQSSHVPPTFGAGTRLEIKGS TSGSGKPGS GE |
| | | GSTKGQVQLQQSGSELKKPGASVKVSCKAS |
| | | GYTFTNYGVNWIKQAPGQGLQWMGWINPN |
| | | TGEPTFDDDFKGRFAF SLDT SVSTAYLQIS SL |
| | | KADDTAVYFCSRSRGKNEAWFAYWGQGTL |
| | | VTVSSEFPKPSTPPGSSGGAPGILGFVFTLKEF |
| | | TLDF STAKTYVD SLNVIRSAIGTPLQ TISSGGT |
| | | SLLMID SGSGDNLFAVDVRGIDPEEGRFNNL |
| | | RLIVERNNLYVTGFVNRTNNVFYRFADF SHV |
| | | TFPGTTAVTLSGDSSYTTLQRVAGISRTGMQI |
| | | NRHSLTTSYLDLMSHSGTSLTQSVARAMLRF |
| | | VTVTAEALRFRQIQRGFRTTLDDLSGRSYVM |
| | | TAEDVDLTLNWGRL SSVLPDYHGQD SVRVG |
| | | RISFGSINAILGSVALILNCHHHASRVARKDE |
| | | L |
| SEQ ID NO: 82 | Protein 11 | MEVQLVESGGGLVQ AGGSLRLSCAASGITF S |
| | | INTMGWYRQAPGKQRELVALIS SIGDTYYAD |
| | | SVKGRFTISRDNAKNTVYLQMNSLKPEDTA |
| | | VYYCKRFRTAAQGTDYWGQGTQVTVSSAH |
| | | HSEDPS SKAPKAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVFYRF ADF SHVTFPGTTA |
| | | VTLSGDSSYTTLQRVAGISRTGMQINRHSLT |
| | | TSYLDLMSHSGTSLTQSVARAMLRFVTVTA |
| | | EALRFRQIQRGFRTTLDDL SGRSYVMTAEDV |
| | | DLTLNWGRLSSVLPDYHGQDSVRVGRISFGS |
| | | INAILGSVALILNCHHHASRVARKDEL |
| SEQ ID NO: 83 | Protein 12 | MEVQLVESGGGLVQ AGGSLRLSC AASGITF S |
| | | INTMGWYRQAPGKQRELVALIS SIGDTYYAD |
| | | SVKGRFTISRDNAKNTVYLQMNSLKPEDTA |
| | | VYYCKRFRTAAQGTDYWGQGTQVTVSSEFP |
| | | KPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGFVNRTNNVF YRF ADF SHVTFPGTTA |
| | | VTLSGDSSYTTLQRVAGISRTGMQrNRHSLT |
| | | TSYLDLMSHSGTSLTQSVARAMLRFVTVTA |
| | | EALRFRQIQRGFRTTLDDL SGRS YVMTAEDV |
| | | DLTLNWGRLSSVLPDYHGQDSVRVGRISFGS |
| | | INAILGSVALILNCHHHASRVARKDEL |
| SEQ ID NO: 84 | Protein 13 | MEVQLVESGGGLVQ AGGSLRLSC AASGITF S |
| | | INTMGWYRQAPGKQRELVALIS SIGDTYYAD |
| | | SVKGRFTISRDNAKNTVYLQMNSLKPEDTA |
| | | VYYCKRFRTAAQGTDYWGQGTQVTVSSAH |
| | | HSEDPSSKAPKAPGILGFVFTLGILGFVFTLK |
| | | EFTLDF STAKTYVDSLNVIRSAIGTPLQTIS SG |
| | | GT SLLMID SGTGDNLFAVDVRGIDPEEGRFN |
| | | NLRLIVERNNLYVTGFVNRTNNVF YRF ADF S |
| | | HVTFPGTTAVTLSGDSSYTTLQRVAGISRTG |
| | | MQINRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASRVA |
| | | RKDEL |
| SEQ ID NO: 85 | Protein 14 | MEVQLVESGGGLVQ AGGSLRLSCAASGITF S |
| | | INTMGWYRQAPGKQRELVALIS SIGDTYYAD |
| | | SVKGRFTISRDNAKNTVYLQMNSLKPEDTA |
| | | VYYCKRFRTAAQGTDYWGQGTQVTVSSEFP |
| | | KPSTPPGSSGGAPGILGFVFTLGILGFVFTLKE |
| | | FTLDF STAKTYVDSLNVIRS AIGTPLQTIS SGG |
| | | TSLLMIDSGTGDNLFAVDVRGIDPEEGRFNN |
| | | LRLIVERNNL YVTGF VNRTNNVF YRF ADF SH |
| | | VTFPGTTAVTLSGDSSYTTLQRVAGISRTGM |
| | | QINRHSLTTSYLDLMSHSGTSLTQSVARAML |
| | | RFVTVTAEALRFRQIQRGFRTTLDDLSGRSY |
| | | VMTAEDVDLTLNWGRLSSVLPDYHGQDSV |
| | | RVGRISFGSINAILGSVALILNSHHHASRVAR |
| | | KDEL |
| SEQ ID NO: 86 | Protein 15 | MAPTS SSTKKTQLQLEHLLLDLQMILNGINN |
| | | YKNPKLTRMLTFKFYMPKKATELKHLQCLE |
| | | EELKPLEE VLNLAQSKNFHLRPRDLI SNINVI |
| | | VLELKGSETTFMCEYADETATIVEFLNRWIT |
| | | FCQSIISTLTEFPKPSTPPGSSGGAPGILGFVFT |
| | | LKEFTLDFSTAKTYVDSLNVIRS AIGTPLQTIS |
| | | SGGT SLLMID SGSGDNLF AVDVRGIDPEEGR |
| | | FNNLRLIVERNNL YVTGF VNRTNNVFYRF AD |
| | | FSHVTFPGTTAVTLSGDSSYTTLQRVAGISRT |
| | | GMQrNRHSLTTSYLDLMSHSGTSLTQSVARA |
| | | MLRFVTVTAEALRFRQIQRGFRTTLDDLSGR |
| | | SYVMTAEDVDLTLNWGRLS SVLPDYHGQD S |
| | | VRVGRISFGSrNAILGSVALILNCHHHASRVA |
| | | RKDEL |
| SEQ ID NO: 87 | Protein 16 | MAPTS SSTKKTQLQLEHLLLDLQMILNGINN |
| | | YKNPKLTRMLTFKFYMPKKATELKHLQCLE |
| | | EELKPLEE VLNLAQSKNFHLRPRDLI SNINVI |
| | | VLELKGSETTFMCEYADETATIVEFLNRWIT |
| | | FCQSIISTLTEFPKPSTPPGSSGGAPGILGFVFT |
| | | LKEF TLDF STAKTYVD SLNVIRS AIGTPLQTI S |
| | | SGGT SLLMID SGTGDNLFAVDVRGIDPEEGR |
| | | FNNLRLIVERNNL YVTGF VNRTNNVF YRF AD |
| | | FSHVTFPGTTAVTLSGDSSYTTLQRVAGISRT |
| | | GMQrNRHSLTTSYLDLMSHSGTSLTQSVARA |
| | | MLRFVTVTAEALRFRQIQRGFRTTLDDLSGR |
| | | SYVMTAED VDLTLNWGRLS SVLPDYHGQD S |
| | | VRVGRISFGSrNAILGSVALILNSHHHASRVA |
| | | RKDEL |
| SEQ ID NO: 88 | Protein 17 | MQVQLVQSGAELVKPGASVKMSCKASGYT |
| | | FTSYNMIIWVKQTPGQGLEWIGAIYPGNGDT |
| | | SYNQKFKGK ATLTADK SSSTAYMQL SSLTSE |
| | | D SAVYYCARAQLRPNYWYFD VWGAGTT VT |
| | | VSSGSTSGSGKPGSGEGSDIVLSQSPAILSASP |
| | | GEKVTMTCRAS SSV SYMFIWYQQKPGS SPKP |
| | | WIYATSNLASGVPARFSGSGSGTSYSLTISRV |
| | | EAEDAATYYCQQWISNPPTFGAGTKLELKEF |
| | | PKPSTPPGS SGGAPGILGFVFTLKEFTLDF STA |
| | | KTYVDSLNVIRSAIGTPLQTISSGGTSLLMIDS |
| | | GSGDNLFAVDVRGIDPEEGRFNNLRLIVERN |
| | | NLYVTGF VNRTNNVF YRF ADF SHVTFPGTTA |
| | | VTLSGDSSYTTLQRVAGISRTGMQrNRHSLT |
| | | TSYLDLMSHSGTSLTQSVARAMLRFVTVTA |
| | | EALRFRQIQRGFRTTLDDL SGRSYVMTAED V |
| | | DLTLNWGRLSSVLPDYHGQDSVRVGRISFGS |
| | | INAILGSVALILNSHHHASRVAR |
| SEQ ID NO: 89 | Protein 18 | MQVQLQQPGAELVKPGASVKMSCKTSGYTF |
| | | TSYNVHWVKQTPGQGLEWIGAIYPGNGDTS |
| | | FNQKFKGK ATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEK VTMTCRAS SSV SYMDWYQQKPGS SPK |
| | | PWIYATSNLASGVPARFSGSGSGTSYSLTISR |
| | | VEAEDAATYYCQQWISNPPTFGAGTKLELK |
| | | EFPKP STPPG SSGGAP GILGFVFTLKEFTLDF S |
| | | TAKTYVDSLNVIRSAIGTPLQTISSGGTSLLMI |
| | | DSGSGDNLFAVDVRGIDPEEGRFNNLRLIVE |
| | | RNNLYVTGFVNRTNNVF YRF ADF SHVTFPG |
| | | TTAVTLSGDSSYTTLQRVAGISRTGMQrNRH |
| | | SLTTSYLDLMSHSGTSLTQSVARAMLRFVTV |
| | | TAEALRFRQIQRGFRTTLDDL SGRS YVMTAE |
| | | DVDLTLNWGRLSSVLPDYHGQDSVRVGRIS |
| | | FGSrNAILGSVALILNCHHHASRVAR |
| SEQ ID NO: 90 | Protein 19 | MQVQLQQPGAELVKPGASVKMSCKTSGYTF |
| | | TSYNVHWVKQTPGQGLEWIGAIYPGNGDTS |
| | | FNQKFKGK ATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEKVTMTCRAS SSVSYMDWYQQKPGS SPK |
| | | PWIYATSNLASGVPARFSGSGSGTSYSLTISR |
| | | VEAEDAATYYCQQWISNPPTFGAGTKLELK |
| | | EFPKP STPP GSSGGAP GILGFVFTLKEFTLDF S |
| | | TAKTYVDSLNVIRSAIGTPLQTISSGGTSLLMI |
| | | DSGSGDNLFAVDVRGIDPEEGRFNNLRLIVE |
| | | RNNLYVTGFVNRTNNVFYRF ADF SHVTFPG |
| | | TTAVTLSGDSSYTTLQRVAGISRTGMQrNRH |
| | | SLTTSYLDLMSHSGTSLTQSVARAMLRFVTV |
| | | TAEALRFRQIQRGFRTTLDDL SGRSYVMTAE |
| | | DVDLTLNWGRLSSVLPDYHGQDSVRVGRIS |
| | | FGSrNAILGSVALILNSHHHASRVAR |
| SEQ ID NO: 91 | Protein 20 | MQVQLQQPGAELVKPGASVKMSCKTSGYTF |
| | | TSYNVHWVKQTPGQGLEWIGAIYPGNGDTS |
| | | FNQKFKGKATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEKVTMTCRAS SSVSYMDWYQQKPGS SPK |
| | | PWIYATSNLASGVPARFSGSGSGTSYSLTISR |
| | | VEAEDAATYYCQQWISNPPTFGAGTKLELK |
| | | EFPKP STPPG SSGGAP GILGFVFTLKEFTLDF S |
| | | TAKTYVDSLNVIRSAIGTPLQTISSGGTSLLMI |
| | | D SGTGDNLF AVDVRGIDPEEGRFNNLRLIVE |
| | | RNNLYVTGFVNRTNNVFYRF ADF SHVTFPG |
| | | TTAVTLSGDSSYTTLQRVAGISRTGMQINRH |
| | | SLTTSYLDLMSHSGTSLTQSVARAMLRFVTV |
| | | TAEALRFRQIQRGFRTTLDDL SGRS YVMTAE |
| | | DVDLTLNWGRLSSVLPDYHGQDSVRVGRIS |
| | | FGSrNAILGSVALILNCHHHASRVAR |
| SEQ ID NO: 92 | Protein 21 | MQVQLQQPGAELVKPGASVKMSCKASGYT |
| | | FTSYNMHWVKQTPGRGLEWIGAIYPGNGDT |
| | | SYNQKFKGKATLTADK SSSTAYMQL SSLTSE |
| | | D SAVYYCARSTYYGGDWYFNVWGAGTTVT |
| | | VSAGSTSGSGKPGSGEGSTKGQIVLSQSPAIL |
| | | SASPGEKVTMTCRAS SSV SYIHWFQQKPGS S |
| | | PKPWIYATSNLASGVPVRFSGSGSGTSYSLTI |
| | | SRVEAEDAATYYCQQWTSNPPTFGGGTKLEI |
| | | KEFPKP STPPG SSGGAP GILGFVFTLKEF TLDF |
| | | STAKTYVDSLNVIRSAIGTPLQTISSGGTSLL |
| | | MIDSGSGDNLFAVDVRGIDPEEGRFNNLRLI |
| | | VERNNLYVTGFVNRTNNVFYRF ADF SHVTF |
| | | PGTTAVTLSGDSSYTTLQRVAGISRTGMQIN |
| | | RHSLTTSYLDLMSHSGTSLTQSVARAMLRFV |
| | | TVTAEALRFRQIQRGFRTTLDDLSGRSYVMT |
| | | AEDVDLTLNWGRLS SVLPDYHGQD SVRVGR |
| | | ISFGSINAILGSVALILNSHHHASRVAR |
| SEQ ID NO: 93 | Protein 22 | MEVQLVE SGGGLVQPGRSLRL SCAA SGF TFN |
| | | DYAMHWVRQAPGKGLEWVSTISWNSGSIG |
| | | YADSVKGRFTISRDNAKKSLYLQMNSLRAE |
| | | DTALYYCAKDIQYGNYYYGMDVWGQGTTV |
| | | TVSSGSTSGSGKPGSGEGSEIVLTQSPATLSLS |
| | | PGERATLSCRASQSVSSYLAWYQQKPGQAP |
| | | RLLIYDASNRATGIPARF SGSGSGTDFTLTIS S |
| | | LEPEDFAVYYCQQRSNWPITFGQGTRLEIKE |
| | | FPKPSTPPGS SGGAPGILGFVFTLKEFTLDF ST |
| | | AKTYVD SLNVIRS AIGTPLQTIS SGGTSLLMI |
| | | D SGTGDNLF AVDVRGIDPEEGRFNNLRLIVE |
| | | RNNLYVTGF VNRTNNVFYRF ADF SHVTFPG |
| | | TTAVTLSGDSSYTTLQRVAGISRTGMQINRH |
| | | SLTTSYLDLMSHSGTSLTQSVARAMLRFVTV |
| | | TAEALRFRQIQRGFRTTLDDL SGRSYVMTAE |
| | | DVDLTLNWGRLSSVLPDYHGQDSVRVGRIS |
| | | FGSINAILGSVALILNSHHHASRVAR |
| SEQ ID NO: 94 | Protein 23 | MEIVLTQSPATLSLSPGERATLSCRASQSVSS |
| | | YLAWYQQKPGQAPRLLI YDASNRATGIPARF |
| | | SGSGSGTDF TLTISSLEPEDFAVYYCQQRSN |
| | | WPITFGQGTRLEIKGGGGSGGGGSGGGGSG |
| | | GGGSGGGGSEVQLVES GGGLVQPGRSLRL S |
| | | CAASGFTFNDYAMHWVRQAPGKGLEWVST |
| | | ISWNSGSIGYADSVKGRFTISRDNAKKSLYL |
| | | QMNSLRAEDTALYYCAKDIQYGNYYYGMD |
| | | VWGQGTTVTVSSEFPKPSTPPGSSGGAPGILG |
| | | FVF TLKEF TLDF STAKTYVD SLNVIRS AIGTP |
| | | LQTIS SGGT SLLMID SGTGDNLFAVDVRGIDP |
| | | EEGRFNNLRLIVERNNLYVTGFVNRTNNVFY |
| | | RFADFSHVTFPGTTAVTLSGDSSYTTLQRVA |
| | | GISRTGMQINRHSLTTSYLDLMSHSGTSLTQS |
| | | VARAMLRFVTVTAEALRFRQIQRGFRTTLDD |
| | | LSGRSYVMTAEDVDLTLNWGRLSSVLPDYH |
| | | GQDSVRVGRISFGSINAILGSVALILNSHHHA |
| | | SRVAR |
| SEQ ID NO: 95 | Protein 24 | MQIVLSQSPAILSASPGEKVTMTCRASSSVSY |
| | | MHWYQQKPGSSPKPWIYAPSNLASGVPARF |
| | | SGSGSGTSY SLTISRVEAEDAATYYCQQWSF |
| | | NPPTF GAGTKLELK SGGGGSGGGGSGGGGS |
| | | GGGGSGGGGSQAYLQQSGAELVRPGASVK |
| | | MSCKASGYTFTSYNMHWVKQTPRQGLEWI |
| | | GAIYPGNGDTSYNQKFKGKATLTVDKS SSTA |
| | | YMQLSSLTSEDSAVYFCARVVYYSNSYWYF |
| | | DVWGTGTTVTVSEFPKPSTPPGSSGGILGFVF |
| | | TLGAPKEFTLDFSTAKTYVDSLNVIRS AIGTP |
| | | LQTISSGGTSLLMIDSGSGDNLF AVDVRGIDP |
| | | EEGRFNNLRLIVERNNLYVTGFVNRTNNVFY |
| | | RFADFSHVTFPGTTAVTLSGDSSYTTLQRVA |
| | | GISRTGMQINRHSLTTSYLDLMSHSGTSLTQS |
| | | VARAMLRFVTVTAEALRFRQIQRGFRTTLDD |
| | | LSGRSYVMTAEDVDLTLNWGRLSSVLPDYH |
| | | GQDSVRVGRISFGSINAILGSVALILNSHHHA |
| | | SRVAR |
| SEQ ID NO: 96 | Protein 25 | MQAYLQQSGAELVRPGASVKMSCKASGYTF |
| | | TSYNMHWVKQTPRQGLEWIGAIYPGNGDTS |
| | | YNQKFKGKATLTVDK SSSTAYMQL SSLTSE |
| | | DSAVYFCARVVYYSNSYWYFDVWGTGTTV |
| | | TVSGSTSGSGKPGSGEGSQIVLSQSPAILSASP |
| | | GEKVTMTCRAS SSV SYMHWYQQKPGS SPKP |
| | | WIYAPSNLASGVPARFSGSGSGTSYSLTISRV |
| | | EAEDAATYYCQQWSFNPPTFGAGTKLELK S |
| | | EFPKP STPPG SSGGAP GILGFVFTLKEFTLDF S |
| | | TAKTYVDSLNVIRSAIGTPLQTISSGGTSLLMI |
| | | DSGSGDNLFAVDVRGIDPEEGRFNNLRLIVE |
| | | RNNLYVTGFVNRTNNVFYRF ADF SHVTFPG |
| | | TTAVTLSGDSSYTTLQRVAGISRTGMQrNRH |
| | | SLTTSYLDLMSHSGTSLTQSVARAMLRFVTV |
| | | TAEALRFRQIQRGFRTTLDDL SGRSYVMTAE |
| | | DVDLTLNWGRLSSVLPDYHGQDSVRVGRIS |
| | | FGSrNAILGSVALILNSHHHASRVAR |
| SEQ ID NO: 97 | Protein 26 | ASVSDVPRDLEVVAATPTSLLISWCRQRCAD |
| | | SYRITYGETGGNSPVQEFTVPGSWKTATISGL |
| | | KPGVDYTITVYVVTHYYGWDRYSFIPISrNYR |
| | | TGSEFPKPSTPPGSSGGAPGILGFVFTLKEFTL |
| | | DF STAKTYVD SLNVIRS AIGTPLQ TISSGGTSL |
| | | LMID SGSGDNLFAVDVRGIDPEEGRFNNLRLI |
| | | VERNNL YVTGF VNRTNNVFYRF ADFSHVTF |
| | | PGTTAVTLSGDSSYTTLQRVAGISRTGMQIN |
| | | RHSLTTSYLDLMSHSGTSLTQSVARAMLRFV |
| | | TVTAEALRFRQIQRGFRTTLDDLSGRSYVMT |
| | | AEDVDLTLNWGRLS SVLPDYHGQD SVRVGR |
| | | ISFGSFNAILGSVALILNSHHHASRVAR |
| SEQ ID NO: 98 | Protein 27 | MQVQLQQPGAELVKPGASVKMSCKTSGYTF |
| | | TSYNVFIWVKQTPGQGLEWIGAIYPGNGDTS |
| | | FNQKFKGKATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEKVTMTCRAS SSV SYMDWYQQKPGS SPK |
| | | PWIYATSNLASGVPARFSGSGSGTSYSLTISR |
| | | VEAEDAATYYCQQWISNPPTFGAGTKLELK |
| | | EFPKP STPPG SSGGAP GILGFVF TLKEF TLDF S |
| | | TAKTYVDSLNVIRSAIGTPLQTISSGGTSLLMI |
| | | DSGSGDNLF AVDVRGIDPEEGRFNNLRLIVE |
| | | RNNLYVTGFVNRTNNVFYRF ADF SHVTFPG |
| | | TTAVTLSGDSSYTTLQRVAGISRTGMQFNRH |
| | | SLTTSYLDLMSHSGTSLTQSVARAMLRFVTV |
| | | TAEALRFRQIQRGFRTTLDDL SGRSYVMTAE |
| | | DVDLTLNWGRLSSVLPDYHGQDSVRVGRIS |
| | | FGSrNAILGSVALILNCHHHASRVARCITGDA |
| | | LVALPEGESVRIADIVPGARPNSDNAIDLKVL |
| | | DRHGNPVLADRLFHSGEHPVYTVRTVEGLR |
| | | VTGTANHPLLCLVDVAGVPTLLWKLIDEIKP |
| | | GDYAVIQRSAFSVDCAGFARGKPEFAPTTYT |
| | | VGVPGLVRFLEAHHRDPDAQAIADELTDGR |
| | | FYYAKVASVTDAGVQPVYSLRVDTADHAFI |
| | | TNGFVSHATGLTGLNSGLTTNPGVSAWQVN |
| | | TAYTAGQLVTYNGKTYKCLQPHTSLAGWEP |
| | | SNVPALWQLQ |
| SEQ ID NO: 99 | Protein 28 | MQVQLQQPGAELVKPGASVKMSCKTSGYTF |
| | | TSYNVHWVKQTPGQGLEWIGAIYPGNGDTS |
| | | FNQKFKGK ATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEK VTMTCRAS SSV SYMDW YQQKPGS SPK |
| | | PWIYATSNLASGVPARFSGSGSGTSYSLTISR |
| | | VEAEDAATYYCQQWISNPPTFGAGTKLELK |
| | | EFPKP STPPG SSGGAP GILGFVFTLKEF TLDF S |
| | | TAKTYVDSLNVIRSAIGTPLQTISSGGTSLLMI |
| | | DSGSGDNLFAVDVRGIDPEEGRFNNLRLIVE |
| | | RNNLYVTGF VNRTNNVF YRF ADF SHVTFPG |
| | | TTAVTLSGDSSYTTLQRVAGISRTGMQINRH |
| | | SLTTSYLDLMSHSGTSLTQSVARAMLRFVTV |
| | | TAEALRFRQIQRGFRTTLDDL SGRSYVMT AE |
| | | DVDLTLNWGRLSSVLPDYHGQDSVRVGRIS |
| | | FGSrNAILGSVALILNSHHHASRVARCITGDA |
| | | LVALPEGESVRIADIVPGARPNSDNAIDLKVL |
| | | DRHGNPVLADRLFHSGEHPVYTVRTVEGLR |
| | | VTGTANHPLLCLVDVAGVPTLLWKLIDEIKP |
| | | GDYAVIQRSAFSVDCAGFARGKPEFAPTTYT |
| | | VGVPGLVRFLEAHHRDPDAQAIADELTDGR |
| | | FYYAKVASVTDAGVQPVYSLRVDTADHAFI |
| | | TNGFVSHATGLTGLNSGLTTNPGVSAWQVN |
| | | TAYTAGQLVTYNGKTYKCLQPHTSLAGWEP |
| | | SNVPALWQLQ |
| SEQ ID NO: 100 | Protein 29 | MQVQLQQPGAELVKPGASVKMSCKTSGYTF |
| | | TSYNVHWVKQTPGQGLEWIGAIYPGNGDTS |
| | | FNQKFKGK ATLTADK SSSTVYMQL SSLTSED |
| | | SAVYYCARSNYYGSSYVWFFDVWGAGTTV |
| | | TVSSGSTSGSGKPGSGEGSQIVLSQSPTILSAS |
| | | PGEK VTMTCRAS SSVS YMDW YQQKPGS SPK |
| | | PWIYATSNLASGVPARFSGSGSGTSYSLTISR |
| | | VEAEDAATYYCQQWISNPPTFGAGTKLELK |
| | | EFPKP STPPG SSGGAP GILGFVFTLKEF TLDF S |
| | | TAKTYVDSLNVIRSAIGTPLQTISSGGTSLLMI |
| | | DSGSGDNLFAVDVRGIDPEEGRFNNLRLIVE |
| | | RNNL YVTGF VNRTNNVF YRF ADF SHVTFPG |
| | | TTAVTLSGDSSYTTLQRVAGISRTGMQINRH |
| | | SLTTSYLDLMSHSGTSLTQSVARAMLRFVTV |
| | | TAEALRFRQIQRGFRTTLDDL SGRSYVMT AE |
| | | DVDLTLNWGRLSSVLPDYHGQDSVRVGRIS |
| | | FGSINAILGSVALILNCHHHASRVARKDEL |
| SEQ ID NO: 101 | Protein 30 | MKEFTLDFSTAKTYVDSLNVIRSAIGTPLQTI |
| | | SSGGT SLLMID SGSGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASA |
| | | VAAEFPKPSTPPGSSGGAPGILGFVFTLMQV |
| | | QLQQPGAELVKPGASVKMSCKTSGYTFTSY |
| | | NVHWVKQTPGQGLEWIGAIYPGNGDTSFNQ |
| | | KFKGKATLTADKSSSTVYMQLSSLTSEDSAV |
| | | YYCARSNYYGS SYVWFFDVWGAGTT VTV S |
| | | SGSTSGSGKPGSGEGSQIVLSQSPTILSASPGE |
| | | KVTMTCRAS SSVSYMDWYQQKPGS SPKPWI |
| | | YATSNLASGVPARFSGSGSGTSYSLTISRVEA |
| | | EDAATYYCQQWISNPPTFGAGTKLELK |
| SEQ ID NO: 102 | Protein 31 | MKEFTLDFSTAKTYVDSLNVIRSAIGTPLQTI |
| | | SSGGTSLLMIDSGSGDNLFAVD VRGIDPEEG |
| | | RFNNLRLIVERNNLYVTGFVNRTNNVFYRFA |
| | | DF SHVTFPGTTAVTLSGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLDIELT |
| | | QSPSSFSVSLGDRVTITCKASEDIYNRLAWY |
| | | QQKPGNAPRLLIS GATSLETGVP SRFSGSGSG |
| | | KDYTLSITSLQTEDVATYYCQQYWSTPTFGG |
| | | GTKLEIKGS TSGSGKPGS GEGSKVQLQES GP S |
| | | LVQPSQRLSITCTVSGFSLISYGVHWVRQSPG |
| | | KGLEWLGVIWRGGS TDYNAAFMSRLSITKD |
| | | NSKSQVFFKMNSLQADDTAIYFCAKTLITTG |
| | | YAMDYWGQGTTVTVSS |
| SEQ ID NO: 103 | Protein 32 | MKEFTLDFSTAKTYVDSLNVIRSAIGTPLQTI |
| | | SSGGTSLLMIDSGTGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNSHHHASR |
| | | VAREFPKPSTPPGSSGGAPGILGFVFTLDIELT |
| | | QSPSSFSVSLGDRVTITCKASEDIYNRLAWY |
| | | QQKPGNAPRLLISGATSLETGVPSRFSGSGSG |
| | | KDYTLSITSLQTEDVATYYCQQYWSTPTFGG |
| | | GTKLEIKGSTSGSGKPGS GEGSKVQLQES GP S |
| | | LVQPSQRLSITCTVSGFSLISYGVHWVRQSPG |
| | | KGLEWLGVIWRGGS TDYNAAFM SRL SITKD |
| | | NSKSQVFFKMNSLQADDTAIYFCAKTLITTG |
| | | YAMDYWGQGTTVTV SS |
| SEQ ID NO: 104 | Protein 33 | MKEFTLDFSTAKTYVDSLNVIRSAIGTPLQTI |
| | | SSGGT SLLMID SGSGDNLFAVD VRGIDPEEG |
| | | RFNNLRLIVERNNLYVTGFVNRTNNVFYRFA |
| | | DF SHVTFPGTTAVTLSGDSSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLDIQM |
| | | TQSP SSL SA SVGDRVTITCRASQD VNTAVAW |
| | | YQQKPGKAPKLLIY SA SFLYSGVP SRF SGSRS |
| | | GTDFTLTISSLQPEDFATYYCQQHYTTPPTFG |
| | | QGTKVEIKRTGS TSGSGKPGSGEGSEVQLVE |
| | | SGGGLVQPGGSLRL SCAA SGFNIKDTYIFtWV |
| | | RQAPGKGLEWVARIYPTNGYTRYAD SVKGR |
| | | FTISADTSKNTAYLQMNSLRAEDTAVYYCSR |
| | | WGGDGF YAMDVWGQGTLVTVSS |
| SEQ ID NO: 105 | Protein 34 | MKEFTLDFSTAKTYVDSLNVIRSAIGTPLQTI |
| | | SSGGTSLLMIDSGTGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNSHHHASR |
| | | VAREFPKPSTPPGSSGGAPGILGFVFTLDIQM |
| | | TQ SP SSL SA SVGDRVTITCRASQD VNTAVAW |
| | | YQQKPGKAPKLLIY SA SFLYSGVP SRF SGSRS |
| | | GTDFTLTISSLQPEDFATYYCQQHYTTPPTFG |
| | | QGTKVEIKRTGS TSGSGKPGS GEGSEVQLVE |
| | | SGGGLVQPGGSLRL SCAA SGFNIKDTYIHWV |
| | | RQAPGKGLEWVARIYPTNGYTRYAD SVKGR |
| | | FTISADTSKNTAYLQMNSLRAEDTAVYYCSR |
| | | WGGDGFYAMDVWGQGTLVTVS S |
| SEQ ID NO: 106 | Protein 35 | MKEFTLDFSTAKTYVDSLNVIRSAIGTPLQTI |
| | | SSGGTSLLMIDSGSGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLDIVM |
| | | TQAAPSIPVTPGESVSISCRSSKSLLNSNGNTY |
| | | LYWFLQRPGQSPQLLIYRMSNLASGVPDRFS |
| | | GSGS GTAF TLRISRVEAEDVGVYYCMQHLE |
| | | YPF TF GAGTKLELKGS TSGSGKPGS GEGSEV |
| | | QLQQSGPELIKPGASVKMSCKASGYTFTSYV |
| | | MHWVKQKPGQGLEWIGYINPYNDGTKYNE |
| | | KFKGKATLTSDKSSSTAYMELSSLTSEDSAV |
| | | YYCARGTYYYGSRVFD YWGQGTTLTV SS |
| SEQ ID NO: 107 | Protein 36 | MKEFTLDFSTAKTYVDSLNVIRSAIGTPLQTI |
| | | SSGGT SLLMID SGTGDNLF AVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNSHHHASR |
| | | VAREFPKPSTPPGSSGGAPGILGFVFTLDIVM |
| | | TQAAPSIPVTPGESVSISCRSSKSLLNSNGNTY |
| | | LYWFLQRPGQSPQLLIYRMSNLASGVPDRFS |
| | | GSGS GTAF TLRISRVEAEDVGVYYCMQULE |
| | | YPF TFGAGTKLELKGS TSGSGKPGS GEGSEV |
| | | QLQQSGPELIKPGASVKMSCKASGYTFTSYV |
| | | MHWVKQKPGQGLEWIGYINPYNDGTKYNE |
| | | KFKGKATLTSDKSSSTAYMELSSLTSEDSAV |
| | | YYCARGTYYYGSRVFD YWGQGTTLTV SS |
| SEQ ID NO: 108 | Protein 37 | MKEFTLDFSTAKTYVDSLNVIRSAIGTPLQTI |
| | | SSGGTSLLMIDSGSGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLDIQLT |
| | | QSPL SLPVTLGQPASISCRSSQSLVHRNGNTY |
| | | LHWFQQRPGQSPRLLIYT VSNRF SGVPDRF S |
| | | GSGS GTDF TLKISRVEAEDVGVYF CSQSSHV |
| | | PPTFGAGTRLEIKGSTSGSGKPGSGEGSTKGQ |
| | | VQLQQSGSELKKPGASVKVSCKASGYTFTN |
| | | YGVNWIKQAPGQGLQWMGWINPNTGEPTF |
| | | DDDFKGRF AF SLDT SVSTAYLQIS SLKADDT |
| | | AVYFCSRSRGKNEAWFAYWGQGTLVTVSS |
| SEQ ID NO: 109 | Protein 38 | MKEFTLDFSTAKTYVDSLNVIRSAIGTPLQTI |
| | | SSGGTSLLMIDSGTGDNLFAVD VRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNSHHHASR |
| | | VAREFPKPSTPPGSSGGAPGILGFVFTLDIQLT |
| | | QSPL SLPVTLGQPASISCRSSQSLVHRNGNTY |
| | | LHWFQQRPGQSPRLLIYT VSNRF SGVPDRF S |
| | | GSGS GTDF TLKISRVEAEDVGVYF CSQSSHV |
| | | PPTFGAGTRLEIKGSTSGSGKPGSGEGSTKGQ |
| | | VQLQQSGSELKKPGASVKVSCKASGYTFTN |
| | | YGVNWIKQAPGQGLQWMGWINPNTGEPTF |
| | | DDDFKGRFAF SLDT SVSTAYLQIS SLKADDT |
| | | AVYFCSRSRGKNEAWFAYWGQGTLVTVSS |
| SEQ ID NO: 110 | Protein 39 | MKEFTLDFSTAKTYVDSLNVIRSAIGTPLQTI |
| | | SSGGT SLLMID SGSGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRL SSVLPDYHGQ |
| | | DSVRVGRISFGSrNAILGSVALILNCHHHASR |
| | | VARAHHSEDPS SKAPKAPGILGFVFTLEVQL |
| | | VESGGGLVQAGGSLRLSCAASGITF SINTMG |
| | | WYRQAPGKQRELVALISSIGDTYYADSVKG |
| | | RFTISRDNAKNTVYLQMNSLKPEDTAVYYC |
| | | KRFRTAAQGTDYWGQGTQVTVSS |
| SEQ ID NO: 111 | Protein 40 | MKEFTLDFSTAKTYVDSLNVIRSAIGTPLQTI |
| | | SSGGTSLLMIDSGSGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLEVQL |
| | | VESGGGLVQAGGSLRLSCAASGITF SINTMG |
| | | WYRQAPGKQRELVALISSIGDTYYADSVKG |
| | | RFTISRDNAKNTVYLQMNSLKPEDTAVYYC |
| | | KRFRTAAQGTDYWGQGTQVTVS S |
| SEQ ID NO: 112 | Protein 41 | MKEFTLDFSTAKTYVDSLNVIRSAIGTPLQTI |
| | | SSGGTSLLMIDSGTGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNSHHHASR |
| | | VARAHHSEDPS SKAPKAPGILGFVFTLEVQL |
| | | VESGGGLVQAGGSLRLSCAASGITF SINTMG |
| | | WYRQAPGKQRELVALISSIGDTYYADSVKG |
| | | RFTISRDNAKNTVYLQMNSLKPEDTAVYYC |
| | | KRFRTAAQGTDYWGQGTQVTVS S |
| SEQ ID NO: 113 | Protein 42 | MKEFTLDFSTAKTYVDSLNVIRSAIGTPLQTI |
| | | SSGGTSLLMIDSGTGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSINAILGSVALILNSHEHASR |
| | | VAREFPKPSTPPGSSGGAPGILGFVFTLEVQL |
| | | VESGGGLVQAGGSLRLSCAASGITF SINTMG |
| | | WYRQAPGKQRELVALISSIGDTYYADSVKG |
| | | RFTISRDNAKNTVYLQMNSLKPEDTAVYYC |
| | | KRFRTAAQGTDYWGQGTQVTVSS |
| SEQ ID NO: 114 | Protein 43 | MKEFTLDFSTAKTYVDSLNVIRSAIGTPLQTI |
| | | SSGGTSLLMIDSGSGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLASVS |
| | | DVPRDLEVVAATPTSLLISWCRQRCADSYRI |
| | | TYGETGGN SPVQEFTVPGSWKTATISGLKPG |
| | | VDYTITVYVVTHYYGWDRYSHPISINYRTGS |
| SEQ ID NO: 115 | Protein 44 | MKEFTLDFSTAKTYVDSLNVIRSAIGTPLQTI |
| | | SSGGTSLLMIDSGTGDNLFAVDVRGIDPEEG |
| | | RFNNLRLIVERNNLYVTGFVNRTNNVFYRFA |
| | | DF SHVTFPGTTAVTL SGD SSYTTLQRVAGISR |
| | | TGMQINRHSLTTSYLDLMSHSGTSLTQSVAR |
| | | AMLRFVTVTAEALRFRQIQRGFRTTLDDLSG |
| | | R SYVMTAEDVDLTLNWGRLS SVLPDYHGQ |
| | | DSVRVGRISFGSrNAILGSVALILNCHHHASR |
| | | VAREFPKPSTPPGSSGGAPGILGFVFTLASVS |
| | | DVPRDLEVVAATPTSLLISWCRQRCADSYRI |
| | | TYGETGGN SPVQEFTVPGSWKTATISGLKPG |
| | | VDYTITVYVVTHYYGWDRYSHPISINYRTGS |

The invention is claimed as follows:

1. A cell-targeting molecule comprising
i) a Shiga toxin effector polypeptide having a Shiga toxin A1 fragment region,
ii) a heterologous binding region capable of specifically binding at least one extracellular target biomolecule, and
iii) a heterologous, CD8+ T-cell epitope which is not embedded in the Shiga toxin A 1 fragment region;
whereby administration of the cell-targeting molecule to a cell results in the internalization of the cell-targeting molecule by the cell and the cell presenting on a cellular surface the CD8+ T-cell epitope complexed with a MHC class I molecule.

2. The cell-targeting molecule of claim 1, wherein the CD8+ T-cell epitope is fused to the Shiga toxin effector polypeptide or the binding region.

3. The cell-targeting molecule of claim 2, wherein the cell-targeting molecule comprises a single-chain polypeptide comprising the binding region, the Shiga toxin effector polypeptide, and the CD8+ T-cell epitope.

4. The cell-targeting molecule of claim 2, wherein the binding region comprises two or more polypeptide chains and the T-cell epitope-peptide is fused to a polypeptide comprising the Shiga toxin effector polypeptide and one of the two or more polypeptide chains.

5. The cell-targeting molecule of any one of claims 1-4, wherein the Shiga toxin effector polypeptide comprises a Shiga toxin A 1 fragment derived region having a carboxy terminus, and the heterologous, CD8+ T-cell epitope is positioned carboxy-terminal to the carboxy terminus of the Shiga toxin A1 fragment derived region.

6. The cell-targeting molecule of any one of claims 1-5, wherein the binding region comprises a polypeptide selected from the group consisting of:
single-domain antibody fragment, single-chain variable fragment, antibody variable fragment, complementary determining region 3 fragment, constrained FR3-CDR3-FR4 polypeptide, Fd fragment, antigen-binding fragment, Armadillo repeat polypeptide, fibronectin-derived lO\hat{ }fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality.

7. The cell-targeting molecule of any one of claims 1-6, wherein the Shiga toxin effector polypeptide comprises or consists essentially of the polypeptide sequence selected from the group consisting of:
(i) amino acids 75 to 251 of SEQ ID NO: 1, SEQ ID NO:2, or SEQ ID NO:3;
(ii) amino acids 1 to 241 of SEQ ID NO: 1, SEQ ID NO:2, or SEQ ID NO:3;
(iii) amino acids 1 to 251 of SEQ ID NO:1, SEQ ID NO:2, or SEQ ID NO:3; and
(iv) amino acids 1 to 261 of SEQ ID NO: 1, SEQ ID NO:2, or SEQ ID NO:3.

8. The cell-targeting molecule of any one of claims 1-6, wherein the carboxy terminus of the Shiga toxin A1 fragment derived region comprises a disrupted furin cleavage motif.

9. The cell-targeting molecule of claim 8, wherein the disrupted furin-cleavage motif comprises one or more mutations, relative to a wild-type Shiga toxin A Subunit, the mutation altering at least one amino acid residue in a region natively positioned at 248-251 of the A Subunit of Shiga-like toxin 1 (SEQ ID NO: 1) or Shiga toxin (SEQ ID NO: 2), or at 247-250 of the A Subunit of Shiga-like toxin 2 (SEQ ID NO:3).

10. The cell-targeting molecule of claim 8 or claim 9, wherein the disrupted furin-cleavage motif comprises an amino acid residue substitution in the furin-cleavage motif relative to a wild-type Shiga toxin A Subunit.

11. The cell-targeting molecule of claim 10, wherein the substitution of the amino acid residue in the furin-cleavage motif is of an arginine residue with a non-positively charged, amino acid residue selected from the group consisting of:
alanine, glycine, proline, serine, threonine, aspartate, asparagine, glutamate, glutamine, cysteine, isoleucine, leucine, methionine, valine, phenylalanine, tryptophan, and tyrosine.

12. The cell-targeting molecule of any one of claims 1-11, wherein the binding region is capable of binding to the extracellular target biomolecule selected from the group consisting of:
CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD 19, CD21, CS1/SLAMF7, CD33, CD52, CD 133, CEA, gpA33, mucin, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gplOO, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gplOO, tyrosinase associated antigen, HPV-E7, Epstein-Barr virus antigen, Bcr-Abl, alpha-fetoprotein antigen, 17-A1, bladder tumor antigen, CD38, CD15, CD23, CD45, CD53, CD88, CD129, CD183, CD191, CD193, CD244, CD294, CD305, C3AR, FceRIa, IL-1R, galectin-9, mrp-14, NKG2D, PD-L1, Siglec-8, Siglec-10, CD49d, CD 13, CD44, CD54, CD63, CD69, CD123, TLR4, FceRIa, IgE, CD107a, CD203c, CD14, CD68, CD80, CD86, CD105, CD115, F4/80, ILT-3, galectin-3, CD11a-c, GITRL, MHC class I molecule, MHC class II molecule, CD284, CD107-Mac3, CD195, HLA-DR, CD16/32, CD282, CD11c, and any immunogenic fragment of any of the foregoing.

13. The cell-targeting molecule of any one of claims 1-12, whereby administration of the cell-targeting molecule to a cell physically coupled with an extracellular target biomolecule of the binding region, the cell-targeting molecule is capable of causing death of the cell.

14. The cell-targeting molecule of claim 13, whereby administration of the cell-targeting molecule to a first population of cells whose members are physically coupled to extracellular target biomolecules of the binding region, and a second population of cells whose members are not physically coupled to any extracellular target biomolecule of the binding region, the cytotoxic effect of the cell-targeting molecule to members of said first population of cells relative to members of said second population of cells is at least 3-fold greater.

15. The cell-targeting molecule of any one of claims 1-14, wherein the Shiga toxin effector polypeptide comprises a mutation relative to a naturally occurring A Subunit of a member of the Shiga toxin family which changes the enzymatic activity of the Shiga toxin effector region, the mutation selected from at least one amino acid residue deletion, insertion, or substitution.

16. The cell-targeting molecule of claim 15, wherein the mutation is selected from at least one amino acid residue deletion, insertion, or substitution that reduces or eliminates cytotoxicity of the toxin effector polypeptide.

17. The cell-targeting molecule of any one of claims 1-16, comprising or consisting essentially of the polypeptide of any one of SEQ ID NOs: 13-61 and 73-115.

18. A pharmaceutical composition comprising the cell-targeting molecule of any one of claims 1-17 and at least one pharmaceutically acceptable excipient or carrier.

19. A polynucleotide capable of encoding the cell-targeting molecule of any one of claims 1-17, or a complement thereof, or a fragment of any of the foregoing.

20. An expression vector comprising the polynucleotide of claim 19.

21. A host cell comprising any one of the polynucleotides or expression vectors of claims 19-20.

22. A method of killing a cell, the method comprising the step of contacting the cell with the cell-targeting molecule of any one of claims 1-17, or the pharmaceutical composition of claim 18.

23. The method of claim 22, wherein the contacting occurs in vitro.

24. The method of claim 22, wherein the contacting occurs in vivo.

25. A method of treating a disease, disorder, or condition in a patient, the method comprising the step of administering to a patient in need thereof a therapeutically effective amount of the cell-targeting molecule of any one of claims 1-17, or the pharmaceutical composition of claim 18.

26. The method of claim 25, wherein the disease, disorder, or condition is selected from the group consisting of: cancer, tumor, growth abnormality, immune disorder, and microbial infection.

27. The method of claim 26, wherein the cancer selected from the group consisting of:
bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer.

28. The method of claim 26, wherein the immune disorder associated is with a disease selected from the group consisting of:
amyloidosis, ankylosing spondylitis, asthma, autism, cardiogenesis, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders, multiple sclerosis, myasthenia gravis, neuroinflammation, polyarteritis nodosa, polyarthritis, psoriasis, psoriatic arthritis, rheumatoid arthritis, scleroderma, septic shock, Sjorgren's syndrome, systemic lupus erythematosus, ulcerative colitis, vasculitis.

29. A composition comprising the cell-targeting molecule of any one of claims 1-17, for the treatment or prevention of a cancer, tumor, growth abnormality, immune disorder, or microbial infection.

30. Use of the composition of matter of any one of claims 1-21 in the manufacture of a medicament for the treatment or prevention of a cancer, tumor, growth abnormality, immune disorder, or microbial infection.

31. A method of “seeding” a tissue locus within a chordate, the method comprising the step of administering to the chordate the cell-targeting molecule of any one of claims 1-17, the pharmaceutical composition of claim 18.

32. The method of claim 31, wherein the T-cell epitope-peptide of the cell-targeting molecule is selected from the group consisting of:
peptides not natively presented by the target cells of the cell-targeting molecule in MHC class I complexes, peptides not natively present within any protein expressed by the target cell, peptides not natively present within the transcriptome or proteome of the target cell, peptides not natively present in the extracellular microenvironment of the site to be seeded, and peptides not natively present in the tumor mass or infect tissue site to be targeted.

33. The method of claim 31, wherein the tissue locus comprises a malignant, diseased, or inflamed tissue.

34. The method of claim 33, wherein the tissue locus comprises the tissue selected from the group consisting of: tumor mass, cancerous growth, tumor, infected tissue, or abnormal cellular mass.

35. A method of treating cancer using immunotherapy, the method comprising the step of administering to a patient in need thereof the cell-targeting molecule of any one of claims 1-17 or the pharmaceutical composition of claim 18.

36. A kit comprising the composition of matter of any one of claims 1-21; and an additional reagent and/or pharmaceutical delivery device.
